(19) United States
(12) Patent Application Publication
(10) Pub. No.: US 2011/0190365 A1
(43) Pub. Date: Aug. 4, 2011

(54) Insecticidal 4-phenyl-1H-pyrazoles

(75) Inventors: Stefan Werner, Monheim (DE); Klaus-Helmut Müller, Dusseldorf (DE); Hans-Georg Schwarz, Dorsten (DE); Tetsuya Murata, Tochigi (JP); Klaus Raming, Leverkusen (DE); Ulrich Görgens, Ratingen (DE); Angela Becker, Dusseldorf (DE); Eva-Maria Franken, Leichlingen (DE); Elichi Shimojo, Tochigi (JP); Katsuhiko Shibuya, Tochigi (JP); Ulrich Ebbinghaus-Kintscher, Dortmund (DE); Teruyuki Chihara, Tochigi (JP); Masashi Ataka, Saitama (JP); Olga Malsam, Rosrath (DE); Arnd Voerste, Koin (DE)

(73) Assignee: Bayer Crop Science AG, Monheim (DE)

(21) Appl. No.: 13/058,767

(22) PCT Filed: Aug. 1, 2009
(86) PCT No.: PCT/EP09/05587

§ 371 Date: Apr. 15, 2011

(30) Foreign Application Priority Data
Aug. 14, 2008 (EP) 08162378.7

(51) Int. Cl. A01N 43/56; C07D 231/12; A01P 15/00

(52) U.S. Cl. 514/406; 548/377.1

(57) ABSTRACT

The present invention relates to novel 4-phenyl-1H-pyrazoles and their use as insecticides and/or parasiticides and also to processes for their preparation and to compositions comprising such phenylpyrazoles.

The present invention relates to novel 4-phenyl-1H-pyrazoles and their use as insecticides and/or parasiticides and also to processes for their preparation and to compositions comprising such phenylpyrazoles.

EP 846686 describes 4-phenyl-1H-pyrazoles (A) having parasiticidal, insecticidal and nematicidal action. The definitions of the substituents R³, R⁵ and R⁷ are as follows:

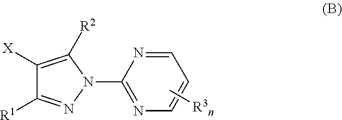

R³ represents halogen;
R⁵ represents H, C₁₋₆-alkyl, optionally substituted by one or more halogen atoms, C₁₋₆-alkoxy, optionally substituted by one or more halogen atoms, or SF₅;
   R⁷ represents halogen.

WO 2008/077483 describes pyrimidinylpyrazoles (B) having insecticidal and/or parasiticidal action. The definitions of the substituents R³ and n are as follows:

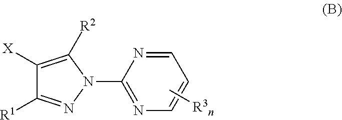

R³ represents halogen, alkyl, haloalkyl, alkoxy or dialkylamino;
n represents 0 or 1.

Meegalla et al. describe the synthesis and insecticidal activity of 3-thiomethyl-4-(hetero)aryl-5-amino-1-phenylpyrazoles as GABA channel blockers (Bioorganic & Medicinal Chemistry Letters (2004), 14, 4949-4953).

WO 2007/048734 describes 5-aminopyrazoles (C) for controlling phytophathogenic harmful fungi. The definitions of the substituents R³, R⁴ and R⁵ are as follows:

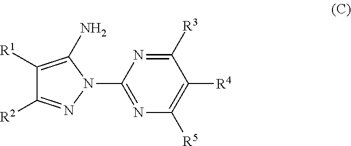

R³, R⁴, R⁵ independently of one another represent hydrogen, halogen, cyano, hydroxyl, mercapto, C₁-C₁₀-alkyl, C₁-C₁₀-haloalkyl, C₃-C₈-cycloalkyl, C₂-C₁₀-alkenyl, C₂-C₁₀-alkynyl, C₁-C₆-alkoxy, C₁-C₆-alkylthio, C₁-C₆-alkoxy-C₁-C₆-alkyl, NR^{A}R^{B}, phenyl, phenoxy or phenylthio;
   R^{A}, R^{B} represent hydrogen or C₁-C₆-alkyl.

The present invention provides novel 4-phenyl-1H-pyrazoles of the formula (I)

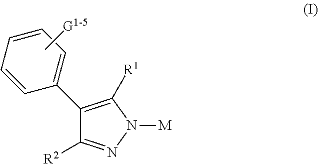

in which
M represents one of the groupings listed below:

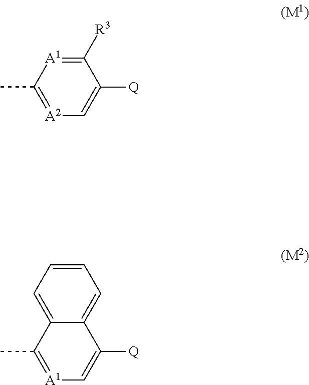

This results in 4-phenyl-1H-pyrazoles of the formulae (I-A) and (I-B)

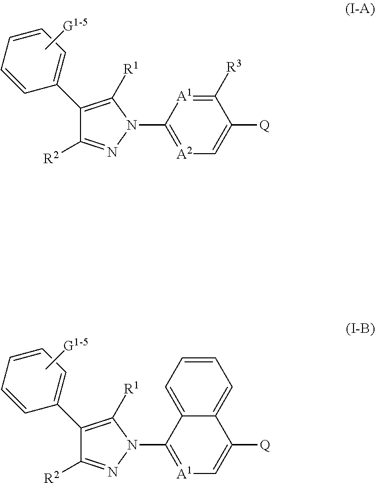

in which
A¹ and A² independently of one another represent nitrogen or C—R⁴;
G¹, G², G³, G⁴ and G⁵ represent hydrogen or a substituent independently of the others selected from the group consisting of: halogen, alkyl, alkenyl, alkynyl, haloalkyl, SF_{S}, hydroxyl, alkoxy, alkoxyalkyl, alkoxyalkoxy, cycloalkyl, alkenyloxy, alkynyloxy, cycloalkylalkoxy, haloalkoxy, haloalkoxyalkyl, alkylsulphanyl, haloalkylsulphanyl, alkylsulphinyl, haloalkylsulphinyl, alkylsulphonyl, haloalkylsulphonyl, cyano, alkylcarbonyl, alkoxycarbonyl, alkoxycarbonylalkyl, carboxyl, aminocarbonyl, aminothiocarbonyl, alkylaminocarbonyl, dialkylaminocarbonyl, trialkylsilyl, nitro, amino, alkylamino, dialkylamino, alkylsulphonylamino, dialkylsulphonylamino, —CR⁵═NO—R⁵, —CR⁵═NO-haloalkyl and formyl, where vicinal alkyl, haloalkyl, alkoxy and/or haloalkoxy groups together with the carbon atoms to which they are attached may form a five- to six-membered cyclic system which contains 0 to 2 oxygen atoms and whose alkyl moiety may optionally be substituted by one or more fluorine atoms;
   Q represents a substituent selected from the group consisting of Q¹, Q², Q³ and Q⁴;
   Q¹ represents one of the heterocyclic groupings listed below;

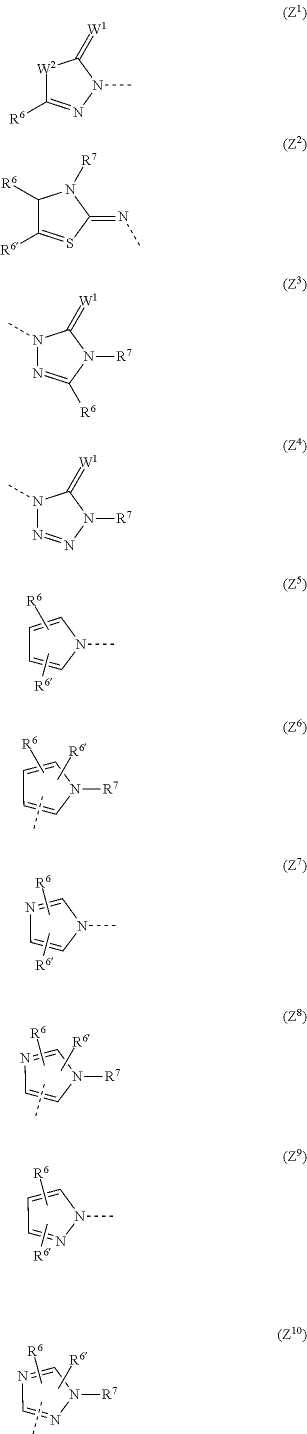

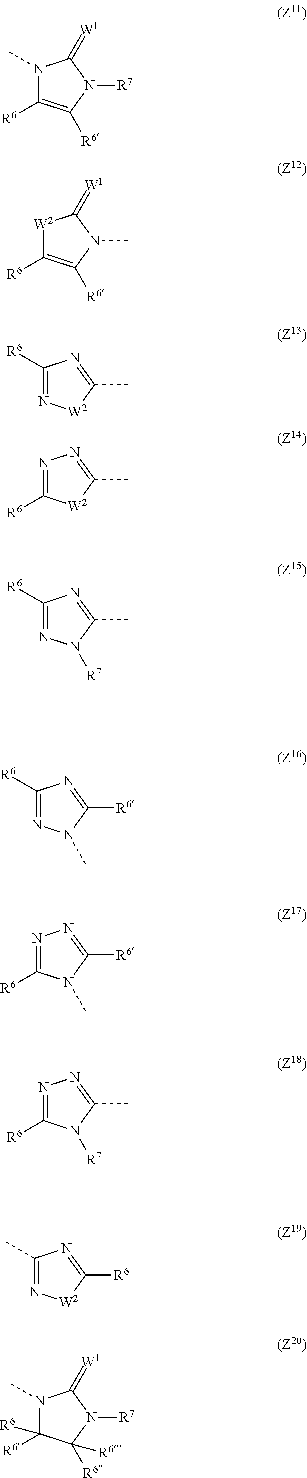

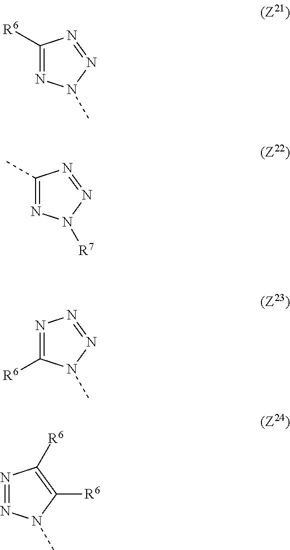

where
W¹ and W² independently of one another represent oxygen or sulphur,
and
R⁶, R^{6′}, R^{6″}, R^{6′″}, and R⁷ have the meaning given below;
Q² represents C(W¹)NR⁸R⁹;
Q³ represents C(R¹⁰R¹¹)NR⁸R⁹;
Q⁴ represents cyano (where R¹ does not represent amino), nitro, amino, COOH, COOR¹², fluorine (if R³ is different from chlorine), chlorine (if R³ is different from chlorine, COOH, CH₂CH₂OMe and OMe), bromine, iodine, SR¹² (where R¹ does not represent amino if R¹² represents alkyl), S(O)R¹², S(O)₂R¹² or S(O)₂NR⁸R⁹;
R¹ represents hydrogen, halogen, alkylsulphanyl, alkylsulphinyl, alkylsulphonyl, cyano, amino, Z¹⁶ or NR¹³R¹⁴;
R² represents hydrogen, halogen, alkyl, alkenyl, alkynyl, haloalkyl, cycloalkyl, hydroxy-alkyl, alkoxyalkyl, haloalkoxyalkyl, alkoxyhaloalkyl, alkylsulphanylalkyl, alkylsulphinylalkyl, alkylsulphonylalkyl, haloalkylsulphanylalkyl, haloalkylsulphinylalkyl, haloalkylsulphonylalkyl cycloalkylalkyl, phenylalkyl, heteroarylalkyl, heterocyclylalkyl, phenyl, or also represents arylhaloalkyl, haloalkyloxyhaloalkyloxyhaloalkyl, heterocyclyl, alkylsulphanyl, alkylsulphinyl, alkylsulphonyl or phenyl;
where a phenyl or heteroaryl radical is optionally mono- or polysubstituted by halogen, alkyl, haloalkyl, alkoxy, nitro or cyano; a heterocyclyl radical is optionally substituted by —OH/═O, —SH/═S, —NH₂, halogen, alkyl, haloalkyl, alkoxy, alkylsulphanyl, alkylsulphinyl, alkylsulphonyl, monoalkylamino, dialkylamino, nitro or cyano; and a phenyl-, heteroaryl- or heterocyclyl-bound alkanediyl radical is optionally mono- or polysubstituted by halogen, alkyl, haloalkyl, alkoxy or cyano;
R³ represents halogen, alkyl, alkenyl, alkynyl, haloalkyl, cycloalkyl, alkoxyalkyl, alkylcarbonyloxyalkyl, alkylsulphanylalkyl, alkylsulphinylalkyl, alkylsulphonylalkyl, hydroxyl, Z¹⁶, alkoxy, acylamino, alkoxycarbonylamino, alkenyloxy, alkinyloxy, cycloalkylalkoxy, halo-alkoxy, alkylsulphanyl, haloalkylsulphanyl, alkylsulphinyl, haloalkylsulphinyl, alkylsulphonyl, haloalkylsulphonyl, cyano, alkylcarbonyl, alkoxycarbonyl, alkoxycarbonylalkyl, carboxyl, aminocarbonyl, aminothiocarbonyl, alkylaminocarbonyl, dialkylaminocarbonyl, alkylsulphonyl-aminocarbonyl, trialkylsilyl, nitro, amino, alkylamino, dialkylamino, alkylsulphonylamino, dialkylsulphonylamino, —CR⁵═NO—R⁵, —CR⁵═NO-haloalkyl or formyl;
   R⁴ represents hydrogen, cyano, nitro, alkyl, haloalkoxy, halogen or alkoxy;
   R⁵ represents hydrogen or alkyl;
R⁶, R^{6′}, R^{6″}, R^{6′″} independently of one another represent hydrogen, amino, hydroxyl, mercapto, nitro, cyano, carboxyl, halogen, alkyl, haloalkyl, alkenyl, alkynyl, cycloalkyl, alkoxy, haloalkoxy, alkenyloxy, alkynyloxy, alkoxycarbonyl, alkylsulphanyl, haloalkylsulphanyl, alkylsulphinyl, haloalkylsulphinyl, alkylsulphonyl, haloalkylsulphonyl, aminocarbonyl, aminothio-carbonyl, CR⁵═NO—R⁵, —CR⁵═NO-haloalkyl, formyl, alkylamino, dialkylamino, phenyl, heteroaryl, heteroarylalkyl or heterocyclylalkyl;
where a phenyl or heteroaryl radical is optionally mono- or polysubstituted by halogen, alkyl, haloalkyl, alkoxy, nitro or cyano; a heterocyclyl radical is optionally substituted by —OH/═O, —SH/═S, —NH₂, halogen, alkyl, haloalkyl, alkoxy, alkylsulphanyl, alkylsulphinyl, alkylsulphonyl, monoalkylamino, dialkylamino, nitro or cyano; and a phenyl-, heteroaryl- or heterocyclyl-bound alkanediyl radical is optionally mono- or polysubstituted by halogen, alkyl, haloalkyl, alkoxy or cyano;
R⁷ represents hydrogen, amino, hydroxyl, cyano, alkyl, haloalkyl, alkenyl, alkynyl, cyclo-alkyl, alkoxy, alkenyloxy, alkynyloxy, alkoxycarbonyl, alkylcarbonyl, alkylaminocarbonyl, dialkylaminocarbonyl, phenyl, phenylalkyl, heteroarylalkyl or heterocyclylalkyl;
where a phenyl or heteroaryl radical is optionally mono- or polysubstituted by halogen, alkyl, haloalkyl, alkoxy, nitro or cyano; a heterocyclyl radical is optionally substituted by —OH/═O, —SH/═S, —NH₂, halogen, alkyl, haloalkyl, alkoxy, alkylsulphanyl, alkylsulphinyl, alkylsulphonyl, monoalkylamino, dialkylamino, nitro or cyano; and a phenyl-, heteroaryl- or heterocyclyl-bound alkanediyl radical is optionally mono- or polysubstituted by halogen, alkyl, haloalkyl, alkoxy or cyano and where optionally two adjacent radicals from the group consisting of R⁶, R^{6′}, R^{6″}, R^{6′″} and R⁷ together represent alkanediyl or alkenediyl, each of which has 2 to 5 carbon atoms and is optionally substituted and/or optionally interrupted at the beginning (or the end) or within the hydrocarbon chain by oxygen or sulphur or a grouping from the group consisting of —S(O)—, —SO₂—, —NH— and —N-alkyl-;
R⁸ and R⁹ independently of one another represent hydrogen, alkyl, haloalkyl, alkenyl (optionally substituted by halogen, alkyl, haloalkyl or cyano), alkynyl, cycloalkyl (optionally mono- or polysubstituted at the cycle by halogen, haloalkyl, alkyl or condensed to an aromatic or heteroaromatic moiety), cycloalkylalkyl (optionally mono- or polysubstituted at the cycle by halogen, haloalkyl, alkyl or condensed to an aromatic or heteroaromatic moiety, optionally mono- or polysubstituted at the alkyl moiety by halogen, alkyl, haloalkyl, alkoxy or cyano), cycloalkylcarbonylheterocyclyl, alkyloxycarbonylheterocyclyl, alkylsulphinylalkyl, alkylsulphanylalkyl, alkylsulphonylalkyl, hydroxyalkyl, heterocyclylcarbonylalkyl, heteroarylcarbonylaminoalkyl, haloalkoxyalkyl, alkylthiocarbonyl, dialkylaminocarbonyl, alkylaminocarbonyl, haloalkylaminocarbonyl, alkoxy, alkenyloxy, alkynyloxy, alkoxyalkyl, alkylcarbonylalkyl, C(R¹⁰R¹¹)CR⁵═NO—R⁵, alkylsulphonyl, alkylcarbonyl, haloalkylcarbonyl, alkenylcarbonyl, alkynylcarbonyl, cycloalkylcarbonyl, alkoxycarbonyl, alkenyloxycarbonyl, alkynyloxycarbonyl, alkoxyalkylcarbonyl, phenylsulphonyl, phenyl, heteroaryl, heterocyclyl, phenylalkyl, heteroarylalkyl, heterocyclylalkyl, phenylcarbonyl, heteroarylcarbonyl, heterocyclylcarbonyl, phenylalkylcarbonyl, heteroarylalkylcarbonyl, heterocyclylalkylcarbonyl, phenoxycarbonyl or phenylalkoxycarbonyl;
where a phenyl or heteroaryl radical is optionally mono- or polysubstituted by halogen, alkyl, haloalkyl, alkoxy, nitro or cyano; a heterocyclyl radical is optionally substituted by —OH/═O, —SH/═S, —NH₂, halogen, alkyl, haloalkyl, alkoxy, alkylsulphanyl, alkylsulphinyl, alkylsulphonyl, monoalkylamino, dialkylamino, nitro or cyano; and a phenyl-, heteroaryl- or heterocyclyl-bound alkanediyl radical is optionally mono- or polysubstituted by halogen, alkyl, haloalkyl, alkoxy or cyano and
where optionally R⁸/R⁹ together represent alkanediyl or alkenediyl, each of which has 2 to 5 carbon atoms and is optionally substituted and/or optionally interrupted at the beginning (or the end) or within the hydrocarbon chain by oxygen or sulphur or a grouping from the group consisting of —S(O)—, —SO₂—, —NH— and —N-alkyl-;
   R¹⁰ and R¹¹ independently of one another represent hydrogen, alkyl, haloalkyl or cyano;
   R¹² represents alkyl or haloalkyl;
   R¹³ and R¹⁴ independently of one another represent hydrogen, alkyl, haloalkyl, alkenyl, alkynyl, alkoxyalkyl, alkylcarbonylalkyl, C(R¹⁰R¹¹)CR⁵═NO—R⁵, alkylsulphonyl, alkylcarbonyl, haloalkyl-carbonyl, alkenylcarbonyl, alkynylcarbonyl, cycloalkylcarbonyl, alkoxycarbonyl, alkenyloxy-carbonyl, alkynyloxycarbonyl, alkoxyalkylcarbonyl, phenylsulphonyl, phenyl, heteroaryl, hetero-cyclyl, phenylalkyl, heteroarylalkyl, heterocyclylalkyl, phenylcarbonyl, heteroarylcarbonyl, heterocyclylcarbonyl, phenylalkylcarbonyl, heteroarylalkylcarbonyl, heterocyclylalkylcarbonyl, phenoxycarbonyl or phenylalkoxycarbonyl;
where a phenyl or heteroaryl radical is optionally mono- or polysubstituted by halogen, alkyl, haloalkyl, alkoxy, nitro or cyano; a heterocyclyl radical is optionally substituted by —OH/═O, —SH/═S, —NH₂, halogen, alkyl, haloalkyl, alkoxy, alkylsulphanyl, alkylsulphinyl, alkylsulphonyl, monoalkylamino, dialkylamino, nitro or cyano; and a phenyl-, heteroaryl- or heterocyclyl-bound alkanediyl radical is optionally mono- or polysubstituted by halogen, alkyl, haloalkyl, alkoxy or cyano
   or
   R¹³ and R¹⁴ as imine form the grouping ═CR⁵—NR¹⁵R¹⁶ or ═CR⁵—OR¹²;
   R¹⁵ and R¹⁶ independently of one another represent alkyl or
R¹⁵ and R¹⁶ together represent alkanediyl or alkenediyl, each of which has 2 to 5 carbon atoms and is optionally substituted and/or optionally interrupted at the beginning (or the end) or within the hydrocarbon chain by oxygen or sulphur or a grouping from the group consisting of —S(O)—, —SO₂—, —NH— and —N-alkyl-;
   and the compounds of the general formula (I) furthermore include N-oxides, salts, tautomers, diastereomers and optical isomers.

Finally, it has been found that the compounds of the formula (I) according to the invention have very good insecticidal and parasiticidal properties and can be used in crop protection, in veterinary hygiene, in the domestic field and in the protection of materials for controlling unwanted pests, such as insects and endo- or ectoparasites.

Halogen-substituted radicals, for example haloalkyl, are mono- or polyhalogenated, up to the maximum number of possible substituents. In the case of polyhalogenation, the halogen atoms can be identical or different. Halogen represents fluorine, chlorine, bromine or iodine, in particular fluorine, chlorine or bromine.

Saturated or unsaturated hydrocarbon radicals, such as alkyl or alkenyl, can in each case be straight-chain or branched as far as this is possible, including in combination with heteroatoms, such as, for example, in alkoxy.

Optionally substituted radicals can be mono- or polysubstituted, where in the case of polysubstitution the substituents can be identical or different.

The formula (I) provides a general definition of the 4-phenyl-1H-pyrazoles according to the invention. Preferred, particularly preferred, very particularly preferred and especially preferred radical definitions of the formulae given above and below are listed below. These definitions apply to the end products of the formula (I) and likewise to all intermediates.

A¹ and A² independently of one another preferably each represent nitrogen, C—H, C-halogen, C—(C₁-C₆-haloalkyl), C—(C₁-C₆-alkoxy), C-cyano or C—(C₁-C₆-alkyl);
A¹ and A² independently of one another particularly preferably represent nitrogen, C—H, C-halogen or C—(C₁-C₄-haloalkyl);
A¹ and A² independently of one another very particularly preferably represent nitrogen or C—H;
A¹ and A² especially preferably represent C—H; or
A¹ and A² especially preferably represent nitrogen; or
A¹ especially preferably represents nitrogen and A² especially preferably represents C—H; or
A¹ especially preferably represents C—H and A² especially preferably represents nitrogen;
G¹, G², G³, G⁴ and G⁵ preferably represent hydrogen or a combination of substituents independently of one another selected from the group consisting of: halogen, C₁-C₆-alkyl, C₂-C₆-alkenyl, C₂-C₆-alkynyl, C₁-C₆-haloalkyl, SF₅, hydroxyl, C₁-C₆-alkoxy, C₁-C₆-alkoxy-C₁-C₆-alkyl, C₁-C₆-alkoxy-C₁-C₆-alkoxy, C₃-C₆-cycloalkyl, C₂-C₆-alkenyloxy, C₂-C₆-alkynyloxy, C₃-C₆-cycloalkyl-C₁-C₆-alkoxy, C₁-C₆-haloalkoxy, C₁-C₆-haloalkoxy-C₁-C₆-alkyl, C₁-C₆-alkylsulphanyl, C₁-C₆-haloalkylsulphanyl, C₁-C₆-alkylsulphinyl, C₁-C₆-haloalkylsulphinyl, C₁-C₆-alkylsulphonyl, C₁-C₆-haloalkylsulphonyl, cyano, C₁-C₆-alkylcarbonyl, C₁-C₆-alkoxycarbonyl, C₁-C₆-alkoxycarbonyl-C₁-C₆-alkyl, carboxyl, aminocarbonyl, aminothiocarbonyl, C₁-C₆-alkylaminocarbonyl, C₁-C₆-dialkylaminocarbonyl, C₁-C₆-trialkylsilyl, nitro, amino, C₁-C₆-alkylamino, C₁-C₆-dialkylamino, C₁-C₆-alkylsulphonylamino, C₁-C₆-dialkylsulphonylamino, —CR⁵═NO—C₁-C₆-alkyl, —CR⁵═NO-halo-C₁-C₆-alkyl or formyl, where vicinal C₁-C₆-alkyl, C₁-C₆-haloalkyl, C₁-C₆-alkoxy and/or C₁-C₆-haloalkoxy groups together with the carbon atoms to which they are attached may faun a five- to six-membered cyclic system which contains 0 to 2 oxygen atoms and whose alkanediyl radical may optionally be substituted by one or more fluorine atoms;
G¹, G², G³, G⁴ and G⁵ particularly preferably represent hydrogen or a combination of up to three substituents independently of one another selected from the group consisting of: halogen, C₁-C₄-alkyl, C₂-C₄-alkenyl, C₂-C₄-alkynyl, C₁-C₄-haloalkyl, SF₅, hydroxyl, C₁-C₄-alkoxy, C₁-C₄-alkoxy-C₁-C₄-alkyl, C₁-C₄-alkoxy-C₁-C₄-alkoxy, C₃-C₄-cycloalkyl, C₂-C₄-alkenyloxy, C₂-C₄-alkynyloxy, C₃-C₄-cycloalkyl-C₁-C₄-alkoxy, C₁-C₄-haloalkoxy, C₁-C₄-haloalkoxy-C₁-C₄-alkyl, C₁-C₄-alkylsulphanyl, C₁-C₄-haloalkylsulphanyl, C₁-C₄-alkylsulphinyl, C₁-C₄-haloalkylsulphinyl, C₁-C₄-alkylsulphonyl, C₁-C₄-haloalkylsulphonyl, cyano, C₁-C₄-alkylcarbonyl, C₁-C₄-alkoxycarbonyl, C₁-C₄-alkoxycarbonyl-C₁-C₄-alkyl, carboxyl, aminocarbonyl, aminothiocarbonyl, C₁-C₄-alkylaminocarbonyl, C₁-C₄-dialkylaminocarbonyl, C₁-C₄-trialkylsilyl, nitro, amino, C₁-C₄-alkylamino, C₁-C₄-dialkylamino, C₁-C₄-alkylsulphonylamino, C₁-C₄-dialkylsulphonylamino, —CR⁵═NO—C₁-C₄-alkyl, —CR⁵═NO-halo-C₁-C₄-alkyl or formyl, where vicinal C₁-C₄-alkyl, C₁-C₄ haloalkyl, C₁-C₄-alkoxy and/or C₁-C₄-haloalkoxy groups together with the carbon atoms to which they are attached may form a five- to six-membered cyclic system which contains 0 to 2 oxygen atoms and whose alkanediyl radical may optionally be substituted by one or more fluorine atoms;
G¹, G², G, G⁴ and G⁵ very particularly preferably represent a 3-substitution, 4-substitution, 3,4-disubstitution, 3,5-disubstitution or 3,4,5-trisubstitution, where the substituents G¹, G², G³, G⁴ and G⁵ independently of one another are selected from the group consisting of: hydrogen, halogen, C₁-C₂-alkyl, C₁-C₂-haloalkyl, C₁-C₂-alkoxy, C₁-C₂-alkoxy-C₁-C₂-alkyl, C₁-C₂-alkoxy-C₁-C₂-alkoxy, C₃-C₆-cycloalkyl-C₁-C₂-alkoxy, C₁-C₂-haloalkoxy, C₁-C₂-alkylsulphanyl, C₁-C₂-haloalkylsulphanyl, C₁-C₂-alkylsulphinyl, C₁-C₂-haloalkylsulphinyl, C₁-C₂-alkylsulphonyl, C₁-C₂-haloalkylsulphonyl, cyano, C₁-C₂-alkylcarbonyl, C₁-C₂-alkoxycarbonyl, carboxyl, aminocarbonyl, aminothiocarbonyl, C₁-C₂-alkylaminocarbonyl, C₁-C₂-dialkylaminocarbonyl, nitro, amino, C₁-C₂-alkylamino, C₁-C₂-dialkylamino, C₁-C₂-alkylsulphonylamino, C₁-C₂-dialkylsulphonylamino;
G¹, G², G³, G⁴ and G⁵ especially preferably represent a 2-chloro substitution, 3-chloro substitution, 3-bromo substitution, 3-fluoro substitution, 4-bromo substitution, 4-chloro substitution, 3,4-dichloro substitution, 3,4-difluoro substitution, 3-chloro-4-fluoro substitution, 3-fluoro-4-chloro substitution, 3-fluoro-5-chloro substitution, 3-chloro-5-trifluoromethyl substitution, 3,5-dichloro substitution, 3,5-dimethyl substitution or 3,5-bistrifluoromethyl substitution;
   Q¹ preferably represents one of the heterocyclic groupings listed below:

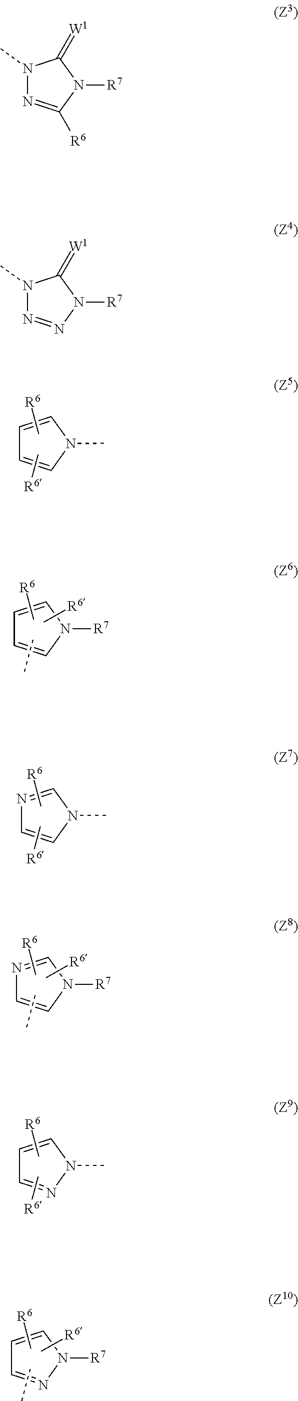

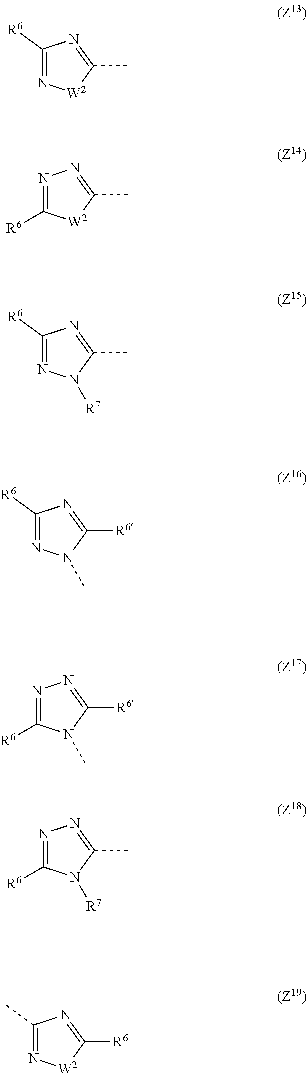

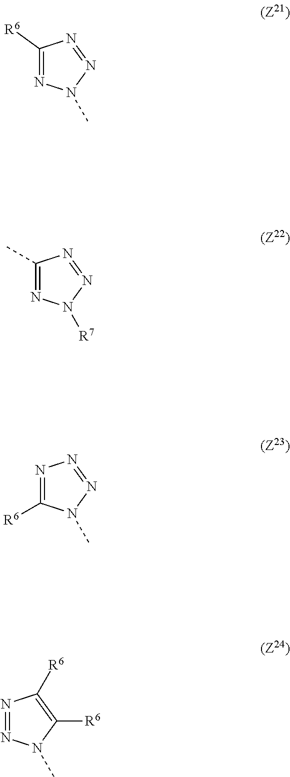

Q¹ particularly preferably represents one of the heterocyclic groupings listed below:

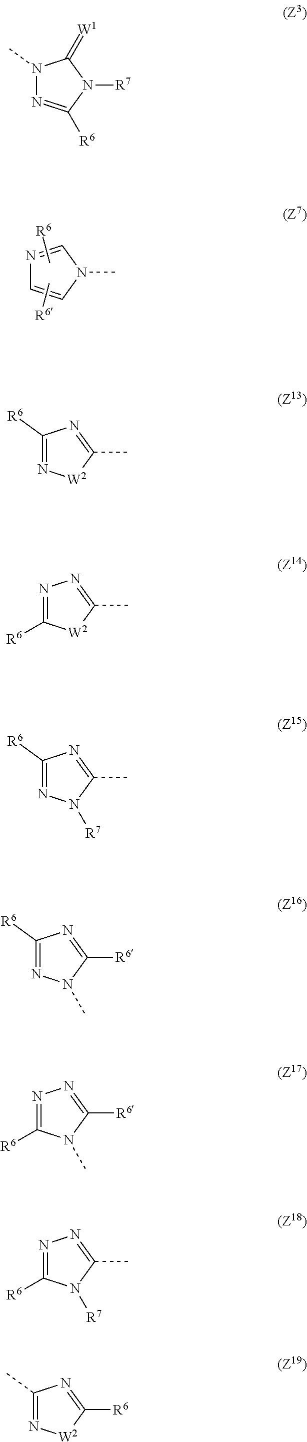

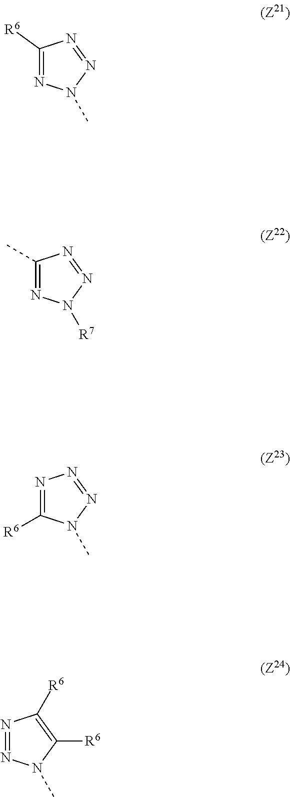

Q¹ very particularly preferably represents one of the heterocyclic groupings listed below:

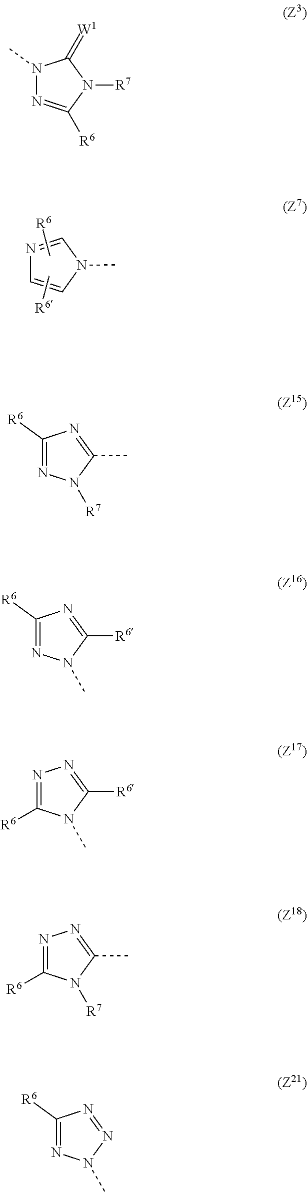

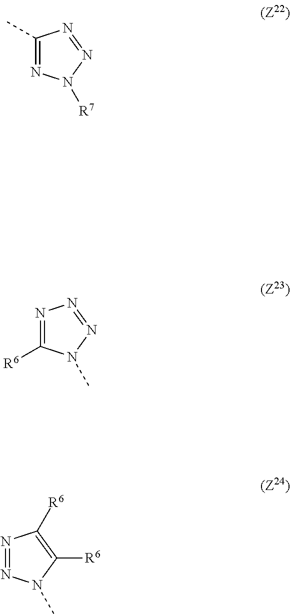

Q¹ especially preferably represents one of the heterocyclic groupings listed below:

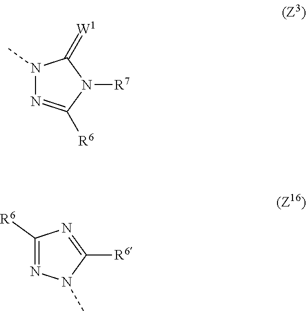

where
W¹ preferably represents oxygen or sulphur;
W¹ very particularly preferably represents oxygen;
W² preferably represents oxygen or sulphur;
W² very particularly preferably represents oxygen; and
R⁶, R^{6′}, R^{6″}, R^{6′″} and R⁷ have the meaning given below;
Q² preferably represents C(O)NR⁸R⁹;
Q³ preferably represents C(R¹⁰R¹¹)NR⁸R⁹;
Q⁴ preferably represents cyano (where R¹ does not represent amino), COOH, COOR¹², fluorine (if R³ is different from chlorine), chlorine (if R³ is different from chlorine, COOH, CH₂CH₂OMe and OMe), bromine, iodine, SR¹² (where R¹ does not represent amino if R¹² represents alkyl), S(O)R¹², S(O)₂R¹² or S(O)₂NR⁸R⁹;
Q⁴ particularly preferably represents cyano (where R¹ does not represent amino), COOH, COOMe, COOEt, fluorine (if R³ is different from chlorine), chlorine (if R³ is different from chlorine, COOH, CH₂CH₂OMe and OMe), bromine, iodine, SR¹² (where R¹ does not represent amino if R¹² represents alkyl), S(O)R¹², S(O)₂R¹² or S(O)₂NR⁸R⁹;
Q⁴ very particularly preferably represents cyano (where R¹ does not represent amino), COOH, COOMe, COOEt, fluorine (if R³ is different from chlorine), bromine, iodine, SR¹² (where R¹ does not represent amino if R¹² represents alkyl), S(O)R¹², S(O)₂R¹² or S(O)₂NR⁸R⁹;

Q⁴ especially preferably represents cyano (where R¹ does not represent amino), COOH, COOMe, COOEt, fluorine (if R³ is different from chlorine), bromine, iodine, SR¹² (where R¹ does not represent amino if R¹² represents alkyl), S(O)R¹² or S(O)₂R¹²;
R¹ preferably represents hydrogen, halogen, C₁-C₄-alkylsulphanyl, C₁-C₄-alkylsulphinyl, C₁-C₄-alkylsulphonyl, Z¹⁶, amino or NR¹³R¹⁴;
R¹ particularly preferably represents hydrogen, fluorine, chlorine, bromine, iodine, Z¹⁶, amino or NR¹³R¹⁴;
   R¹ very particularly preferably represents chlorine, bromine, iodine, Z¹⁶, amino or NR¹³R¹⁴;
   R¹ especially preferably represents amino or NR¹³R¹⁴;
R² preferably represents hydrogen, halogen, C₁-C₆-alkyl, C₁-C₆-haloalkyl, C₁-C₆-alkoxy-C₁-C₆-alkyl, C₁-C₆-haloalkoxy-C₁-C₆-alkyl, C₁-C₆-alkoxy-C₁-C₆-haloalkyl, C₁-C₆-alkylsulphanyl-C₁-C₆-alkyl, C₁-C₆-alkylsulphinyl-C₁-C₆-alkyl, C₁-C₆-alkylsulphonyl-C₁-C₆-alkyl, C₁-C₆-haloalkylsulphanyl-C₁-C₆-alkyl, C₁-C₆-haloalkylsulphinyl-C₁-C₆-alkyl, C₁-C₆-haloalkylsulphonyl-C₁-C₆-alkyl, C₃-C₆-cycloalkyl-C₁-C₆-alkyl, phenyl-C₁-C₆-alkyl, heteroaryl-C₁-C₆-alkyl, hetero-C₃-C₆-cyclyl-C₁-C₆-alkyl, aryl-C₁-C₆-haloalkyl, C₁-C₆-haloalkyloxy-C₁-C₆-haloalkyloxy-C₁-C₆-haloalkyl, C₃-C₆-heterocyclyl, C₁-C₆-alkylsulphanyl, C₁-C₆-alkylsulphinyl, C₁-C₆-alkylsulphonyl or phenyl;
where a phenyl or heteroaryl radical is optionally mono- or polysubstituted by halogen, C₁-C₆-alkyl, C₁-C₆-haloalkyl, C₁-C₆-alkoxy, nitro or cyano; a heterocyclyl radical is optionally substituted by —OH/═O, —SH/═S, —NH₂, halogen, C₁-C₆-alkyl, C₁-C₆-haloalkyl, C₁-C₆-alkoxy, C₁-C₆-alkylsulphanyl, C₁-C₆-alkylsulphinyl, C₁-C₆-alkylsulphonyl, C₁-C₆-monoalkylamino, C₁-C₆-dialkylamino, nitro or cyano; and a phenyl-, heteroaryl- or heterocyclyl-bound alkanediyl radical is optionally mono- or polysubstituted by halogen, C₁-C₆-alkyl, C₁-C₆-haloalkyl, C₁-C₆-alkoxy or cyano;
R² particularly preferably represents hydrogen, halogen, C₁-C₄-alkyl, C₁-C₄-haloalkyl, C₁-C₄-alkoxy-C₁-C₄-alkyl, C₁-C₄-haloalkoxy-C₁-C₄-alkyl, C₁-C₄-alkoxy-C₁-C₄-haloalkyl, C₁-C₄-alkylsulphanyl-C₁-C₄-alkyl, C₁-C₄-alkylsulphinyl-C₁-C₄-alkyl, C₁-C₄-alkylsulphonyl-C₁-C₄-alkyl, C₁-C₄-haloalkylsulphanyl-C₁-C₄-alkyl, C₁-C₄-haloalkylsulphinyl-C₁-C₄-alkyl, C₁-C₄-haloalkylsulphonyl-C₁-C₄-alkyl, C₃-C₄-cycloalkyl-C₁-C₄-alkyl, phenyl-C₁-C₄-alkyl, heteroaryl-C₁-C₄-alkyl, hetero-C₃-C₄-cyclyl-C₁-C₄-alkyl, aryl-C₁-C₄-haloalkyl, C₁-C₄-haloalkyloxy-C₁-C₄-haloalkyloxy-C₁-C₄-haloalkyl, C₃-C₄-heterocyclyl, C₁-C₄-alkylsulphanyl, C₁-C₄-alkylsulphinyl, C₁-C₄-alkylsulphonyl or phenyl;
where a phenyl or heteroaryl radical is optionally mono- or polysubstituted by halogen, C₁-C₄-alkyl, C₁-C₄-haloalkyl, C₁-C₄-alkoxy, nitro or cyano; a heterocyclyl radical is optionally substituted by —OH/═O, —SH/═S, —NH₂, halogen, C₁-C₄-alkyl, C₁-C₄-haloalkyl, C₁-C₄-alkoxy, C₁-C₄-alkylsulphanyl, C₁-C₄-alkylsulphinyl, C₁-C₄-alkylsulphonyl, C₁-C₄-monoalkylamino, C₁-C₄-dialkylamino, nitro or cyano; and a phenyl-, heteroaryl- or heterocyclyl-bound alkanediyl radical is optionally mono- or polysubstituted by halogen, C₁-C₄-alkyl, C₁-C₄-haloalkyl, C₁-C₄-alkoxy or cyano;
R² very particularly preferably represents fluorine, chlorine, bromine, iodine, C₁-C₄-alkoxy-C₁-C₄-alkyl, C₁₋₄-alkyl, aryl-C₁-C₄-haloalkyl, C₁-C₆-alkylsulphanyl, C₁-C₆-alkylsulphinyl, C₁-C₆-alkylsulphonyl or C₁₋₄-haloalkyl;
   R² especially preferably represents C₁₋₂-haloalkyl;
R³ preferably represents halogen, C₁-C₆-alkyl, C₂-C₆-alkenyl, C₂-C₆-alkynyl, C₁-C₆-haloalkyl, C₃-C₆-cycloalkyl, C₁-C₆-alkoxy-C₁-C₆-alkyl, C₁-C₆-alkylcarbonyloxy-C₁-C₆-alkyl, C₁-C₆-alkylsulphanyl-C₁-C₆-alkyl, C₁-C₆-alkylsulphinyl-C₁-C₆-alkyl, C₁-C₆-alkylsulphonyl-C₁-C₆-alkyl, hydroxyl, C₁-C₆-alkoxy, C₁-C₆-acylamino, C₁-C₆-alkoxycarbonylamino, C₂-C₆-alkenyloxy, C₂-C₆-alkynyloxy, C₃-C₆-cycloalkyl-C₁-C₆-alkoxy, C₁-C₆-haloalkoxy, C₁-C₆-alkylsulphanyl, C₁-C₆-haloalkylsulphanyl, C₁-C₆-alkylsulphinyl, C₁-C₆-haloalkylsulphinyl, C₁-C₆-alkylsulphonyl, C₁-C₆-haloalkylsulphonyl, cyano, C₁-C₆-alkylcarbonyl, C₁-C₆-alkoxycarbonyl, C₁-C₆-alkoxycarbonyl-C₁-C₆-alkyl, carboxyl, aminocarbonyl, aminothiocarbonyl, C₁-C₆-alkylaminocarbonyl, C₁-C₆-dialkylaminocarbonyl, C₁-C₆-alkylsulphonylaminocarbonyl, C₁-C₆-trialkylsilyl, nitro, amino, C₁-C₆-alkylamino, C₁-C₆-dialkylamino, C₁-C₆-alkylsulphonylamino, C₁-C₆-dialkylsulphonylamino, —CR⁵═NO—R⁵, —CR⁵═NO-halo-C₁-C₆-alkyl, Z¹⁶ or formyl;
R³ especially preferably represents halogen, C₁-C₄-alkyl, C₂-C₄-alkenyl, C₂-C₄-alkynyl, C₁-C₄-haloalkyl, C₃-C₄-cycloalkyl, C₁-C₄-alkoxy-C₁-C₄-alkyl, C₁-C₄-alkylcarbonyloxy-C₁-C₄-alkyl, C₁-C₄-alkylsulphanyl-C₁-C₄-alkyl, C₁-C₄-alkylsulphinyl-C₁-C₄-alkyl, C₁-C₄-alkylsulphonyl-C₁-C₄-alkyl, hydroxyl, C₁-C₄-alkoxy, C₁-C₄-acylamino, C₁-C₄-alkoxycarbonylamino, C₂-C₄-alkenyloxy, C₂-C₄-alkynyloxy, C₃-C₄-cycloalkyl-C₁-C₄-alkoxy, C₁-C₄-haloalkoxy, C₁-C₄-alkylsulphanyl, C₁-C₄-haloalkylsulphanyl, C₁-C₄-alkylsulphinyl, C₁-C₄-haloalkylsulphinyl, C₁-C₄-alkylsulphonyl, C₁-C₄-haloalkylsulphonyl, cyano, C₁-C₄-alkylcarbonyl, C₁-C₄-alkoxycarbonyl, C₁-C₄-alkoxycarbonyl-C₁-C₄-alkyl, carboxyl, aminocarbonyl, aminothiocarbonyl, C₁-C₄-alkylaminocarbonyl, C₁-C₄-dialkylaminocarbonyl, C₁-C₄-alkylsulphonylaminocarbonyl, C₁-C₄-trialkylsilyl, nitro, amino, C₁-C₄-alkylamino, C₁-C₄-dialkylamino, C₁-C₄-alkylsulphonylamino, C₁-C₄-dialkylsulphonylamino, —CR⁵═NO—R⁵, —CR⁵═NO-halo-C₁-C₄-alkyl, Z¹⁶ or formyl;
R³ very particularly preferably represents halogen, C₁-C₂-alkyl, C₁-C₂-haloalkyl, cyclopropyl, C₁-C₂-alkoxy-C₁-C₂-alkyl, C₁-C₂-alkylcarbonyloxy-C₁-C₂-alkyl, C₁-C₂-alkylsulphanyl-C₁-C₂-alkyl, C₁-C₂-alkylsulphinyl-C₁-C₂-alkyl, C₁-C₄-alkylsulphonyl-C₁-C₂-alkyl, C₁-C₂-alkoxy, C₁-C₂-acylamino, C₁-C₂-alkoxycarbonylamino, cyclopropyl-C₁-C₂-alkoxy, C₁-C₂-haloalkoxy, C₁-C₂-alkylsulphanyl, C₁-C₂-haloalkylsulphanyl, C₁-C₂-alkylsulphinyl, C₁-C₂-haloalkylsulphinyl, C₁-C₂-alkylsulphonyl, C₁-C₂-haloalkylsulphonyl, cyano, C₁-C₂-alkylcarbonyl, C₁-C₂-alkoxycarbonyl, C₁-C₂-alkoxycarbonyl-C₁-C₂-alkyl, carboxyl, aminocarbonyl, aminothiocarbonyl, C₁-C₂-alkylaminocarbonyl, C₁-C₂-dialkylaminocarbonyl, C₁-C₂-alkylsulphonylaminocarbonyl, C₁-C₂-trialkylsilyl, nitro, amino, C₁-C₂-alkylamino, C₁-C₂-dialkylamino, C₁-C₂-alkylsulphonylamino, C₁-C₂-dialkylsulphonylamino, —CR⁵═NO—R⁵, —CR⁵═NO-halo-C₁-C₂-alkyl, Z¹⁶ or formyl;
R³ especially preferably represents fluorine, chlorine, bromine, iodine, C₁-C₂-alkyl, C₁-C₂-haloalkyl, C₁-C₂-haloalkylsulphanyl, C₁-C₂-haloalkylsulphinyl, C₁-C₂-haloalkylsulphonyl or cyano;
R⁴ preferably represents hydrogen, cyano, nitro, C₁-C₆-alkyl, C₁-C₆-haloalkyl, halogen or C₁-C₆-alkoxy;
R⁴ particularly preferably represents hydrogen, cyano, nitro, C₁-C₄-alkyl, C₁-C₄-haloalkyl, halogen or C₁-C₄-alkoxy;
   R⁴ very particularly preferably represents hydrogen, fluorine, chlorine, bromine or iodine;
   R⁴ especially preferably represents hydrogen;
   R⁵ preferably represents hydrogen or C₁-C₆-alkyl;
   R⁵ particularly preferably represents hydrogen or C₁-C₄-alkyl;
   R⁵ very particularly preferably represents hydrogen or C₁-C₃-alkyl;
   R⁵ especially preferably represents hydrogen, methyl or ethyl;
R⁶, R^{6′}, R^{6″} and R^{6′″} independently of one another preferably represent hydrogen, amino, hydroxyl, mercapto, nitro, cyano, carboxyl, halogen, C₁-C₆-alkyl, C₁-C₆-haloalkyl, C₂-C₆-alkenyl, C₂-C₆-alkynyl, C₃-C₆-cycloalkyl, C₁-C₆-alkoxy, C₁-C₆-haloalkoxy, C₂-C₆-alkenyloxy, C₂-C₆-alkynyloxy, C₁-C₆-alkoxycarbonyl, C₁-C₆-alkylsulphanyl, C₁-C₆-haloalkylsulphanyl, C₁-C₆-alkylsulphinyl, C₁-C₆-haloalkylsulphinyl, C₁-C₆-alkylsulphonyl, C₁-C₆-haloalkylsulphonyl, amino-carbonyl, aminothiocarbonyl, CR⁵═NO—C₁-C₆-alkyl, —CR⁵═NO-halo-C₁-C₆-alkyl, formyl, C₁-C₆-alkylamino, C₁-C₆-dialkylamino, phenyl, heteroaryl, Hheteroaryl-C₁-C₆-alkyl or hetero-C₃-C₆-cyclyl-C₁-C₆-alkyl;
where a phenyl or heteroaryl radical is optionally mono- or polysubstituted by halogen, C₁-C₆-alkyl, C₁-C₆-haloalkyl, C₁-C₆-alkoxy, nitro or cyano; a heterocyclyl radical is optionally substituted by —OH/═O, —SH/═S, —NH₂, halogen, C₁-C₆-alkyl, C₁-C₆-haloalkyl, C₁-C₆-alkoxy, C₁-C₆-alkylsulphanyl, C₁-C₆-alkylsulphinyl, C₁-C₆-alkylsulphonyl, C₁-C₆-monoalkylamino, C₁-C₆-dialkylamino, nitro or cyano; and a phenyl-, heteroaryl- or heterocyclyl-bound alkanediyl radical is optionally mono- or polysubstituted by halogen, C₁-C₆-alkyl, C₁-C₆-haloalkyl, C₁-C₆-alkoxy or cyano;
R⁶, R^{6′}, R^{6″} and R^{6′″} independently of one another particularly preferably represent hydrogen, amino, hydroxyl, mercapto, nitro, cyano, carboxyl, halogen, C₁-C₄-alkyl, C₁-C₄-haloalkyl, C₂-C₄-alkenyl, C₂-C₄-alkynyl, C₃-C₄-cycloalkyl, C₁-C₄-alkoxy, C₁-C₄-haloalkoxy, C₂-C₄-alkenyloxy, C₂-C₄-alkynyloxy, C₁-C₄-alkoxycarbonyl, C₁-C₄-alkylsulphanyl, C₁-C₄-haloalkylsulphanyl, C₁-C₄-alkylsulphinyl, C₁-C₄-haloalkylsulphinyl, C₁-C₄-alkylsulphonyl, C₁-C₄-haloalkylsulphonyl, aminocarbonyl, aminothiocarbonyl, CR⁵═NO—C₁-C₄-alkyl, —CR⁵═NO-halo-C₁-C₄-alkyl, formyl, C₁-C₄-alkylamino, C₁-C₄-dialkylamino, phenyl, heteroaryl, Hheteroaryl-C₁-C₄-alkyl or hetero-C₃-C₄-cyclyl-C₁-C₄-alkyl;
where a phenyl or heteroaryl radical is optionally mono- or polysubstituted by halogen, C₁-C₄-alkyl, C₁-C₄-haloalkyl, C₁-C₄-alkoxy, nitro or cyano; a heterocyclyl radical is optionally substituted by —OH/═O, —SH/═S, —NH₂, halogen, C₁-C₄-alkyl, C₁-C₄-haloalkyl, C₁-C₄-alkoxy, C₁-C₄-alkylsulphanyl, C₁-C₄-alkylsulphinyl, C₁-C₄-alkylsulphonyl, C₁-C₄-monoalkylamino, C₁-C₄-dialkylamino, nitro or cyano; and a phenyl-, heteroaryl- or heterocyclyl-bound alkanediyl radical is optionally mono- or polysubstituted by halogen, C₁-C₁-C₄-haloalkyl, C₁-C₄-alkoxy or cyano;
R⁶, R^{6′}, R^{6″} and R^{6′″} independently of one another very particularly preferably represent hydrogen, amino, cyano, fluorine, chlorine, bromine, iodine, C₁-C₂-alkyl, C₁-C₂-haloalkyl, C₁-C₄ alkoxy, C₁-C₂-haloalkoxy, phenyl or heteroaryl;
where a phenyl or heteroaryl radical is optionally mono- or polysubstituted by halogen, C₁-C₂-alkyl, C₁-C₂-haloalkyl, C₁-C₂-alkoxy, nitro or cyano;
R⁶, R^{6′}, R^{6″} and R^{6′″} independently of one another especially preferably represent hydrogen, amino, cyano, fluorine, chlorine, methyl, ethyl, C₁-C₂-haloalkyl, methoxy, ethoxy or C₁-C₄ haloalkoxy;

R⁷ preferably represents hydrogen, amino, hydroxyl, cyano, C₁-C₆-alkyl, C₁-C₆-haloalkyl, C₂-C₆-alkenyl, C₂-C₆-alkynyl, C₃-C₆-cycloalkyl, C₁-C₆-alkoxy, C₂-C₆-alkenyloxy, C₂-C₆-alkynyloxy, C₁-C₆-alkoxycarbonyl, C₁-C₆-alkylcarbonyl, C₁-C₆-alkylaminocarbonyl, C₁-C₆-dialkylaminocarbonyl, phenyl, phenyl-C₁-C₆-alkyl, heteroaryl-C₁-C₆-alkyl or hetero-C₃-C₆-cyclyl-C₁-C₆-alkyl;
where a phenyl or heteroaryl radical is optionally mono- or polysubstituted by halogen, C₁-C₆-alkyl, C₁-C₆-haloalkyl, C₁-C₆-alkoxy, nitro or cyano; a heterocyclyl radical is optionally substituted by —OH/═O, —SH/═S, —NH₂, halogen, C₁-C₆-alkyl, C₁-C₆-haloalkyl, C₁-C₆-alkoxy, C₁-C₆-alkylsulphanyl, C₁-C₆-alkylsulphinyl, C₁-C₆-alkylsulphonyl, C₁-C₆-monoalkylamino, C₁-C₆-dialkylamino, nitro or cyano; and a phenyl-, heteroaryl- or heterocyclyl-bound alkanediyl radical is optionally mono- or polysubstituted by halogen, C₁-C₆-alkyl, C₁-C₆-haloalkyl, C₁-C₆-alkoxy or cyano and
where optionally two adjacent radicals from the group consisting of R⁶, and R⁷ together represent alkanediyl or alkenediyl, each of which has 2 to 5 carbon atoms and is optionally substituted and/or optionally interrupted at the beginning (or the end) or within the hydrocarbon chain by oxygen or sulphur or a grouping from the group consisting of —S(O)—, —SO₂—, —NH— and —N—C₁-C₆-alkyl-;
R⁷ particularly preferably represents hydrogen, amino, hydroxyl, cyano, C₁-C₄-alkyl, C₁-C₄-haloalkyl, C₂-C₄-alkenyl, C₂-C₄-alkynyl, C₃-C₄-cycloalkyl, C₁-C₄-alkoxy, C₂-C₄-alkenyloxy, C₂-C₄-alkynyloxy, C₁-C₄-alkoxycarbonyl, C₁-C₄-alkylcarbonyl, C₁-C₄-alkylaminocarbonyl, C₁-C₄-dialkylaminocarbonyl, phenyl, phenyl-C₁-C₄-alkyl, heteroaryl-C₁-C₄-alkyl or hetero-C₃-C₄-cyclyl-C₁-C₄-alkyl;
where a phenyl or heteroaryl radical is optionally mono- or polysubstituted by halogen, C₁-C₄-alkyl, C₁-C₄-haloalkyl, C₁-C₄-alkoxy, nitro or cyano; a heterocyclyl radical is optionally substituted by —OH/═O, —SH/═S, —NH₂, halogen, C₁-C₄-alkyl, C₁-C₄-haloalkyl, C₁-C₄-alkoxy, C₁-C₄-alkylsulphanyl, C₁-C₄-alkylsulphinyl, C₁-C₄-alkylsulphonyl, C₁-C₄-monoalkylamino, C₁-C₄-dialkylamino, nitro or cyano; and a phenyl-, heteroaryl- or heterocyclyl-bound alkanediyl radical is optionally mono- or polysubstituted by halogen, C₁-C₄-alkyl, C₁-C₄-haloalkyl, C₁-C₄-alkoxy or cyano and
where optionally two adjacent radicals from the group consisting of R⁶, R^{6′}, R^{6″}, R^{6′″} and R⁷ together represent alkanediyl or alkenediyl, each of which has 2 to 5 carbon atoms and is optionally substituted and/or optionally interrupted at the beginning (or the end) or within the hydrocarbon chain by oxygen or sulphur or a grouping from the group consisting of —S(O)—, —SO₂—, —NH— and —N—C₁-C₄-alkyl-;
R⁷ very particularly preferably represents hydrogen, amino, hydroxyl, cyano, C₁-C₂-alkyl, C₁-C₂-haloalkyl, cyclopropyl, C₁-C₂-alkoxy, C₁-C₂-alkoxycarbonyl, C₁-C₂-alkylcarbonyl, C₁-C₂-alkylaminocarbonyl, C₁-C₂-dialkylaminocarbonyl, phenyl, phenyl-C₁-C₂-alkyl or heteroaryl-C₁-C₂-alkyl;
where a phenyl or heteroaryl radical is optionally mono- or polysubstituted by halogen, C₁-C₂-alkyl, C₁-C₂-haloalkyl, C₁-C₂-alkoxy, nitro or cyano; and a phenyl- or heteroaryl-bound alkanediyl radical is optionally mono- or polysubstituted by halogen, C₁-C₂-alkyl, C₁-C₄-haloalkyl, C₁-C₂-alkoxy or cyano and where optionally two adjacent radicals R⁶/R⁶, R⁶/R⁷ or R⁷/R⁷ together represent alkanediyl or alkenediyl, each of which has 2 to 4 carbon atoms and is optionally substituted and/or optionally interrupted at the beginning (or the end) or within the hydrocarbon chain by oxygen or sulphur or a grouping from the group consisting of —S(O)—, —SO₂—, —NH— and —N—C₁-C₂-alkyl-;
R⁷ especially preferably represents hydrogen, amino, cyano, C₁-C₂-alkyl, C₁-C₂-haloalkyl or C₁-C₂-alkoxy;
R⁸ and R⁹ preferably and independently of one another represent hydrogen, C₁-C₆-alkyl, C₁-C₆-haloalkyl, C₂-C₆-alkenyl (optionally substituted by halogen, C₁-C₆-alkyl, C₁-C₆-haloalkyl or cyano), C₂-C₆-alkynyl, C₃-C₆-cycloalkyl (optionally mono- or polysubstituted at the cycle by halogen, C₁-C₆-haloalkyl, C₁-C₆-alkyl or condensed to an aromatic or heteroaromatic moiety), C₃-C₆-cycloalkyl-C₁-C₆-alkyl (optionally mono- or polysubstituted at the cycle by halogen, C₁-C₆-haloalkyl, C₁-C₆-alkyl or condensed to an aromatic or heteroaromatic moiety, optionally mono- or polysubstituted at the C₁-C₆-alkyl moiety by halogen, C₁-C₆-alkyl, C₁-C₆-haloalkyl, C₁-C₆-alkoxy or cyano), C₃-C₆-cycloalkylcarbonyl-C₃-C₆-heterocyclyl, C₁-C₆-alkyloxycarbonyl-C₃-C₆-heterocyclyl, C₁-C₆-alkylsulphinyl-C₁-C₆-alkyl, C₁-C₆-alkylsulphanyl-C₁-C₆-alkyl, C₁-C₆-alkylsulphonyl-C₁-C₆-alkyl, hydroxy-C₁-C₆-alkyl, C₃-C₆-heterocyclylcarbonyl-C₁-C₆-alkyl, heteroarylcarbonylamino-C₁-C₆-alkyl, C₁-C₆-haloalkoxy-C₁-C₆-alkyl, C₁-C₆-alkylthiocarbonyl, C₁-C₆-dialkylaminocarbonyl, C₁-C₆-alkylaminocarbonyl, C₁-C₆-haloalkylaminocarbonyl, C₁-C₆-alkoxy, C₂-C₆-alkenyloxy, C₂-C₆-alkynyloxy, C₁-C₆-alkoxy-C₁-C₆-alkyl, C₁-C₆-alkylcarbonyl-C₁-C₆-alkyl, C(R¹⁰R¹¹)CR⁵═NO—C₁-C₆-alkyl, C₁-C₆-alkylsulphonyl, C₁-C₆-alkylcarbonyl, C₁-C₆-haloalkylcarbonyl, C₂-C₆-alkenylcarbonyl, C₂-C₆-alkynylcarbonyl, C₃-C₆-cycloalkylcarbonyl, C₁-C₆-alkoxycarbonyl, C₂-C₆-alkenyloxycarbonyl, C₂-C₆-alkynyloxycarbonyl, C₁-C₆-alkoxyalkylcarbonyl, phenylsulphonyl, phenyl, heteroaryl, hetero-C₃-C₆-cyclyl, phenyl-C₁-C₆-alkyl, heteroaryl-C₁-C₆-alkyl, hetero-C₃-C₆-cyclyl-C₁-C₆-alkyl, phenylcarbonyl, heteroarylcarbonyl, hetero-C₃-C₆-cyclylcarbonyl, phenyl-C₁-C₆-alkylcarbonyl, heteroaryl-C₁-C₆-alkylcarbonyl, hetero-C₃-C₆-cyclyl-C₁-C₆-alkylcarbonyl, phenoxycarbonyl or phenyl-C₁-C₆-alkoxycarbonyl;
where a phenyl or heteroaryl radical is optionally mono- or polysubstituted by halogen, C₁-C₆-alkyl, C₁-C₆-haloalkyl, C₁-C₆-alkoxy, nitro or cyano; a heterocyclyl radical is optionally substituted by —OH/═O, —SH/═S, —NH₂, halogen, C₁-C₆-alkyl, C₁-C₆-haloalkyl, C₁-C₆-alkoxy, C₁-C₆-alkylsulphanyl, C₁-C₆-alkylsulphinyl, C₁-C₆-alkylsulphonyl, C₁-C₆-monoalkylamino, C₁-C₆-dialkylamino, nitro or cyano; and a phenyl-, heteroaryl- or heterocyclyl-bound alkanediyl radical is optionally mono- or polysubstituted by halogen, C₁-C₆-alkyl, C₁-C₆-haloalkyl, C₁-C₆-alkoxy or cyano and
where optionally R⁸/R⁹ together represent alkanediyl or alkenediyl, each of which has 2 to 5 carbon atoms and is optionally substituted and/or optionally interrupted at the beginning (or the end) or within the hydrocarbon chain by oxygen or sulphur or a grouping from the group consisting of —S(O)—, —SO₂—, —NH— or —N—C₁-C₆-alkyl-;
R⁸ and R⁹ particularly preferably and independently of one another represent hydrogen, C₁-C₄-alkyl, C₁-C₄-haloalkyl, C₂-C₄-alkenyl (optionally substituted by halogen, C₁-C₄-alkyl, C₁-C₄-haloalkyl or cyano), C₂-C₄-alkynyl, C₃-C₅-cycloalkyl (optionally mono- or polysubstituted at the cycle by halogen, C₁-C₄-haloalkyl, C₁-C₄-alkyl or condensed to an aromatic or heteroaromatic moiety), C₃-C₅-cycloalkyl-C₁-C₄-alkyl (optionally mono- or polysubstituted at the cycle by halogen, C₁-C₄-haloalkyl, C₁-C₄-alkyl or condensed to an aromatic or heteroaromatic moiety, optionally mono- or polysubstituted at the C₁-C₄-alkyl moiety by halogen, C₁-C₄-alkyl, C₁-C₄-haloalkyl C₁-C₄-alkoxy or cyano), C₁-C₄-alkylsulphinyl-C₁-C₄-alkyl, C₁-C₄-alkylsulphanyl-C₁-C₄-alkyl, C₁-C₄-alkylsulphonyl-C₁-C₄-alkyl, C₁-C₄-haloalkoxy-C₁-C₄-alkyl, C₁-C₄-alkylthiocarbonyl, C₁-C₄-dialkylaminocarbonyl, C₁-C₄-alkylaminocarbonyl, C₁-C₄-haloalkylaminocarbonyl, C₁-C₄-alkoxy, C₂-C₄-alkenyloxy, C₂-C₄-alkynyloxy, C₁-C₄-alkoxy-C₁-C₄-alkyl, C₁-C₄-alkylcarbonyl-C₁-C₄-alkyl, C(R¹⁰R¹¹)CR⁵═NO—C₁-C₄-alkyl, C₁-C₄-alkylsulphonyl, C₁-C₄-alkylcarbonyl, C₁-Cᵣ haloalkylcarbonyl, C₂-C₄-alkenylcarbonyl, C₂-C₄-alkynylcarbonyl, C₃-C₄-cycloalkylcarbonyl, C₁-C₄-alkoxycarbonyl, C₂-C₄-alkenyloxycarbonyl, C₂-C₄-alkynyloxycarbonyl, C₁-C₄-alkoxyalkylcarbonyl, phenylsulphonyl, phenyl, heteroaryl, hetero-C₃-C₄-cyclyl, phenyl-C₁-C₄-alkyl, heteroaryl-C₁-C₄-alkyl, hetero-C₃-C₄-cyclyl-C₁-C₄-alkyl, phenylcarbonyl, heteroarylcarbonyl, hetero-C₃-C₄-cyclylcarbonyl, phenyl-C₁-C₄-alkylcarbonyl, heteroaryl-C₁-C₄-alkylcarbonyl, hetero-C₃-C₄-cyclyl-C₁-C₄-alkylcarbonyl, phenoxycarbonyl or phenyl-C₁-C₄-alkoxycarbonyl;
where a phenyl or heteroaryl radical is optionally mono- or polysubstituted by halogen, C₁-C₄-alkyl, C₁-C₄-haloalkyl, C₁-C₄-alkoxy, nitro or cyano; a heterocyclyl radical is optionally substituted by —OH/═O, —SH/═S, —NH₂, halogen, C₁-C₄-alkyl, C₁-C₄-haloalkyl, C₁-C₄-alkoxy, C₁-C₄-alkylsulphanyl, C₁-C₄-alkylsulphinyl, C₁-C₄-alkylsulphonyl, C₁-C₄-monoalkylamino, C₁-C₄-dialkylamino, nitro or cyano; and a phenyl-, heteroaryl- or heterocyclyl-bound alkanediyl radical is optionally mono- or polysubstituted by halogen, C₁-C₄-alkyl, C₁-C₄-haloalkyl, C₁-C₄-alkoxy or cyano and
where optionally R⁸/R⁹ together represent alkanediyl or alkenediyl, each of which has 2 to 5 carbon atoms and is optionally substituted and/or optionally interrupted at the beginning (or the end) or within the hydrocarbon chain by oxygen or sulphur or a grouping from the group consisting of —S(O)—, —SO₂—, —NH— or —N—C₁-C₄-alkyl-;
R⁸ and R⁹ very particularly preferably and independently of one another represent hydrogen, C₁-C₄-alkyl, C₁-C₄-haloalkyl, C₃-C₅-cycloalkyl (optionally mono- or polysubstituted at the cycle by halogen, C₁-C₂-haloalkyl, C₁-C₂-alkyl or condensed to an aromatic or heteroaromatic moiety), C₃-C₅-cycloalkyl-C₁-C₂-alkyl (optionally mono- or polysubstituted at the cycle by halogen, C₁-C₂-haloalkyl, C₁-C₂-alkyl or condensed to an aromatic or heteroaromatic moiety, optionally mono- or polysubstituted at the C₁-C₂-alkyl moiety by halogen, C₁-C₂-alkyl, C₁-C₂-haloalkyl, C₁-C₂-alkoxy or cyano), C₁-C₂-alkylsulphinyl-C₁-C₃-alkyl, C₁-C₂-alkylsulphanyl-C₁-C₃-alkyl, C₁-C₂-alkylsulphonyl-C₁-C₃-alkyl, phenyl-C₁-C₄-alkyl, heteroaryl-C₁-C₄-alkyl, C₁-C₄-dialkylaminocarbonyl, C₁-C₄-alkylaminocarbonyl, C₁-C₄-haloalkylaminocarbonyl, C₁-C₂-alkylsulphonyl, C₁-C₂-alkylcarbonyl, C₁-C₂-haloalkylcarbonyl, C₃-C₅-cycloalkylcarbonyl, C₁-C₂-alkoxycarbonyl, C₁-C₂-alkoxy-C₁-C₂-alkylcarbonyl, phenylsulphonyl, phenyl, heteroaryl, hetero-C₃-C₅-cyclyl, phenylcarbonyl, heteroarylcarbonyl, hetero-C₃-C₅-cyclylcarbonyl, phenyl-C₁-C₂-alkylcarbonyl, heteroaryl-C₁-C₂-alkylcarbonyl, hetero-C₃-C₅-cyclyl-C₁-C₂-alkylcarbonyl, phenoxycarbonyl or phenyl-C₁-C₂-alkoxycarbonyl;
where a phenyl or heteroaryl radical is optionally mono- or polysubstituted by halogen, C₁-C₂-alkyl, C₁-C₂-haloalkyl, C₁-C₂-alkoxy, nitro or cyano; and a phenyl- or heteroaryl-bound alkanediyl radical is optionally mono- or polysubstituted by halogen, C₁-C₄-alkyl, C₁-C₄-haloalkyl, C₁-C₂-alkoxy or cyano and
where optionally R⁸/R⁹ together represent alkanediyl or alkenediyl, each of which has 2 to 5 carbon atoms and is optionally substituted and/or optionally interrupted at the beginning (or the end) or within the hydrocarbon chain by oxygen or sulphur or a grouping from the group consisting of —S(O)—, —SO₂—, —NH— or —N—C₁-C₄-alkyl-;
R⁸ and R⁹ especially preferably and independently of one another represent hydrogen, C₁-C₄ alkyl, C₁-C₄-haloalkyl, C₃-C₅-cycloalkyl, C₃-C₅-cycloalkyl-C₁-C₂-alkyl, C₃-C₅-cyclohaloalkyl-C₁-C₂-alkyl, C₃-C₄-halocycloalkyl, methylsulphanyl-C₁-C₃-alkyl, methylsulphinyl-C₁-C₃-alkyl, methylsulphonyl-C₁-C₃-alkyl, phenylmethyl (optionally mono- or polysubstituted at the aromatic moiety by fluorine, chlorine, bromine, methyl, ethyl, trifluoromethyl, methoxy, cyano or nitro, optionally mono- or polysubstituted at the methyl moiety by methyl, ethyl, propyl, isopropyl, methoxy or trifluoromethyl), pyridylmethyl (optionally mono- or polysubstituted at the pyridyl moiety by fluorine, chlorine, bromine, methyl, ethyl, trifluoromethyl, methoxy, cyano or nitro, optionally mono- or polysubstituted at the methyl moiety by methyl, ethyl, propyl, isopropyl, methoxy or trifluoromethyl) or pyrimidylmethyl (optionally mono- or polysubstituted at the pyrimidyl moiety by fluorine, chlorine, bromine, methyl, ethyl, trifluoromethyl, methoxy, cyano or nitro, optionally mono- or polysubstituted at the methyl moiety by methyl, ethyl, propyl, isopropyl, methoxy or trifluoromethyl);
R¹⁰ and R¹¹ preferably and independently of one another represent hydrogen, C₁-C₆-alkyl, C₁-C₆-haloalkyl or cyano;
R¹⁰ and R¹¹ particularly preferably and independently of one another represent hydrogen, C₁-C₄-alkyl, C₁-C₄-haloalkyl or cyano;

R¹⁰ and R¹¹ very particularly preferably and independently of one another represent hydrogen, C₁-C₂-alkyl or C₁-C₂-haloalkyl;

R¹⁰ and R¹¹ especially preferably represent hydrogen;
R¹² preferably represents C₁-C₆-alkyl or C₁-C₆-haloalkyl;
R¹² particularly preferably represents C₁-C₄-alkyl or C₁-C₄-haloalkyl;
R¹² very particularly preferably represents C₁-C₄-alkyl;
R¹² especially preferably represents methyl or ethyl;
R¹³ and R¹⁴ preferably and independently of one another represent hydrogen, C₁-C₆-alkyl, C₁-C₆-haloalkyl, C₂-C₆-alkenyl, C₂-C₆-alkynyl, C₁-C₆-alkoxy-C₁-C₆-alkyl, C₁-C₆-alkylcarbonyl-C₁-C₆-alkyl, C(R¹⁰R¹¹)CR⁵═NO—C₁-C₆-alkyl, C₁-C₆-alkylsulphonyl, C₁-C₆-alkylcarbonyl, C₁-C₆-haloalkylcarbonyl, C₂-C₆-alkenylcarbonyl, C₂-C₆-alkynylcarbonyl, C₃-C₆-cycloalkylcarbonyl, C₁-C₆-alkoxycarbonyl, C₂-C₆-alkenyloxycarbonyl, C₂-C₆-alkynyloxycarbonyl, C₁-C₆-alkoxy-C₁-C₆-alkylcarbonyl, phenylsulphonyl, phenyl, heteroaryl, hetero-C₃-C₆-cyclyl, phenyl-C₁-C₆-alkyl, heteroaryl-C₁-C₆-alkyl, hetero-C₃-C₆-cyclyl-C₁-C₆-alkyl, phenylcarbonyl, heteroarylcarbonyl, hetero-C₃-C₆-cyclylcarbonyl, phenyl-C₁-C₆-alkylcarbonyl, heteroaryl-C₁-C₆-alkylcarbonyl, hetero-C₃-C₆-cyclyl-C₁-C₆-alkylcarbonyl, phenoxycarbonyl or phenyl-C₁-C₆-alkoxycarbonyl;
where a phenyl or heteroaryl radical is optionally mono- or polysubstituted by halogen, C₁-C₆-alkyl, C₁-C₆-haloalkyl, C₁-C₆-alkoxy, nitro or cyano; a heterocyclyl radical is optionally substituted by —OH/═O, —SH/═S, —NH₂, halogen, C₁-C₆-alkyl, C₁-C₆-haloalkyl, C₁-C₆-alkoxy, C₁-C₆-alkylsulphanyl, C₁-C₆-alkylsulphinyl, C₁-C₆-alkylsulphonyl, C₁-C₆-monoalkylamino, C₁-C₆-dialkylamino, nitro or cyano; and a phenyl-, heteroaryl- or heterocyclyl-bound alkanediyl radical is optionally mono- or polysubstituted by halogen, C₁-C₆-alkyl, C₁-C₆-haloalkyl, C₁-C₆-alkoxy or cyano;
   or
   R¹³ and R¹⁴ as imine form the grouping ═CR⁵—NR¹⁵R¹⁶ or ═CR^{S}—OR¹²;
   R¹⁵ and R¹⁶ independently of one another represent alkyl or
R¹⁵ and R¹⁶ together represent alkanediyl or alkenediyl, each of which has 2 to 5 carbon atoms and is optionally substituted and/or optionally interrupted at the beginning (or the end) or within the hydrocarbon chain by oxygen or sulphur or a grouping from the group consisting of —S(O)—, —SO₂—, —NH— or —N-alkyl-;
R¹³ and R¹⁴ particularly preferably and independently of one another represent hydrogen, C₁-C₄-alkyl, C₁-C₄-haloalkyl, C₂-C₄-alkenyl, C₂-C₄-alkynyl, C₁-C₄-alkoxy-C₁-C₄-alkyl, C₁-C₄-alkylcarbonyl-C₁-C₄-alkyl, C(R¹⁰R¹¹)CR⁵═NO—C₁-C₄-alkyl, C₁-C₄-alkylsulphonyl, C₁-C₄-alkylcarbonyl, C₁-C₄-haloalkylcarbonyl, C₂-C₄-alkenylcarbonyl, C₂-C₄-alkynylcarbonyl, C₃-C₄-cycloalkylcarbonyl, C₁-C₄-alkoxycarbonyl, C₂-C₄-alkenyloxycarbonyl, C₂-C₄-alkynyloxycarbonyl, C₁-C₄-alkoxy-C₁-C₄-alkylcarbonyl, phenylsulphonyl, phenyl, heteroaryl, hetero-C₃-C₄-cyclyl, phenyl-C₁-C₄-alkyl, heteroaryl-C₁-C₄-alkyl, hetero-C₁-C₄-cyclyl-C₁-C₄-alkyl, phenylcarbonyl, heteroarylcarbonyl, hetero-C₃-C₄-cyclylcarbonyl, phenyl-C₁-C₄-alkylcarbonyl, heteroaryl-C₁-C₄-alkylcarbonyl, hetero-C₃-C₄-cyclyl-C₁-C₄-alkylcarbonyl, phenoxycarbonyl or phenyl-C₁-C₄-alkoxycarbonyl;
where a phenyl or heteroaryl radical is optionally mono- or polysubstituted by halogen, C₁-C₄-alkyl, C₁-C₄-haloalkyl, C₁-C₄-alkoxy, nitro or cyano; a heterocyclyl radical is optionally substituted by —OH/═O, —SH/═S, —NH₂, halogen, C₁-C₄-alkyl, C₁-C₄-haloalkyl, C₁-C₄-alkoxy, C₁-C₄-alkylsulphanyl, C₁-C₄-alkylsulphinyl, C₁-C₄-alkylsulphonyl, C₁-C₄-monoalkylamino, C₁-C₄-dialkylamino, nitro or cyano; and a phenyl-, heteroaryl- or heterocyclyl-bound alkanediyl radical is optionally mono- or polysubstituted by halogen, C₁-C₄-alkyl, C₁-C₄-haloalkyl, C₁-C₄-alkoxy or cyano;
   or
   R¹³ and R¹⁴ as imine form the grouping ═CR⁵—NR¹⁵R¹⁶ or ═CR⁵—OR¹²;
   R¹⁵ and R¹⁶ independently of one another represent alkyl or
R¹⁵ and R¹⁶ together represent alkanediyl or alkenediyl, each of which has 2 to 5 carbon atoms and is optionally substituted and/or optionally interrupted at the beginning (or the end) or within the hydrocarbon chain by oxygen or sulphur or a grouping from the group consisting of —S(O)—, —SO₂—, —NH— or —N-alkyl-;

R¹³ and R¹⁴ very particularly preferably and independently of one another represent hydrogen, C₁-C₂-alkyl, C₁-C₂-haloalkyl, C₁-C₂-alkoxy-C₁-C₂-alkyl, C₁-C₂-alkylsulphonyl, C₁-C₂-alkylcarbonyl, C₁-C₂-haloalkylcarbonyl, C₁-C₂-alkoxycarbonyl, C₁-C₂-alkoxy-C₁-C₂-alkylcarbonyl, phenylsulfonyl, phenyl, heteroaryl, phenyl-C₁-C₂-alkyl, heteroaryl-C₁-C₂-alkyl, phenylcarbonyl, heteroarylcarbonyl, phenyl-C₁-C₂-alkylcarbonyl, phenoxycarbonyl or phenyl-C₁-C₂-alkoxycarbonyl;
where a phenyl or heteroaryl radical is optionally mono- or polysubstituted by halogen, C₁-C₂-alkyl, C₁-C₂-haloalkyl, C₁-C₂-alkoxy, nitro or cyano; and a phenyl- or heteroaryl-bound alkanediyl radical is optionally mono- or polysubstituted by halogen, C₁-C₂-alkyl, C₁-C₂-haloalkyl, C₁-C₂-alkoxy or cyano
   or
   R¹³ and R¹⁴ as imine form the grouping ═CR⁵—NR¹⁵R¹⁶ or ═CR⁵—OR¹²;
   R¹⁵ and R¹⁶ independently of one another represent alkyl or
R¹⁵ and R¹⁶ together represent alkanediyl or alkenediyl, each of which has 2 to 5 carbon atoms and is optionally substituted and/or optionally interrupted at the beginning (or the end) or within the hydrocarbon chain by oxygen or sulphur or a grouping from the group consisting of —S(O)—, —SO₂—, —NH— or —N-alkyl-;
R¹³ and R¹⁴ especially preferably and independently of one another represent hydrogen, methyl, ethyl, propyl, isopropyl, methylcarbonyl, methoxycarbonyl, trifluoromethylcarbonyl, methylsulphanyl, trifluoromethylsulphanyl, benzyl or pyridylmethyl.
   Depending on their substituent R¹, the compounds of the formula (I) can be divided into the compounds of the formula (II), (III), (IV) and (V):

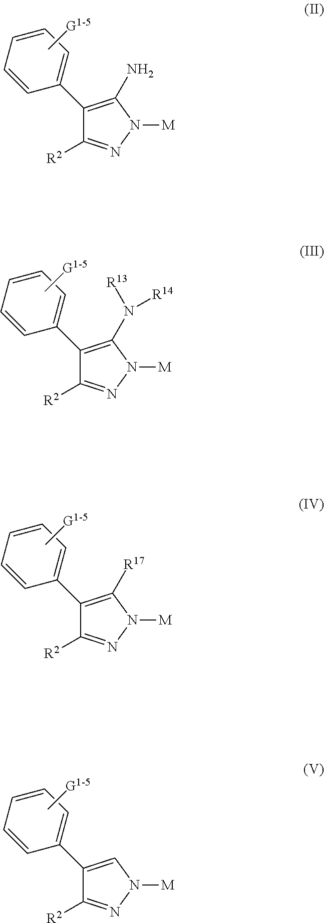

where
G¹⁻⁵, R², R¹³, R¹⁴, and M have the meanings given above, but R¹³ and R¹⁴ do not simultaneously represent hydrogen and
   R¹⁷ represents halogen, alkylsulphonyl, alkylsulphinyl, alkylsulphonyl or cyano.
   This results in 4-phenyl-1H-pyrazoles of the formulae (II-A), (II-B), (III-A), (III-B), (IV-A), (IV-B), (V-A) and (V-B),

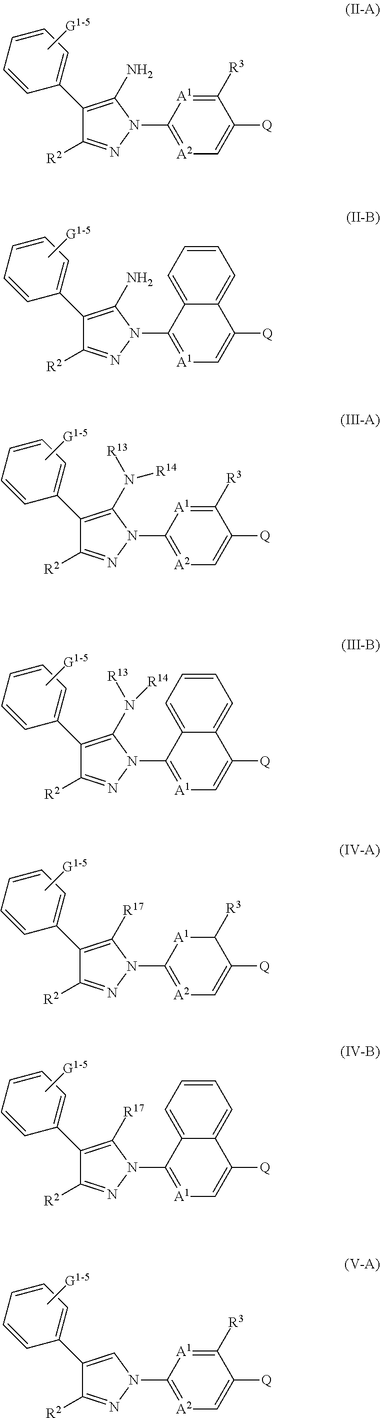

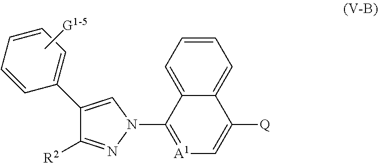

where
G¹⁻⁵, R², R³, R¹³, R¹⁴, A¹⁻² and Q have the meanings given above, but R¹³ and R¹⁴ do not simultaneously represent hydrogen and
   R¹⁷ represents halogen, alkylsulphanyl, alkylsulphinyl, alkylsulphonyl or cyano.
   In an embodiment 1, the invention relates to compounds of the formula (II-A)

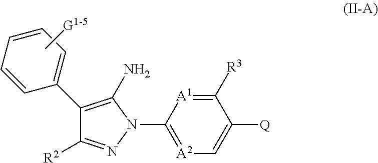

where
G¹, G² and G³ independently of one another represent hydrogen, halogen, methyl or CF₃;
G⁴ and G⁵ represent hydrogen;
R² represents fluorine, chlorine, bromine, iodine, C₁-C₄-alkoxy-C₁-C₄-alkyl, C₁₋₄-alkyl, aryl-C₁-C₄-haloalkyl, C₁-C₆-alkylsulphanyl, C₁-C₆-alkylsulphinyl, C₁-C₆-alkylsulphonyl or C₁₋₄-haloalkyl;
   A¹ and A² independently of one another represent nitrogen or CH;
R³ represents fluorine, chlorine, bromine, iodine, C₁₋₂-alkyl, C₁₋₂-haloalkyl, C₁-C₂-haloalkylsulphanyl, C₁-C₂-haloalkylsulphonyl, C₁-C₂-haloalkylsulphinyl or cyano and
   Q represents Q¹, Q², Q³ or Q⁴.

In an embodiment 2, the invention relates to compounds according to embodiment 1 in which Q represents Q¹.

In an embodiment 3, the invention relates to compounds according to embodiment 2 in which Q¹ represents Z³, Z⁷, Z¹⁵, Z¹⁶, Z¹⁷, Z¹⁸, Z²¹, Z²², Z²³ or Z²⁴; R⁶, R^{6′}, R^{6″}, R^{6′″} represent hydrogen, amino, cyano, fluorine, chlorine, methyl, ethyl, C₁-C₂-haloalkyl, methoxy, ethoxy or C₁-C₂-haloalkoxy; and R⁷ represents hydrogen, amino, cyano, C₁-C₂-alkyl, C₁-C₂-haloalkyl or C₁-C₂-alkoxy.

In an embodiment 4, the invention relates to compounds according to embodiment 3 in which Q¹ represents Z¹⁶ or Z³.

In an embodiment 5, the invention relates to compounds according to embodiment 1 in which Q represents Q².

In an embodiment 6, the invention relates to compounds according to embodiment 5 in which Q² represents C(O)NR⁸R⁹ and R⁸ and R⁹ independently of one another represent hydrogen, C₁-C₄-alkyl, C₁-C₄-haloalkyl, C₃-C₅-cycloalkyl (optionally mono- or polysubstituted at the cycle by halogen, C₁-C₂-haloalkyl, C₁-C₂-alkyl or condensed to an aromatic or heteroaromatic moiety), C₃-C₅-cycloalkyl-C₁-C₂-alkyl (optionally mono- or polysubstituted at the cycle by halogen, C₁-C₂-haloalkyl, C₁-C₂-alkyl or condensed to an aromatic or heteroaromatic moiety, optionally mono- or polysubstituted at the C₁-C₂-alkyl moiety by halogen, C₁-C₂-alkyl, C₁-C₂-haloalkyl, C₁-C₂-alkoxy or cyano), C₁-C₂-alkylsulphinyl-C₁-C₃-alkyl, C₁-C₃-alkylsulphanyl-C₁-C₃-alkyl, C₁-C₂-alkylsulphonyl-C₁-C₃-alkyl, phenyl-C₁-C₄-alkyl or heteroaryl-C₁-C₄-alkyl; where a phenyl or heteroaryl radical is optionally mono- or polysubstituted by halogen, C₁-C₂-alkyl, C₁-C₂-haloalkyl, C₁-C₂-alkoxy, nitro or cyano; and an alkanediyl radical attached to phenyl or heteroaryl is optionally mono- or polysubstituted by halogen, C₁-C₄-alkyl, C₁-C₄-haloalkyl, C₁-C₂-alkoxy or cyano.

In an embodiment 7, the invention relates to compounds according to embodiment 6 in which R⁸ and R⁹ independently of one another represent hydrogen, C₁-C₄-alkyl, C₁-C₄-haloalkyl, C₃-C₄-cycloalkyl, C₃-C₄-halocycloalkyl, C₃-C₅-cycloalkyl-C₁-C₂-alkyl, C₃-C₅-halocycloalkyl-C₁-C₂-alkyl, methylsulphinyl-C₁-C₃-alkyl, methylsulphanyl-C₁-C₃-alkyl, methylsulphonyl-C₁-C₃-alkyl, phenylmethyl (optionally mono- or polysubstituted at the aromatic moiety by fluorine, chlorine, bromine, methyl, ethyl, trifluoromethyl, methoxy, cyano or nitro, optionally mono- or polysubstituted at the methyl moiety by methyl, ethyl, propyl, isopropyl, methoxy or trifluoromethyl), pyridylmethyl (optionally mono- or polysubstituted at the pyridyl moiety by fluorine, chlorine, bromine, methyl, ethyl, trifluoromethyl, methoxy, cyano or nitro, optionally mono- or polysubstituted at the methyl moiety by methyl, ethyl, propyl, isopropyl, methoxy or trifluoromethyl) or pyrimidylmethyl (optionally mono- or polysubstituted at the pyrimidyl moiety by fluorine, chlorine, bromine, methyl, ethyl, trifluoromethyl, methoxy, cyano or nitro, optionally mono- or polysubstituted at the methyl moiety by methyl, ethyl, propyl, isopropyl, methoxy or trifluoromethyl).

In an embodiment 8, the invention relates to compounds according to embodiment 1 in which Q represents Q³.

In an embodiment 9, the invention relates to compounds according to embodiment 8 in which Q³ represents C(R¹⁰R¹¹)NR⁸R⁹; R¹⁰ and R¹¹ independently of one another represent hydrogen, C₁-C₂-alkyl or C₁-C₂-haloalkyl and R⁸ and R⁹ independently of one another represent hydrogen, C₁-C₄-alkyl, C₁-C₄-haloalkyl, C₃-C₅-cycloalkyl (optionally mono- or polysubstituted at the cycle by halogen, C₁-C₂-haloalkyl, C₁-C₂-alkyl or condensed to an aromatic or heteroaromatic moiety), C₃-C₅-cycloalkyl-C₁-C₂-alkyl (optionally mono- or polysubstituted at the cycle by halogen, C₁-C₂-haloalkyl, C₁-C₂-alkyl or condensed to an aromatic or heteroaromatic moiety, optionally mono- or polysubstituted at the C₁-C₂-alkyl moiety by halogen, C₁-C₂-alkyl, C₁-C₂-haloalkyl, C₁-C₂-alkoxy or cyano), C₁-C₂-alkylsulphinyl-C₁-C₃-alkyl, C₁-C₂-alkylsulfanyl-C₁-C₃-alkyl, C₁-C₂-alkylsulfonyl-C₁-C₃-alkyl, phenyl-C₁-C₄-alkyl, heteroaryl-C₁-C₄-alkyl, C₁-C₄-dialkylaminocarbonyl, C₁-C₄-alkylaminocarbonyl, alkylaminocarbonyl, C₁-C₂-alkylsulfonyl, C₁-C₂-alkylcarbonyl, C₁-C₂-haloalkylcarbonyl, C₃-C₅-cycloalkylcarbonyl, C₁-C₂-alkoxycarbonyl, C₁-C₂-alkoxy-C₁-C₂-alkylcarbonyl, phenylsulfonyl, phenyl, heteroaryl, hetero-C₃-C₅-cyclyl, phenylcarbonyl, heteroarylcarbonyl, hetero-C₃-C₅-cyclylcarbonyl, phenyl-C₁-C₂-alkylcarbonyl, heteroaryl-C₁-C₂-alkylcarbonyl, hetero-C₃-C₅-cyclyl-C₁-C₂-alkylcarbonyl, hhenoxycarbonyl or phenyl-C₁-C₂-alkoxycarbonyl; where a phenyl or heteroaryl radical is optionally mono- or polysubstituted by halogen, C₁-C₂-alkyl, C₁-C₂-haloalkyl, C₁-C₂-alkoxy, nitro or cyano; and a phenyl- or heteroaryl-bound alkanediyl radical is optionally mono- or polysubstituted by halogen, C₁-C₄-alkyl, C₁-C₄-haloalkyl, C₁-C₂-alkoxy or cyano.

In an embodiment 10, the invention relates to compounds according to embodiment 9 in which R⁸ and R⁹ independently of one another represent hydrogen, C₁-C₂-alkyl, C₁-C₂-haloalkyl, C₃-C₅-cycloalkyl, C₃-C₅-cycloalkyl-C₁-C₂-alkyl, C₃-C₅-halocycloalkyl, C₃-C₅-halocycloalkyl-C₁-C₂-alkyl, methylsulphinyl-C₁-C₃-alkyl, methylsulphanyl-C₁-C₃-alkyl, methylsulphonyl-C₁-C₃-alkyl, phenyl-C₁-C₂-alkyl, heteroaryl-C₁-C₂-alkyl, C₁-C₂-dialkylaminocarbonyl, C₁-C₂-alkylaminocarbonyl, C₁-C₂-haloalkylaminocarbonyl, C₁-C₂-alkylsulphonyl, C₁-C₂-alkylcarbonyl, C₁-C₂-haloalkylcarbonyl, C₃-C₅-cycloalkylcarbonyl, C₁-C₂-alkoxycarbonyl, phenylsulphonyl, phenylcarbonyl, heteroarylcarbonyl, hetero-C₃-C₅-cyclylcarbonyl, phenyl-C₁-C₂-alkylcarbonyl, heteroaryl-C₁-C₂-alkylcarbonyl, hetero-C₃-C₅-cyclyl-C₁-C₂-alkylcarbonyl or phenoxycarbonyl; where a phenyl or heteroaryl radical is optionally mono- or polysubstituted by halogen, C₁-C₂-alkyl, C₁-C₂-haloalkyl, C₁-C₂-alkoxy, nitro or cyano; and a phenyl- or heteroaryl-bound alkanediyl radical is optionally mono- or polysubstituted by halogen, C₁-C₄-alkyl, C₁-C₄ haloalkyl, C₁-C₂-alkoxy or cyano.

In an embodiment 11, the invention relates to compounds according to embodiment 1 in which Q represents Q⁴.

In an embodiment 12, the invention relates to compounds according to embodiment 11 in which Q⁴ represents cyano (where R¹ does not represent amino), nitro, amino, COOH, COOR¹², fluorine (if R³ is different from chlorine), chlorine (if R³ is different from chlorine, COOH, CH₂CH₂OMe and OMe), bromine, iodine, SR¹² (where R¹ does not represent amino if R¹² represents alkyl), S(O)R¹² or S(O)₂R¹² and R¹² represents C₁-C₄-alkyl or C₁-C₄-haloalkyl.

In an embodiment 13, the invention relates to compounds according to embodiment 12 in which Q⁴ represents cyano (where R¹ does not represent amino), COOH, COOMe, COOEt, fluorine (if R³ is different from chlorine), bromine, iodine, SR¹² (where R¹ does not represent amino if R¹² represents alkyl), S(O)R¹² or S(O)₂R¹².

In an embodiment 14, the invention relates to compounds of the formula (III-A)

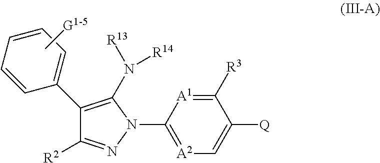

where
G¹, G² and G³ independently of one another represent hydrogen, halogen, methyl or CF₃;
G⁴ and G⁵ represent hydrogen;
R² represents fluorine, chlorine, bromine, iodine, C₁-C₄-alkoxy-C₁-C₄-alkyl, C₁₋₄-alkyl, aryl-C₁-C₄-haloalkyl, C₁-C₆-alkylsulphanyl, C₁-C₆-alkylsulphinyl, C₁-C₆-alkylsulphonyl or C₁₋₄-haloalkyl;
   A¹ and A² independently of one another represent nitrogen or CH;
R³ represents fluorine, chlorine, bromine, iodine, C₁₋₂-alkyl, C₁₋₂-haloalkyl, C₁-C₂-haloalkylsulphanyl, C₁-C₂-haloalkylsulphonyl, C₁-C₂-haloalkylsulphinyl or cyano;
R¹³ and R¹⁴ independently of one another represent hydrogen, methyl, ethyl, propyl, isopropyl, methylcarbonyl, methoxycarbonyl, trifluoromethylcarbonyl, methylsulphanyl, trifluoromethylsulphanyl, benzyl or pyridylmethyl, but R¹³ and R¹⁴ do not simultaneously represent hydrogen, and
   Q represents Q¹, Q², Q³ or Q⁴.

In an embodiment 15, the invention relates to compounds according to embodiment 14 in which Q represents Q¹.

In an embodiment 16, the invention relates to compounds according to embodiment 15 in which Q¹ represents Z³, Z⁷, Z¹⁵, Z¹⁶, Z¹⁷, Z¹⁸, Z²¹, Z²², Z²³ or R^{6′}, R^{6″}, R^{6′″} represent hydrogen, amino, cyano, fluorine, chlorine, methyl, ethyl, C₁-C₂-haloalkyl, methoxy, ethoxy or C₁-C₂-haloalkoxy; and R⁷ represents hydrogen, amino, cyano, C₁-C₂-alkyl, C₁-C₂-haloalkyl or C₁-C₂-alkoxy.

In an embodiment 17, the invention relates to compounds according to embodiment 16 in which Q¹ represents Z¹⁶ or Z³.

In an embodiment 18, the invention relates to compounds according to embodiment 14 in which Q represents Q².

In an embodiment 19, the invention relates to compounds according to embodiment 18 in which Q² represents C(O)NR⁸R⁹ and R⁸ and R⁹ independently of one another represent hydrogen, C₁-C₄-alkyl, C₁-C₄-haloalkyl, C₃-C₅-cycloalkyl (optionally mono- or polysubstituted at the cycle by halogen, C₁-C₂-haloalkyl, C₁-C₂-alkyl or condensed to an aromatic or heteroaromatic moiety), C₃-C₅-cycloalkyl-C₁-C₂-alkyl (optionally mono- or polysubstituted at the cycle by halogen, C₁-C₂-haloalkyl, C₁-C₂-alkyl or condensed to an aromatic or heteroaromatic moiety, optionally mono- or polysubstituted at the C₁-C₂-alkyl moiety by halogen, C₁-C₂-alkyl, C₁-C₂-haloalkyl, C₁-C₂-alkoxy or cyano), C₁-C₂-alkylsulphinyl-C₁-C₃-alkyl, C₁-C₂-alkylsulphanyl-C₁-C₃-alkyl, C₁-C₂-alkylsulphonyl-C₁-C₃-alkyl, phenyl-C₁-C₄-alkyl or heteroaryl-C₁-C₄-alkyl; where a phenyl or heteroaryl radical is optionally mono- or polysubstituted by halogen, C₁-C₂-alkyl, C₁-C₂-haloalkyl, C₁-C₂-alkoxy, nitro or cyano; and an alkanediyl radical attached to phenyl or heteroaryl is optionally mono- or polysubstituted by halogen, C₁-C₄-alkyl, C₁-C₄-haloalkyl, C₁-C₂-alkoxy or cyano.

In an embodiment 20, the invention relates to compounds according to embodiment 19 in which R⁸ and R⁹ independently of one another represent hydrogen, C₁-C₄-alkyl, C₁-C₄-haloalkyl, C₃-C₄-cycloalkyl, C₃-C₄-halocycloalkyl, C₃-C₅-cycloalkyl-C₁-C₂-alkyl, C₃-C₅-halocycloalkyl-C₁-C₂-alkyl, methylalkylsulphinyl-C₁-C₃-alkyl, methylalkylsulfanyl-C₁-C₃-alkyl, methylalkylsulphonyl-C₁-C₃-alkyl, phenylmethyl (optionally mono- or polysubstituted at the aromatic moiety by fluorine, chlorine, bromine, methyl, ethyl, trifluoromethyl, methoxy, cyano or nitro, optionally mono- or polysubstituted at the methyl moiety by methyl, ethyl, propyl, isopropyl, methoxy or trifluoromethyl), pyridylmethyl (optionally mono- or polysubstituted at the pyridyl moiety by fluorine, chlorine, bromine, methyl, ethyl, trifluoromethyl, methoxy, cyano or nitro, optionally mono- or polysubstituted at the methyl moiety by methyl, ethyl, propyl, isopropyl, methoxy or trifluoromethyl) or pyrimidylmethyl (optionally mono- or polysubstituted at the pyrimidyl moiety by fluorine, chlorine, bromine, methyl, ethyl, trifluoromethyl, methoxy, cyano or nitro, optionally mono- or polysubstituted at the methyl moiety by methyl, ethyl, propyl, isopropyl, methoxy or trifluoromethyl).

In an embodiment 21, the invention relates to compounds according to embodiment 14 in which Q represents Q³.

In an embodiment 22, the invention relates to compounds according to embodiment 21 in which Q³ represents C(R¹⁰R¹¹)NR⁸R⁹; R¹⁰ and R¹¹ independently of one another represent hydrogen, C₁-C₂-alkyl or C₁-C₂-haloalkyl and R⁸ and R⁹ independently of one another represent hydrogen, C₁-C₄-alkyl, C₁-C₄-haloalkyl, C₃-C₅-cycloalkyl (optionally mono- or polysubstituted at the cycle by halogen, C₁-C₂-haloalkyl, C₁-C₂-alkyl or condensed to an aromatic or heteroaromatic moiety), C₃-C₄-cycloalkyl-C₁-C₂-alkyl (optionally mono- or polysubstituted at the cycle by halogen, C₁-C₂-haloalkyl, C₁-C₂-alkyl or condensed to an aromatic or heteroaromatic moiety, optionally mono- or polysubstituted at the C₁-C₂-alkyl moiety by halogen, C₁-C₂-alkyl, C₁-C₂-haloalkyl, C₁-C₂-alkoxy or cyano), C₁-C₂-alkylsulfinyl-C₁-C₂-alkyl, C₁-C₂-alkylsulfanyl-C₁-C₂-alkyl, C₁-C₂-alkylsulfonyl-C₁-C₂-alkyl, phenyl-C₁-C₄-alkyl, heteroaryl-C₁-C₄-alkyl, C₁-C₄-dialkylaminocarbonyl, C₁-C₄-alkylaminocarbonyl, C₁-C₄-haloalkylaminocarbonyl, C₁-C₂-alkylsulfonyl, C₁-C₂-alkylcarbonyl, C₁-C₂-haloalkylcarbonyl, C₃-C₅-cycloalkylcarbonyl, C₁-C₂-alkoxycarbonyl, C₁-C₂-alkoxy-C₁-C₂-alkylcarbonyl, phenylsulfonyl, phenyl, heteroaryl, hetero-C₃-C₅-cyclyl, phenylcarbonyl, heteroarylcarbonyl, hetero-C₃-C₅-cyclylcarbonyl, phenyl-C₁-C₂-alkylcarbonyl, heteroaryl-C₁-C₂-alkylcarbonyl, hetero-C₃-C₅-cyclyl-C₁-C₂-alkylcarbonyl, phenoxycarbonyl or phenyl-C₁-C₂-alkoxycarbonyl; where a phenyl or heteroaryl radical is optionally mono- or polysubstituted by halogen, C₁-C₂-alkyl, C₁-C₂-haloalkyl, C₁-C₂-alkoxy, nitro or cyano; and a phenyl- or heteroaryl-bound alkanediyl radical is optionally mono- or polysubstituted by halogen, C₁-C₄-alkyl, C₁-C₄-haloalkyl, C₁-C₂-alkoxy or cyano.

In an embodiment 23, the invention relates to compounds according to embodiment 22 in which R⁸ and R⁹ independently of one another represent hydrogen, C₁-C₂-alkyl, C₁-C₂-haloalkyl, C₃-C₅-cycloalkyl, C₃-C₅-cycloalkyl-C₁-C₂-alkyl, C₃-C₅-halocycloalkyl, C₃-C₅-halocycloalkyl-C₁-C₂-alkyl, methylsulphinyl-C₁-C₃-alkyl, methylsulphanyl-C₁-C₃-alkyl, methylsulphonyl-C₁-C₃-alkyl, phenyl-C₁-C₂-alkyl, heteroaryl-C₁-C₂-alkyl, C₁-C₂-dialkylaminocarbonyl, C₁-C₂-alkylaminocarbonyl, C₁-C₂-haloalkylaminocarbonyl, C₁-C₂-alkylsulphonyl, C₁-C₂-alkylcarbonyl, C₁-C₂-haloalkylcarbonyl, C₃-C₅-cycloalkylcarbonyl, C₁-C₂-alkoxycarbonyl, phenylsulphonyl, phenylcarbonyl, heteroarylcarbonyl, hetero-C₃-C₅-cyclylcarbonyl, phenyl-C₁-C₂-alkylcarbonyl, heteroaryl-C₁-C₂-alkylcarbonyl, hetero-C₃-C₅-cyclyl-C₁-C₂-alkylcarbonyl or phenoxycarbonyl; where a phenyl or heteroaryl radical is optionally mono- or polysubstituted by halogen, C₁-C₂-alkyl, C₁-C₂-haloalkyl, C₁-C₂-alkoxy, nitro or cyano; and a phenyl- or heteroaryl-bound alkanediyl radical is optionally mono- or polysubstituted by halogen, C₁-C₄-alkyl, C₁-C₄-haloalkyl, C₁-C₂-alkoxy or cyano. In an embodiment 24, the invention relates to compounds according to embodiment 14 in which Q represents Q⁴.

In an embodiment 25, the invention relates to compounds according to embodiment 24 in which Q⁴ represents cyano (where R¹ does not represent amino), nitro, amino, COOH, COOR¹², fluorine (if R³ is different from chlorine), chlorine (if R³ is different from chlorine, COOH, CH₂CH₂OMe and OMe), bromine, iodine, SR¹² (where R¹ does not represent amino if R¹² represents alkyl), S(O)R¹² or S(O)₂R¹² and R¹² represents C₁-C₄-alkyl or C₁-C₄-haloalkyl.

In an embodiment 26, the invention relates to compounds according to embodiment 25 in which Q⁴ represents cyano (where R¹ does not represent amino), COOH, COOMe, COOEt, fluorine (if R³ is different from chlorine), bromine, iodine, SR¹² (where R¹ does not represent amino if R¹² represents alkyl), S(O)R¹² or S(O)₂R¹².

In an embodiment 27, the invention relates to compounds of the formula (IV-A)

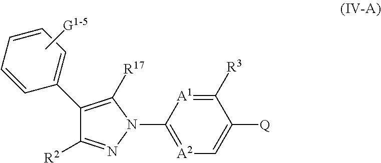

where
G¹, G² and G³ independently of one another represent hydrogen, halogen, methyl or CF₃;
G⁴ and G⁵ represent hydrogen;
R² represents fluorine, chlorine, bromine, iodine, C₁-C₄-alkoxy-C₁-C₄-alkyl, C₁₋₄-alkyl, aryl-C₁-C₄-haloalkyl, C₁-C₆-alkylsulphanyl, C₁-C₆-alkylsulphinyl, C₁-C₆-alkylsulphonyl or C₁₋₄-haloalkyl;
   A¹ and A² independently of one another represent nitrogen or CH;
R³ represents fluorine, chlorine, bromine, iodine, C₁₋₂-alkyl, C₁₋₂-haloalkyl, C₁-C₂-haloalkylsulphanyl, C₁-C₂-haloalkylsulphonyl, C₁-C₂-haloalkylsulphinyl or cyano;
   R¹⁷ represents halogen, alkylsulphanyl, alkylsulphinyl, alkylsulphonyl or cyano and
   Q represents Q¹, Q², Q³ or Q⁴.

In an embodiment 28, the invention relates to compounds according to embodiment 27 in which Q represents Q¹.

In an embodiment 29, the invention relates to compounds according to embodiment 28 in which Q¹ represents Z³, Z⁷, Z¹⁵, Z¹⁶, Z¹⁷, Z¹⁸, Z²¹, Z²², Z²³ or Z²⁴; R⁶, R^{6′}, R^{6″}, R^{6′″} represent hydrogen, amino, cyano, fluorine, chlorine, methyl, ethyl, C₁-C₂-haloalkyl, methoxy, ethoxy or C₁-C₂-haloalkoxy; and R⁷ represents hydrogen, amino, cyano, C₁-C₂-alkyl, C₁-C₂-haloalkyl or C₁-C₂-alkoxy.

In an embodiment 30, the invention relates to compounds according to embodiment 29 in which Q¹ represents Z¹⁶ or Z³.

In an embodiment 31, the invention relates to compounds according to embodiment 27 in which Q represents Q².

In an embodiment 32, the invention relates to compounds according to embodiment 31 in which Q² represents C(O)NR⁸R⁹ and R⁸ and R⁹ independently of one another represent hydrogen, C₁-C₄ alkyl, C₁-C₄-haloalkyl, C₃-C₅-cycloalkyl (optionally mono- or polysubstituted at the cycle by halogen, C₁-C₂-haloalkyl, C₁-C₂-alkyl or condensed to an aromatic or heteroaromatic moiety), C₃-C₅-cycloalkyl-C₁-C₂-alkyl (optionally mono- or polysubstituted at the cycle by halogen, C₁-C₂-haloalkyl, C₁-C₂-alkyl or condensed to an aromatic or heteroaromatic moiety, optionally mono- or polysubstituted at the C₁-C₂-alkyl moiety by halogen, C₁-C₂-alkyl, C₁-C₂-haloalkyl, C₁-C₂-alkoxy or cyano), C₁-C₂-alkylsulphinyl-C₁-C₃-alkyl, C₁-C₂-alkylsulphanyl-C₁-C₃-alkyl, C₁-C₂-alkylsulphonyl-C₁-C₃-alkyl, phenyl-C₁-C₄-alkyl or heteroaryl-C₁-C₄-alkyl; where a phenyl or heteroaryl radical is optionally mono- or polysubstituted by halogen, C₁-C₂-alkyl, C₁-C₂-haloalkyl, C₁-C₂-alkoxy, nitro or cyano; and an alkanediyl radical attached to phenyl or heteroaryl is optionally mono- or polysubstituted by halogen, C₁-C₄-alkyl, C₁-C₄-haloalkyl, C₁-C₂-alkoxy or cyano.

In an embodiment 33, the invention relates to compounds according to embodiment 32 in which R⁸ and R⁹ independently of one another represent hydrogen, C₁-C₄-alkyl, C₁-C₄-haloalkyl, C₃-C₄-cycloalkyl, C₃-C₄-halocycloalkyl, C₃-C₅-cycloalkyl-C₁-C₂-alkyl, C₃-C₅-halocycloalkyl-C₁-C₂-alkyl, methylalkylsulphinyl-C₁-C₃-alkyl, methylalkylsulfanyl-C₁-C₃-alkyl, methylalkylsulphonyl-C₁-C₃-alkyl, phenylmethyl (optionally mono- or polysubstituted at the aromatic moiety by fluorine, chlorine, bromine, methyl, ethyl, trifluoromethyl, methoxy, cyano or nitro, optionally mono- or polysubstituted at the methyl moiety by methyl, ethyl, propyl, isopropyl, methoxy or trifluoromethyl), pyridylmethyl (optionally mono- or polysubstituted at the pyridyl moiety by fluorine, chlorine, bromine, methyl, ethyl, trifluoromethyl, methoxy, cyano or nitro, optionally mono- or polysubstituted at the methyl moiety by methyl, ethyl, propyl, isopropyl, methoxy or trifluoromethyl) or pyrimidylmethyl (optionally mono- or polysubstituted at the pyrimidyl moiety by fluorine, chlorine, bromine, methyl, ethyl, trifluoromethyl, methoxy, cyano or nitro, optionally mono- or polysubstituted at the methyl moiety by methyl, ethyl, propyl, isopropyl, methoxy or trifluoromethyl).

In an embodiment 34, the invention relates to compounds according to embodiment 27 in which Q represents Q³.

In an embodiment 35, the invention relates to compounds according to embodiment 34 in which Q³ represents C(R¹⁰R¹¹)NR⁸R⁹; R¹⁰ and R¹¹ independently of one another represent hydrogen, C₁-C₂-alkyl or C₁-C₂-haloalkyl and R⁸ and R⁹ independently of one another represent hydrogen, C₁-C₄-alkyl, C₁-C₄-haloalkyl, C₃-C₅-cycloalkyl (optionally mono- or polysubstituted at the cycle by halogen, C₁-C₂-haloalkyl, C₁-C₂-alkyl or condensed to an aromatic or heteroaromatic moiety), C₃-C₄-cycloalkyl-C₁-C₂-alkyl (optionally mono- or polysubstituted at the cycle by halogen, C₁-C₂-haloalkyl, C₁-C₂-alkyl or condensed to an aromatic or heteroaromatic moiety, optionally mono- or polysubstituted at the C₁-C₂-alkyl moiety by halogen, C₁-C₂-alkyl, C₁-C₂-haloalkyl, C₁-C₂-alkoxy or cyano), C₁-C₂-alkylsulfinyl-C₁-C₂-alkyl, C₁-C₂-alkylsulfanyl-C₁-C₂-alkyl, C₁-C₂-alkylsulfonyl-C₁-C₂-alkyl, phenyl-C₁-C₄-alkyl, heteroaryl-C₁-C₄-alkyl, C₁-C₄-dialkylaminocarbonyl, C₁-Cᵣ alkylaminocarbonyl, C₁-C₄-haloalkylaminocarbonyl, C₁-C₂-alkylsulfonyl, C₁-C₂-alkylcarbonyl, C₁-C₂-haloalkylcarbonyl, C₃-C₅-cycloalkylcarbonyl, C₁-C₂-alkoxycarbonyl, C₁-C₂-alkoxy-C₁-C₂-alkylcarbonyl, phenylsulfonyl, phenyl, heteroaryl, hetero-C₃-C₅-cyclyl, phenylcarbonyl, heteroarylcarbonyl, hetero-C₃-C₅-cyclylcarbonyl, phenyl-C₁-C₂-alkylcarbonyl, heteroaryl-C₁-C₂-alkylcarbonyl, hetero-C₃-C₅-cyclyl-C₁-C₂-alkylcarbonyl, phenoxycarbonyl or phenyl-C₁-C₂-alkoxycarbonyl; where a phenyl or heteroaryl radical is optionally mono- or polysubstituted by halogen, C₁-C₂-alkyl, C₁-C₂-haloalkyl, C₁-C₂-alkoxy, nitro or cyano; and a phenyl- or heteroaryl-bound alkanediyl radical is optionally mono- or polysubstituted by halogen, C₁-C₄-alkyl, C₁-C₄ haloalkyl, C₁-C₂-alkoxy or cyano.

In an embodiment 36, the invention relates to compounds according to embodiment 35 in which R⁸ and R⁹ independently of one another represent hydrogen, C₁-C₂-alkyl, C₁-C₂-haloalkyl, C₃-C₅-cycloalkyl, C₃-C₅-cycloalkyl-C₁-C₂-alkyl, C₃-C₅-halocycloalkyl, C₃-C₅-halocycloalkyl-C₁-C₂-alkyl, methylsulphinyl-C₁-C₃-alkyl, methylsulphanyl-C₁-C₃-alkyl, methylsulphonyl-C₁-C₃-alkyl, phenyl-C₁-C₂-alkyl, heteroaryl-C₁-C₂-alkyl, C₁-C₂-dialkylaminocarbonyl, C₁-C₂-alkylaminocarbonyl, C₁-C₂-haloalkylaminocarbonyl, C₁-C₂-alkylsulphonyl, C₁-C₂-alkylcarbonyl, C₁-C₂-haloalkylcarbonyl, C₃-C₅-cycloalkylcarbonyl, C₁-C₂-alkoxycarbonyl, phenylsulphonyl, phenylcarbonyl, heteroarylcarbonyl, hetero-C₃-C₅-cyclylcarbonyl, phenyl-C₁-C₂-alkylcarbonyl, heteroaryl-C₁-C₂-alkylcarbonyl, hetero-C₃-C₅-cyclyl-C₁-C₂-alkylcarbonyl or phenoxycarbonyl; where a phenyl or heteroaryl radical is optionally mono- or polysubstituted by halogen, C₁-C₂-alkyl, C₁-C₂-haloalkyl, C₁-C₂-alkoxy, nitro or cyano; and a phenyl- or heteroaryl-bound alkanediyl radical is optionally mono- or polysubstituted by halogen, C₁-C₄-alkyl, C₁-C₄-haloalkyl, C₁-C₂-alkoxy or cyano. In an embodiment 37, the invention relates to compounds according to embodiment 27 in which Q represents Q⁴.

In an embodiment 38, the invention relates to compounds according to embodiment 37 in which Q⁴ represents cyano (where R¹ does not represent amino), nitro, amino, COOH, COOR¹², fluorine (if R³ is different from chlorine), chlorine (if R³ is different from chlorine, COOH, CH₂CH₂OMe and OMe), bromine, iodine, SR¹² (where R¹ does not represent amino if R¹² represents alkyl), S(O)R¹² or S(O)₂R¹² and R¹² represents C₁-C₄-alkyl or C₁-C₄-haloalkyl.

In an embodiment 39, the invention relates to compounds according to embodiment 38 in which Q⁴ represents cyano (where R¹ does not represent amino), COOH, COOMe, COOEt, fluorine (if R³ is different from chlorine), bromine, iodine, SR¹² (where R¹ does not represent amino if R¹² represents alkyl), S(O)R¹² or S(O)₂R¹².

In an embodiment 40, the invention relates to compounds of the formula (V-A)

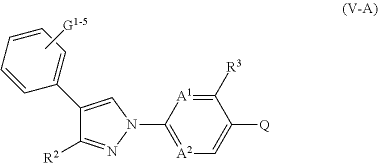

where
G¹, G² and G³ independently of one another represent hydrogen, halogen, methyl or CF₃;
G⁴ and G⁵ represent hydrogen;
R² represents fluorine, chlorine, bromine, iodine, C₁-C₄-alkoxy-C₁-C₄-alkyl, C₁₋₄-alkyl, aryl-C₁-C₄-haloalkyl, C₁-C₆-alkylsulphanyl, C₁-C₆-alkylsulphinyl, C₁-C₆-alkylsulphonyl or C₁₋₄-haloalkyl;
   A¹ and A² independently of one another represent nitrogen or CH;
R³ represents, fluorine, chlorine, bromine, iodine, C₁₋₂-alkyl, C₁₋₂-haloalkyl, C₁-C₄ haloalkylsulphanyl, C₁-C₂-haloalkylsulphonyl, C₁-C₂-haloalkylsulphinyl or cyano and
   Q represents Q¹, Q², Q³ or Q⁴.

In an embodiment 41, the invention relates to compounds according to embodiment 40 in which Q represents Q¹.

In an embodiment 42, the invention relates to compounds according to embodiment 41 in which Q¹ represents Z³, Z⁷, Z¹⁵, Z¹⁶, Z¹⁷, Z¹⁸, Z²¹, Z²², Z²³ or Z²⁴; R^{6′″}, R^{6′}, R^{6″}, R^{6′″} represent hydrogen, amino, cyano, fluorine, chlorine, methyl, ethyl, C₁-C₂-haloalkyl, methoxy, ethoxy or C₁-C₂-haloalkoxy; and R⁷ represents hydrogen, amino, cyano, C₁-C₂-alkyl, C₁-C₂-haloalkyl or C₁-C₂-alkoxy.

In an embodiment 43, the invention relates to compounds according to embodiment 42 in which Q¹ represents Z¹⁶ or Z³.

In an embodiment 44, the invention relates to compounds according to embodiment 40 in which Q represents Q².

In an embodiment 45, the invention relates to compounds according to embodiment 44 in which Q² represents C(O)NR⁸R⁹ and R⁸ and R⁹ independently of one another represent hydrogen, C₁-C₄-alkyl, C₁-C₄-haloalkyl, C₃-C₅-cycloalkyl (optionally mono- or polysubstituted at the cycle by halogen, C₁-C₂-haloalkyl, C₁-C₂-alkyl or condensed to an aromatic or heteroaromatic moiety), C₃-C₅-cycloalkyl-C₁-C₂-alkyl (optionally mono- or polysubstituted at the cycle by halogen, C₁-C₂-haloalkyl, C₁-C₂-alkyl or condensed to an aromatic or heteroaromatic moiety, optionally mono- or polysubstituted at the C₁-C₂-alkyl moiety by halogen, C₁-C₂-alkyl, C₁-C₂-haloalkyl, C₁-C₂-alkoxy or cyano), C₁-C₂-alkylsulphinyl-C₁-C₃-alkyl, C₁-C₃-alkylsulphanyl-C₁-C₃-alkyl, C₁-C₂-alkylsulphonyl-C₁-C₃-alkyl, phenyl-C₁-C₄-alkyl or heteroaryl-C₁-C₄-alkyl; where a phenyl or heteroaryl radical is optionally mono- or polysubstituted by halogen, C₁-C₂-alkyl, C₁-C₂-haloalkyl, C₁-C₂-alkoxy, nitro or cyano; and an alkanediyl radical attached to phenyl or heteroaryl is optionally mono- or polysubstituted by halogen, C₁-C₄-alkyl, C₁-C₄-haloalkyl, C₁-C₂-alkoxy or cyano.

In an embodiment 46, the invention relates to compounds according to embodiment 45 in which R⁸ and R⁹ independently of one another represent hydrogen, C₁-C₄-alkyl, C₁-C₄-haloalkyl, C₃-C₄-cycloalkyl, C₃-C₄-halocycloalkyl, C₃-C₅-cycloalkyl-C₁-C₂-alkyl, C₃-C₅-halocycloalkyl-C₁-C₂-alkyl, methylalkylsulphinyl-C₁-C₃-alkyl, methylalkylsulfanyl-C₁-C₃-alkyl, methylalkylsulphonyl-C₁-C₃-alkyl, phenylmethyl (optionally mono- or polysubstituted at the aromatic moiety by fluorine, chlorine, bromine, methyl, ethyl, trifluoromethyl, methoxy, cyano or nitro, optionally mono- or polysubstituted at the methyl moiety by methyl, ethyl, propyl, isopropyl, methoxy or trifluoromethyl), pyridylmethyl (optionally mono- or polysubstituted at the pyridyl moiety by fluorine, chlorine, bromine, methyl, ethyl, trifluoromethyl, methoxy, cyano or nitro, optionally mono- or polysubstituted at the methyl moiety by methyl, ethyl, propyl, isopropyl, methoxy or trifluoromethyl) or pyrimidylmethyl (optionally mono- or polysubstituted at the pyrimidyl moiety by fluorine, chlorine, bromine, methyl, ethyl, trifluoromethyl, methoxy, cyano or nitro, optionally mono- or polysubstituted at the methyl moiety by methyl, ethyl, propyl, isopropyl, methoxy or trifluoromethyl).

In an embodiment 47, the invention relates to compounds according to embodiment 40 in which Q represents Q³.

In an embodiment 48, the invention relates to compounds according to embodiment 47 in which Q³ represents C(R¹⁰R¹¹)NR⁸R⁹; R¹⁰ and R¹¹ independently of one another represent hydrogen, C₁-C₂-alkyl or C₁-C₂-haloalkyl and R⁸ and R⁹ independently of one another represent hydrogen, C₁-C₄-alkyl, C₁-C₄-haloalkyl, C₃-C₄-cycloalkyl (optionally mono- or polysubstituted at the cycle by halogen, C₁-C₂-haloalkyl, C₁-C₂-alkyl or condensed to an aromatic or heteroaromatic moiety), C₃-C₄-cycloalkyl-C₁-C₂-alkyl (optionally mono- or polysubstituted at the cycle by halogen, C₁-C₂-haloalkyl, C₁-C₂-alkyl or condensed to an aromatic or heteroaromatic moiety, optionally mono- or polysubstituted at the C₁-C₂-alkyl moiety by halogen, C₁-C₂-alkyl, C₁-C₂-haloalkyl, C₁-C₂-alkoxy or cyano), C₁-C₂-alkylsulfinyl-C₁-C₂-alkyl, C₁-C₂-alkylsulfanyl-C₁-C₂-alkyl, C₁-C₂-alkylsulfonyl-C₁-C₂-alkyl, phenyl-C₁-C₄-alkyl, heteroaryl-C₁-C₄-alkyl, C₁-C₄-dialkylaminocarbonyl, C₁-C₄-alkylaminocarbonyl, C₁-C₄-haloalkylaminocarbonyl, C₁-C₂-alkylsulfonyl, C₁-C₂-alkylcarbonyl, C₁-C₂-haloalkylcarbonyl, C₃-C₅-cycloalkylcarbonyl, C₁-C₂-alkoxycarbonyl, C₁-C₂-alkoxy-C₁-C₂-alkylcarbonyl, phenylsulfonyl, phenyl, heteroaryl, hetero-C₃-C₅-cyclyl, phenylcarbonyl, heteroarylcarbonyl, hetero-C₃-C₅-cyclylcarbonyl, phenyl-C₁-C₂-alkylcarbonyl, heteroaryl-C₁-C₂-alkylcarbonyl, hetero-C₃-C₅-cyclyl-C₁-C₂-alkylcarbonyl, phenoxycarbonyl or phenyl-C₁-C₂-alkoxycarbonyl; where a phenyl or heteroaryl radical is optionally mono- or polysubstituted by halogen, C₁-C₂-alkyl, C₁-C₂-haloalkyl, C₁-C₂-alkoxy, nitro or cyano; and a phenyl- or heteroaryl-bound alkanediyl radical is optionally mono- or polysubstituted by halogen, C₁-C₄-alkyl, C₁-C₄-haloalkyl, C₁-C₂-alkoxy or cyano.

In an embodiment 49, the invention relates to compounds according to embodiment 48 in which R⁸ and R⁹ independently of one another represent hydrogen, C₁-C₂-alkyl, C₁-C₂-haloalkyl, C₃-C₅-cycloalkyl, C₃-C₅-cycloalkyl-C₁-C₂-alkyl, C₃-C₅-halocycloalkyl, C₃-C₅-halocycloalkyl-C₁-C₂-alkyl, methylsulphinyl-C₁-C₃-alkyl, methylsulphanyl-C₁-C₃-alkyl, methylsulphonyl-C₁-C₃-alkyl, phenyl-C₁-C₂-alkyl, heteroaryl-C₁-C₂-alkyl, C₁-C₂-dialkylaminocarbonyl, C₁-C₂-alkylaminocarbonyl, C₁-C₂-haloalkylaminocarbonyl, C₁-C₂-alkylsulphonyl, C₁-C₂-alkylcarbonyl, C₁-C₂-haloalkylcarbonyl, C₃-C₅-cycloalkylcarbonyl, C₁-C₂-alkoxycarbonyl, phenylsulphonyl, phenylcarbonyl, heteroarylcarbonyl, hetero-C₃-C₅-cyclylcarbonyl, phenyl-C₁-C₂-alkylcarbonyl, heteroaryl-C₁-C₂-alkylcarbonyl, hetero-C₃-C₅-cyclyl-C₁-C₂-alkylcarbonyl or phenoxycarbonyl; where a phenyl or heteroaryl radical is optionally mono- or polysubstituted by halogen, C₁-C₂-alkyl, C₁-C₂-haloalkyl, C₁-C₂-alkoxy, nitro or cyano; and a phenyl- or heteroaryl-bound alkanediyl radical is optionally mono- or polysubstituted by halogen, C₁-C₄-alkyl, C₁-C₄ haloalkyl, C₁-C₂-alkoxy or cyano.

In an embodiment 50, the invention relates to compounds according to embodiment 40 in which Q represents Q⁴.

In an embodiment 51, the invention relates to compounds according to embodiment 50 in which Q⁴ represents cyano (where R¹ does not represent amino), nitro, amino, COOH, COOR¹², fluorine (if R³ is different from chlorine), chlorine (if R³ is different from chlorine, COOH, CH₂CH₂OMe and OMe), bromine, iodine, SR¹² (where R¹ does not represent amino if R¹² represents alkyl), S(O)R¹² or S(O)₂R¹² and R¹² represents C₁-C₄-alkyl or C₁-C₄-haloalkyl.

In an embodiment 52, the invention relates to compounds according to embodiment 51 in which Q⁴ represents cyano (where R¹ does not represent amino), COOH, COOMe, COOEt, fluorine (if R³ is different from chlorine), bromine, iodine, SR¹² (where R¹ does not represent amino if R¹² represents alkyl), S(O)R¹² or S(O)₂R¹².

The present invention also provides the use of the compounds of the formula (I) for controlling animal pests, where the radicals are as defined above.

The present invention also provides the use of the compounds of the formula (I) for controlling animal pests, where M represents M¹, Q represents Q⁴, Q⁴ represents hydrogen and all other radcials are as defined above, with the proviso that A¹ and A² do not simultaneously represent nitrogen.

The present invention also provides the use of the compounds of the formula (I) for controlling animal pests, where M represents M¹, R³ represent hydrogen and all other radcials are as defined above, with the proviso that A¹ and A² do not simultaneously represent nitrogen.

The compounds according to the invention can be prepared in the manner specified below, or in a manner analogous thereto.

### Process (A)

Compounds of the formula (II) are synthesized, for example, by process (A) below:

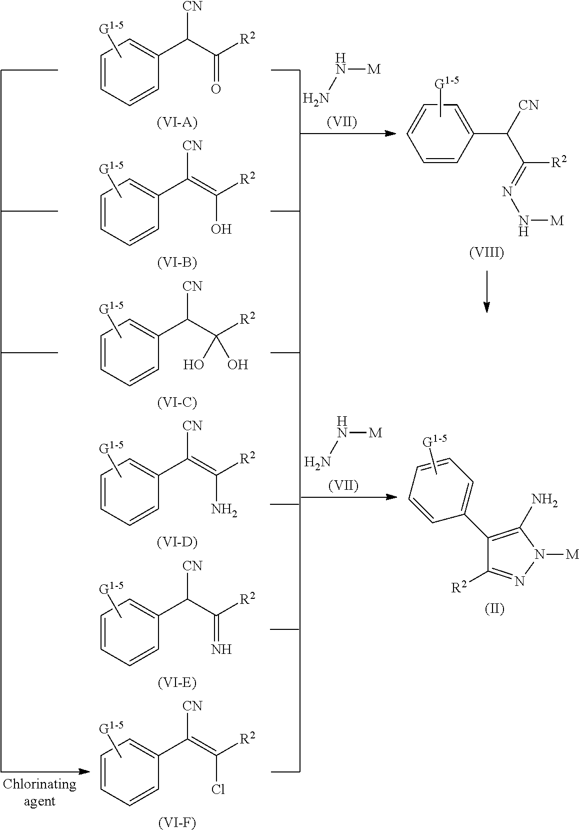

where
G¹⁻⁵, R² and M have the meanings given above.

In process (A), to prepare the compounds of the formula (II), ketonitriles, their tautomers or hydrates of the formulae (VI-A), (VI-B) and (VI-C) or aminoacrylonitriles and their tautomers of the formulae (VI-D) and (IV-E) are condensed with aryl- or heteroarylhydrazines of the formula (VII), where initially hydrazones of the formula (VIII) are formed as intermediate and after prolonged reaction time and elevated temperature ring closure to the aminopyrazole of the formula (II) occurs. Here, it is possible to add acids as catalyst, possible suitable acids being inorganic acids such as hydrochloric acid and organic acids such as sulphonic acids or acetic acid.

Alternatively, ketonitriles, their tautomers or hydrates of the formulae (VI-A), (VI-B) and (VI-C) are initially reacted with a chlorinating agent, for example phosphoryl chloride, phosphorus pentachloride, thionyl chloride, phosgene, chlorine or oxalyl chloride, if appropriate diluted in an inert organic solvent, to give chloroacrylonitriles (VI-F), where the reaction can be carried out in a temperature range of from −20° C. to 120° C. In a subsequent step, the condensation with aryl or heteroarylhydrazines of the formula (VII) is carried out in a suitable organic solvent in the presence of basic auxiliary reagents, for example alkoxides or nitrogen bases, where the reaction can be carried out in the temperature range from −20° C. to 120° C.

Synthesis methods for structurally related aminopyrazoles are known and described, for example, in J. Med. Chem. 2006, 14(6), 1785-1791; Eur. J. Med. Chem. 2005, 40, 922-927; J. Chem. Res. Synopses 1985, 5, 198-199; J. Mol. Cat. A 2006, 258(1-2), 371-375; J. Med. Chem. 2006, 49, 3332-3344; Bioorg. Med. Chem. 2006, 14(6), 1785-1791; Eur. J. Med. Chem. 2005, 40(9), 922-927; Tetrahedron 2004, 60(4), 901-906; J. Heterocyclic Chem. 1975, 12(5), 899-901, Molecular Div. 2003, 7(2-4), 161-164; DE-A-2643640; and U.S. Pat. No. 3,041,342.

The ketonitriles can be present in the tautomeric forms (VI-A) and (VI-B) and as hydrate (VI-C). The starting compounds can also be employed in the form of their salts; the ketonitriles, for example, can be used in the form of their alkali metal salts.

The aminoacrylonitriles can be present in the tautomeric forms (VI-D) and (VI-E). The starting compounds can also be employed in the form of their salts; the aminoacrylonitriles, for example, can be used in the form of their alkali metal salts.

Ketonitriles of the formulae (VI-A, VI-B and VI-C) can be prepared by known methods. Such methods are described in: J. Amer. Chem. Soc. 1950, 72, 5409-5413; Tetrahedron 2007, 63(47), 11626-11635, Org. Lett. 2007, 9(18), 3575-3578, J. Med. Chem. 2007, 50(2), 399-403, Bioorg. Med. Chem. Lett. 2006, 16(3), 695-700.

Acrylonitriles of the formulae (VI-D and VI-E) can be prepared by known methods. Such methods are described in: J. Med. Chem. 2006, 49, 3332-3344.

Chloroacrylonitriles of the formula (VI-F) can be prepared by known methods. Such methods are described in: J. Org Chem (UdSSR) 1981, 1441, JP 08-208620, J. Med. Chem. 2005, 48(22), 6843-6854, Nucleosides, Nucleotides Nucleic Acids 2004, 23(5), 805-812, J. Med. Chem. 2001, 44(3), 350-361.

Some of the aryl-, pyridyl- and pyrimidinylhydrazines of the formula (VII) are commercially available or can be prepared by methods known to the person skilled in the art, such as described, for example, in process (H).

The amidation, acylation and substitution reactions required for the construction of Q¹⁻³ are carried out by methods known to the person skilled in the art as described, for example, in processes (F), (G) and (J) and can be carried out either at the end of the synthesis sequence or, preferably, at the stage of suitable intermediates.

### Process (B)

Compounds of the formula (I) according to the invention are synthesized, for example, by process (B) below:

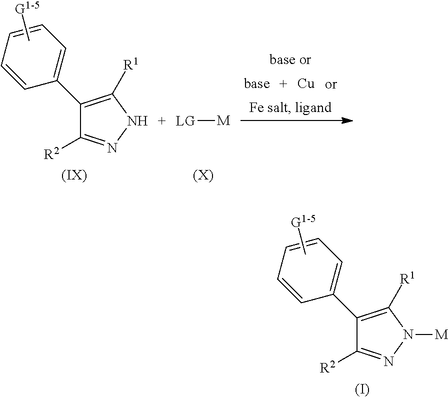

where
LG=halogen, alkylsulphonyl, boronic acid or boronic ester

The intermediates (IX) are prepared by process (A) using hydrazine hydrate:

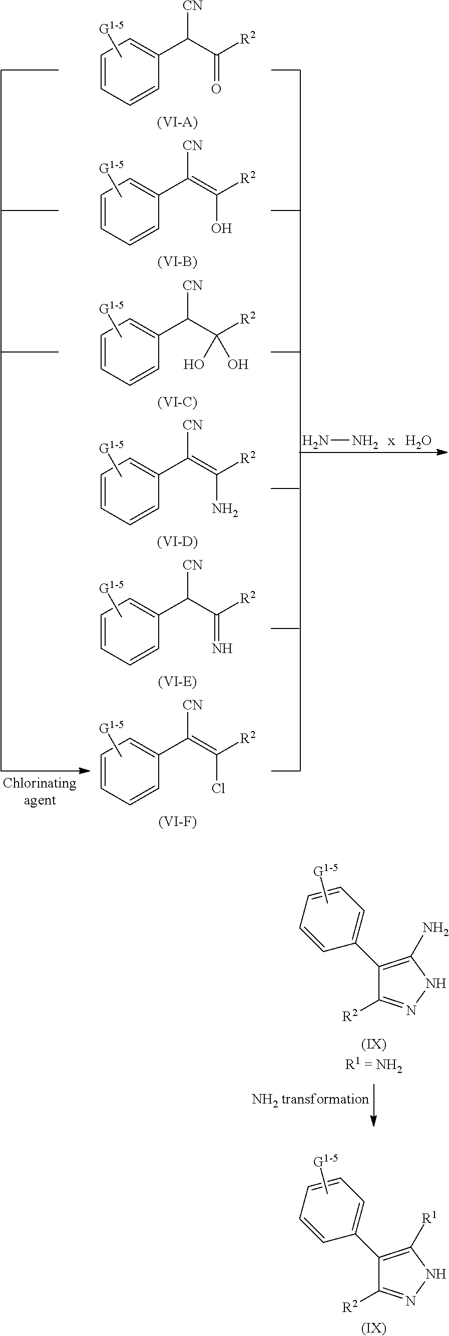

In process (B), 1-H-aminopyrazoles of the formula (IX) are reacted with aryl halides, heteroaryl halides, arylalkylsulphones, heteroarylalkylsulphones, arylboronic acids, heteroarylboronic acids, arylboronic esters or heteroarylboronic esters (LG-M) in the presence of a base and, if appropriate, a copper or iron salt and a ligand in a suitable organic solvent, where preferably a particular isomer, the aminopyrazole of the formula (I), is formed.

The synthesis of structurally related aminopyrazoles of the formula (IX) is generally known and described in: DE-A 2643640, Bioorg. Med. Chem. Lett. 2004, 14, 3669, Bioorg. Med. Chem. 2000, 8(1), 181-190, J. Comb. Chem. 2007, 9(3), 507-512, J. Med. Chem. 2004, 47(24), 5872-5893, EP-A-269859, J. Prakt. Chem. 1979, 321(2), 341-344,

The Cu- and Fe-catalyzed arylation of pyrazoles is known and described in: J. Org. Chem. 2004, 69(17), 5578-5587, Angew. Chem. Int. Ed. 2007, 46, 934-936, Bioorg. Med. Chem. Lett. 2007, 17(15), 4303-4307, IN 178903, Bioorg. Med. Chem. 2007, 17(2), 354-357, J. Med. Chem. 2004, 47(19), 4645-4648.

The synthesis of aminopyrazoles by nucleophilic aromatic and heteroaromatic substitution is known and described in: J. Med. Chem. 2004, 47, 2995-3008, JP 2007-169575, Synth. Commun 2006, 36(11), 1601-1612, Can. J. Chem. 1988, 66(3), 420-428.

The ketonitriles can be present in the tautomeric forms (VI-A) and (VI-B) and as hydrate (VI-C). The starting compounds can also be employed in the form of their salts; the ketonitriles, for example, can be used in the form of their alkali metal salts.

The aminoacrylonitriles can be present in the tautomeric forms (VI-D) and (VI-E). The starting compounds can also be employed in the form of their salts; the aminoacrylonitriles, for example, can be used in the form of their alkali metal salts.

The ketonitriles of the formulae (VI-A, VI-B and VI-C) can be prepared by known methods. J. Amer. Chem. Soc. 1950, 72, 5409-5413; Tetrahedron 2007, 63(47), 11626-11635; Org. Lett. 2007, 9(18), 3575-3578; J. Med. Chem. 2007, 50(2), 399-403; Bioorg. Med. Chem. Lett. 2006, 16(3), 695-700.

The acrylonitriles of the formulae (VI-D and VI-E) can be prepared by known methods: J. Med. Chem. 2006, 49, 3332-3344.

The chloroacrylonitriles of the formula (VI-F) can be prepared by known methods: J. Org. Chem. (UdSSR) 1981, 1441 JP 08208620, J. Med. Chem., 2005, 48(22), 6843-6854, Nucleosides, Nucleotides and Nucleic Acids, 2004, 23(5), 805-812, J. Med. Chem., 2001, 44(3), 350-361

Some of the aryl- and heteroaryl compounds LG-M are commercially available or can be prepared by methods known to the person skilled in the art.

The amidation, acylation and substitution reactions required for the construction of Q¹⁻³ are carried out by methods known to the person skilled in the art as described, for example, in processes (F), (G) and (I) and can be carried out either at the end of the synthesis sequence or, preferably, at the stage of suitable intermediates.

The present invention also relates to compounds of the formula (IX)

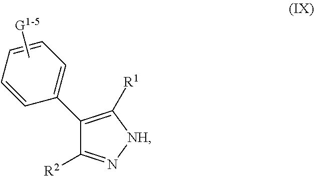

where G¹, G², G³, G⁴, G⁵, R¹ and R² are as defined above, with the proviso, that R¹ does not represent amino.

### Process (C)

Compounds of the formulae (IIIa, IIIb, IIIc and IIId) according to the invention can be prepared by process (C):

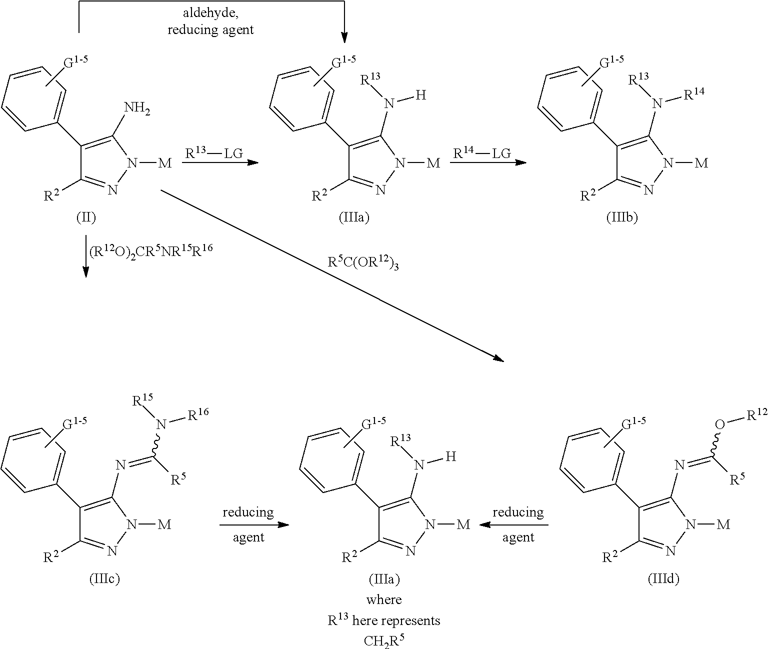

where
G¹⁻⁵, R², R¹³, R¹⁴, and M have the meanings given above, but R¹³ and R¹⁴ do not both simultaneously represent hydrogen;
   LG represents halogen or alkylsulphonyl.

In process (C) according to the invention for preparing compounds of the formulae (IIIa) and (IIIb), compounds of the formula (II) are reacted with one or two alkylating agents, acylating agents or sulphonylating agents R¹³-LG or R¹⁴-LG, where aminopyrazoles of the formulae (IIIa) and (Mb) are formed by monosubstitution and disubstitution, respectively. Suitable alkylating agents are alkyl bromides, alkyl dibromides, alkyl iodides, alkyl diiodides, dialkyl sulphates and alkyl sulphonates. The acylating agents used are carboxylic anhydrides and carbonyl chlorides, the sulphonylating agents used are sulphonyl chlorides. Alternatively, the mono-N-substituted aminopyrazoles of the formula (IIIa) can be obtained by reductive amination from the aminopyrazoles of the formula (II), an aldehyde and a reducing agent, for example hydrogen in the presence of a hydrogenation catalyst, alkali metal borohydrides or borane.

Alternatively, compounds of the formula (IIIa) can be obtained by converting compounds of the formula (II) into amidines of the formula (IIIc) or imidoesters of the formula (IIId), followed by a reduction step.

The alkylation of structurally related aminopyrazoles is described in: WO 2006/000312, WO 2005/09312, WO 2005/023761, WO 2004/099156.

The reductive amination of structurally related aminopyrazoles is described in: J. Med. Chem. 2002, 45(14), 2994-3008, WO 2002/010153, Tetrahedron Lett. 2001, 42(21), 3587-3590.

The acylation of structurally related aminopyrazoles is described in: J. Med. Chem. 2006, 49(11), 3332-3344, J. Med. Chem. 2007, 50(21), 5161-5167, Bioorg. Med. Chem. 2006, 14(22), 7501-7511, Org. Biomol. Chem. 2004, 2(11), 1603-1611.

The sulphonylation of structurally related aminopyrazoles is described in: Acta Poloniae Pharmaceutica 2003, 60(1), 51-60, Biochemistry 2003, 42(21), 6363-6369, J. Med. Chem. 2001, 44(22), 3622-3631.

The synthesis of structurally related imines of the formula (IIIc) is described in: Tetrahedron 2006, 62(24), 5617-5625, Tetrahedron 1987, 53(18), 4185-4193.

The synthesis of structurally related amidines (IIIc) and imidoesters (IIId) and their reduction is described in: WO 2005/060749.

### Process (D)

Compounds of the formulae (III) and (IVa to IVe) according to the invention are synthesized, for example, by process (D):

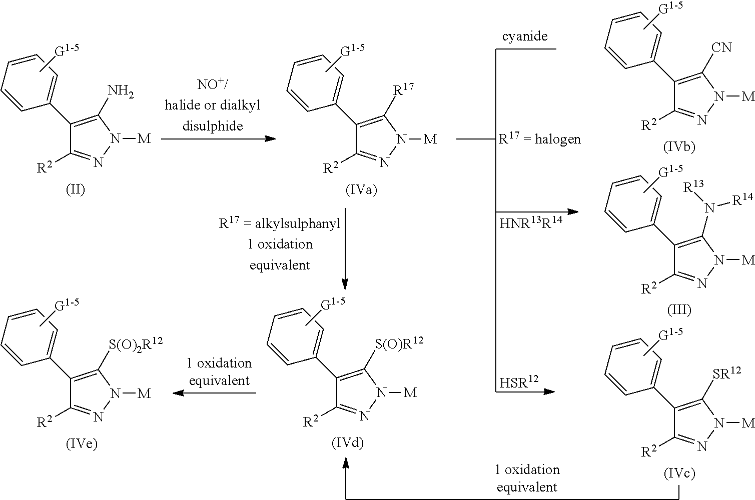

where
G¹⁻⁵, R², R¹², R¹³, R¹⁴ and M have the meanings described above;
R¹⁷ represents halogen or alkylsulphanyl.

In process (D) according to the invention for preparing the compounds of the formulae (III) and (IV), aminopyrazoles of the formula (II) are reacted with nitrosyl species in the presence of suitable halides, where after the formation of a diazo inter mediate a 5-halopyrazole of the formula (IVa, R¹⁷=halogen) is formed. Suitable sources for nitrosyl species are alkali metal nitrites plus acids and also esters of nitrous acid, for example butyl nitrite and tert-butyl nitrite. Suitable for use as halides are metal halides and also organic halides, for example bromoform or iodoform. These 5-halopyrazoles can be converted with cyanides, for example CuCN in suitable solvents, for example NMP with input of heat, into 5-cyanopyrazoles of the formula (IVb) or with amines in suitable solvents into 5-aminopyrazoles of the formula (III), where R¹³ or R¹⁴ does not represent hydrogen. The reaction of the diazo intermediates with alkylsulphanyl sources, for example dialkyl disulphides, leads to 5-alkylsulphanylpyrazoles of the formula (IVa, R¹⁷=alkylsulphanyl). These thioethers can be oxidized in the presence of a suitable oxidizing agent, for example with H₂O₂, sodium periodate, tert-butyl hypochlorite, calcium hypochlorite Ca(OCl)₂, sodium chlorite NaClO₂, sodium hypochlorite NaOCl, peracids or O₂ and catalytic cerium ammonium nitrate to give 5-alkylsulphinylpyrazoles of the formula (IVd) and 5-alkylsulphonylpyrazoles of the formula (IVe).

The synthesis of structurally related 5-halo- and 5-alkylsulphanylpyrazoles is described in WO 2003/074492, J. Heterocycl. Chem. 1987, 24, 267-270, GB 2149402A, J. Med. Chem. 2006, 49, 1562-1575.

The synthesis of structurally related 5-cyanopyrazoles is described in: J. Med. Chem. 2006, 49, 1562-1575

The synthesis of N-substituted aminopyrazoles of the formula (III) is described in: DE 3520327, WO 2005/023776.

The oxidation of thioethers is described in: J. March “Advanced Organic Chemistry” 4. edition, John Wiley & Sons, 1992, p. 1202 and the literature cited therein. Process (E)

Compounds of the formula (I) according to the invention are also synthesized, for example, by process (E) below:

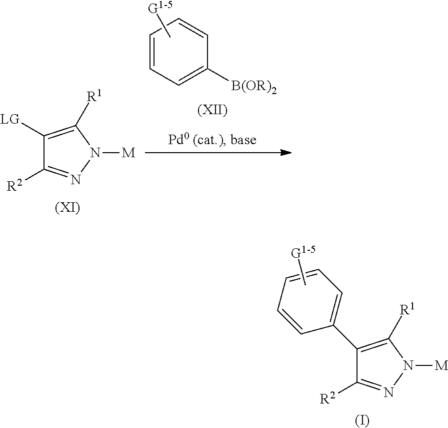

where G¹⁻⁵, R¹, R² and M have the meanings given above and LG represents bromine, iodine or alkylsulphonyl.

In the process (E) according to the invention for preparing the compounds of the formula (I), bromides or iodides of the formula (XI) are reacted with boronic acids or boronic esters of the formula (XII) in the presence of suitable palladium catalysts, ligands and bases (Suzuki reaction) in the temperature range from −20° C. to 120° C. in suitable solvents.

The bromides or iodides of the formula (XI) can be prepared by known methods described, for example in: WO 2005/11292, Chemistry of Heterocyclic Compounds (New York, N.Y., United States), 2005, 41(1), 105-110, Bioorg. & Med. Chem. 2004, 12(12), 3345-3355, Bioorg. Med. Chem. Lett. 2004, 14, 4949, J. Med. Chem. 1977, 20(12), 1562-1569.

Some of the boronic acids or boronic esters of the formula (XII) are commercially available or can be prepared easily by known methods. This is described, for example, in: WO 1999/64428.

The present invention also provides compounds of the formulae (XI-A) and (XI-B)

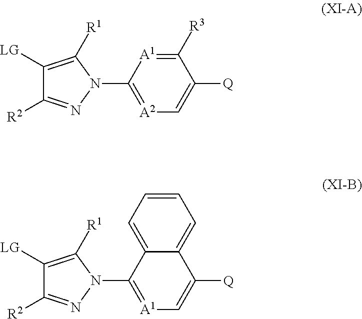

where R¹, R², R³, A′, A² and Q are as defined above and LG represents chlorine, bromine, iodine or alkylsulphonyl.

### Process (F)

Compounds of the formulae (I-A) and (I-B) according to the invention where Q=Q′ are synthesized, for example, by process (F) below:

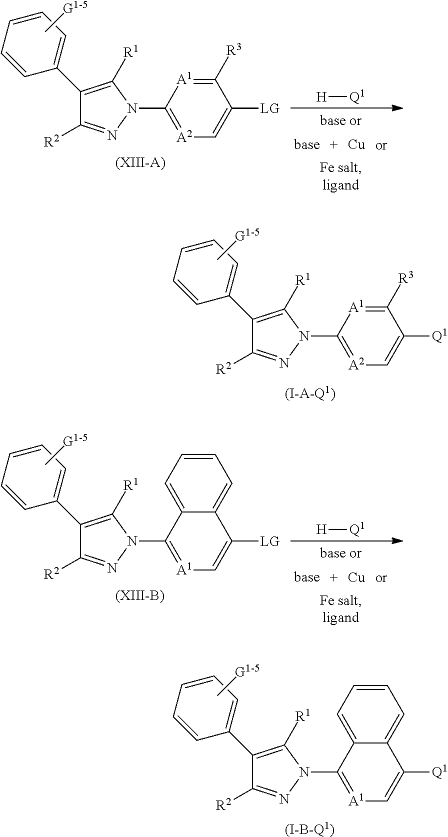

where
G¹⁻⁵, R¹, R², R³, A¹ and A² have the meanings given above and LG=halogen, alkylsulphonyl, boronic acid or boronic ester.

In the process (F) according to the invention for preparing the compounds of the formulae (I-A-Q1) and (I-B-Q1), compounds of the formula (XIII-A) or (XIII-B) are reacted with N-heterocycles in the presence of a base and, if appropriate, a copper or iron salt and a ligand in a suitable organic solvent, with formation of a compound of the formula (I-A-Q1) or (I-B-Q1).

The Cu-catalyzed arylation of heterocycles such as triazoles is described in: J. Org. Chem. 2007, 72(22), 8535, J. Am. Chem. Soc. 2007, 129 (45), 13879, J. Org. Chem. 2007, 72 (8), 2737, Angew. Chem. 2007, 46 (6), 934.

The arylation of heterocycles such as triazoles by nucleophilic substitution is described in: Bioorg. Med. Chem. Lett. 2007, 17(24), 6707, J. Heterocycl. Chem. 2007, 44 (6), 1323, Chem. Pharm. Bull. 2005, 53 (7), 764.

### Process (G)

Compounds of the formulae (I-A) and (I-B) according to the invention where Q=Q² are synthesized, for example, by process (G) below:

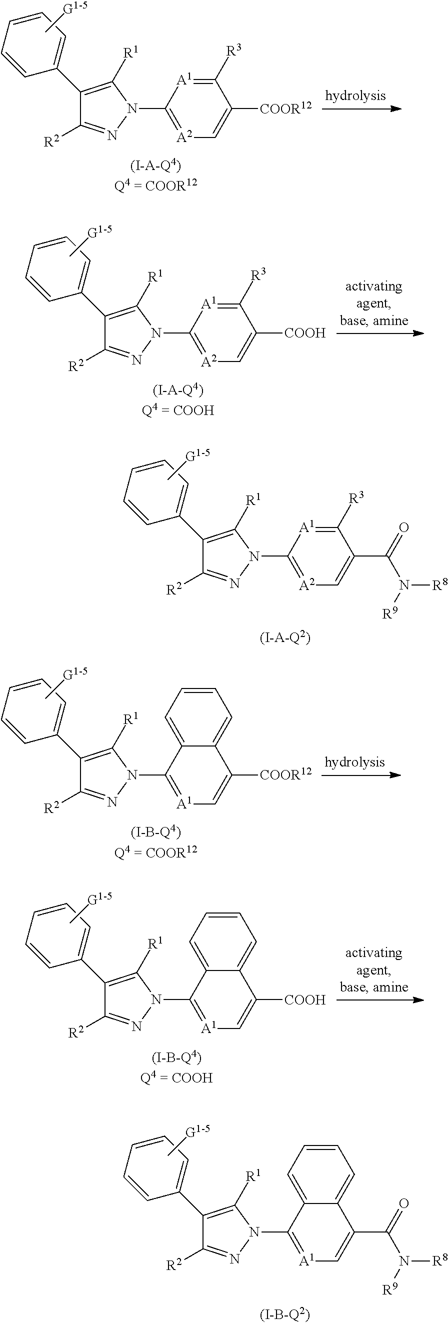

where
G¹⁻⁵, R¹, R², R³, R⁸, R⁹, R¹², A¹ and A² have the meanings given above.

In the process (G) according to the invention for preparing the compounds (I-A-Q2) and (I-B-Q2), compounds of the formula (I-A-Q4) or (I-B-Q4) [Q4=ester] are hydrolyzed by methods known to the person skilled in the art to give (I-A-Q4) or (I-B-Q4) [Q4=COOH] and then converted with the aid of an activating agent with a primary or secondary amine HNR⁸R⁹ into amides of the formula (I-A-Q2) or (I-B-Q2). Activation can be via acid chlorides, mixed anhydrides, pentafluorophenyl esters or with the aid of substituted carbodiimides, for example DCC(N,N′-dicyclohexylcarbodiimide), DIC (diisopropylcarbodiimide) or EDC (N-ethyl-N-(3-dimethylaminopropyl)carbodiimide HCl), and a benzotriazole such as HOBt (1-hydroxybenzotriazole) or azabenzotriazole such as HOAt (7-aza-1-hydroxybenzotriazole).

### Process Routes for the Preparation of Intermediates

### Process (H)

Intermediates of the formula (VII) are synthesized, for example, by process (H) below:

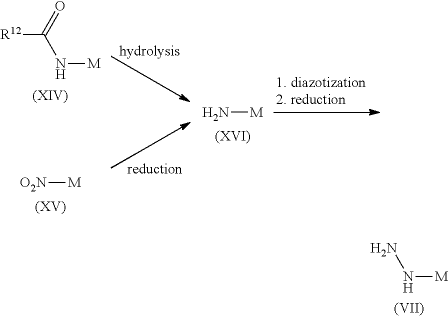

where
R¹² and M have the meanings given above.

In process (H) according to the invention for preparing the hydrazines (VII), the amines (XVI) are converted by methods known to the person skilled in the art, for example in a sequence of diazotization and reduction, into the corresponding hydrazines (cf., for example, J. Med. Chem. 1993, 36 (11) pp. 1529-1538 and WO 2006/081034). Some of the hydrazines of the general formula (VII) are additionally commercially available (for example ethyl 4-hydrazinylbenzenecarboxylate or methyl 4-hydrazinyl-2-methoxybenzenecarboxylate). Some amines of the formula (XVI) are commercially available or can be prepared by methods known to the person skilled in the art, for example by hydrolysis of compounds of the formula (XIV) or reduction of compounds of the formula (XV).

The present invention also provides compounds of the formulae (VII-A) and (VII-B)

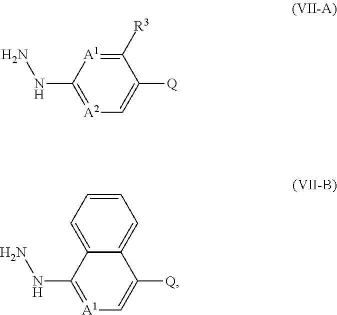

where A¹, A², R³ and Q are as defined above.

### Process (J)

Intermediates of the formulae (X-A)/(X-B) and (XVI-A)/(XVI-B) are synthesized, for example, by process (J) below:

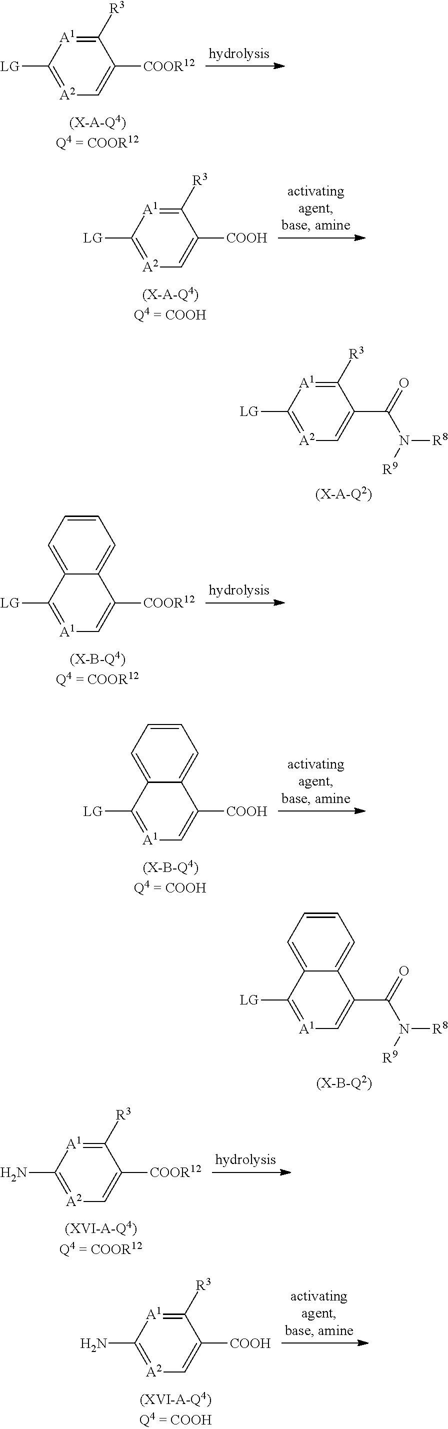

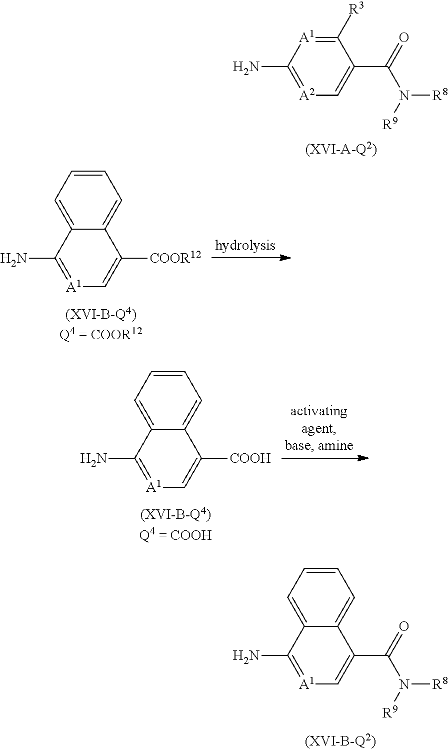

where
R³, R⁸, R⁹, R¹², A¹ and A² have the meanings given above.

In process (J) according to the invention for preparing the compounds (X-A)/(X-B) and (XVI-A)/(XVI-B), compounds of the formula (X-A-Q4/X-B-Q4) or (XVI-A-Q4)/(XVI-B-Q4) (Q⁴=ester) are hydrolyzed by methods known to the person skilled in the art (Q⁴═COOH) and then converted with the aid of an activating agent with a primary or secondary amine HNR⁸R⁹ into amides of the formula (X-A-Q2)/(X-B-Q2) or (XVI-A-Q2)/(XVI-B-Q2). Activation can be via acid chlorides, mixed anhydrides, pentafluorophenyl esters or with the aid of substituted carbodiimides, for example DCC(N,N′-dicyclohexylcarbodiimide), DIC (diisopropylcarbodiimide) or EDC (N-ethyl-N′-(3-dimethylaminopropyl)carbodiimide HCl), and a benzotriazole such as HOBt (1-hydroxybenzotriazole) or azabenzotriazole such as HOAt (7-aza-1-hydroxybenzotriazole).

The present invention also provides compounds of the formulae (X-A) and (X-B)

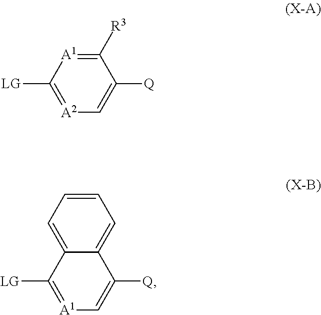

where A¹, A², R³ and Q are as defined above and LG represents halogen.
Synthesis Procedure for the Preparation of Compounds of the Formula (I-AB-Q1 where R¹═NH₂) by Nucleophilic Substitution

### 5-[5-Amino-4-(3-chloro-5-trifluoromethylphenyl)-3-trifluoromethylpyrazol-1-yl]-2-fluorobenzonitrile

### (I-A-Q4-009)

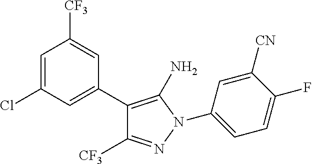

2-Fluoro-5-hydrazinobenzonitrile (120 mg, 0.79 mmol) and a spatula tip of p-toluenesulphonic acid are added to a solution of 2-(3-chloro-5-trifluoromethylphenyl)-4,4,4-trifluoro-3-oxobutyronitrile (250 mg, 0.79 mmol) in toluene (5 ml). The reaction vessel is closed and stirred in a CEM Discover microwave reactor at 170° C. for 12 min. The solvent is removed under reduced pressure. Purification by chromatography on silica gel using cyclohexane/ethyl acetate 8/1 gives 5-[5-amino-4-(3-chloro-5-trifluoromethylphenyl)-3-trifluoromethylpyrazol-1-yl]-2-fluorobenzonitrile (265 mg, 0.59 mmol, 72%).

### log P(HCOOH): 4.54

¹H NMR (DMSO-d6): 8.18, (dd, 1H, J=5.6 and 2.8 Hz), 8.01 (ddd, 1H, J=9.2 and 4.8 and 2.8 Hz), 7.78 (s, 1H), 7.71 (d, 1H, J=9.2 Hz), 7.67 (s, 1H), 7.59 (s, 1H), 5.86 (s, 2H)
5-[5-Amino-4-(3-chloro-5-trifluoromethylphenyl)-3-trifluoromethylpyrazol-1-yl]-2-[1,2,4]triazol-1-ylbenzonitrile (I-A-Q1-001)

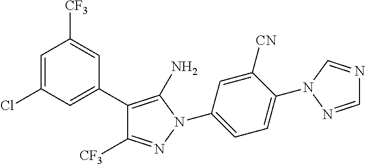

1,2,4-Triazole (18 mg, 0.27 mmol) and potassium carbonate (37 mg, 0.27 mmol) are added to a solution of 5-[5-amino-4-(3-chloro-5-trifluoromethylphenyl)-3-trifluoromethylpyrazol-1-yl]-2-fluorobenzonitrile (100 mg, 0.22 mmol) in DMF (2 ml). The reaction mixture is stirred at 90° C. for 15 min, cooled to room temperature, poured into water (5 ml) and extracted with ethyl acetate (5 ml). The organic phase is washed with water (5 ml), dried over sodium sulphate and concentrated using a rotary evaporator. Purification by chromatography on silica gel using cyclohexane/ethyl acetate 2/1 to 1/1 gives 5-[5-amino-4-(3-chloro-5-trifluoromethylphenyl)-3-trifluoromethylpyrazol-1-yl]-2-[1,2,4]triazol-1-ylbenzonitrile (50 mg, 0.10 mmol, 45%).

### log P(HCOOH): 3.92

¹H NMR (DMSO-d6): 9.19 (s, 1H), 8.36 (s, 1H), 8.33 (d, 1H, J=2.4 Hz), 8.15 (dd, 1H, J=8.8 and 2.4 Hz), 8.05 (d, 1H, J=8.8 Hz), 7.80 (s, 1H), 7.69 (s, 1H), 7.61 (s, 1H), 5.98 (s, 2H)

### 5-[5-Amino-4-(3-chloro-5-trifluoromethylphenyl)-3-trifluoromethylpyrazol-1-yl]-2-(5-oxo-4,5-dihydro[1,2,4]triazol-1-yl)benzonitrile (I-A-Q1-002)

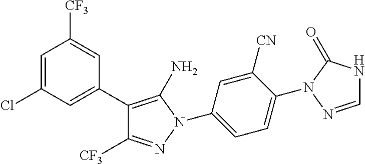

2,4-Dihydro[1,2,4]triazol-3-one (11 mg, 0.13 mmol) and potassium carbonate (18 mg, 0.13 mmol) are added to a solution of 5-[5-amino-4-(3-chloro-5-trifluoromethylphenyl)-3-trifluoromethylpyrazol-1-yl]-2-fluorobenzonitrile (50 mg, 0.11 mmol) in DMF (2 ml). The reaction mixture is stirred at 80° C. for 15 min, cooled to room temperature, poured into water (5 ml) and extracted with ethyl acetate (5 ml). The organic phase is washed with water (5 ml), dried over sodium sulphate and concentrated using a rotary evaporator. Purification by chromatography on silica gel using cyclohexane/ethyl acetate 2/1 to 1/3 gives 5-[5-amino-4-(3-chloro-5-trifluoromethylphenyl)-3-trifluoromethylpyrazol-1-yl]-2-(5-oxo-4,5-dihydro[1,2,4]triazol-1-yl)benzonitrile (8 mg, 0.016 mmol, 12%).

### log P(HCOOH): 3.45

¹H NMR (DMSO-d6): 8.28 (d, 1H, J=2.3 Hz), 8.24 (d, 1H, J=1.2 Hz), 8.12 (dd, 1H, J=8.8 and 2.4 Hz), 7.87 (d, 1H, J=8.8 Hz), 7.79 (s, 1H), 7.69 (s, 1H), 7.60 (s, 1H), 5.96 (s, 2H)

Synthesis Procedure for the Preparation of Compounds of the Formula (I-AB-Q1 where R¹═NH₂) Using Hydrazines

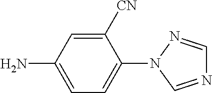

### 5-Amino-2-[1,2,4]triazol-1-ylbenzonitrile

[1,2,4]Triazole (10.1 g, 73.5 mmol) is initially charged in NMP (50 ml), and potassium carbonate (30.5 g, 220 mmol) is added. After 10 min of stirring at room temperature 5-amino-2-fluorobenzonitrile (10.0 g, 73.5 mmol) is added, and the mixture is stirred at 170° C. for 5 h. The reaction mixture is cooled and poured into water (800 ml). Removal of the precipitate by filtration with suction and washing with water gives 5-amino-2-[1,2,4]triazol-1-ylbenzonitrile (7.50 g, 40.5 mmol, 53%) which is used without purification for the next step.

### log P(HCOOH): 1.01

¹H NMR (DMSO-d6): 8.82 (s, 1H), 8.16 (s, 1H), 7.37 (d, 1H, J=8.4 Hz), 7.03 (d, 1H, J=2.8 Hz), 6.97 (dd, 1H, J=8.8 and 2.4 Hz), 5.85 (s, 2H)

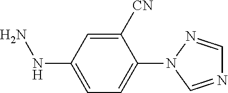

### 5-Hydrazino-2-[1,2,4]triazol-1-ylbenzonitrile (VII-A-103)

5-Amino-2-[1,2,4]triazol-1-ylbenzonitrile (5.0 g, 27.0 mmol) is initially charged in water (25 ml), concentrated hydrochloric acid (50 ml) is added and the mixture is stirred at medium speed at room temperature. The reaction mixture is cooled to 0° C., and a solution of sodium nitrite (3.73 g, 54.0 mmol) in water (25 ml) is added. The mixture is stirred at 0° C. for 1 h, and a solution of tin(II) chloride dihydrate (18.3 g, 81.0 mmol) in concentrated hydrochloric acid (100 ml) is then added dropwise. After the end of the addition, the mixture is stirred at 0° C. for 30 min, warmed to room temperature and stirred for a further 1.5 h. With ice cooling, the reaction mixture is made alkaline using concentrated aqueous sodium hydroxide solution, ethyl acetate is added and the mixture is filtered with suction through Celite. The organic phase is dried over sodium sulphate and concentrated using a rotary evaporator. Crystallisation of the residue from methanol gives 5-hydrazino-2-[1,2,4]triazol-1-ylbenzonitrile (1.2 g, 6.0 mmol, 22%).

¹H NMR (DMSO-d6): 8.84 (s, 1H), 8.17 (s, 1H), 7.46 (s, 1H), 7.43 (d, 1H, J=8.8 Hz), 7.23 (d, 1H, J=2.4 Hz), 7.13 (dd, 1H, J=9.2 and 2.8 Hz), 4.20 (, 2H)

Synthesis Procedure for the Preparation of Compounds of the Formula (I-A/B-Q2 where R¹═NH₂) Using Hydrazines

### Methyl 4-hydrazino-2-methylbenzoate (VII-A-008)

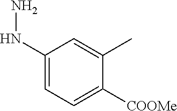

4-Acetamido-2-methylbenzoic acid (46 g, 238 mmol) is initially charged in methanol (460 ml), and concentrated sulphuric acid (63 g, 643 mmol) is added. The reaction mixture is heated under reflux for 3 h, cooled, carefully added to ice-water and made alkaline using 5N aqueous sodium hydroxide solution. Extraction with ethyl acetate, drying of the organic phase over sodium sulphate and removal of the solvent under reduced pressure gives ethyl 4-amino-2-methylbenzoate (38.3 g, 232 mmol, 94%) which is used without further purification for the next step.

Methyl 4-amino-2-methylbenzoate (5.0 g, 30 mmol) is initially charged in water (25 ml), concentrated hydrochloric acid (50 ml) is added and the mixture is stirred at room temperature for 30 min. The reaction mixture is cooled to 0° C., and a solution of sodium nitrite (2.72 g, 39.3 mmol) in water (25 ml) is slowly added dropwise. The mixture is stirred at 0° C. for one hour, and tin(II) chloride dihydrate (20.5 g, 90.8 mmol) in concentrated hydrochloric acid (100 ml) is then added dropwise at 0 C. The mixture is stirred initially at 0° C. for 30 min and then at room temperature for 1.5 h. The reaction mixture is diluted with water (50 ml), and the precipitate is filtered off and washed with a little water. The filtrate is poured into ice-water, adjusted to pH 8-9 using semiconcentrated aqueous sodium hydroxide solution and extracted twice with dichloromethane. The organic phases are combined and dried over sodium sulfate. Removal of the solvent under reduced pressure gives methyl 4-hydrazino-2-methylbenzoate (4.1 g, 22.8 mmol, 70%) which is used without further purification for the next step.

### log P(HCOOH): 0.40

¹H NMR (DMSO-d6): 7.67 (d, 1H, J=8.8 Hz), 7.19 (s, 1H), 6.61-6.58 (m, 2H), 4.03 (s, 2H), 3.71 (s, 3H), 2.44 (s, 3H)

### Methyl 4-[5-amino-4-(3,5-dichlorophenyl)-3-trifluoromethylpyrazol-1-yl]-2-methylbenzoate (I-A-Q4-011)

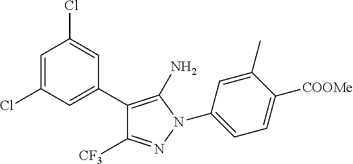

Methyl 4-hydrazino-2-methylbenzoate (43 mg, 0.24 mmol) and triethylamine (24 mg, 0.24 mmol) are added to a solution of 3-chloro-2-(3,5-dichlorophenyl)-4,4,4-trifluorobut-2-enenitrile (100 mg, 0.24 mmol) in ethanol (3 ml), and the mixture is stirred at 80° C. overnight. The reaction mixture is cooled to room temperature, poured into water (5 ml) and extracted with ethyl acetate (5 ml). The organic phase is washed with water (5 ml), dried over sodium sulphate and concentrated using a rotary evaporator. Purification by chromatography on silica gel using cyclohexane/ethyl acetate 6/1 gives methyl 4-[5-amino-4-(3,5-dichlorophenyl)-3-trifluoromethylpyrazol-1-yl]-2-methyl-benzoate (55 mg, 0.12 mmol, 52%).

### log P(HCOOH): 5.08

¹H NMR (DMSO-d6): 7.96 (d, 1H, J=8.4 Hz), 7.59-7.55 (m, 2H), 7.53 (t, 1H, J=2.0 Hz), 7.34 (d, 2H, J=2.0 Hz), 5.70 (s, 2H), 3.87 (s, 3H), 2.60 (s, 3H)

### 4-[5-Amino-4-(3,5-dichlorophenyl)-3-trifluoromethylpyrazol-1-yl]-2-methylbenzoic acid (I-A-Q4-010)

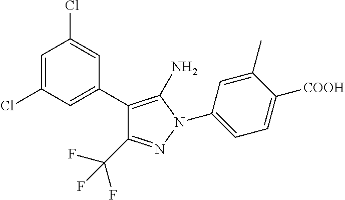

Methyl 4-[5-amino-4-(3,5-dichlorophenyl)-3-trifluoromethylpyrazol-1-yl]-2-methylbenzoate (300 mg, 0.51 mmol) is initially charged in water (10 ml) and ethanol (10 ml), and sodium hydroxide (200 mg, 5.1 mmol) is added. The reaction mixture is stirred at room temperature for two days, poured into water (50 ml), acidified (pH3-4) with 2N hydrochloric acid, extracted twice with ethyl acetate and dried over sodium sulphate. Removal of the solvent on a rotary evaporator gives methyl 4-[5-amino-4-(3,5-dichlorophenyl)-3-trifluoromethylpyrazol-1-yl]-2-methylbenzoate in quantitative yield.

### log P(HCOOH): 4.04

¹H NMR (DMSO-d6): 7.97 (d, 1H, J=8.4 Hz), 7.55-7.52 (m, 3H), 7.34 (d, 2H, J=2.0 Hz), 5.68 (s, 2H), 2.61 (s, 3H)
4-[5-Amino-4-(3,5-dichlorophenyl)-3-trifluoromethylpyrazol-1-yl]-2-methyl-N-pyridin-2-ylmethylbenzamide (I-A-Q2-012)

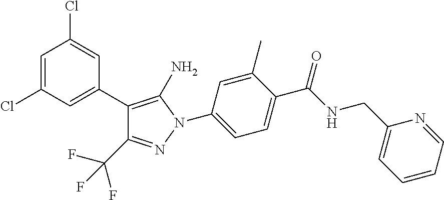

A solution of 4-[5-amino-4-(3,5-dichlorophenyl)-3-trifluoromethylpyrazol-1-yl]-2-methylbenzoic acid (100 mg, 0.23 mmol), 1-ethyl-3-[3-dimethylaminopropyl]carbodiimide hydrochloride (58 mg, 0.30 mmol), 1-hydroxy-1H-benzotriazole (41 mg, 0.30 mmol) and triethylamine (28 mg, 0.28 mmol) in DMF (4 ml) are added to a solution of 2-aminomethylpyridine (25 mg, 0.23 mmol) in DMF (1 ml), and the mixture is stirred at room temperature overnight. Water (5 ml) is added, the reaction mixture is extracted twice with ethyl acetate (5 ml each) and the extracts are washed with NaHCO₃ solution (5 ml) and NaCl solution (5 ml), dried over sodium sulphate and concentrated on a rotary evaporator. Purification by column chromatography on silica gel using cyclohexane/ethyl acetate ½ to ⅓ gives 4-[5-amino-4-(3,5-dichlorophenyl)-3-trifluoromethylpyrazol-1-yl]-2-methyl-N-pyridin-2-ylmethylbenzamide (75 mg, 0.14 mmol, 61%).

### log P(HCOOH): 3.35

¹H NMR (DMSO-d6): 8.71 (t, 1H, J=5.6 Hz), 8.52 (d, 1H, J=4.0 Hz), 7.78 (td, 1H, J=8.0 and 2.0 Hz), 7.60 (d, 1H, J=8.0 Hz), 7.53 (t, 1H, J=2.0 Hz), 7.50-7.47 (m, 2H), 7.40 (d, 1 H, J=7.6 Hz), 7.34 (d, 2H, J=2.0 Hz), 7.26 (dd, 1H, J=7.6 and 5.6 Hz), 5.59 (s, 2H), 4.58 (d, 2 H, J=6.0 Hz), 2.46 (s, 3H)

Synthesis Procedure for the Preparation of Compounds of the Formula (I-A/B-Q2 where R¹═NH₂) by N-arylation of Pyrazoles

### Ethyl 4-[5-amino-4-(3,4-dichlorophenyl)-3-(trifluoromethyl)-1H-pyrazol-1-yl]-2-methylbenzoate (I-A-Q4-013)

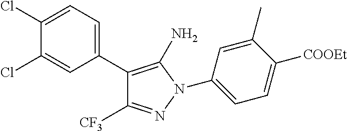

Under argon, 4-(3,4-dichlorophenyl)-3-(trifluoromethyl)-1H-pyrazol-5-amine (70% pure) (1.94 g, 6.55 mmol) is initially charged in toluene (39 ml), ethyl 4-iodo-2-methylbenzoate (90% pure) (1.90 g), potassium carbonate (3.10 g), copper(I) iodide (62 mg) and trans-dimethylaminocyclohexane (186 mg) are added and the mixture is stirred under argon at 110° C. for 16 hours.

The reaction mixture is concentrated on a rotary evaporator, taken up in water (20 ml) and ethyl acetate (39 ml), adjusted to pH=1 using conc. HCl, the org. phase is separated off, the aqueous phase is once more extracted with ethyl acetate (30 ml) and the combined organic phases are dried over sodium sulphate and concentrated on a rotary evaporator. After extraction of organic impurities with hexane, a brown solid remains. Purification by HPLC affords ethyl 4-[5-amino-4-(3,4-dichlorophenyl)-3-(trifluoromethyl)-1H-pyrazol-1-yl]-2-methylbenzoate (630 mg, 24%).

### log P(HCOOH): 5.24

¹H NMR (DMSO-d6): 1.35 (t, 3H); 2.56 (s, 3H); 4.34 (q, 2H); 5.62 (br. s, 2H); 7.32 (dd, 1 H); 7.54-7.59 (m, 3H); 7.67 (d, 1H); 7.95 (d, 1H)

### 4-[5-Amino-4-(3,4-dichlorophenyl)-3-(trifluoromethyl)-1H-pyrazol-1-yl]-2-methylbenzoic acid

### (I-A-Q4-012)

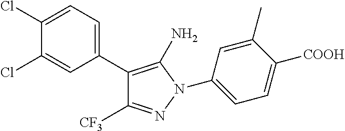

At 20 C., ethyl 4-[5-amino-4-(3,4-dichlorophenyl)-3-(trifluoromethyl)-1H-pyrazol-1-pyrazol-1-yl]-2-methylbenzoate (600 mg) is stirred in isopropanol (9 ml) and 1N aqueous sodium hydroxide solution (6 ml) for two days.

The isopropanol is removed under reduced pressure on a rotary evaporator and the aqueous phase is adjusted to pH=1 with concentrated hydrochloric acid. The resulting precipitate is filtered off with suction, washed with water and dried.

This gives 4-[5-amino-4-(3,4-dichlorophenyl)-3-(trifluoromethyl)-1H-pyrazol-1-yl]-2-methylbenzenecarboxylic acid (495 mg, 86%) as a yellowish solid, content according to HPLC: 98.8%.

### log P(HCOOH): 3.92

¹H NMR (DMSO-d6): 2.61 (s, 3H); 5.61 (br. S, 2H); 7.32 (dd, 1H); 7.52-7.56 (m, 3H); 7.67 (d, 1H); 7.96 (d, 1H)

Synthesis Procedure for the Preparation of Compounds of the Formula (I-A/B-Q2 where R¹═I)

### 4-[4-(3,5-Dichlorophenyl)-5-iodo-3-trifluoromethylpyrazol-1-yl]-2-methyl-N-pyridin-2-ylmethylbenzamide (I-A-Q2-025)

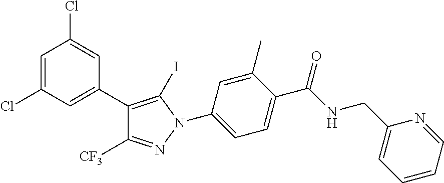

Iodoform (121 mg, 0.31 mmol) is added to a solution of 4-[5-amino-4-(3,5-dichlorophenyl)-3-trifluoromethylpyrazol-1-yl]-2-methyl-N-pyridin-2-ylmethylbenzamide (80 mg, 0.15 mmol) in chloroform (1 ml). tert-Butyl nitrite (35 mg, 0.34 mmol) is slowly added dropwise, and the reaction mixture is stirred at room temperature overnight. Purification by chromatography on silica gel using cyclohexane/ethyl acetate 1/1 gives 4-[4-(3,5-dichlorophenyl)-5-iodo-3-trifluoromethylpyrazol-1-yl]-2-methyl-N-pyridin-2-ylmethylbenzamide (45 mg, 0.071 mmol, 46%). Minor impurities in the 4-[4-(3,5-dichlorophenyl)-3-trifluoromethylpyrazol-1-yl]-2-methyl-N-pyridin-2-ylmethylbenzamide can be removed by HPLC.

### log P(HCOOH): 4.29

¹H NMR (DMSO-d6): 8.85 (t, 1H, J=5.8 Hz), 8.52 (d, 1H, J=4.0 Hz), 7.78 (td, 1H, J=8.0 and 2.0 Hz), 7.71 (t, 1H, J=2.0 Hz), 7.64 (d, (1H, J=8.8 Hz), 7.53-7.50 (m, 2F), 7.45 (d, 2 H, J=1.6 Hz), 7.42 (d, 1H, J=8.0 Hz), 7.27 (dd, 1H, J=6.4 and 5.2 Hz), 4.58 (d, 2H, J=6.0 Hz), Me under DMSO-d6 signal

Synthesis Procedure for the Preparation of Compounds of the Formula (I-A/B-Q2 where R¹═Br)

### 4-[5-Bromo-4-(3,5-dichlorophenyl)-3-trifluoromethylpyrazol-1-yl]-2-methyl-N-(2,2,2-trifluoro-1-mmethylethyl)benzamide (I-A-Q2-024)

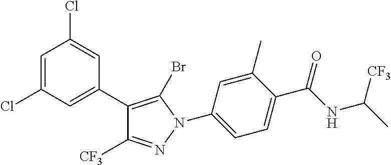

tert-Butyl nitrite (86 mg, 0.84 mmol) is slowly added dropwise to a solution of 4-[5-amino-4-(3,5-dichlorophenyl)-3-trifluoromethylpyrazol-1-yl]-2-methyl-N-(2,2,2-trifluoro-1-methylethyl)benzamide (200 mg, 0.38 mmol) in bromoform (0.5 ml), and the mixture is stirred at room temperature overnight. Purification by chromatography on silica gel using cyclohexane/ethyl acetate 1/1 gives 4-[5-bromo-4-(3,5-dichlorophenyl)-3-trifluoromethylpyrazol-1-yl]-2-methyl-N-(2,2,2-trifluoro-1-methylethyl)benzamide (62 mg, 0.11 mmol, 24%).

### log P(HCOOH): 5.53

¹H NMR (DMSO-d6): 8.90 (d, 1H, J=8.8 Hz), 7.72 (t, 1H, J=2.0 Hz), 7.57-7.54 (m, 3H), 7.50 (d, 2H, J=2.0 Hz), 4.84-4.78 (m, 1H), 2.43 (s, 3H), 1.34 (d, 3H, J=6.4 Hz)

General Synthesis Procedure for the Preparation of Compounds of the Formula (I-AB-Q2 where R¹═Cl)

### 4-[5-Chloro-4-(3,5-dichlorophenyl)-3-trifluoromethylpyrazol-1-yl]-2-methyl-N-(2,2,2-trifluoro-1-methylethyl)benzamide (I-A-Q2-022)

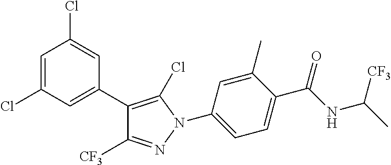

Copper(II) chloride (31 mg, 0.23 mmol) and (4-[5-amino-4-(3,5-dichlorophenyl)-3-trifluoromethylpyrazol-1-yl]-2-methyl-N-(2,2,2-trifluoro-1-methylethyl)benzamide (80 mg, 0.15 mmol) are added to a solution of tert-butyl nitrite (24 mg, 0.23 mmol) in acetonitrile (2 ml), and the mixture is stirred at 80° C. for 30 min and then concentrated using a rotary evaporator. Purification by chromatography on silica gel using cyclohexane/ethyl acetate 1/1 gives 4-[5-chloro-4-(3,5-dichlorophenyl)-3-trifluoromethylpyrazol-1-yl]-2-methyl-N-(2,2,2-trifluoro-1-methylethyl)benzamide (30 mg, 0.055 mmol, 33%).

### log P(HCOOH): 5.59

¹H NMR (DMSO-d6): 8.88 (d, 1H, J=8.4 Hz), 7.72 (t, 1H, J=1.8 Hz), 7.59-7.55 (m, 3H), 7.52 (d, 2H, J=2.0 Hz), 4.84-4.78 (m, 1H), 2.43 (s, 3H), 1.34 (d, 3H, J=6.4 Hz)

Synthesis Procedure for the Preparation of Compounds of the Formula (I-A/B-Q2 where R¹═SMe or SOMe)

### 4-[4-(3,5-Dichlorophenyl)-5-methylsulphanyl-3-trifluoromethylpyrazol-1-yl]-2-methyl-N-(2,2,2-trifluoro-1-methylethyl)benzamide (I-A-Q2-027)

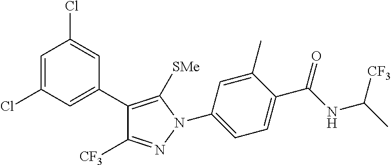

Dimethyl disulphide (36 mg, 0.38 mmol) is added to a solution of 4-[5-amino-4-(3,5-dichlorophenyl)-3-trifluoromethylpyrazol-1-yl]-2-methyl-N-(2,2,2-trifluoro-1-methylethyl)benzamide (100 mg, 0.19 mmol) in chloroform (1 ml). The reaction mixture and tert-butyl nitrite (29 mg, 0.29 mmol) are slowly brought to reflux for 3 min, cooled, poured into water (5 ml) and extracted with ethyl acetate (5 ml). The organic phase is washed with water (5 ml), dried over sodium sulphate and concentrated using a rotary evaporator. Purification by chromatography on silica gel using cyclohexane/ethyl acetate 8/1 gives 4-[4-(3,5-dichlorophenyl)-5-methylsulphanyl-3-trifluoromethylpyrazol-1-yl]-2-methyl-N-(2,2,2-trifluoro-1-methylethyl)benzamide (43 mg, 0.77 mmol, 39%).

### log P(HCOOH): 5.48

¹H NMR (DMSO-d6): 8.87 (d, 1H, J=8.8 Hz), 7.69 (t, 1H, J=1.8 Hz), 7.58-7.51 (m, 3H), 7.50 (d, 2H, J=2.0 Hz), 4.84-4.77 (m, 1H), 2.43 (s, 3H), 2.04 (s, 3H), 1.34 (d, 3H, J=6.4 Hz)

### 4-[4-(3,5-Dichlorophenyl)-5-methanesulphinyl-3-trifluoromethylpyrazol-1-yl]-2-methyl-N-pyridin-2-ylmethylbenzamide (I-A-Q2-063)

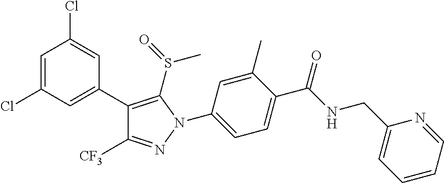

Chloroperbenzoic acid (29 mg, 0.12 mmol) is added to a solution of 4-[4-(3,5-dichlorophenyl)-5-methanesulphanyl-3-trifluoromethylpyrazol-1-yl]-2-methyl-N-pyridin-2-ylmethylbenzamide (50 mg, 0.09 mmol) in dichloromethane (1 ml), and the mixture is stirred overnight. The reaction mixture is washed with water, and the organic phase is dried over sodium sulphate and concentrated using a rotary evaporator. Purification by chromatography on silica gel using cyclohexane/ethyl acetate 9/1 gives 4-[4-(3,5-dichlorophenyl)-5-methanesulphanyl-3-trifluoromethylpyrazol-1-yl]-2-methyl-N-pyridin-2-ylmethylbenzamide (20 mg, 0.035 mmol, 39%).

### log P(HCOOH): 3.11

¹H NMR (DMSO-d6): 8.54 (d, 1H, J=4.8 Hz), 7.77 (td, 1H, J=7.6 and 2.0 Hz), 7.66-7.48 (m, 7H), 7.41 (d, 1H, J=8.0 Hz), 7.26 (dd, 7.2 and 4.8 Hz), 4.66 (d, 2H, J=6.0 Hz), 2.65 (s, 3 H), 2.50 (s, 3H)

Synthesis Procedure for the Preparation of Compounds of the Formula (I-AB-Q2 where R¹═NH₂ and A¹=A²═N)

### N-(Pyridin-2-ylmethyl)-2-[5-amino-4-(3,5-dichlorophenyl)-3-trifluoromethylpyrazol-1-yl]-4-trifluoromethylpyrimidine-5-carboxamide (I-A-Q2-020)

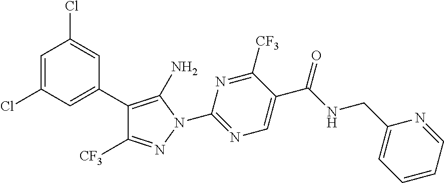

N-Ethyldiisopropylamine (174 mg, 1.35 mmol) and 2-aminomethylpyridine (140 mg, 1.29 mmol) in THF (2 ml) are added to a solution of 2-chloro-4-trifluoromethylpyrimidine-5-carbonyl chloride (300 mg, 1.23 mmol) in dichloromethane (3 ml), and the mixture is stirred at room temperature overnight. Removal of the solvent under reduced pressure gives 650 mg of crude product, 95 mg of which are dissolved in DMF (2 ml), and 4-(3,5-dichlorophenyl)-5-trifluoromethyl-2H-pyrazol-3-ylamine (107 mg, 0.36 mmol) and potassium carbonate (58 mg, 0.42 mmol) are added. The mixture is stirred at 100° C. for 1.5 h, cooled to room temperature, poured into water (5 ml) and extracted with ethyl acetate (5 ml). The organic phase is washed with water (5 ml), dried over sodium sulphate and concentrated using a rotary evaporator. Purification by chromatography on silica gel using cyclohexane/ethyl acetate 3/1 gives N-(pyridin-2-ylmethyl)-2-[5-amino-4-(3,5-dichlorophenyl)-3-trifluoromethylpyrazol-1-yl]-4-trifluoromethylpyrimidine-5-carboxamide (55 mg, 0.095 mmol, 53%).

### log P(HCOOH): 3.54

¹H NMR (DMSO-d6): 9.30 (s, 1H), 9.27 (t, 1H, J=6.0 Hz), 8.54 (d, 1H, J=4.8 Hz), 7.80 (td, 1H, J=7.8 and 2.0 Hz), 7.57 (t, 1H, J=1.8 Hz), 7.42 (d, 1H, J=7.6 Hz), 7.37 (d, 2H, J=1.6 Hz), 7.30 (dd, 1H, J=6.4 and 3.6 Hz), 7.00 (s, 2H), 4.62 (d, 2H, J=5.6 Hz)

Synthesis Procedure for the Preparation of Compounds of the Formula (I-A/B-02 where R¹═H)

### Ethyl 4-(4-iodo-3-trifluoromethylpyrazol-1-yl)-2-trifluoromethylbenzoate (XI-A-001)

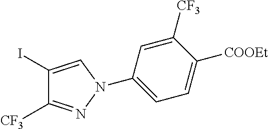

4-Iodo-3-trifluoromethyl-1H-pyrazole (2.22 g, 8.47 mmol) and potassium carbonate (1.40 g, 10.2 mmol) are added to a solution of ethyl 4-fluoro-2-trifluoromethylbenzoate (2.00 g, 8.47 mmol) in DMF (30 ml), and the mixture is stirred at 60° C. for 1 h. The reaction mixture is diluted with ethyl acetate, washed with water and saturated sodium chloride solution, dried over magnesium sulphate and concentrated under reduced pressure. Purification by chromatography on silica gel gives ethyl 4-(4-iodo-3-trifluoromethylpyrazol-1-yl)-2-trifluoromethylbenzoate (3.65 g, 7.63 mmol, 90%).

¹H NMR (CDCl₃): 1.41 (t, 3H, J=7.1 Hz), 4.43 (q, 2H, J=7.1 Hz), 7.92-7.99 (m, 2H), 8.08 (s, 1H), 8.14-8.14 (m, 1H).

### Ethyl 4-[4-(3,5-dichlorophenyl)-3-trifluoromethylpyrazol-1-yl]-2-trifluoromethylbenzoate

### (I-A-Q4-001)

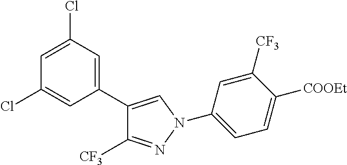

3,5-Dichlorophenylboronic acid (0.60 g, 3.14 mmol) and sodium carbonate (0.69 g, 6.48 mmol) in water (2 ml) are added to a solution of ethyl 4-(4-iodo-3-trifluoromethylpyrazol-1-yl)-2-trifluoromethylbenzoate (1.00 g, 2.09 mmol) in DME (10 ml). The reaction vessel is degassed and filled with nitrogen. Tetrakis(triphenylphosphine)palladium (0.73 g, 0.63 mmol) is added, and the reaction mixture is stirred at 85° C. for 9 h. After cooling, the mixture is poured into water and extracted with ethyl acetate. The organic phase is washed with water and saturated sodium chloride solution, dried over magnesium sulphate and concentrated using a rotary evaporator. Purification by chromatography on silica gel gives ethyl 4-[4-(3,5-dichlorophenyl)-3-trifluoromethylpyrazol-1-yl]-2-trifluoromethylbenzoate (0.30 g, 0.60 mmol, 29%).

¹H NMR (CDCl₃): 1.42 (t, 3H, J=7.1 Hz), 4.44 (q, 2H, J=7.1 Hz), 7.37-7.42 (m, 3H), 8.00-8.01 (m, 2H), 8.14-8.15 (m, 2H)

### 4-[4-(3,5-Dichlorophenyl)-3-trifluoromethylpyrazol-1-yl]-2-trifluoromethylbenzoate

### (I-A-Q4-002)

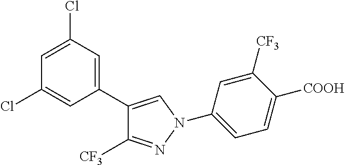

Sodium hydroxide (48 mg, 1.2 mmol) in water (10 ml) is added to a solution of ethyl 4-[4-(3,5-dichlorophenyl)-3-trifluoromethylpyrazol-1-yl]-2-trifluoromethylbenzoate (300 mg, 0.60 mmol) in ethanol (10 ml), and the mixture is stirred at room temperature overnight. The organic solvent is removed under reduced pressure and the aqueous residue is treated with dilute hydrochloric acid. The precipitate formed is filtered off and dried at 50° C. under reduced pressure, giving 4-[4-(3,5-dichlorophenyl)-3-trifluoromethylpyrazol-1-yl]-2-trifluoromethylbenzoic acid (259 mg, 0.53 mmol, 88%).

### 4-[4-(3,5-Dichlorophenyl)-3-trifluoromethylpyrazol-1-yl]-N-(2,2,2-trifluoroethyl)-2-trifluoromethylbenzamide (I-A-Q2-002)

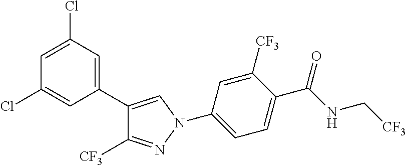

2,2,2-Trifluoroethylamine (40 mg, 0.41 mmol), and 1-ethyl-3-[3-dimethylaminopropyl]carbodiimide hydrochloride (60 mg, 0.33 mmol) are added to a solution of 4-[4-(3,5-dichlorophenyl)-3-trifluoromethylpyrazol-1-yl]-2-trifluoromethylbenzoic acid (130 mg, 0.28 mmol) in DMF (10 ml). The reaction mixture is stirred at room temperature for 8 h, diluted with tert-butyl methyl ether, washed with water and saturated sodium chloride solution and dried over magnesium sulphate, and the solvent is removed under reduced pressure. Purification by chromatography on silica gel gives 4-[4-(3,5-dichlorophenyl)-3-trifluoromethylpyrazol-1-yl]-N-(2,2,2-trifluoroethyl)-2-trifluoromethylbenzamide (100 mg, 0.18 mmol, 66%).

### log P (pH 2): 5.16

### log P (pH 7.5): 5.14

¹H NMR (CDCl₃): 4.10-4.14 (m, 2H), 6.33-6.35 (m, 1H), 7.34-7.40 (m, 3H), 7.59-7.67 (m, 1H), 7.86-8.15 (m, 3H).

The stated log P values were determined in accordance with EEC Directive 79/831 Annex V.A8 by HPLC (High Performance Liquid Chromatography) using a reversed-phase column (C18).

Agilent 1100 LC-System; 50*4.6 Zorbax Eclipse Plus C18 1.8 microm; mobile phase A: acetonitrile (0.1% formic acid); mobile phase B: water (0.09% formic acid); linear gradient from 10% acetonitrile to 95% acetonitrile in 4.25 min, then 95% acetonitrile for a further 1.25 min; oven temperature 55° C.; flow rate: 2.0 ml/min

When using the active compounds according to the invention as insecticides and acaricides, the application rates can be varied within a relatively wide range, depending on the kind of application. The application rate of the active compounds according to the invention is when treating plant parts, e.g. leaves: from 0.1 to 10 000 g/ha, preferably from 10 to 1000 g/ha, particularly preferably from 50 to 300 g/ha (when the application is carried out by watering or dripping, it may even be possible to reduce the application rate, in particular when inert substrates such as rock wool or perlite are used); when treating seed: from 2 to 200 g per 100 kg of seed, preferably from 3 to 150 g per 100 kg of seed, particularly preferably from 2.5 to 25 g per 100 kg of seed, very particularly preferably from 2.5 to 12.5 g per 100 kg of seed; when treating the soil: from 0.1 to 10 000 g/ha, preferably from 1 to 5000 g/ha. These application rates are mentioned only by way of example and are not limiting in the sense of the invention.

The active compounds according to the invention can be used to protect plants for a certain period after the treatment against attack by the animal pests mentioned. The period for which protection is provided extends generally for 1 to 28 days, preferably for 1 to 14 days, particularly preferably for 1 to 10 days, very particularly preferably for 1 to 7 days after the treatment of the plants with the active compounds, or for up to 200 days after a seed treatment.

The active compounds according to the invention, in combination with good plant tolerance and favourable toxicity to warm-blooded animals and being tolerated well by the environment, are suitable for protecting plants and plant organs, for increasing the harvest yields, for improving the quality of the harvested material and for controlling animal pests, in particular insects, arachnids, helminths, nematodes and molluscs, which are encountered in agriculture, in horticulture, in animal husbandry, in forests, in gardens and leisure facilities, in the protection of stored products and of materials, and in the hygiene sector. They may be preferably employed as crop protection agents. They are active against normally sensitive and resistant species and also against all or some stages of development. The abovementioned pests include:

From the order of the Anoplura (Phthiraptera), for example, Damalinia spp., Haematopinus spp., Linognathus spp., Pediculus spp., Trichodectes spp.

From the class of the Arachnida, for example, Acarus siro, Aceria sheldoni, Aculops spp., Aculus spp., Amblyomma spp., Argas spp., Boophilus spp., Brevipalpus spp., Bryobia praetiosa, Chorioptes spp., Dermanyssus gallinae, Eotetranychus spp., Epitrimerus pyri, Eutetranychus spp., Eriophyes spp., Hemitarsonemus spp., Hyalomma spp., Ixodes spp., Latrodectus mactans, Metatetranychus spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vasates lycopersici.

From the class of the Bivalva, for example, Dreissena spp.

From the order of the Chilopoda, for example, Geophilus spp., Scutigera spp.

From the order of the Coleoptera, for example, Acanthoscelides obtectus, Adoretus spp., Agelastica alni, Agriotes spp., Amphimallon solstitialis, Anobium punctatum, Anoplophora spp., Anthonomus spp., Anthrenus spp., Apogonia spp., Atomaria spp., Attagenus spp., Bruchidius obtectus, Bruchus spp., Ceuthorhynchus spp., Cleonus mendicus, Conoderus spp., Cosmopolites spp., Costelytra zealandica, Curculio spp., Cryptorhynchus lapathi, Dermestes spp., Diabrotica spp., Epilachna spp., Faustinus cubae, Gibbium psylloides, Heteronychus arator, Hylamorpha elegans, Hylotrupes bajulus, Hypera postica, Hypothenemus spp., Lachnosterna consanguinea, Leptinotarsa decemlineata, Lissorhoptrus oryzophilus, Lixus spp., Lyctus spp., Meligethes aeneus, Melolontha melolontha, Migdolus spp., Monochamus spp., Naupactus xanthographus, Niptus hololeucus, Oryctes rhinoceros, Oryzaephilus surinamensis, Otiorrhynchus sulcatus, Oxycetonia jucunda, Phaedon cochleariae, Phyllophaga spp., Popillia japonica, Premnotrypes spp., Psylliodes chrysocephala, Ptinus spp., Rhizobius ventralis, Rhizopertha dominica, Sitophilus spp., Sphenophorus spp., Sternechus spp., Symphyletes spp., Tenebrio molitor, Tribolium spp., Trogodei spp., Tychius spp., Xylotrechus spp., Zabrus spp.

From the order of the Collembola, for example, Onychiurus armatus.

From the order of the Dermaptera, for example, Forficula auricularia.

From the order of the Diplopoda, for example, Blaniulus guttulatus.

From the order of the Diptera, for example, Aedes spp., Anopheles spp., Bibio hortulanus, Calliphora erythrocephala, Ceratitis capitata, Chrysomyia spp., Cochliomyia spp., Cordylobia anthropophaga, Culex spp., Cuterebra spp., Dacus oleae, Dermatobia hominis, Drosophila spp., Fannia spp., Gastrophilus spp., Hylemyia spp., Hyppobosca spp., Hypoderma spp., Liriomyza spp. Lucilia spp., Musca spp., Nezara spp., Oestrus spp., Oscinella frit, Pegomyia hyoscyami, Phorbia spp., Stomoxys spp., Tabanus spp., Tannia spp., Tipula paludosa, Wohlfahrtia spp.

From the class of the Gastropoda, for example, Arion spp., Biomphalaria spp., Bulinus spp., Deroceras spp., Galba spp., Lymnaea spp., Oncomelania spp., Succinea spp.

From the class of the helminths, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp, Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp., Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.

It is furthermore possible to control protozoa, such as Eimeria.

From the order of the Heteroptera, for example, Anasa tristis, Antestiopsis spp., Blissus spp., Calocoris spp., Campylomma livida, Cavelerius spp., Cimex spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptoglossus phyllopus, Lygus spp., Macropes excavatus, Miridae, Nezara spp., Oebalus spp., Pentomidae, Piesma quadrata, Piezodorus spp., Psallus seriatus, Pseudacysta persea, Rhodnius spp., Sahlbergella singularis, Scotinophora spp., Stephanitis nashi, Tibraca spp., Triatoma spp.

From the order of the Homoptera, for example, Acyrthosipon spp., Aeneolamia spp., Agonoscena spp., Aleurodes spp., Aleurolobus barodensis, Aleurothrixus spp., Amrasca spp., Anuraphis cardui, Aonidiella spp., Aphanostigma piri, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona marginata, Carneocephala fulgida, Ceratovacuna lanigera, Cercopidae, Ceroplastes spp., Chaetosiphon fragaefolii, Chionaspis tegalensis, Chlorita onukii, Chromaphis juglandicola, Chrysomphalus ficus, Cicadulina mbila, Coccomytilus halli, Coccus spp., Cryptomyzus ribis, Dalbulus spp., Dialeurodes spp., Diaphorina spp., Diaspis spp., Doralis spp., Drosicha spp., Dysaphis spp., Dysmicoccus spp., Empoasca spp., Eriosoma spp., Erythroneura spp., Euscelis bilobatus, Geococcus coffeae, Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Mahanarva fimbriolata, Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Protopulvinaria pyriformis, Pseudaulacaspis pentagona, Pseudococcus spp., Psylla spp., Pteromalus spp., Pyrilla spp., Quadraspidiotus spp., Quesada gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes vaporariorum, Trioza spp., Typhlocyba spp., Unaspis spp., Viteus vitifolii.

From the order of the Hymenoptera, for example, Diprion spp., Hoplocampa spp., Lasius spp., Monomorium pharaonis and Vespa spp.

From the order of the Isopoda, for example, Armadillidium vulgare, Oniscus asellus and Porcellio scaber.

From the order of the Isoptera, for example, Reticulitermes spp. and Odontotermes spp.

From the order of the Lepidoptera, for example, Acronicta major, Aedia leucomelas, Agrotis spp., Alabama argillacea, Anticarsia spp., Barathra brassicae, Bucculatrix thurberiella, Bupalus piniarius, Cacoecia podana, Capua reticulana, Carpocapsa pomonella, Chematobia brumata, Chilo spp., Choristoneura fumiferana, Clysia ambiguella, Cnaphalocerus spp., Earias insulana, Ephestia kuehniella, Euproctis chrysorrhoea, Euxoa spp., Feltia spp., Galleria mellonella, Helicoverpa spp., Heliothis spp., Hofmannophila pseudospretella, Homona magnanima, Hyponomeuta padella, Laphygma spp., Lithocolletis blancardella, Lithophane antennata, Loxagrotis albicosta, Lymantria spp., Malacosoma neustria, Mamestra brassicae, Mocis repanda, Mythimna separata, Oria spp., Oulema oryzae, Panolis flammea, Pectinophora gossypiella, Phyllocnistis citrella, Pieris spp., Plutella xylostella, Prodenia spp., Pseudaletia spp., Pseudoplusia includens, Pyrausta nubilalis, Spodoptera spp., Thermesia gemmatalis, Tinea pellionella, Tineola bisselliella, Tortrix viridana, Trichoplusia spp.

From the order of the Orthoptera, for example, Acheta domesticus, Blatta orientalis, Blattella germanica, Gryllotalpa spp., Leucophaea maderae, Locusta spp., Melanoplus spp., Periplaneta americana, Schistocerca gregaria.

From the order of the Siphonaptera, for example, Ceratophyllus spp. and Xenopsylla cheopis.

From the order of the Symphyla, for example, Scutigerella immaculata.

From the order of the Thysanoptera, for example, Baliothrips bifoimis, Enneothrips flavens, Frankliniella spp., Heliothrips spp., Hercinothrips femoralis, Kakothrips spp., Rhipiphorothrips cruentatus, Scirtothrips spp., Taeniothrips cardamoni and Thrips spp.

From the order of the Thysanura, for example, Lepisma saccharina.

The phytoparasitic nematodes include, for example, Anguina spp., Aphelenchoides spp., Belonoaimus spp., Bursaphelenchus spp., Ditylenchus dipsaci, Globodera spp., Heliocotylenchus spp., Heterodera spp., Longidorus spp., Meloidogyne spp., Pratylenchus spp., Radopholus similis, Rotylenchus spp., Trichodorus spp., Tylenchorhynchus spp., Tylenchulus spp., Tylenchulus semipenetrans and Xiphinema spp.

If appropriate, the compounds according to the invention can, at certain concentrations or application rates, also be used as herbicides, safeners, growth regulators or agents to improve plant properties, or as microbicides, for example as fungicides, antimycotics, bactericides, viricides (including agents against viroids) or as agents against MLO (mycoplasma-like organisms) and RLO (rickettsia-like organisms). If appropriate, they can also be used as intermediates or precursors for the synthesis of other active compounds.

The active compounds can be converted into the customary formulations, such as solutions, emulsions, wettable powders, water- and oil-based suspensions, powders, dusts, pastes, soluble powders, soluble granules, granules for broadcasting, suspoemulsion concentrates, natural compounds impregnated with active compound, synthetic substances impregnated with active compound, fertilizers and also microencapsulations in polymeric substances.

These formulations are produced in a known manner, for example by mixing the active compounds with extenders, that is, liquid solvents and/or solid carriers, optionally with the use of surfactants, that is to say emulsifiers and/or dispersants and/or foam-formers. The formulations are prepared either in suitable facilities or else before or during application.

Suitable for use as auxiliaries are substances which are suitable for imparting to the composition itself and/or to preparations derived therefrom (for example spray liquors, seed dressings) particular properties such as certain technical properties and/or also particular biological properties. Typical suitable auxiliaries are: extenders, solvents and carriers.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

If the extender used is water, it is also possible to employ, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethyl sulphoxide, and also water.

Suitable carriers are:

for example ammonium salts and ground natural minerals such as kaolins, clays, talc, chalk, quartz, attapulgite, montmorillonite or diatomaceous earth, and ground synthetic materials such as highly disperse silica, alumina and silicates; suitable solid carriers for granules are: for example, crushed and fractionated natural rocks such as calcite, marble, pumice, sepiolite and dolomite, and also synthetic granules of inorganic and organic meals, and also granules of organic material such as paper, sawdust, coconut shells, maize cobs and tobacco stalks; suitable emulsifiers and/or foam-formers are: for example, nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulfonates and also protein hydrolysates; suitable dispersants are nonionic and/or ionic substances, for example from the classes of the alcohol-POE and/or —POP ethers, acid and/or POP POE esters, alkylaryl and/or POP POE ethers, fat and/or POP POE adducts, POE- and/or POP-polyol derivatives, POE- and/or POP-sorbitan or -sugar adducts, alkyl or aryl sulphates, alkyl- or arylsulphonates and alkyl or aryl phosphates or the corresponding PO-ether adducts. Furthermore, suitable oligomers or polymers, for example those derived from vinylic monomers, from acrylic acid, from EO and/or PO alone or in combination with, for example, (poly)alcohols or (poly)amines. It is also possible to employ lignin and its sulphonic acid derivatives, unmodified and modified celluloses, aromatic and/or aliphatic sulphonic acids and also their adducts with formaldehyde.

Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations.

It is possible to use colorants such as inorganic pigments, for example iron oxide, titanium oxide and Prussian Blue, and organic colorants such as alizarin colorants, azo colorants and metal phthalocyanine colorants, and trace nutrients such as salts of iron, manganese, boron, copper, cobalt, molybdenum and zinc.

Other possible additives are perfumes, mineral or vegetable oils which are optionally modified, waxes and nutrients (including trace nutrients), such as salts of iron, manganese, boron, copper, cobalt, molybdenum and zinc.

Stabilizers, such as low-temperature stabilizers, preservatives, antioxidants, light stabilizers or other agents which improve chemical and/or physical stability, may also be present.

The active compound according to the invention can be present in its commercially available formulations and in the use fauns, prepared from these formulations, as a mixture with other active compounds, such as insecticides, attractants, sterilizing agents, bactericides, acaricides, nematicides, fungicides, growth-regulating substances, herbicides, safeners, fertilizers, semiochemicals or else agents for improving plant properties.

When used as insecticides, the active compounds according to the invention can furthermore be present in their commercially available formulations and in the use forms, prepared from these formulations, as a mixture with synergistic agents. Synergistic agents are compounds which increase the action of the active compounds, without it being necessary for the synergistic agent added to be active itself.

When used as insecticides, the active compounds according to the invention can furthermore be present in their commercially available formulations and in the use forms, prepared from these formulations, as a mixture with inhibitors which reduce degradation of the active compound after use in the environment of the plant, on the surface of parts of plants or in plant tissues.

The active compound content of the use forms prepared from the commercially available formulations can vary within wide limits. The total active compound concentration, or the active compound concentration of the individual active compounds of the use forms is in the range of from 0.00000001 to 97% by weight of active compound, preferably in the range of from 0.0000001 to 97% by weight, particularly preferably in the range of from 0.000001 to 83% by weight or 0.000001 to 5% by weight, and very particularly preferably in the range of from 0.0001 to 1% by weight.

The compounds are employed in a customary manner appropriate for the use forms.

All plants and plant parts can be treated in accordance with the invention. By plants are understood here all plants and plant populations such as desired and undesired wild plants or crop plants (including naturally occurring crop plants). Crop plants can be plants which can be obtained by conventional breeding and optimization methods or by biotechnological and genetic engineering methods or combinations of these methods, including the transgenic plants and including the plant varieties which can or cannot be protected by varietal property rights. Parts of plants are to be understood as meaning all above-ground and below-ground parts and organs of plants, such as shoot, leaf, flower and root, examples which may be mentioned being leaves, needles, stems, trunks, flowers, fruit-bodies, fruits and seeds and also roots, tubers and rhizomes. The plant parts also include harvested material and also vegetative and generative propagation material, for example cuttings, tubers, rhizomes, slips and seed.

Treatment according to the invention of the plants and plant parts with the active compounds is carried out directly or by allowing the compounds to act on their surroundings, environment or storage space by the customary treatment methods, for example by immersion, spraying, evaporation, fogging, scattering, painting on, injection and, in the case of propagation material, in particular in the case of seeds, also by applying one or more coats.

The following plants may be mentioned as plants which can be treated according to the invention: cotton, flax, grapevine, fruit, vegetables, such as Rosaceae sp. (for example pome fruits such as apples and pears, but also stone fruits such as apricots, cherries, almonds and peaches, and soft fruits such as strawberries), Ribesioidae sp., Juglandaceae sp., Betulaceae sp., Anacardiaceae sp., Fagaceae sp., Moraceae sp., Oleaceae sp., Actimidaceae sp., Lauraceae sp., Musaceae sp. (for example banana plants and banana plantations), Rubiaceae sp. (for example coffee), Theaceae sp., Sterculiceae sp., Rutaceae sp. (for example lemons, oranges and grapefruit); Solanaceae sp. (for example tomatoes), Liliaceae sp., Asteraceae sp. (for example lettuce), Umbelliferae sp., Cruciferae sp., Chenopodiaceae sp., Cucurbitaceae sp. (for example cucumber), Alliaceae sp. (for example leeks, onions), Papilionaceae sp. (for example peas); major crop plants such as Gramineae sp. (for example maize, turf, cereals such as wheat, rye, rice, barley, oats, millet and triticale), Asteraceae sp. (for example sunflower), Brassicaceae sp. (for example white cabbage, red cabbage, broccoli, cauliflower, Brussels sprouts, pak Choi, kohlrabi, radishes, and also http://de.wikipedia.org/wiki/Rapsoil seed rape, mustard, horseradish and cress), Fabacae sp. (for example beans, peanuts), Papilionaceae sp. (for example soya beans), Solanaceae sp. (for example potatoes), Chenopodiaceae sp. (for example sugar beet, fodder beet, Swiss chard, beetroot); useful plants and ornamental plants in gardens and forests; and in each case genetically modified types of these plants.

The active compounds according to the invention are particularly suitable for the treatment of seed. Here, particular mention may be made of the active compounds according to the invention mentioned above as preferred or particularly preferred. Thus, most of the damage to crop plants which is caused by pests occurs as early as when the seed is infested during storage and after the seed is introduced into the soil, and during and immediately after germination of the plants. This phase is particularly critical since the roots and shoots of the growing plant are particularly sensitive and even minor damage can lead to the death of the whole plant. Protecting the seed and the germinating plant by the use of suitable compositions is therefore of particularly great interest.

The control of pests by treating the seed of plants has been known for a long time and is the subject of continuous improvements. However, the treatment of seed entails a series of problems which cannot always be solved in a satisfactory manner. Thus, it is desirable to develop methods for protecting the seed and the germinating plant which dispense with the additional application of crop protection products after sowing or after emergence of the plants. It is furthermore desirable to optimize the amount of active compound employed in such a way as to provide optimum protection for the seed and the germinating plant from attack by pests, but without damaging the plant itself by the active compound employed. In particular, methods for the treatment of seed should also take into consideration the intrinsic insecticidal properties of transgenic plants in order to achieve optimum protection of the seed and also the germinating plant with a minimum of crop protection products being employed.

The present invention therefore in particular also relates to a method for the protection of seed and germinating plants, from attack by pests, by treating the seed with an active compound according to the invention. The invention likewise relates to the use of the active compounds according to the invention for the treatment of seed for protecting the seed and the resulting plant from pests. Furthermore, the invention relates to seed which has been treated with an active compound according to the invention so as to afford protection from pests. The invention also relates to seed where an active compound of the formula I has been applied as component of a coating or as a further layer or further layers in addition to a coating.

One of the advantages of the present invention is that the particular systemic properties of some of the active compounds according to the invention mean that treatment of the seed with these active compounds not only protects the seed itself, but also the resulting plants after emergence, from pests. In this manner, the immediate treatment of the crop at the time of sowing or shortly thereafter can be dispensed with.

Furthermore, it must be considered as advantageous that the active compounds according to the invention can also be employed in particular in transgenic seed, the plants arising from this seed being capable of expressing a protein directed against pests. By treating such seed with the active compounds according to the invention, certain pests can be controlled merely by the expression of the, for example, insecticidal protein, and additionally damage to the seed may be averted by the active compounds according to the invention.

The active compounds according to the invention are suitable for protecting seed of any plant variety as already mentioned above which is employed in agriculture, in the greenhouse, in forests or in horticulture. In particular, this takes the form of seed of maize, peanut, canola, oilseed rape, poppy, soya beans, cotton, beet (for example sugar beet and fodder beet), rice, millet, wheat, barley, oats, rye, sunflower, tobacco, potatoes or vegetables (for example tomatoes, cabbage species). The active compounds according to the invention are likewise suitable for treating the seed of fruit plants and vegetables as already mentioned above. The treatment of the seed of maize, soya beans, cotton, wheat and canola or oilseed rape is of particular importance.

As already mentioned above, the treatment of transgenic seed with an active compound according to the invention is also of particular importance. This takes the form of seed of plants which, as a rule, comprise at least one heterologous gene which governs the expression of a polypeptide with in particular insecticidal properties. In this context, the heterologous genes in transgenic seed may be derived from microorganisms such as Bacillus, Rhizobium, Pseudomonas, Serratia, Trichodeima, Clavibacter, Glomus or Gliocladium. The present invention is particularly suitable for the treatment of transgenic seed which comprises at least one heterologous gene originating from Bacillus sp. and whose gene product shows activity against the European corn borer and/or the corn root worm. It is particularly preferably a heterologous gene derived from Bacillus thuringiensis.

Within the context of the present invention, the active compound according to the invention is applied to the seed either alone or in a suitable formulation. Preferably, the seed is treated in a state in which it is stable enough to avoid damage during treatment. In general, the seed may be treated at any point in time between harvest and sowing. The seed usually used has been separated from the plant and freed from cobs, shells, stalks, coats, hairs or the flesh of the fruits.

When treating the seed, care must generally be taken that the amount of the active compound according to the invention applied to the seed and/or the amount of further additives is chosen in such a way that the germination of the seed is not adversely affected, or that the resulting plant is not damaged. This must be borne in mind in particular in the case of active compounds which can have phytotoxic effects at certain application rates.

The compositions according to the invention can be applied directly, i.e. without containing any other components and undiluted. In general, it is preferred to apply the compositions to the seed in the form of a suitable formulation. Suitable formulations and methods for treating seed are known to the person skilled in the art and are described, for example, in the following documents: U.S. Pat. No. 4,272,417 A, U.S. Pat. No. 4,245,432 A, U.S. Pat. No. 4,808,430 A, U.S. Pat. No. 5,876,739 A, US 2003/0176428 A1, WO 2002/080675 A1, WO 2002/028186 A2.

The active compounds which can be used in accordance with the invention can be converted into the customary seed-dressing formulations, such as solutions, emulsions, suspensions, powders, foams, slurries or other coating compositions for seed, and also ULV formulations.

These formulations are prepared in a known manner, by mixing the active compounds with customary additives such as, for example, customary extenders and also solvents or diluents, colorants, wetting agents, dispersants, emulsifiers, antifoams, preservatives, secondary thickeners, adhesives, gibberellins and also water.

Colorants which may be present in the seed-dressing formulations which can be used in accordance with the invention are all colorants which are customary for such purposes. In this context, not only pigments, which are sparingly soluble in water, but also dyes, which are soluble in water, may be used. Examples which may be mentioned are the colorants known by the names Rhodamin B, C.I. Pigment Red 112 and C.I. Solvent Red 1.

Suitable wetting agents which may be present in the seed-dressing formulations which can be used in accordance with the invention are all substances which promote wetting and which are conventionally used for the formulation of agrochemical active compounds. Preference is given to using alkylnaphthalenesulphonates, such as diisopropyl- or diisobutylnaphthalenesulphonates.

Suitable dispersants and/or emulsifiers which may be present in the seed-dressing formulations which can be used in accordance with the invention are all nonionic, anionic and cationic dispersants conventionally used for the formulation of agrochemical active compounds. Preference is given to using nonionic or anionic dispersants or mixtures of nonionic or anionic dispersants. Suitable nonionic dispersants which may be mentioned are, in particular, ethylene oxide/propylene oxide block polymers, alkylphenol polyglycol ethers and tristryrylphenol polyglycol ether, and their phosphated or sulphated derivatives. Suitable anionic dispersants are, in particular, lignosulphonates, polyacrylic acid salts and arylsulphonate/formaldehyde condensates.

Antifoams which may be present in the seed-dressing formulations which can be used in accordance with the invention are all foam-inhibiting substances conventionally used for the formulation of agrochemical active compounds. Silicone antifoams and magnesium stearate can preferably be used.

Preservatives which may be present in the seed-dressing formulations which can be used in accordance with the invention are all substances which can be employed for such purposes in agrochemical compositions. Dichlorophene and benzyl alcohol hemiformal may be mentioned by way of example.

Secondary thickeners which may be present in the seed-dressing formulations which can be used in accordance with the invention are all substances which can be employed for such purposes in agrochemical compositions. Cellulose derivatives, acrylic acid derivatives, xanthan, modified clays and finely divided silica are preferred.

Adhesives which may be present in the seed-dressing formulations which can be used in accordance with the invention are all customary binders which can be employed in seed-dressing products. Polyvinylpyrrolidone, polyvinyl acetate, polyvinyl alcohol and tylose may be mentioned as being preferred.

Gibberellins which can be present in the seed-dressing formulations which can be used in accordance with the invention are preferably the gibberellins A1, A3 (=gibberellic acid), A4 and A7; gibberellic acid is especially preferably used. The gibberellins are known (cf. R. Wegler “Chemie der Pflanzenschutz- and Schädlingsbekampfungsmittel” [Chemistry of crop protection agents and pesticides], vol. 2, Springer Verlag, 1970, p. 401-412).

The seed-dressing formulations which can be used in accordance with the invention can be employed for the treatment of a wide range of seed, including the seed of transgenic plants, either directly or after previously having been diluted with water. In this context, additional synergistic effects may also occur in cooperation with the substances formed by expression.

All mixers which can conventionally be employed for the seed-dressing operation are suitable for treating seed with the seed-dressing formulations which can be used in accordance with the invention or with the preparations prepared therefrom by addition of water. Specifically, a procedure is followed during the seed-dressing operation in which the seed is placed into a mixer, the specific desired amount of seed-dressing formulations, either as such or after previously having been diluted with water, is added, and everything is mixed until the formulation is distributed uniformly on the seed. If appropriate, this is followed by a drying process.

The method of treatment according to the invention can be used in the treatment of genetically modified organisms (GMOs), e.g. plants or seeds. Genetically modified plants (or transgenic plants) are plants in which a heterologous gene has been stably integrated into the genome. The expression “heterologous gene” essentially means a gene which is provided or assembled outside the plant and when introduced in the nuclear, chloroplastic or mitochondrial genome gives the transformed plant new or improved agronomic or other properties by expressing a protein or polypeptide of interest or by downregulating or silencing other gene(s) which are present in the plant (using for example antisense technology, cosuppression technology or RNAi technology [RNA interference]). A heterologous gene that is located in the genome is also called a transgene. A transgene that is defined by its particular location in the plant genome is called a transformation or transgenic event.

Plants and plant varieties which are preferably treated according to the invention include all plants which have genetic material which imparts particularly advantageous, useful traits to these plants (whether obtained by breeding and/or biotechnological means).

Plants and plant varieties which are also preferably treated according to the invention are resistant against one or more biotic stress factors, i.e. said plants have a better defence against animal and microbial pests, such as against nematodes, insects, mites, phytopathogenic fungi, bacteria, viruses and/or viroids.

Plants and plant varieties which may also be treated according to the invention are those plants which are resistant to one or more abiotic stress factors. Abiotic stress conditions may include, for example, drought, cold temperature exposure, heat exposure, osmotic stress, waterlogging, increased soil salinity, increased exposure to minerals, exposure to ozone, exposure to strong light, limited availability of nitrogen nutrients, limited availability of phosphorus nutrients or shade avoidance.

Plants and plant varieties which may also be treated according to the invention are those plants characterized by enhanced yield characteristics. Enhanced yield in said plants can be the result of, for example, improved plant physiology, growth and development, such as water use efficiency, water retention efficiency, improved nitrogen use, enhanced carbon assimilation, improved photosynthesis, increased germination efficiency and accelerated maturation. Yield can furthermore be affected by improved plant architecture (under stress and non-stress conditions), including early flowering, flowering control for hybrid seed production, seedling vigour, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield traits include seed composition, such as carbohydrate content, protein content, oil content and composition, nutritional value, reduction in anti-nutritional compounds, improved processability and better storage stability.

Plants that may be treated according to the invention are hybrid plants that already express the characteristics of heterosis, or hybrid vigour, which results in generally higher yield, increased vigour, better health and better resistance towards biotic and abiotic stress factors. Such plants are typically made by crossing an inbred male-sterile parent line (the female parent) with another inbred male-fertile parent line (the male parent). Hybrid seed is typically harvested from the male-sterile plants and sold to growers. Male-sterile plants can sometimes (e.g. in maize) be produced by detasseling (i.e. the mechanical removal of the male reproductive organs or male flowers) but, more typically, male sterility is the result of genetic determinants in the plant genome. In that case, and especially when seed is the desired product to be harvested from the hybrid plants, it is typically useful to ensure that male fertility in hybrid plants, which contain the genetic determinants responsible for male sterility, is fully restored. This can be accomplished by ensuring that the male parents have appropriate fertility restorer genes which are capable of restoring the male fertility in hybrid plants that contain the genetic determinants responsible for male sterility. Genetic determinants for male sterility may be located in the cytoplasm. Examples of cytoplasmic male sterility (CMS) were for instance described in Brassica species (WO 1992/005251, WO 1995/009910, WO 1998/27806, WO 2005/002324, WO 2006/021972 and U.S. Pat. No. 6,229,072). However, genetic determinants for male sterility can also be located in the nuclear genome. Male-sterile plants can also be obtained by plant biotechnology methods such as genetic engineering. A particularly useful means of obtaining male-sterile plants is described in WO 89/10396 in which, for example, a ribonuclease such as a barnase is selectively expressed in the tapetum cells in the stamens. Fertility can then be restored by expression in the tapetum cells of a ribonuclease inhibitor such as barstar (e.g. WO 1991/002069).

Plants or plant varieties (obtained by plant biotechnology methods such as genetic engineering) which may be treated according to the invention are herbicide-tolerant plants, i.e. plants made tolerant to one or more given herbicides. Such plants can be obtained either by genetic transformation, or by selection of plants containing a mutation imparting such herbicide tolerance.

Herbicide-tolerant plants are for example glyphosate-tolerant plants, i.e. plants made tolerant to the herbicide glyphosate or salts thereof. For example, glyphosate-tolerant plants can be obtained by transforming the plant with a gene encoding the enzyme 5-enolpyruvylshikimate-3-phosphate synthase (EPSPS). Examples of such EPSPS genes are the AroA gene (mutant CT7) of the bacterium Salmonella typhimurium (Comai et al., Science (1983), 221, 370-371), the CP4 gene of the bacterium Agrobacterium sp. (Barry et al., Curr. Topics Plant Physiol. (1992), 7, 139-145), the genes encoding a petunia EPSPS (Shah et al., Science (1986), 233, 478-481), a tomato EPSPS (Gasser et al., J. Biol. Chem. (1988), 263, 4280-4289) or an Eleusine EPSPS (WO 2001/66704). It can also be a mutated EPSPS, as described, for example, in EP-A 0837944, WO 2000/066746, WO 2000/066747 or WO 2002/026995. Glyphosate-tolerant plants can also be obtained by expressing a gene that encodes a glyphosate oxidoreductase enzyme as described in U.S. Pat. No. 5,776,760 and U.S. Pat. No. 5,463,175. Glyphosate-tolerant plants can also be obtained by expressing a gene that encodes a glyphosate acetyltransferase enzyme as described, for example, in WO 2002/036782, WO 2003/092360, WO 2005/012515 and WO 2007/024782. Glyphosate-tolerant plants can also be obtained by selecting plants containing naturally occurring mutations of the abovementioned genes as described, for example, in WO 2001/024615 or WO 2003/013226.

Other herbicide-resistant plants are for example plants which have been made tolerant to herbicides inhibiting the enzyme glutamine synthase, such as bialaphos, phosphinothricin or glufosinate. Such plants can be obtained by expressing an enzyme detoxifying the herbicide or a mutant glutamine synthase enzyme that is resistant to inhibition. One such efficient detoxifying enzyme is, for example, an enzyme encoding a phosphinothricin acetyltransferase (such as the bar or pat protein from Streptomyces species for example). Plants expressing an exogenous phosphinothricin acetyltransferase have been described, for example, in U.S. Pat. No. 5,561,236; U.S. Pat. No. 5,648,477; U.S. Pat. No. 5,646,024; U.S. Pat. No. 5,273,894; U.S. Pat. No. 5,637,489; U.S. Pat. No. 5,276,268; U.S. Pat. No. 5,739,082; U.S. Pat. No. 5,908,810 and U.S. Pat. No. 7,112,665.

Further herbicide-tolerant plants are also plants that have been made tolerant to the herbicides inhibiting the enzyme hydroxyphenylpyruvatedioxygenase (HPPD). Hydroxyphenylpyruvatedioxygenases are enzymes that catalyse the reaction in which para-hydroxyphenylpyruvate (HPP) is transformed into homogentisate. Plants tolerant to HPPD-inhibitors can be transformed with a gene encoding a naturally-occurring resistant HPPD enzyme, or a gene encoding a mutated HPPD enzyme according to WO 1996/038567, WO 1999/024585 and WO 1999/024586. Tolerance to HPPD-inhibitors can also be obtained by transforming plants with genes encoding certain enzymes enabling the formation of homogentisate despite the inhibition of the native HPPD enzyme by the HPPD-inhibitor. Such plants and genes are described in WO 1999/034008 and WO 2002/36787. Tolerance of plants to HPPD inhibitors can also be improved by transforming plants with a gene encoding an enzyme prephenate dehydrogenase in addition to a gene encoding an HPPD-tolerant enzyme, as described in WO 2004/024928.

Further herbicide-resistant plants are plants that have been made tolerant to acetolactate synthase (ALS) inhibitors. Known ALS inhibitors include, for example, sulphonylurea, imidazolinone, triazolopyrimidines, pyrimidinyl oxy(thio)benzoates, and/or sulphonylaminocarbonyltriazolinone herbicides. Different mutations in the ALS enzyme (also known as acetohydroxy acid synthase, AHAS) are known to confer tolerance to different herbicides and groups of herbicides, as described, for example, in Tranel and Wright, Weed Science (2002), 50, 700-712, and also in U.S. Pat. No. 5,605,011, U.S. Pat. No. 5,378,824, U.S. Pat. No. 5,141,870 and U.S. Pat. No. 5,013,659. The production of sulphonylurea-tolerant plants and imidazolinone-tolerant plants has been described in U.S. Pat. No. 5,605,011; U.S. Pat. No. 5,013,659; U.S. Pat. No. 5,141,870; U.S. Pat. No. 5,767,361; U.S. Pat. No. 5,731,180; U.S. Pat. No. 5,304,732; U.S. Pat. No. 4,761,373; U.S. Pat. No. 5,331,107; U.S. Pat. No. 5,928,937; and U.S. Pat. No. 5,378,824; and also in the international publication WO 1996/033270. Further imidazolinone-tolerant plants have also been described, for example in WO 2004/040012, WO 2004/106529, WO 2005/020673, WO 2005/093093, WO 2006/007373, WO 2006/015376, WO 2006/024351 and WO 2006/060634. Further sulphonylurea- and imidazolinone-tolerant plants have also been described, for example in WO 2007/024782.

Other plants tolerant to imidazolinone and/or sulphonylurea can be obtained by induced mutagenesis, by selection in cell cultures in the presence of the herbicide or by mutation breeding, as described, for example, for soya beans in U.S. Pat. No. 5,084,082, for rice in WO 1997/41218, for sugar beet in U.S. Pat. No. 5,773,702 and WO 1999/057965, for lettuce in U.S. Pat. No. 5,198,599 or for sunflower in WO 2001/065922.

Plants or plant varieties (obtained by plant biotechnology methods such as genetic engineering) which may also be treated according to the invention are insect-resistant transgenic plants, i.e. plants made resistant to attack by certain target insects. Such plants can be obtained by genetic transformation, or by selection of plants containing a mutation imparting such insect resistance.

In the present context, the term “insect-resistant transgenic plant” includes any plant containing at least one transgene comprising a coding sequence encoding:
1) an insecticidal crystal protein from Bacillus thuringiensis or an insecticidal portion thereof, such as the insecticidal crystal proteins listed by Crickmore et al., Microbiology and Molecular Biology Reviews (1998), 62, 807-813, updated by Crickmore et al. (2005) in the Bacillus thuringiensis toxin nomenclature, online at: http://www.lifesci.sussex.ac.uk/Home/Neil_Crickmore/Bt/), or insecticidal portions thereof, for example proteins of the Cry protein classes Cry1Ab, Cry1Ac, Cry1F, Cry2Ab, Cry3Ae or Cry3Bb or insecticidal portions thereof; or
2) a crystal protein from Bacillus thuringiensis or a portion thereof which is insecticidal in the presence of a second other crystal protein than Bacillus thuringiensis or a portion thereof, such as the binary toxin made up of the Cy34 and Cy35 crystal proteins (Moellenbeck et al., Nat. Biotechnol. (2001), 19, 668-72; Schnepf et al., Applied Environm. Microb. (2006), 71, 1765-1774); or
3) a hybrid insecticidal protein comprising parts of two different insecticidal crystal proteins from Bacillus thuringiensis, such as a hybrid of the proteins of 1) above or a hybrid of the proteins of 2) above, for example the Cry1A.105 protein produced by maize event MON98034 (WO 2007/027777); or
4) a protein of any one of points 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation, such as the Cry3Bb1 protein in maize events MON863 or MON88017, or the Cry3A protein in maize event MIR604; or
5) an insecticidal secreted protein from Bacillus thuringiensis or Bacillus cereus, or an insecticidal portion thereof, such as the vegetative insecticidal proteins (VIP) listed at: http://www.lifesci.sussex.ac.uk/home/Neil_Crickmore/Bt/vip.html, for example proteins from the VIP3Aa protein class; or
6) a secreted protein from Bacillus thuringiensis or Bacillus cereus which is insecticidal in the presence of a second secreted protein from Bacillus thuringiensis or B. cereus, such as the binary toxin made up of the VIP1A and VIP2A proteins (WO 1994/21795); or
7) a hybrid insecticidal protein comprising parts from different secreted proteins from Bacillus thuringiensis or Bacillus cereus, such as a hybrid of the proteins in 1) above or a hybrid of the proteins in 2) above; or
8) a protein of any one of points 1) to 3) above wherein some, particularly 1 to 10, amino acids have been replaced by another amino acid to obtain a higher insecticidal activity to a target insect species, and/or to expand the range of target insect species affected, and/or because of changes induced in the encoding DNA during cloning or transformation (while still encoding an insecticidal protein), such as the VIP3Aa protein in cotton event COT 102.

Of course, insect-resistant transgenic plants, as used herein, also include any plant comprising a combination of genes encoding the proteins of any one of the above classes 1 to 8. In one embodiment, an insect-resistant plant contains more than one transgene encoding a protein of any one of the above classes 1 to 8, to expand the range of target insect species affected or to delay insect resistance development to the plants, by using different proteins insecticidal to the same target insect species but having a different mode of action, such as binding to different receptor binding sites in the insect.

Plants or plant varieties (obtained by plant biotechnology methods such as genetic engineering) which may also be treated according to the invention are tolerant to abiotic stress factors. Such plants can be obtained by genetic transformation, or by selection of plants containing a mutation imparting such stress resistance. Particularly useful stress-tolerant plants include the following:
a. plants which contain a transgene capable of reducing the expression and/or the activity of the poly(ADP-ribose)polymerase (PARP) gene in the plant cells or plants, as described in WO 2000/004173 or EP 04077984.5 or EP 06009836.5.
b. plants which contain a stress tolerance-enhancing transgene capable of reducing the expression and/or the activity of the PARG encoding genes of the plants or plant cells, as described, for example, in WO 2004/090140;
c. plants which contain a stress tolerance-enhancing transgene coding for a plant-functional enzyme of the nicotinamide adenine dinucleotide salvage biosynthesis pathway, including nicotinamidase, nicotinate phosphoribosyltransferase, nicotinic acid mononucleotide adenyl transferase, nicotinamide adenine dinucleotide synthetase or nicotinamide phosphoribosyltransferase, as described, for example, in EP 04077624.7 or WO 2006/133827 or PCT/EP07/002,433.

Plants or plant varieties (obtained by plant biotechnology methods such as genetic engineering) which may also be treated according to the invention show altered quantity, quality and/or storage stability of the harvested product and/or altered properties of specific ingredients of the harvested product such as, for example:
1) Transgenic plants which synthesize a modified starch which is altered with respect to its chemophysical traits, in particular the amylose content or the amylose/amylopectin ratio, the degree of branching, the average chain length, the distribution of the side chains, the viscosity behaviour, the gel resistance, the grain size and/or grain morphology of the starch in comparison to the synthesized starch in wild-type plant cells or plants, such that this modified starch is better suited for certain applications. These transgenic plants synthesizing a modified starch are described, for example, in EP 0571427, WO 1995/004826, EP 0719338, WO 1996/15248, WO 1996/19581, WO 1996/27674, WO 1997/11188, WO 1997/26362, WO 1997/32985, WO 1997/42328, WO 1997/44472, WO 1997/45545, WO 1998/27212, WO 1998/40503, WO 99/58688, WO 1999/58690, WO 1999/58654, WO 2000/008184, WO 2000/008185, WO 2000/28052, WO 2000/77229, WO 2001/12782, WO 2001/12826, WO 2002/101059, WO 2003/071860, WO 2004/056999, WO 2005/030942, WO 2005/030941, WO 2005/095632, WO 2005/095617, WO 2005/095619, WO 2005/095618, WO 2005/123927, WO 2006/018319, WO 2006/103107, WO 2006/108702, WO 2007/009823, WO 2000/22140, WO 2006/063862, WO 2006/072603, WO 2002/034923, EP 06090134.5, EP 06090228.5, EP 06090227.7, EP 07090007.1, EP 07090009.7, WO 2001/14569, WO 2002/79410, WO 2003/33540, WO 2004/078983, WO 2001/19975, WO 1995/26407, WO 1996/34968, WO 1998/20145, WO 1999/12950, WO 1999/66050, WO 1999/53072, U.S. Pat. No. 6,734,341, WO 2000/11192, WO 1998/22604, WO 1998/32326, WO 2001/98509, WO 2001/98509, WO 2005/002359, U.S. Pat. No. 5,824,790, U.S. Pat. No. 6,013,861, WO 1994/004693, WO 1994/009144, WO 1994/11520, WO 1995/35026 and WO 1997/20936.
2) Transgenic plants which synthesize non-starch carbohydrate polymers or which synthesize non-starch carbohydrate polymers with altered properties in comparison to wild-type plants without genetic modification. Examples are plants which produce polyfructose, especially of the inulin and levan type, as described in EP 0663956, WO 1996/001904, WO 1996/021023, WO 1998/039460 and WO 1999/024593, plants which produce alpha-1,4-glucans, as described in WO 1995/031553, US 2002/031826, U.S. Pat. No. 6,284,479, U.S. Pat. No. 5,712,107, WO 1997/047806, WO 1997/047807, WO 1997/047808 and WO 2000/14249, plants which produce alpha-1,6-branched alpha-1,4-glucans, as described in WO 2000/73422, and plants which produce alternan, as described in WO 2000/047727, EP 06077301.7, U.S. Pat. No. 5,908,975 and EP 0728213.
3) Transgenic plants which produce hyaluronan, as described, for example, in WO 2006/032538, WO 2007/039314, WO 2007/039315, WO 2007/039316, JP 2006/304779 and WO 2005/012529.

Plants or plant varieties (obtained by plant biotechnology methods such as genetic engineering) which may also be treated according to the invention are plants, such as cotton plants, with altered fibre characteristics. Such plants can be obtained by genetic transformation, or by selection of plants containing a mutation imparting such altered fibre characteristics and include:
a) plants, such as cotton plants, which contain an altered form of cellulose synthase genes, as described in WO 1998/000549;
b) plants, such as cotton plants, which contain an altered form of rsw2 or rsw3 homologous nucleic acids, as described in WO 2004/053219;
c) plants, such as cotton plants, with an increased expression of sucrose phosphate synthase, as described in WO 2001/017333;
d) plants, such as cotton plants, with an increased expression of sucrose synthase, as described in WO 02/45485;
e) plants, such as cotton plants, wherein the timing of the plasmodesmatal gating at the basis of the fibre cell is altered, for example through downregulation of fibre-selective β-1,3-glucanase, as described in WO 2005/017157;
f) plants, such as cotton plants, which have fibres with altered reactivity, for example through the expression of the N-acetylglucosaminetransferase gene including nodC and chitin synthase genes, as described in WO 2006/136351.

Plants or plant cultivars (obtained by plant biotechnology methods such as genetic engineering) which may also be treated according to the invention are plants, such as oilseed rape or related Brassica plants, with altered oil profile characteristics. Such plants can be obtained by genetic transformation or by selection of plants containing a mutation imparting such altered oil characteristics and include:
a) plants, such as oilseed rape plants, which produce oil having a high oleic acid content, as described, for example, in U.S. Pat. No. 5,969,169, U.S. Pat. No. 5,840,946 or U.S. Pat. No. 6,323,392 or U.S. Pat. No. 6,063,947;
b) plants, such as oilseed rape plants, which produce oil having a low linolenic acid content, as described in U.S. Pat. No. 6,270,828, U.S. Pat. No. 6,169,190 or U.S. Pat. No. 5,965,755;
c) plants, such as oilseed rape plants, which produce oil having a low level of saturated fatty acids, as described, for example, in U.S. Pat. No. 5,434,283.

Particularly useful transgenic plants which may be treated according to the invention are plants which comprise one or more genes which encode one or more toxins and are the transgenic plants available under the following trade names: YIELD GARD® (for example maize, cotton, soya beans), KnockOut® (for example maize), BiteGard® (for example maize), BT-Xtra® (for example maize), StarLink® (for example maize), Bollgard® (cotton), Nucotn® (cotton), Nucotn 33B® (cotton), NatureGard® (for example maize), Protecta® and NewLeaf® (potato). Examples of herbicide-tolerant plants which may be mentioned are maize varieties, cotton varieties and soya bean varieties which are available under the following trade names: Roundup Ready® (tolerance to glyphosate, for example maize, cotton, soya beans), Liberty Link® (tolerance to phosphinothricin, for example oilseed rape), IMI® (tolerance to imidazolinone) and SCS® (tolerance to sulphonylurea, for example maize). Herbicide-resistant plants (plants bred in a conventional manner for herbicide tolerance) which may be mentioned include the varieties sold under the name Clearfield® (for example maize).

Particularly useful transgenic plants which may be treated according to the invention are plants containing transformation events, or a combination of transformation events, and that are listed for example in the databases for various national or regional regulatory agencies (see for example http://gmoinfo.jrc.it/gmp_browse.aspx and http://www.agbios.com/dbase.php).

The active compounds according to the invention act not only against plant, hygiene and stored product pests, but also in the veterinary medicine sector against animal parasites (ecto- and endoparasites), such as hard ticks, soft ticks, mange mites, leaf mites, flies (biting and licking), parasitic fly larvae, lice, hair lice, feather lice and fleas. These parasites include:

From the order of the Anoplurida, for example, Haematopinus spp., Linognathus spp., Pediculus spp., Phtirus spp. and Solenopotes spp.

From the order of the Mallophagida and the suborders Amblycerina and Ischnocerina, for example, Trimenopon spp., Menopon spp., Trinoton spp., Bovicola spp., Werneckiella spp., Lepikentron spp., Damalina spp., Trichodectes spp. and Felicola spp.

From the order of the Diptera and the suborders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypodei ma spp., Gasterophilus spp., Hippobosca spp., Lipoptena spp. and Melophagus spp.

From the order of the Siphonapterida, for example Pulex spp., Ctenocephalides spp., Xenopsylla spp. and Ceratophyllus spp.

From the order of the Heteropterida, for example, Cimex spp., Triatoma spp., Rhodnius spp. and Panstrongylus spp.

From the order of the Blattarida, for example Blatta orientalis, Periplaneta americana, Blattella germanica and Supella spp.

From the subclass of the Acari (Acarina) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp. and Varroa spp.

From the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp. and Laminosioptes spp.

The active compounds of the formula (I) according to the invention are also suitable for controlling arthropods which infest agricultural productive livestock, such as, for example, cattle, sheep, goats, horses, pigs, donkeys, camels, buffalo, rabbits, chickens, turkeys, ducks, geese and bees, other pets, such as, for example, dogs, cats, caged birds and aquarium fish, and also so-called test animals, such as, for example, hamsters, guinea pigs, rats and mice. By controlling these arthropods, cases of death and reduction in productivity (for meat, milk, wool, hides, eggs, honey etc.) should be diminished, so that more economic and easier animal husbandry is possible by use of the active compounds according to the invention.

The active compounds according to the invention are used in the veterinary sector and in animal husbandry in a known manner by enteral administration in the form of, for example, tablets, capsules, potions, drenches, granules, pastes, boluses, the feed-through process and suppositories, by parenteral administration, such as, for example, by injection (intramuscular, subcutaneous, intravenous, intraperitoneal and the like), implants, by nasal administration, by dermal use in the form, for example, of dipping or bathing, spraying, pouring on and spotting on, washing and powdering, and also with the aid of moulded articles containing the active compound, such as collars, ear marks, tail marks, limb bands, halters, marking devices and the like.

When used for livestock, poultry, domestic animals and the like, the active compounds of the formula (I) can be used as formulations (for example powders, emulsions, flowables) comprising the active compounds in an amount of from 1 to 80% by weight, either directly or after 100 to 10 000-fold dilution, or they may be used as a chemical bath.

It has furthermore been found that the compounds according to the invention have a strong insecticidal action against insects which destroy industrial materials.

The following insects may be mentioned as examples and as preferred—but without limitation:

beetles, such as Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec. Tryptodendron spec. Apate monachus, Bostrychus capucins, Heterobostrychus brunneus, Sinoxylon spec. Dinoderus minutus;
dermapterans, such as Sirex juvencus, Urocerus gigas, Urocerus gigas taignus, Urocerus augur;
termites, such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus;
bristletails, such as Lepisma saccarina.

Industrial materials in the present connection are to be understood as meaning non-living materials, such as, preferably, plastics, adhesives, sizes, papers and cards, leather, wood and processed wood products and coating compositions.

The ready-to-use compositions can also comprise other insecticides, if appropriate, and also one or more fungicides, if appropriate.

With respect to possible additional partners for mixing, reference is made to the insecticides and fungicides mentioned above.

The compounds according to the invention can at the same time be employed for protecting objects which come into contact with saltwater or brackish water, such as hulls, screens, nets, buildings, moorings and signalling systems in particular, against fouling.

Furthermore, the compounds according to the invention can be used alone or in combinations with other active compounds as antifouling compositions.

The active compounds are also suitable for controlling animal pests in the domestic field, in hygiene and in the protection of stored products, in particular insects, arachnids and mites, which are found in enclosed spaces such as, for example, dwellings, factory halls, offices, vehicle cabins and the like. They can be employed alone or in combination with other active compounds and auxiliaries in domestic insecticide products for controlling these pests. They are active against sensitive and resistant species and against all developmental stages. These pests include:

From the order of the Scorpionidea, for example, Buthus occitanus.

From the order of the Acarina, for example, Argas persicus, Argas reflexus, Bryobia spp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae.

From the order of the Araneae, for example, Aviculariidae, Araneidae.

From the order of the Opiliones, for example, Pseudoscorpiones chelifer, Pseudoscorpiones cheiridium, Opiliones phalangium.

From the order of the Isopoda, for example, Oniscus asellus, Porcellio scaber.

From the order of the Diplopoda, for example, Blaniulus guttulatus, Polydesmus spp.

From the order of the Chilopoda, for example, Geophilus spp.

From the order of the Zygentoma, for example, Ctenolepisma spp., Lepisma saccharina, Lepismodes inquilinus.

From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.

From the order of the Saltatoria, for example, Acheta domesticus.

From the order of the Dermaptera, for example, Forficula auricularia.

From the order of the Isoptera, for example, Kalotermes spp., Reticulitermes spp.

From the order of the Psocoptera, for example, Lepinatus spp., Liposcelis spp.

From the order of the Coleoptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum.

From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans, Tipula paludosa.

From the order of the Lepidoptera, for example, Achroia grisella, Galleria mellonella, Plodia interpunctella, Tinea cloacella, Tinea pellionella, Tineola bisselliella.

From the order of the Siphonaptera, for example, Ctenocephalides canis, Ctenocephalides felis, Pulex irritans, Tunga penetrans, Xenopsylla cheopis.

From the order of the Hymenoptera, for example, Camponotus herculeanus, Lasius fuliginosus, Lasius niger, Lasius umbratus, Monomorium pharaonic, Paravespula spp., Tetramorium caespitum.

From the order of the Anoplura, for example, Pediculus humanus capitis, Pediculus humanus corporis, Pemphigus spp., Phylloera vastatrix, Phthirus pubis.

From the order of the Heteroptera, for example, Cimex hemipterus, Cimex lectularius, Rhodinus prolixus, Triatoma infestans.

In the field of household insecticides, they are used alone or in combination with other suitable active compounds, such as phosphoric acid esters, carbamates, pyrethroids, neonicotinoids, growth regulators or active compounds from other known classes of insecticides.

They are used in aerosols, pressure-free spray products, for example pump and atomizer sprays, automatic fogging systems, foggers, foams, gels, evaporator products with evaporator tablets made of cellulose or plastic, liquid evaporators, gel and membrane evaporators, propeller-driven evaporators, energy-free, or passive, evaporation systems, moth papers, moth bags and moth gels, as granules or dusts, in baits for spreading or in bait stations.

### SYNTHESIS EXAMPLES

The following abbreviations are used in the tables below:

| | | | |
|---|---|---|---|
| Me = methyl | Et = ethyl | Pr = propyl | iPr = isopropyl |
| tBu = tert-butyl | Pen = pentyl | Ph = phenyl | tBu = tert-buty |
| Cypr = cyclopropyl | Cybu = cyclobutyl | Cypen = cyclopentyl | Cyhex = cyclohexyl |
| Ac = C(O)Me | | | |
| 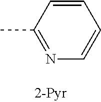 | 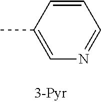 | 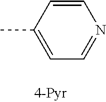 | 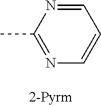 |
| 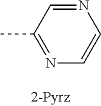 | 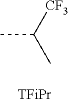 | 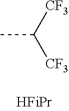 | |

¹H-NMR data (400 MHz., internal reference: tetramethylsilane δ = 0.00 ppm; s = singlet, br.
s = broad singlet, d = doublet, dd = doublet of doublets, m = multiplet, q = quartet, t = triplet)

TABLE 1
In accordance with the general methods described above, it is possible to prepare the compounds of the formula (I-A where Q = Q¹) listed in the
table below.
(I-A-Q¹)

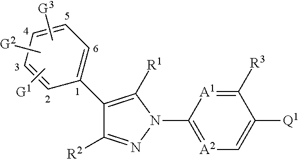

| | | | | | | | | | | Phys. |
|---|---|---|---|---|---|---|---|---|---|---|
| Example | | | | | | | | | | chem. |
| no. | G¹ | G² | G³ | R¹ | R² | R³ | A¹ | A² | Q¹ | data |
| I-A-Q1- 001 | 3-CF₃ | H | 5-Cl | NH₂ | CF₃ | CN | CH | CH | 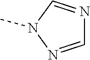 | log P ¹H-NMR |
| I-A-Q1- 002 | 3-CF₃ | H | 5-Cl | NH₂ | CF₃ | CN | CH | CH | 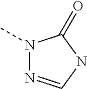 | log P ¹H-NMR |
| I-A-Q1- 003 | 3-Cl | H | 5-Cl | H | CF₃ | Me | CH | CH | 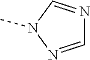 | |
| I-A-Q1- 004 | 3-CF₃ | H | 5-CF₃ | H | CF₃ | CN | CH | CH | 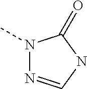 | |
| I-A-Q1- 005 | 3-CF₃ | H | H | NH₂ | CF₃ | NO₂ | CH | CH | 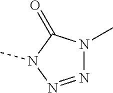 | |
| I-A-Q1- 006 | 3-Br | H | 5-Br | NH₂ | CF₃ | CF₃ | CH | CH | 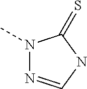 | |
| I-A-Q1- 007 | 3-Cl | 4-Cl | 5-Cl | Cl | CF₃ | Br | CH | CH | 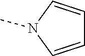 | |
| I-A-Q1- 008 | 3-Br | 4-Cl | 5-Br | Cl | C₂F₅ | Cl | CH | CH | 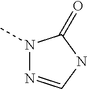 | |
| I-A-Q1- 009 | 3-CF₃ | 4-Cl | 5-Cl | Br | C₂F₅ | CH₂OMe | CH | CH | 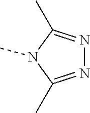 | |
| I-A-Q1- 010 | 3-CF₃ | 4-F | H | Br | C₂F₅ | CH₂NHMe | CH | CH | 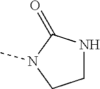 | |
| I-A-Q1- 011 | 3-Cl | 4-CF₃ | 5-Cl | I | C₂F₅ | Me | CH | CH | 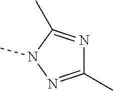 | |
| I-A-Q1- 012 | 3-CF₃ | H | 5-Cl | I | CF₃ | CN | CH | CH | 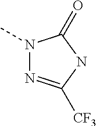 | |
| I-A-Q1- 013 | 3-Cl | 4-CF₃ | 5-Cl | CN | CF₃ | NO₂ | N | N | 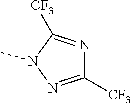 | |
| I-A-Q1- 014 | 3-Cl | 4-Br | 5-Cl | CN | CF₃ | CF₃ | N | N | 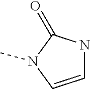 | |
| I-A-Q1- 015 | 3-F | 4-F | 5-F | NHCH₂Ph | C₂F₅ | Br | CH | CH | 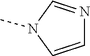 | |
| I-A-Q1- 016 | 2-Cl | 3-CF₃ | 4-Cl | NHAc | CF₃ | Cl | CH | CH | 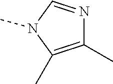 | |
| I-A-Q1- 017 | 3-Cl | 4-CF₃ | H | NAc₂ | CF₃ | CH₂OAc | CH | CH | 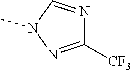 | |
| I-A-Q1- 018 | 3-Br | 4-NH₂ | 5-Br | NHMe | CF₂Ph | CH₂NHAc | CH | CH | 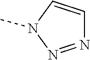 | |
| I-A-Q1- 019 | 3-Br | H | H | NMe₂ | CF₂OMe | Me | CH | CH | 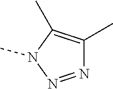 | |
| I-A-Q1- 020 | 3-Cl | 4-Cl | H | SMe | CF₂OPh | CN | CH | CH | 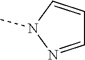 | |
| I-A-Q1- 021 | 2-Cl | 3-Cl | 4-Cl | SOMe | CF₂(2-Pyr) | NO₂ | N | CH | 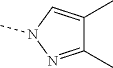 | |
| I-A-Q1- 022 | 3-NO₂ | H | H | SO₂Me | CF₂O(2-Pyr) | CF₃ | CH | N | 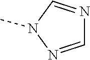 | |
| I-A-Q1- 023 | 3-Cl | H | 5-CF₃ | NH₂ | CHMe(OMe) | CN | CH | CH | 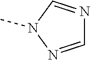 | |
| I-A-Q1- 024 | 3-Cl | H | 5-Cl | NH₂ | CF₃ | CN | CH | CH | 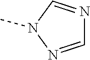 | log P |
| I-A-Q1- 025 | 3-Cl | 4-Cl | H | NH₂ | Me | CN | CH | CH | 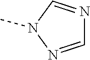 | log P |
| I-A-Q1- 026 | 3-Cl | H | 5-Cl | NH₂ | CF₃ | CN | CH | CH | 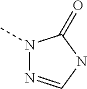 | log P |
| I-A-Q1- 027 | 3-CF₃ | H | 5-CF₃ | NH₂ | CF₃ | CN | CH | CH | 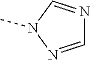 | log P |
| I-A-Q1- 028 | 2-Cl | H | 5-Cl | NH₂ | Me | CN | CH | CH | 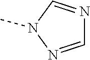 | log P |
| I-A-Q1- 029 | H | 4-Cl | H | NH₂ | Me | CN | CH | CH | 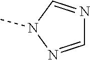 | log P |
| I-A-Q1- 030 | H | 4-Cl | H | NH₂ | CHF₂ | CN | CH | CH | 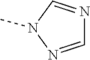 | log P |
| I-A-Q1- 031 | 3-Cl | 4-Cl | H | NH₂ | CF₃ | CN | CH | CH | 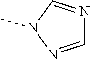 | log P |
| I-A-Q1- 032 | 3-F | 4-Cl | H | NH₂ | CF₃ | CN | CH | CH | 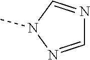 | log P |
| I-A-Q- 033 | H | 4-Cl | H | NH₂ | CF₃ | CN | CH | CH | 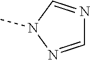 | log P |
| I-A-Q1- 034 | 2-F | 4-F | 6-F | NH₂ | CF₃ | CN | CH | CH | 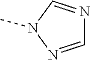 | log P |
| I-A-Q1- 035 | 2-Cl | H | H | NH₂ | CF₃ | CN | CH | CH | 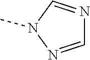 | log P |
| I-A-Q1- 036 | 3-Cl | 4-F | H | NH₂ | CF₃ | CN | CH | CH | 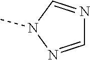 | log P |
| I-A-Q1- 037 | 3-F | H | 5-Cl | NH₂ | CF₃ | CN | CH | CH | 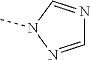 | log P |
| I-A-Q1- 038 | 3-Me | H | 5-Me | NH₂ | CF₃ | CN | CH | CH | 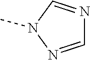 | log P |
| I-A-Q1- 039 | 2-Me | H | H | NH₂ | CF₃ | CN | CH | CH | 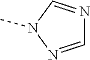 | log P |
| I-A-Q1- 040 | H | 4-Br | H | NH₂ | CF₃ | CN | CH | CH | 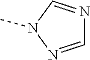 | log P |
| I-A-Q1- 041 | 3-F | 4-F | 5-F | NH₂ | CF₃ | CN | CH | CH | 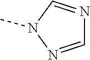 | log P |
| I-A-Q1- 042 | 3-F | H | H | NH₂ | CF₃ | CN | CH | CH | 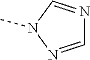 | log P |
| I-A-Q1- 043 | H | 4-I | H | NH₂ | CF₃ | CN | CH | CH | 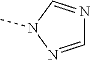 | log P |
| I-A-Q1- 044 | 3-F | 4-F | H | NH₂ | CF₃ | CN | CH | CH | 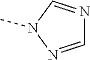 | log P |
| I-A-Q1- 045 | 2-Cl | 4-Cl | H | NH₂ | CF₃ | CN | CH | CH | 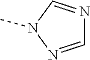 | log P |
| I-A-Q1- 046 | 3-Br | H | H | NH₂ | CF₃ | CN | CH | CH | 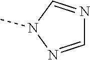 | log P |
| I-A-Q1- 047 | H | 4-CN | H | NH₂ | CF₃ | CN | CH | CH | 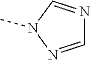 | log P |
| I-A-Q1- 048 | 2-F | 4-F | H | NH₂ | CHF₂ | CN | CH | CH | 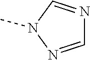 | log P |
| I-A-Q1- 049 | 3-Cl | 5-CF₃ | H | NH₂ | CF₂Cl | CN | CH | CH | 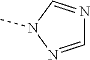 | log P |
| I-A-Q1- 050 | 3-CF₃ | H | 5-CF₃ | NH₂ | C₂F₅ | CN | CH | CH | 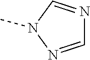 | log P |
| I-A-Q1- 051 | 2-Cl | H | 6-F | NH₂ | Me | CN | CH | CH | 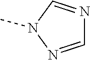 | log P |
| I-A-Q1- 052 | 2-Cl | 4-F | H | NH₂ | Me | CN | CH | CH | 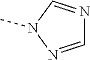 | log P |
| I-A-Q1- 053 | 3-Cl | H | H | NH₂ | Me | CN | CH | CH | 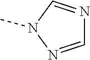 | log P |
| I-A-Q1- 054 | 2-F | H | 6-F | NH₂ | Me | CN | CH | CH | 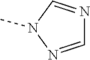 | log P |
| I-A-Q1- 055 | 2-Cl | H | 6-Cl | NH₂ | Me | CN | CH | CH | 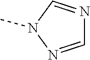 | log P |
| I-A-Q1- 056 | 2-Cl | 4-Cl | H | NH₂ | Me | CN | CH | CH | 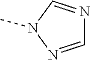 | log P |
| I-A-Q1- 057 | 2-F | 4-F | H | NH₂ | Me | CN | CH | CH | 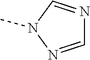 | log P |
| I-A-Q1- 058 | H | 4-MeO | H | NH₂ | Me | CN | CH | CH | 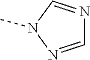 | log P |
| I-A-Q1- 059 | H | 4-F | H | NH₂ | Me | CN | CH | CH | 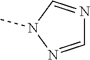 | log P |
| I-A-Q1- 060 | 2-Cl | H | H | NH₂ | Me | CN | CH | CH | 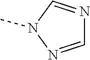 | log P |
| I-A-Q1- 061 | 2-F | H | 5-F | NH₂ | Me | CN | CH | CH | 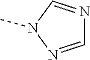 | log P |
| I-A-Q1- 062 | H | 4-Cl | H | NH₂ | Et | CN | CH | CH | 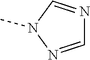 | log P |
| I-A-Q1- 063 | H | 4-Cl | H | NH₂ | Ph | CN | CH | CH | 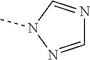 | log P |
| I-A-Q1- 064 | H | 4-Cl | H | NH₂ | Cypr | CN | CH | CH | 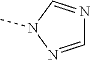 | log P |
| I-A-Q1- 065 | H | 4-Cl | H | NH₂ | Pr | CN | CH | CH | 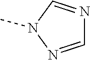 | log P |
| I-A-Q1- 066 | H | H | H | NH₂ | Me | CN | CH | CH | 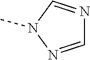 | log P |
| I-A-Q1- 067 | 3-Cl | H | 5-Cl | NH₂ | CF₃ | H | CBr | CH | 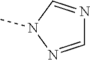 | log P |
| I-A-Q1- 068 | 3-Cl | H | 5-Cl | NH₂ | CF₃ | CF₃ | CH | CH | 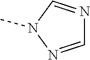 | log P |
| I-A-Q1- 069 | 3-Cl | H | 5-Cl | NH₂ | CF₃ | Cl | CH | CH | 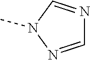 | log P |
| I-A-Q1- 070 | 3-Cl | H | 5-Cl | NH₂ | CF₃ | H | OCF₃ | CH | 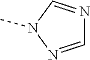 | log P |
| I-A-Q1- 071 | 3-Cl | H | 5-CF₃ | NH₂ | (4-ClC₆H₄)OCF₂ | CN | CH | CH | 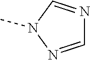 | log P |
| I-A-Q1- 072 | 3-Cl | H | 5-CF₃ | NH₂ | (3-ClC₆H₄)OCF₂ | CN | CH | CH | 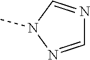 | log P |
| I-A-Q1- 073 | 3-Cl | H | 5-CF₃ | NH₂ | (2-ClC₆H₄)OCF₂ | CN | CH | CH | 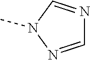 | log P |
| I-A-Q1- 074 | 3-Cl | H | 5-CF₃ | NH₂ | PhOCF₂ | CN | CH | CH | 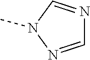 | log P |
| I-A-Q1- 075 | 3-Cl | H | 5-Cl | NH₂ | CF₃ | 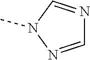 | CH | CH | 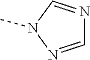 | log P |
| I-A-Q1- 076 | 3-Cl | H | 5-Cl | NH₂ | CF₃ | CN | CH | CH | 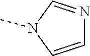 | log P |
| I-A-Q1- 077 | 3-Cl | H | 5-Cl | NH₂ | CF₃ | CN | CH | CH | 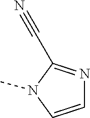 | log P |
| I-A-Q1- 078 | 3-Cl | H | 5-Cl | NH₂ | CF₃ | CN | CH | CH | 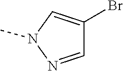 | log P |
| I-A-Q1- 079 | 3-Cl | H | 5-Cl | NH₂ | CF₃ | CN | CH | CH | 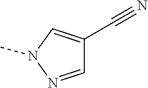 | log P |
| I-A-Q1- 080 | 3-Cl | H | 5-Cl | NH₂ | CF₃ | CN | CH | CH | 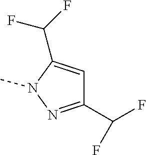 | log P |
| I-A-Q1- 081 | 3-Cl | H | 5-Cl | NH₂ | CF₃ | CN | CH | CH | 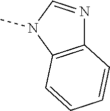 | log P |
| I-A-Q1- 082 | 3-Cl | H | 5-Cl | NH₂ | CF₃ | CN | CH | CH | 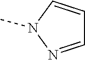 | log P |
| I-A-Q1- 083 | 3-Cl | H | 5-Cl | NH₂ | CF₃ | CN | CH | CH | 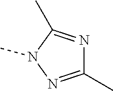 | log P |
| I-A-Q1- 084 | 3-Cl | H | 3-Cl | NH₂ | CF₃ | CN | CH | CH | 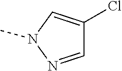 | log P |
| I-A-Q1- 085 | 3-Cl | H | 5-Cl | NH₂ | CF₃ | CN | CH | CH | 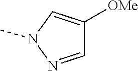 | log P |
| I-A-Q1- 086 | 3-Cl | H | 5-Cl | NH₂ | CF₃ | CN | CH | CH | 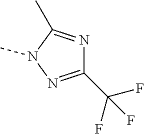 | log P |
| I-A-Q1- 087 | 3-Cl | H | 5-Cl | NH₂ | CF₃ | CN | CH | CH | 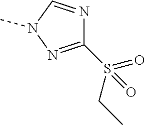 | log P |
| I-A-Q1- 088 | 3-Cl | H | 5-Cl | NH₂ | CF₃ | CN | CH | CH | 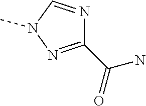 | log P |
| I-A-Q1- 089 | 3-Cl | H | 5-Cl | NH₂ | CF₃ | CN | CH | CH | 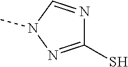 | log P |
| I-A-Q1- 090 | 3-Cl | H | 5-Cl | NH₂ | CF₃ | CN | CH | CH | 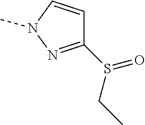 | log P |
| I-A-Q1- 091 | 3-Cl | H | 5-Cl | NH₂ | CF₃ | CN | CH | CH | 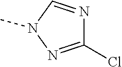 | log P |
| I-A-Q1- 092 | 3-Cl | H | 5-Cl | NH₂ | CF₃ | CN | CH | CH | 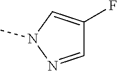 | log P |
| I-A-Q1- 093 | 3-Cl | H | 5-Cl | NH₂ | CF₃ | CN | CH | CH | 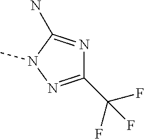 | log P |

Characteristic data for synthesis examples:
I-A-Q1-001: spectroscopic data see protocol under general synthesis procedures
I-A-Q1-002: spectroscopic data see protocol under general synthesis procedures
I-A-Q1-023: log P (HCOOH): 2.76
I-A-Q1-024: log P (HCOOH): 3.90
I-A-Q1-025: log P (HCOOH): 3.02
I-A-Q1-026: log P (HCOOH): 3.37
I-A-Q1-027: log P (HCOOH): 4.07
I-A-Q1-028: log P (HCOOH): 2.65
I-A-Q1-029: log P (HCOOH): 2.65
I-A-Q1-030: log P (HCOOH): 2.92
I-A-Q1-031: log P (HCOOH): 3.60
I-A-Q1-032: log P (HCOOH): 3.28
I-A-Q1-033: log P (HCOOH): 3.30
I-A-Q1-034: log P (HCOOH): 3.10
I-A-Q1-035: log P (HCOOH): 2.80
I-A-Q1-036: log P (HCOOH): 3.24
I-A-Q1-037: log P (HCOOH): 3.33
I-A-Q1-038: log P (HCOOH): 3.33
I-A-Q1-039: log P (HCOOH): 2.89
I-A-Q1-040: log P (HCOOH): 3.44
I-A-Q1-041: log P (HCOOH): 3.19
I-A-Q1-042: log P (HCOOH): 2.84
I-A-Q1-043: log P (HCOOH): 3.61
I-A-Q1-044: log P (HCOOH): 3.10
I-A-Q1-045: log P (HCOOH): 2.64
I-A-Q1-046: log P (HCOOH): 3.24
I-A-Q1-047: log P (HCOOH): 2.64
I-A-Q1-048: log P (HCOOH): 2.39
I-A-Q1-049: log P (HCOOH): 4.11
I-A-Q1-050: log P (HCOOH): 4.34
I-A-Q1-051: log P (HCOOH): 2.27
I-A-Q1-052: log P (HCOOH): 2.43
I-A-Q1-053: log P (HCOOH): 2.66
I-A-Q1-054: log P (HCOOH): 2.18
I-A-Q1-055: log P (HCOOH): 2.47
I-A-Q1-056: log P (HCOOH): 2.80
I-A-Q1-057: log P (HCOOH): 2.35
I-A-Q1-058: log P (HCOOH): 2.11
I-A-Q1-059: log P (HCOOH): 2.30
I-A-Q1-060: log P (HCOOH): 2.27
I-A-Q1-061: log P (HCOOH): 2.29
I-A-Q1-062: log P (HCOOH): 3.09
I-A-Q1-063: log P (HCOOH): 3.53
I-A-Q1-064: log P (HCOOH): 3.31
I-A-Q1-065: log P (HCOOH): 3.33
I-A-Q1-066: log P (HCOOH): 2.14
I-A-Q1-067: log P (HCOOH): 3.97
I-A-Q1-068: log P (HCOOH): 4.32
I-A-Q1-069: log P (HCOOH): 4.14
I-A-Q1-070: log P (HCOOH): 4.10
I-A-Q1-071: log P (HCOOH): 4.84
I-A-Q1-072: log P (HCOOH): 4.82
I-A-Q1-073: log P (HCOOH): 4.51
I-A-Q1-074: log P (HCOOH): 4.33
I-A-Q1-075: log P (HCOOH): 3.28
I-A-Q1-076: log P (HCOOH): 2.98
I-A-Q1-077: log P (HCOOH): 4.05
I-A-Q1-078: log P (HCOOH): 4.83
I-A-Q1-079: log P (HCOOH): 4.28
I-A-Q1-080: log P (HCOOH): 4.64
I-A-Q1-081: log P (HCOOH): 4.28
I-A-Q1-082: log P (HCOOH): 4.34
I-A-Q1-083: log P (HCOOH): 3.84
I-A-Q1-084: log P (HCOOH): 4.70
I-A-Q1-085: log P (HCOOH): 4.45
I-A-Q1-086: log P (HCOOH): 4.57
I-A-Q1-087: log P (HCOOH): 3.78
I-A-Q1-088: log P (HCOOH): 3.06
I-A-Q1-089: log P (HCOOH): 3.53
I-A-Q1-090: log P (HCOOH): 3.47
I-A-Q1-091: log P (HCOOH): 4.17
I-A-Q1-092: log P (HCOOH): 4.40
I-A-Q1-093: log P (HCOOH): 4.00

TABLE 2
In accordance with the general methods described above, it is possible to prepare the compounds of the formula (I-A where Q = Q²) listed in the table below.
(I-A-Q²)

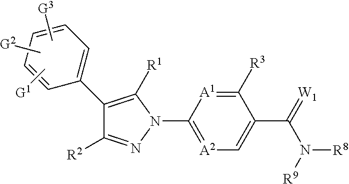

| Example no. | G¹ | G² | G³ | R¹ | R² | R³ | A¹ | A² | W¹ | R⁸ | R⁹ | Phys. chem. data |
|---|---|---|---|---|---|---|---|---|---|---|---|---|
| I-A-Q2-001 | 3-Cl | H | 5-Cl | H | CF₃ | CF₃ | CH | CH | O | H | CH₂-2-Pyr | log P |
| | | | | | | | | | | | | ¹H-NMR |
| I-A-Q2-002 | 3-Cl | H | 5-Cl | H | CF₃ | CF₃ | CH | CH | O | H | CH₂CF₃ | log P |
| | | | | | | | | | | | | ¹H-NMR |
| I-A-Q2-003 | 3-Cl | H | 5-Cl | H | CF₃ | NO₂ | CH | CH | O | H | CH₂-2-Pyr | ¹H-NMR |
| I-A-Q2-004 | 3-Cl | H | 5-Cl | H | CF₃ | NO₂ | CH | CH | O | H | CH₂CF₃ | ¹H-NMR |
| I-A-Q2-005 | 3-Cl | H | 5-Cl | H | CF₃ | CF₃ | CH | CH | O | H | CHMeCF₃ | ¹H-NMR |
| I-A-Q2-006 | 3-Cl | H | 5-Cl | H | H | CF₃ | CH | CH | O | H | CHMeCF₃ | ¹H-NMR |
| I-A-Q2-007 | 3-Cl | H | 5-Cl | H | H | CF₃ | CH | CH | O | H | CH₂CF₃ | |
| I-A-Q2-008 | 3-Cl | H | 5-Cl | H | H | CF₃ | CH | CH | O | H | CH₂-2-Pyr | ¹H-NMR |
| I-A-Q2-009 | 3-CF₃ | H | 5-CF₃ | H | CF₃ | CF₃ | CH | CH | O | H | CH₂-2-Pyr | ¹H-NMR |
| I-A-Q2-010 | 3-CF₃ | H | 5-CF₃ | H | CF₃ | CF₃ | CH | CH | O | H | CH₂CF₃ | ¹H-NMR |
| I-A-Q2-011 | 3-Cl | H | 5-Cl | NH₂ | CF₃ | Me | CH | CH | O | H | TFiPr | log P |
| | | | | | | | | | | | | ¹H-NMR |
| I-A-Q2-012 | 3-Cl | H | 5-Cl | NH₂ | CF₃ | Me | CH | CH | O | H | CH₂-2-Pyr | log P |
| | | | | | | | | | | | | ¹H-NMR |
| I-A-Q2-013 | 3-Cl | H | 5-Cl | NH₂ | CF₃ | Me | CH | CH | O | H | Me | log P |
| I-A-Q2-014 | 3-Cl | H | 5-Cl | NH₂ | CF₃ | Me | CH | CH | O | H | CH₂CF₃ | log P |
| | | | | | | | | | | | | ¹H-NMR |
| I-A-Q2-015 | 3-Cl | H | 5-Cl | NH₂ | CF₃ | Me | CH | CH | O | H | CHMe-2-Pyr | log P |
| | | | | | | | | | | | | ¹H-NMR |
| I-A-Q2-016 | 3-Cl | H | 5-Cl | NH₂ | CF₃ | Me | CH | CH | O | H | CHMePh | log P |
| | | | | | | | | | | | | ¹H-NMR |
| I-A-Q2-017 | 3-Cl | H | 5-Cl | NH₂ | CF₃ | Me | CH | CH | O | H | CH₂CHF₂ | log P |
| | | | | | | | | | | | | ¹H-NMR |
| I-A-Q2-018 | 3-Cl | H | 5-Cl | NH₂ | CF₃ | Me | CH | CH | O | H | CH₂-2-Pyrm | log P |
| | | | | | | | | | | | | ¹H-NMR |
| I-A-Q2-019 | 3-Cl | 4-Cl | H | NH₂ | CF₃ | Me | CH | CH | O | H | TFiPr | log P |
| | | | | | | | | | | | | ¹H-NMR |
| I-A-Q2-020 | 3-Cl | H | 5-Cl | NH₂ | CF₃ | CF₃ | N | N | O | H | CH₂-2-Pyr | log P |
| | | | | | | | | | | | | ¹H-NMR |
| I-A-Q2-021 | 3-Cl | H | 5-Cl | NH₂ | CF₃ | CF₃ | N | N | O | H | CHMe-2-Pyr | log P |
| | | | | | | | | | | | | ¹H-NMR |
| I-A-Q2-022 | 3-Cl | H | 5-Cl | Cl | CF₃ | Me | CH | CH | O | H | TFiPr | log P |
| | | | | | | | | | | | | ¹H-NMR |
| I-A-Q2-023 | 3-Cl | H | 5-Cl | Br | CF₃ | Me | CH | CH | O | H | CH₂-2-Pyr | log P |
| | | | | | | | | | | | | ¹H-NMR |
| I-A-Q2-024 | 3-Cl | H | 5-Cl | Br | CF₃ | Me | CH | CH | O | H | TFiPr | log P |
| | | | | | | | | | | | | ¹H-NMR |
| I-A-Q2-025 | 3-Cl | H | 5-Cl | I | CF₃ | Me | CH | CH | O | H | CH₂-2-Pyr | log P |
| | | | | | | | | | | | | ¹H-NMR |
| I-A-Q2-026 | 3-Cl | H | 5-Cl | I | CF₃ | Me | CH | CH | O | H | TFiPr | log P |
| | | | | | | | | | | | | ¹H-NMR |
| I-A-Q2-027 | 3-Cl | H | 5-Cl | SMe | CF₃ | Me | CH | CH | O | H | TFiPr | log P |
| | | | | | | | | | | | | ¹H-NMR |
| I-A-Q2-028 | 3-Cl | H | 5-Cl | SMe | CF₃ | Me | CH | CH | O | H | CH₂-2-Pyr | log P |
| | | | | | | | | | | | | ¹H-NMR |
| I-A-Q2-029 | 3-Cl | H | 5-Cl | NH₂ | CF₃ | Me | CH | CH | O | H | 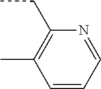 | log P |
| I-A-Q2-030 | 3-Cl | H | 5-Cl | NH₂ | CF₃ | Me | CH | CH | O | H | CH₂COOMe | log P |
| I-A-Q2-031 | 3-Cl | H | 5-Cl | NH₂ | CF₃ | Me | CH | CH | O | H | 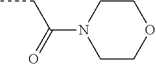 | log P |
| I-A-Q2-032 | 3-Cl | H | 5-Cl | NH₂ | CF₃ | Me | CH | CH | O | H | 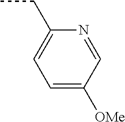 | log P |
| I-A-Q2-033 | 3-Cl | H | 5-Cl | NH₂ | CF₃ | Me | CH | CH | O | Me | CH₂CF₂CHF₂ | log P |
| I-A-Q2-034 | 3-Cl | H | 5-Cl | NH₂ | CF₃ | Me | CH | CH | O | H | 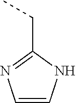 | log P |
| I-A-Q2-035 | 3-Cl | H | 5-Cl | NH₂ | CF₃ | Me | CH | CH | O | H | CH₂CF₂CHF₂ | log P |
| I-A-Q2-036 | 3-Cl | H | 5-Cl | NH₂ | CF₃ | Me | CH | CH | O | H | 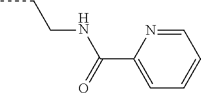 | log P |
| I-A-Q2-037 | 3-Cl | H | 5-Cl | NH₂ | CF₃ | Me | CH | CH | O | H | 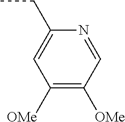 | log P |
| I-A-Q2-038 | 3-Cl | H | 5-Cl | NH₂ | CF₃ | Me | CH | CH | O | H | 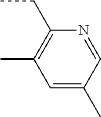 | log P |
| I-A-Q2-039 | 3-Cl | H | 5-Cl | NH₂ | CF₃ | Me | CH | CH | O | H | 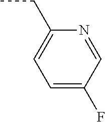 | log P |
| I-A-Q2-040 | 3-Cl | H | 5-Cl | NH₂ | CF₃ | Me | CH | CH | O | H | 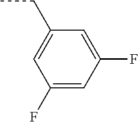 | log P |
| I-A-Q2-041 | 3-Cl | H | 5-Cl | NH₂ | CF₃ | Me | CH | CH | O | H | 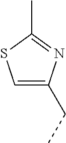 | log P |
| I-A-Q2-042 | 3-Cl | H | 5-Cl | NH₂ | CF₃ | Me | CH | CH | O | H | 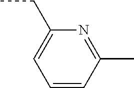 | log P |
| I-A-Q2-043 | 3-Cl | H | 5-Cl | NH₂ | CF₃ | Me | CH | CH | O | H | 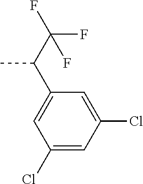 | log P |
| I-A-Q2-044 | 3-Cl | H | 5-Cl | NH₂ | CF₃ | Me | CH | CH | O | H | 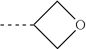 | log P |
| I-A-Q2-045 | 3-Cl | H | 5-Cl | NH₂ | CF₃ | Me | CH | CH | O | H | 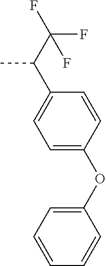 | log P |
| I-A-Q2-046 | 3-Cl | H | 5-Cl | NH₂ | CF₃ | Me | CH | CH | O | H | 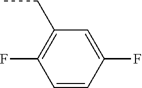 | log P |
| I-A-Q2-047 | 3-Cl | H | 5-Cl | NH₂ | CF₃ | Me | CH | CH | O | H | 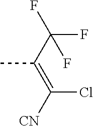 | log P |
| I-A-Q2-048 | 3-Cl | H | 5-Cl | NH₂ | CF₃ | Me | CH | CH | O | H | 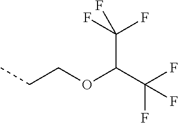 | log P |
| I-A-Q2-049 | 3-Cl | H | 5-Cl | NH₂ | CF₃ | Me | CH | CH | O | H | 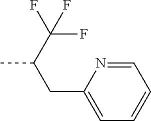 | log P |
| I-A-Q2-050 | 3-Cl | H | 5-Cl | NH₂ | CF₃ | Me | CH | CH | O | H | 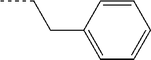 | log P |
| I-A-Q2-051 | 3-Cl | H | 5-Cl | NH₂ | CF₃ | Me | CH | CH | O | H | 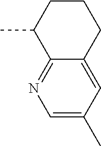 | log P |
| I-A-Q2-052 | 3-Cl | H | 5-Cl | NH₂ | CF₃ | Me | CH | CH | O | H | 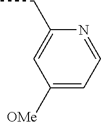 | log P |
| I-A-Q2-053 | 3-Cl | H | 5-Cl | NH₂ | CF₃ | Me | CH | CH | O | H | 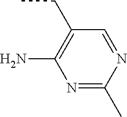 | log P |
| I-A-Q2-054 | 3-Cl | H | 5-Cl | NH₂ | CF₃ | Me | CH | CH | O | H | 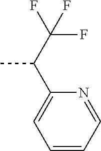 | log P |
| I-A-Q2-055 | 3-Cl | H | 5-Cl | NH₂ | CF₃ | Me | CH | CH | O | H | 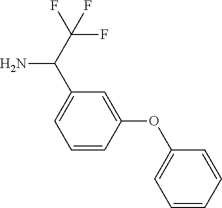 | log P |
| I-A-Q2-056 | 3-Cl | H | 5-Cl | NH₂ | CF₃ | Me | CH | CH | O | Me | Me | log P |
| I-A-Q2-057 | 3-Cl | H | 5-Cl | NH₂ | CF₃ | Me | CH | CH | O | H | 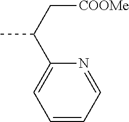 | log P |
| I-A-Q2-058 | 3-Cl | H | 5-Cl | NH₂ | CF₃ | Me | CH | CH | O | H | 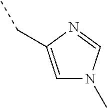 | log P |
| I-A-Q2-059 | 3-Cl | H | 5-Cl | NH₂ | CF₃ | Me | CH | CH | O | H | 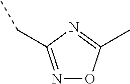 | log P |
| I-A-Q2-060 | 3-Cl | H | 5-Cl | NH₂ | CF₃ | Me | CH | CH | O | H | 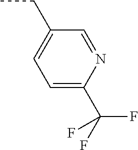 | log P |
| I-A-Q2-061 | 3-Cl | H | 5-Cl | NH₂ | CF₃ | Me | CH | CH | O | H | 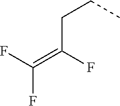 | log P |
| I-A-Q2-062 | 3-Cl | H | 5-Cl | NH₂ | CF₃ | Me | CH | CH | O | H | CH₂CHCH₂ | log P |
| I-A-Q2-063 | 3-Cl | H | 5-Cl | SOMe | CF₃ | Me | CH | CH | O | H | CH₂-2-Pyr | log P |
| | | | | | | | | | | | | ¹H-NMR |
| I-A-Q2-064 | 3-Cl | H | 5-Cl | N═CHNMe₂ | CF₃ | Me | CH | CH | O | H | 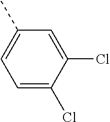 | log P |
| I-A-Q2-065 | 3-Cl | H | 5-Cl | NH₂ | CF₃ | Me | CH | CH | O | H | 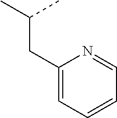 | log P |
| I-A-Q2-066 | 3-Cl | H | 5-Cl | NH₂ | CF₃ | Me | CH | CH | O | H | 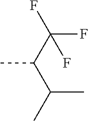 | log P |
| I-A-Q2-067 | 3-Cl | H | 5-Cl | NH₂ | CF₃ | H | CH | CH | O | H | 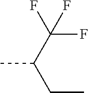 | log P |
| I-A-Q2-068 | 3-Cl | H | 5-CF₃ | SMe | CF₃ | H | CH | CH | O | H | TFiPr | log P |
| I-A-Q2-069 | 3-Cl | H | 5-Cl | NH₂ | CF₃ | H | CH | CH | O | H | CMe₂CH₂CF₃ | log P |
| I-A-Q2-070 | 3-Cl | H | 5-Cl | NH₂ | CF₃ | H | CH | CH | O | H | 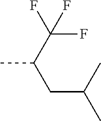 | log P |
| I-A-Q2-071 | 3-Cl | H | 5-Cl | NH₂ | CF₃ | H | CH | CH | O | H | 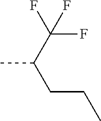 | log P |
| I-A-Q2-072 | 3-Cl | H | 5-Cl | NH₂ | CF₃ | H | CH | CH | O | H | 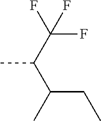 | log P |
| I-A-Q2-073 | 3-Cl | H | 5-Cl | NH₂ | CF₃ | H | CH | CH | O | H | 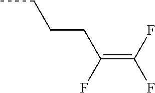 | log P |
| I-A-Q2-074 | H | 4-Cl | H | NH₂ | iPr | H | CH | CH | O | H | Me | log P |
| I-A-Q2-075 | H | 4-Cl | H | NH₂ | Ph | H | CH | CH | O | H | TFiPr | log P |
| I-A-Q2-076 | H | 4-Cl | H | NH₂ | Ph | H | CH | CH | O | H | CH₂-2-Pyrm | log P |
| I-A-Q2-077 | H | 4-Cl | H | NH₂ | Ph | H | CH | CH | O | H | CHMePh | log P |
| I-A-Q2-078 | H | 4-Cl | H | NH₂ | Ph | H | CH | CH | O | H | Me | log P |
| I-A-Q2-079 | 2-Cl | H | H | NH₂ | CF₃ | H | CH | CH | O | H | TFiPr | log P |
| I-A-Q2-080 | 2-Cl | H | H | NH₂ | CF₃ | H | CH | CH | O | H | CH₂-2-Pyrm | log P |
| I-A-Q2-081 | 2-Cl | H | H | NH₂ | CF₃ | H | CH | CH | O | H | CHMePh | log P |
| I-A-Q2-082 | 2-Cl | H | H | NH₂ | CF₃ | H | CH | CH | O | H | Me | log P |
| I-A-Q2-083 | 3-Cl | H | H | NH₂ | CF₃ | H | CH | CH | O | H | TFiPr | log P |
| I-A-Q2-084 | 3-F | H | 5-Cl | NH₂ | CF₃ | H | CH | CH | O | H | TFiPr | log P |
| I-A-Q2-085 | 3-Cl | H | H | NH₂ | CF₃ | H | CH | CH | O | H | CH₂CF₃ | log P |
| I-A-Q2-086 | 3-F | H | 5-Cl | NH₂ | CF₃ | H | CH | CH | O | H | CH₂CF₃ | log P |
| I-A-Q2-087 | 3-Cl | H | H | NH₂ | CF₃ | H | CH | CH | O | H | CHMe-2-Pyr | log P |
| I-A-Q2-088 | 3-F | H | 5-Cl | NH₂ | CF₃ | H | CH | CH | O | H | CHMe-2-Pyr | log P |
| I-A-Q2-089 | 3-Cl | H | H | NH₂ | CF₃ | H | CH | CH | O | H | CHMePh | log P |
| I-A-Q2-090 | 3-F | H | 5-Cl | NH₂ | CF₃ | H | CH | CH | O | H | CHMePh | log P |
| I-A-Q2-091 | 3-Cl | H | H | NH₂ | CF₃ | H | CH | CH | O | H | Cypr | log P |
| I-A-Q2-092 | 3-F | H | 5-Cl | NH₂ | CF₃ | H | CH | CH | O | H | Cypr | log P |
| I-A-Q2-093 | 3-Cl | H | H | NH₂ | CF₃ | H | CH | CH | O | H | Cyhex | log P |
| I-A-Q2-094 | 3-Cl | H | H | NH₂ | CF₃ | H | CH | CH | O | H | Me | log P |
| I-A-Q2-095 | 3-Cl | H | 5-CF₃ | NH₂ | CF₃ | H | CH | CH | O | H | TFiPr | log P |
| I-A-Q2-096 | 3-Cl | H | 5-Cl | Br | CF₃ | H | CH | CH | O | H | TFiPr | log P |
| I-A-Q2-097 | 3-Cl | H | 5-Cl | NH₂ | CF₃ | H | N | CH | O | H | iPr | log P |
| I-A-Q2-098 | 3-Cl | H | H | Br | Me | H | CH | CH | O | H | TFiPr | log P |
| I-A-Q2-099 | 3-Cl | H | 5-Cl | NH₂ | CF₃ | H | N | CH | O | H | CMe₂CH₂CF₃ | log P |
| I-A-Q2-100 | 3-Cl | H | 5-Cl | NH₂ | CF₃ | H | N | CH | O | H | TFiPr | log P |
| I-A-Q2-101 | 3-Cl | H | 5-Cl | NH₂ | CF₃ | H | N | CH | O | H | CH₂CF₃ | log P |
| I-A-Q2-102 | 3-Cl | H | 5-Cl | NH₂ | CF₃ | H | N | CH | O | H | CHMe-2-Pyr | log P |
| I-A-Q2-103 | 3-Cl | H | 5-Cl | NH₂ | CF₃ | H | N | CH | O | H | CHMePh | log P |
| I-A-Q2-104 | 3-Cl | H | 5-Cl | NH₂ | CF₃ | H | N | CH | O | H | CH₂CHCH₂ | log P |
| I-A-Q2-105 | 3-Cl | H | 5-Cl | NH₂ | CF₃ | H | N | CH | O | H | CH₂-2-Pyr | log P |
| I-A-Q2-106 | 3-Cl | H | 5-Cl | NH₂ | CF₃ | H | N | CH | O | H | Cypr | log P |
| I-A-Q2-107 | 3-Cl | H | 5-Cl | NH₂ | CF₃ | H | N | CH | O | H | CH₂-3-Pyr | log P |
| I-A-Q2-108 | 3-Cl | H | 5-Cl | NH₂ | CF₃ | H | N | CH | O | H | CH₂-2-Pyrm | log P |
| I-A-Q2-109 | 3-Cl | H | 5-Cl | Br | CF₃ | H | N | CH | O | H | Me | log P |
| I-A-Q2-110 | 3-Cl | H | 5-Cl | NH₂ | CF₃ | Me | CH | CH | O | Me | CH₂-2-Pyr | log P |
| I-A-Q2-111 | 3-Cl | H | 5-Cl | NH₂ | CF₃ | Me | CH | CH | O | Cypr | CH₂-2-Pyr | log P |
| I-A-Q2-112 | 3-Cl | H | 5-Cl | NH₂ | CF₃ | Me | CH | CH | O | H | 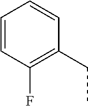 | log P |
| I-A-Q2-113 | 3-Cl | H | 5-Cl | H | CF₃ | Me | CH | CH | O | H | CH₂Cypr | |
| I-A-Q2-114 | 3-CF₃ | H | 5-CF₃ | H | CF₃ | CN | CH | CH | O | H | CH₂C₂F₅ | |
| I-A-Q2-115 | 3-CF₃ | H | H | NH₂ | CF₃ | NO₂ | CH | CH | O | H | CHMe₂ | |
| I-A-Q2-116 | 3-Br | H | 5-Br | NH₂ | CF₃ | CF₃ | CH | CH | O | H | CH₂CH₂SMe | |
| I-A-Q2-117 | 3-Cl | 4-Cl | 5-Cl | Cl | CF₃ | Br | CH | CH | O | H | TFiPr | |
| I-A-Q2-118 | 3-Br | 4-Cl | 5-Br | Cl | C₂F₅ | Cl | CH | CH | O | H | HFiPr | |
| I-A-Q2-119 | 3-CF₃ | 4-Cl | 5-Cl | Br | C₂F₅ | CH₂OMe | CH | CH | O | H | CH₂Ph | |
| I-A-Q2-120 | 3-CF₃ | 4-F | H | Br | C₂F₅ | CH₂NHMe | CH | CH | O | H | CH₂-2-Pyr | |
| I-A-Q2-121 | 3-Cl | 4-CF₃ | 5-Cl | I | C₂F₅ | Me | CH | CH | O | H | CH₂-2-Pyr | |
| I-A-Q2-122 | 3-CF₃ | H | 5-Cl | I | CF₃ | CN | CH | CH | O | H | CHMe-2-Pyr | |
| I-A-Q2-123 | 3-Cl | 4-CF₃ | 5-Cl | CN | CF₃ | NO₂ | N | N | O | H | CHMe-2-Pyr | |
| I-A-Q2-124 | 3-Cl | 4-Br | 5-Cl | CN | CF₃ | CF₃ | N | N | O | H | CH₂-3-Pyr | |
| I-A-Q2-125 | 3-F | 4-F | 5-F | NHCH₂Ph | Cl | Br | CH | CH | O | H | CH₂-3-Pyr | |
| I-A-Q2-126 | 2-Cl | 3-CF₃ | 4-Cl | NHAc | Br | Cl | CH | CH | S | H | CHMe-3-Pyr | |
| I-A-Q2-127 | 3-Cl | 4-CF₃ | H | NAc₂ | CF₃ | CH₂OAc | CH | CH | S | H | CHMe-3-Pyr | |
| I-A-Q2-128 | 3-Br | 4-NH₂ | 5-Br | NHMe | CF₂Ph | CH₂NHAc | CH | CH | O | Me | CH₂-4-Pyr | |
| I-A-Q2-129 | 3-Br | H | H | NMe₂ | CF₂OMe | Me | CH | CH | O | CH₂CF₃ | CH₂-4-Pyr | |
| I-A-Q2-130 | 3-Cl | 4-Cl | H | SMe | CF₂OPh | CN | CH | CH | O | CH₂Ph | CHMe-4-Pyr | |
| I-A-Q2-131 | 2-Cl | 3-Cl | 4-Cl | SOMe | CF₂(2-Pyr) | NO₂ | N | CH | O | CH₂-4-Pyr | CHMe-4-Pyr | |
| I-A-Q2-132 | 3-NO₂ | H | H | SO₂Me | CF₂O(2-Pyr) | CF₃ | CH | N | O | Cypr | CH₂-2-Pyrm | |
| I-A-Q2-133 | 3-Cl | H | 5-Cl | NH₂ | CF₃ | H | CH | CH | O | H | TFiPr | log P |
| I-A-Q2-134 | 3-Cl | H | 5-Cl | NH₂ | CF₃ | H | CH | CH | O | H | CH₂-2-Pyr | log P |
| I-A-Q2-135 | 3-Cl | H | 5-Cl | NH₂ | CF₃ | H | CH | CH | O | H | iPr | log P |
| I-A-Q2-136 | 3-Cl | H | 5-Cl | NH₂ | CF₃ | H | CH | CH | O | H | Cypr | log P |
| I-A-Q2-137 | 3-Cl | H | 5-Cl | NH₂ | CF₃ | H | CH | CH | O | H | CH₂-2-Pyrm | log P |
| I-A-Q2-138 | 3-Cl | H | 5-Cl | NH₂ | CF₃ | H | CH | CH | O | H | CH₂—CF₃ | log P |
| I-A-Q2-139 | 3-Cl | H | 5-Cl | NH₂ | CF₃ | H | CH | CH | O | H | CH₂—CH₂—CF₃ | log P |
| I-A-Q2-140 | 3-Cl | H | 5-Cl | NH₂ | CF₃ | H | CH | CH | O | H | CH₂—CHF₂ | log P |
| I-A-Q2-141 | 3-Cl | H | 5-Cl | NH₂ | CF₃ | H | CH | CH | O | H | 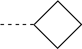 | log P |
| I-A-Q2-142 | 3-Cl | H | 5-Cl | NH₂ | CF₃ | H | CH | CH | O | H | Et | log P |
| I-A-Q2-143 | 3-Cl | H | 5-Cl | NH₂ | CF₃ | H | CH | CH | O | H | CH₂-3-Pyr | log P |
| I-A-Q2-144 | 3-Cl | H | 5-Cl | NH₂ | CF₃ | H | CH | CH | O | H | CH₂—CH═CH₂ | log P |
| I-A-Q2-145 | 3-Cl | H | 5-Cl | NH₂ | CF₃ | H | CH | CH | O | H | CH(Me)Et | log P |
| I-A-Q2-146 | 3-Cl | H | 5-Cl | NH₂ | CF₃ | H | CH | CH | O | H | tBu | log P |
| I-A-Q2-147 | 3-Cl | H | 5-Cl | NH₂ | CF₃ | H | CH | CH | O | H | CHMe-2-Pyr | log P |
| I-A-Q2-148 | 3-Cl | H | 5-Cl | NH₂ | CF₃ | H | CH | CH | O | H | 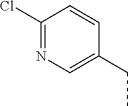 | log P |
| I-A-Q2-149 | 3-Cl | H | 5-Cl | NH₂ | CF₃ | H | CH | CH | O | H | CH₂-Cypr | log P |
| I-A-Q2-150 | 3-Cl | H | 5-Cl | NH₂ | CF₃ | H | CH | CH | O | H | CHMe—Ph | log P |
| I-A-Q2-151 | 3-Cl | H | 5-Cl | NH₂ | CF₃ | H | CH | CH | O | H | Pr | log P |
| I-A-Q2-152 | 3-Cl | H | 5-Cl | NH₂ | CF₃ | H | CH | CH | O | H | CH₂—Ph | log P |
| I-A-Q2-153 | H | 4-Cl | H | NH₂ | CF₃ | H | CH | CH | O | H | TFiPr | log P |
| I-A-Q2-154 | 3-Cl | 4-Cl | H | NH₂ | CF₃ | H | CH | CH | O | H | iPr | log P |
| I-A-Q2-155 | 3-Cl | 4-Cl | H | NH₂ | CF₃ | H | CH | CH | O | H | TFiPr | log P |
| I-A-Q2-156 | 3-Cl | 4-Cl | H | NH₂ | CF₃ | H | CH | CH | O | H | CH₂—CF₃ | log P |
| I-A-Q2-157 | 3-Cl | 4-Cl | H | NH₂ | CF₃ | H | CH | CH | O | H | Cypr | log P |
| I-A-Q2-158 | 3-Cl | 4-Cl | H | NH₂ | CF₃ | H | CH | CH | O | H | CH₂-2-Pyr | log P |
| I-A-Q2-159 | 3-Cl | 4-Cl | H | NH₂ | CF₃ | H | CH | CH | O | H | CH₂—CHF₂ | log P |
| I-A-Q2-160 | 3-Cl | 4-Cl | H | NH₂ | CF₃ | H | CH | CH | O | H | CHMe-2-Pyr | log P |
| I-A-Q2-161 | H | 4-Cl | H | NH₂ | CF₃ | H | CH | CH | O | H | iPr | log P |
| I-A-Q2-162 | H | 4-Cl | H | NH₂ | CF₃ | H | CH | CH | O | H | CH₂—CF₃ | log P |
| I-A-Q2-163 | H | 4-Cl | H | NH₂ | CF₃ | H | CH | CH | O | H | CyPr | log P |
| I-A-Q2-164 | H | 4-Cl | H | NH₂ | CF₃ | H | CH | CH | O | H | CH₂—CHF₂ | log P |
| I-A-Q2-165 | H | 4-Cl | H | NH₂ | CF₃ | H | CH | CH | O | H | CHMe-2-Pyr | log P |
| I-A-Q2-166 | 3-F | 4-Cl | H | NH₂ | CF₃ | H | CH | CH | O | H | TFiPr | log P |
| I-A-Q2-167 | 3-F | 4-Cl | H | NH₂ | CF₃ | H | CH | CH | O | H | Cypr | log P |
| I-A-Q2-168 | 3-F | 4-Cl | H | NH₂ | CF₃ | H | CH | CH | O | H | CH₂-2-Pyr | log P |
| I-A-Q2-169 | 4-Cl | 3-F | H | NH₂ | CF₃ | H | CH | CH | O | H | TFiPr | log P |
| I-A-Q2-170 | 4-Cl | 3-F | H | NH₂ | CF₃ | H | CH | CH | O | H | Cypr | log P |
| I-A-Q2-171 | 4-Cl | 3-F | H | NH₂ | CF₃ | H | CH | CH | O | H | CH₂-2-Pyr | log P |
| I-A-Q2-172 | 3-Br | H | H | NH₂ | CF₃ | H | CH | CH | O | H | TFiPr | log P |
| I-A-Q2-173 | 3-Br | H | H | NH₂ | CF₃ | H | CH | CH | O | H | Cypr | log P |
| I-A-Q2-174 | 3-Br | H | H | NH₂ | CF₃ | H | CH | CH | O | H | CH₂-2-Pyr | log P |
| I-A-Q2-175 | 3-F | 4-F | H | NH₂ | CF₃ | H | CH | CH | O | H | TFiPr | log P |
| I-A-Q2-176 | 3-F | 4-F | H | NH₂ | CF₃ | H | CH | CH | O | H | Cypr | log P |
| I-A-Q2-177 | 3-F | 4-F | H | NH₂ | CF₃ | H | CH | CH | O | H | CH₂-2-Pyr | log P |
| I-A-Q2-178 | 3-Me | H | 5-Me | NH₂ | CF₃ | H | CH | CH | O | H | TFiPr | log P |
| I-A-Q2-179 | 3-Me | H | 5-Me | NH₂ | CF₃ | H | CH | CH | O | H | Cypr | log P |
| I-A-Q2-180 | 3-Me | H | 5-Me | NH₂ | CF₃ | H | CH | CH | O | H | CH₂-2-Pyr | log P |
| I-A-Q2-181 | 3-F | H | H | NH₂ | CF₃ | H | CH | CH | O | H | iPr | log P |
| I-A-Q2-182 | 3-F | H | H | NH₂ | CF₃ | H | CH | CH | O | H | TFiPr | log P |
| I-A-Q2-183 | 3-F | H | H | NH₂ | CF₃ | H | CH | CH | O | H | CH₂—CF₃ | log P |
| I-A-Q2-184 | 3-F | H | H | NH₂ | CF₃ | H | CH | CH | O | H | Cypr | log P |
| I-A-Q2-185 | 3-F | H | H | NH₂ | CF₃ | H | CH | CH | O | H | CH₂-2-Pyr | log P |
| I-A-Q2-186 | 3-F | H | H | NH₂ | CF₃ | H | CH | CH | O | H | CHMe-2-Pyr | log P |
| I-A-Q2-187 | H | 4-Br | H | NH₂ | CF₃ | H | CH | CH | O | H | iPr | log P |
| I-A-Q2-188 | H | 4-Br | H | NH₂ | CF₃ | H | CH | CH | O | H | TFiPr | log P |
| I-A-Q2-189 | H | 4-Br | H | NH₂ | CF₃ | H | CH | CH | O | H | CH₂—CF₃ | log P |
| I-A-Q2-190 | H | 4-Br | H | NH₂ | CF₃ | H | CH | CH | O | H | Cypr | log P |
| I-A-Q2-191 | H | 4-Br | H | NH₂ | CF₃ | H | CH | CH | O | H | CH₂-2-Pyr | log P |
| I-A-Q2-192 | H | 4-Br | H | NH₂ | CF₃ | H | CH | CH | O | H | CHMe-2-Pyr | log P |
| I-A-Q2-193 | 3-Cl | H | 5-Cl | NH₂ | CF₃ | H | CH | CH | O | H | Me | log P |
| I-A-Q2-194 | 3-Cl | 4-Cl | H | NH₂ | CF₃ | H | CH | CH | O | H | Me | log P |
| I-A-Q2-195 | H | 4-Cl | H | NH₂ | CF₃ | H | CH | CH | O | H | Me | log P |
| I-A-Q2-196 | 3-F | 4-F | 5-F | NH₂ | CF₃ | H | CH | CH | O | H | CHMe-2-Pyr | log P |
| I-A-Q2-197 | 3-Cl | H | 5-Cl | NHAc | CF₃ | Me | CH | CH | O | H | CH₂-2-Pyr | log P |
| I-A-Q2-198 | 3-Cl | H | 5-Cl | NH₂ | CF₃ | Me | CH | CH | O | H | CH(CF₃)(CH₂iPr) | log P |
| I-A-Q2-199 | 3-Cl | H | 5-Cl | NH₂ | CF₃ | Me | CH | CH | O | CH₂-2-Pyr | CH₂-2-Pyr | log P |
| I-A-Q2-200 | 3-Cl | H | 5-Cl | NH₂ | CF₃ | Me | CH | CH | O | H | CH(CF₃)(Et) | log P |
| I-A-Q2-201 | 3-Cl | H | 5-Cl | NH₂ | CF₃ | Me | CH | CH | O | H | CH(CF₃)(Pr) | log P |
| I-A-Q2-202 | 3-Cl | H | 5-Cl | NH₂ | CF₃ | Me | CH | CH | O | H | Pr | log P |
| I-A-Q2-203 | 3-Cl | H | 5-Cl | NH₂ | CF₃ | Me | CH | CH | O | H | 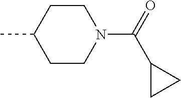 | log P |
| I-A-Q2-204 | 3-Cl | H | 5-Cl | NH₂ | CF₃ | Me | CH | CH | O | H | 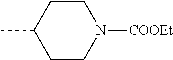 | log P |
| I-A-Q2-205 | 3-Cl | H | 5-Cl | NH₂ | CF₃ | Me | CH | CH | O | H | CH₂CH₂SMe | log P |
| I-A-Q2-206 | 3-Cl | H | 5-Cl | NH₂ | CF₃ | Me | CH | CH | O | H | 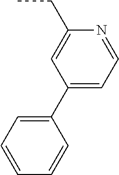 | log P |
| I-A-Q2-207 | 3-Cl | H | 5-Cl | NH₂ | CF₃ | Me | CH | CH | O | H | 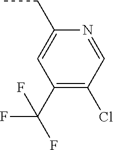 | log P |
| I-A-Q2-208 | 3-Cl | H | 5-Cl | NH₂ | CF₃ | Me | CH | CH | O | Me | CHMe-3-Pyr | log P |
| I-A-Q2-209 | 3-Cl | H | 5-Cl | NH₂ | CF₃ | Me | CH | CH | O | Me | CH₂-3-Pyr | log P |
| I-A-Q2-210 | 3-Cl | H | 5-Cl | NH₂ | CF₃ | Me | CH | CH | O | Me | CHMe-2-Pyr | log P |
| I-A-Q2-211 | 3-Cl | H | 5-Cl | NH₂ | CF₃ | Me | CH | CH | O | Me | CHMe-4-Pyr | log P |
| I-A-Q2-212 | 3-Cl | H | 5-Cl | NH₂ | CF₃ | Me | CH | CH | O | H | CH₂-4-Pyr | log P |
| I-A-Q2-213 | 3-Cl | H | 5-Cl | NH₂ | CF₃ | Me | CH | CH | O | H | CHMe-(4-Cl—Ph) | log P |
| I-A-Q2-214 | 3-Cl | H | 5-Cl | NH₂ | CF₃ | Me | CH | CH | O | H | CH₂-(3-F—Ph) | log P |
| I-A-Q2-215 | 3-Cl | H | 5-Cl | NH₂ | CF₃ | Me | CH | CH | O | H | CH₂-(3-Cl—Ph) | log P |
| I-A-Q2-216 | 3-Cl | H | 5-Cl | NH₂ | CF₃ | Me | CH | CH | O | H | CH₂-3-Pyr | log P |
| I-A-Q2-217 | 3-Cl | H | 5-Cl | NH₂ | CF₃ | Me | CH | CH | O | H | 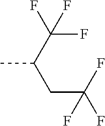 | log P |
| I-A-Q2-218 | 3-Cl | H | 5-Cl | NH₂ | CF₃ | Me | CH | CH | O | H | 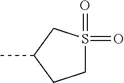 | log P |
| I-A-Q2-219 | 3-Cl | H | 5-Cl | NH₂ | CF₃ | Me | CH | CH | O | H | 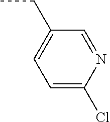 | log P |
| I-A-Q2-220 | 3-Cl | H | 5-Cl | NH₂ | CF₃ | Me | CH | CH | O | H | CH₂-Cypr | log P |
| I-A-Q2-221 | 3-Cl | H | 5-Cl | NH₂ | CF₃ | Me | CH | CH | O | H | tBu | log P |
| I-A-Q2-222 | 3-Cl | H | 5-Cl | NH₂ | CF₃ | Me | CH | CH | O | H | Cyhex | log P |
| I-A-Q2-223 | 3-Cl | H | 5-Cl | NH₂ | CF₃ | Me | CH | CH | O | H | Cypen | log P |
| I-A-Q2-224 | 3-Cl | H | 5-Cl | NH₂ | CF₃ | Me | CH | CH | O | H | CH₂CH(OMe)₂ | log P |
| I-A-Q2-225 | 3-Cl | H | 5-Cl | NH₂ | CF₃ | Me | CH | CH | O | H | CHEt₂ | log P |
| I-A-Q2-226 | 3-Cl | H | 5-Cl | NH₂ | CF₃ | Me | CH | CH | O | H | CH₂CH₂-2-Pyr | log P |
| I-A-Q2-227 | 3-Cl | H | 5-Cl | NH₂ | CF₃ | Me | CH | CH | O | H | CH₂—iPr | log P |
| I-A-Q2-228 | 3-Cl | H | 5-Cl | NH₂ | CF₃ | Me | CH | CH | O | H | Cypr | log P |
| I-A-Q2-229 | 3-Cl | H | 5-Cl | NH₂ | CF₃ | Me | CH | CH | O | H | iPr | log P |
| I-A-Q2-230 | 3-Cl | H | 5-Cl | NH₂ | CF₃ | Me | CH | CH | O | H | 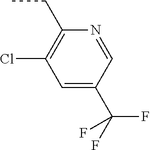 | log P |
| I-A-Q2-231 | 3-Cl | H | 5-Cl | NH₂ | CF₃ | Me | CH | CH | O | H | CHMe-3-Pyr | log P |
| I-A-Q2-232 | 3-Cl | H | 5-Cl | NH₂ | CF₃ | Me | CH | CH | O | H | 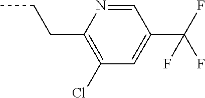 | log P |
| I-A-Q2-233 | 3-Cl | H | 5-Cl | NH₂ | CF₃ | Me | CH | CH | O | H | 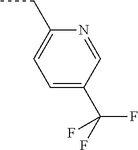 | log P |
| I-A-Q2-234 | 3-Cl | H | 5-Cl | NH₂ | CF₃ | Me | CH | CH | O | H | CHMe-4-Pyr | log P |
| I-A-Q2-235 | 3-Cl | H | 5-Cl | NH₂ | CF₃ | Me | CH | CH | O | H | 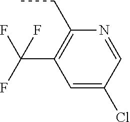 | log P |
| I-A-Q2-236 | 3-Cl | H | 5-Cl | NH₂ | CF₃ | Me | CH | CH | O | Me | CH₂CF₃ | log P |
| I-A-Q2-237 | 3-Cl | H | 5-Cl | NH₂ | CF₃ | Me | CH | CH | O | H | Pen | log P |
| I-A-Q2-238 | 3-Cl | H | 5-Cl | NH₂ | CF₃ | Me | CH | CH | O | H | CH₂CCH | log P |
| I-A-Q2-239 | 3-Cl | H | 5-Cl | NH₂ | CF₃ | Me | CH | CH | O | H | Cybu | log P |
| I-A-Q2-240 | 3-Cl | H | 5-Cl | NH₂ | CF₃ | Me | CH | CH | O | H | 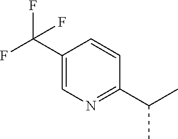 | log P |
| I-A-Q2-241 | 3-Cl | H | 5-Cl | NH₂ | CF₃ | Me | CH | CH | O | H | CH(CF₃)Ph | log P |
| I-A-Q2-242 | 3-Cl | H | 5-Cl | NH₂ | CF₃ | Me | CH | CH | O | H | CH(CF₃)(4-CF₃Ph) | log P |
| I-A-Q2-243 | 3-Cl | H | 5-Cl | NH₂ | CF₃ | Me | CH | CH | O | H | 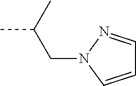 | log P |
| I-A-Q2-244 | 3-Cl | H | 5-Cl | NH₂ | CF₃ | Me | CH | CH | O | H | 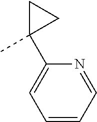 | log P |
| I-A-Q2-245 | 3-Cl | H | 5-Cl | NH₂ | CF₃ | Me | CH | CH | O | H | 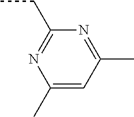 | log P |
| I-A-Q2-246 | 3-Cl | H | 5-Cl | NH₂ | CF₃ | Me | CH | CH | O | H | CH(CF₃)(4-NMe₂—Ph) | log P |
| I-A-Q2-247 | 3-Cl | H | 5-Cl | NH₂ | CF₃ | Me | CH | CH | O | H | CHMe-(4-F—Ph) | log P |
| I-A-Q2-248 | 3-Cl | H | 5-Cl | NH₂ | CF₃ | Me | CH | CH | O | H | 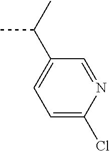 | log P |
| I-A-Q2-249 | 3-Cl | H | 5-Cl | NH₂ | CF₃ | Me | CH | CH | O | H | CH₂-(4-NO₂—Ph) | log P |
| I-A-Q2-250 | 3-Cl | H | 5-Cl | NH₂ | CF₃ | Me | CH | CH | O | H | 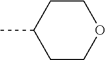 | log P |
| I-A-Q2-251 | 3-Cl | H | 5-Cl | NH₂ | CF₃ | Me | CH | CH | O | H | 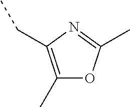 | log P |
| I-A-Q2-252 | 3-Cl | H | 5-Cl | NH₂ | CF₃ | Me | CH | CH | O | H | 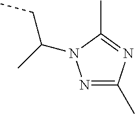 | log P |
| I-A-Q2-253 | 3-Cl | H | 5-Cl | NH₂ | CF₃ | Me | CH | CH | O | H | 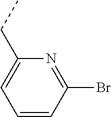 | log P |
| I-A-Q2-254 | 3-Cl | H | 5-Cl | NH₂ | CF₃ | Me | CH | CH | O | H | 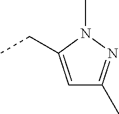 | log P |
| I-A-Q2-255 | 3-Cl | H | 5-Cl | NH₂ | CF₃ | Me | CH | CH | O | H | 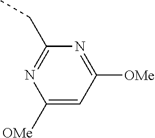 | log P |
| I-A-Q2-256 | 3-Cl | H | 5-Cl | NH₂ | CF₃ | Me | CH | CH | O | H | 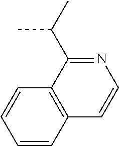 | log P |
| I-A-Q2-257 | 3-Cl | H | 5-Cl | NH₂ | CF₃ | Me | CH | CH | O | H | CH(CF₃)(3-CF₃—Ph) | log P |
| I-A-Q2-258 | 3-Cl | H | 5-Cl | NH₂ | CF₃ | Me | CH | CH | O | H | 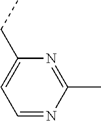 | log P |
| I-A-Q2-259 | 3-Cl | H | 5-Cl | NH₂ | CF₃ | Me | CH | CH | O | H | 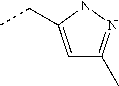 | log P |
| I-A-Q2-260 | 3-Cl | H | 5-Cl | NH₂ | CF₃ | Me | CH | CH | O | H | 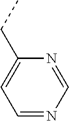 | log P |
| I-A-Q2-261 | 3-Cl | H | 5-Cl | NH₂ | CF₃ | Me | CH | CH | O | H | 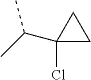 | log P |
| I-A-Q2-262 | 3-Cl | H | 5-Cl | NH₂ | CF₃ | Me | CH | CH | O | H | CH₂CHMe(OH) | log P |
| I-A-Q2-263 | 3-Cl | H | 5-Cl | NH₂ | CF₃ | Me | CH | CH | O | H | CH₂-Cyhex | log P |
| I-A-Q2-264 | 3-Cl | H | 5-Cl | NH₂ | CF₃ | Me | CH | CH | O | H | 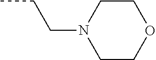 | log P |
| I-A-Q2-265 | 3-Cl | H | 5-Cl | NH₂ | CF₃ | Me | CH | CH | O | H | 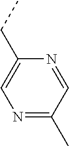 | log P |
| I-A-Q2-266 | 3-Cl | H | 5-Cl | NH₂ | CF₃ | Me | CH | CH | O | H | CH₂CF₂CF₂CF₃ | log P |
| I-A-Q2-267 | 3-Cl | H | 5-Cl | NH₂ | CF₃ | Me | CH | CH | O | H | CH₂(CF₂)₆CF₃ | log P |
| I-A-Q2-268 | 3-Cl | H | 5-Cl | NH₂ | CF₃ | Me | CH | CH | O | H | CH₂CF₂Br | log P |
| I-A-Q2-269 | 3-Cl | H | 5-Cl | NH₂ | CF₃ | Me | CH | CH | O | H | CH₂CF₂CF₃ | log P |
| I-A-Q2-270 | 3-Cl | H | 5-Cl | NH₂ | CF₃ | Me | CH | CH | O | H | Et | log P |
| I-A-Q2-271 | 3-Cl | H | 5-Cl | NH₂ | CF₃ | Me | CH | CH | O | H | CH₂Ph | log P |
| I-A-Q2-272 | 3-Cl | H | 5-Cl | NH₂ | CF₃ | Me | CH | CH | O | H | CH₂CH₂OH | log P |
| I-A-Q2-273 | 3-Cl | H | 5-Cl | NH₂ | CF₃ | Me | CH | CH | O | H | CH₂CH₂S(O)Me | log P |
| I-A-Q2-274 | 3-Cl | H | 5-Cl | NH₂ | CF₃ | Me | CH | CH | O | H | 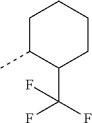 | log P |
| I-A-Q2-275 | 3-Cl | H | 5-Cl | I | CF₃ | Me | CH | CH | O | H | CH₂-2-Pyrm | log P |
| I-A-Q2-276 | 3-Cl | H | 5-Cl | SMe | CF₃ | Me | CH | CH | O | H | CH₂-2-Pyrm | log P |
| I-A-Q2-277 | 3-Cl | H | 5-Cl | Cl | CF₃ | Me | CH | CH | O | H | CH₂-2-Pyrm | log P |
| I-A-Q2-278 | 3-Cl | H | 5-Cl | NH₂ | CF₃ | Me | CH | CH | O | H | 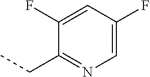 | log P |
| I-A-Q2-279 | 3-Cl | H | 5-Cl | NH₂ | CF₃ | Me | CH | CH | O | H | 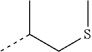 | log P |
| I-A-Q2-280 | 3-Cl | H | 5-Cl | NH₂ | CF₃ | Me | CH | CH | O | H | 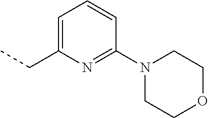 | log P |
| I-A-Q2-281 | 3-Cl | H | 5-Cl | NH₂ | CF₃ | Me | CH | CH | O | H | 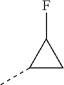 | log P |
| I-A-Q2-282 | 3-Cl | H | 5-Cl | SMe | CF₃ | Me | CH | CH | O | H | CH₂CF₂CF₂H | log P |
| I-A-Q2-283 | 3-Cl | H | 5-Cl | SOMe | CF₃ | Me | CH | CH | O | H | CH₂CF₂CF₂H | log P |
| I-A-Q2-284 | 3-Cl | H | 5-Cl | SOMe | CF₃ | Me | CH | CH | O | H | CH₂CF₂CF₂H | log P |
| I-A-Q2-285 | 3-Cl | H | 5-CF₃ | NH₂ | (3-BrC₆H₄)CF₂ | Me | CH | CH | O | H | CH₂CF₂CF₂H | log P |
| I-A-Q2-286 | 3-Cl | H | 5-CF₃ | NH₂ | (3-CF₃C₆H₄)CF₂ | Me | CH | CH | O | H | CH₂CF₂CF₂H | log P |
| I-A-Q2-287 | 3-Cl | H | 5-CF₃ | NH₂ | CF₃OCF₂CF₂OCF₂ | Me | CH | CH | O | H | CH₂CF₂CF₂H | log P |
| I-A-Q2-288 | 3-Cl | H | 5-CF₃ | NH₂ | (3-MeOC₆H₄)CF₂ | Me | CH | CH | O | H | CH₂CF₂CF₂H | log P |
| I-A-Q2-289 | 3-Cl | H | 5-CF₃ | NH₂ | (4-CF₃C₆H₄)CF₂ | Me | CH | CH | O | H | CH₂CF₂CF₂H | log P |
| I-A-Q2-290 | 3-Cl | H | 5-CF₃ | NH | 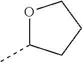 | Me | CH | CH | O | H | CH₂CF₂CF₂H | log P |
| I-A-Q2-291 | 3-Cl | H | 5-CF₃ | NH₂ | 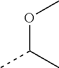 | Me | CH | CH | O | H | CH₂CF₂CF₂H | log P |
| I-A-Q2-292 | 3-Cl | 4-MeO | 5-MeO | NH₂ | CF₃ | Me | CH | CH | O | H | CH₂CF₂CF₂H | log P |
| I-A-Q2-293 | 3,4-OCH₂O— | | 5-Cl | NH₂ | CF₃ | Me | CH | CH | O | H | CH₂CF₂CF₂H | log P |
| I-A-Q2-294 | 3-Cl | H | 5-CF₃ | NH₂ | (4-Cl—C₆H₄)OCF₂ | Me | CH | CH | O | H | CH₂CF₂CF₂H | log P |
| I-A-Q2-295 | 3-Cl | H | 5-CF₃ | NH₂ | (2,4-Cl,Cl—C₆H₄)OCF₂ | Me | CH | CH | O | H | CH₂CF₂CF₂H | log P |
| I-A-Q2-296 | 3-Cl | H | 5-CF₃ | NH₂ | (2,5-Cl,Cl—C₆H₄)OCF₂ | Me | CH | CH | O | H | CH₂CF₂CF₂H | log P |
| I-A-Q2-297 | 3-Cl | H | 5-CF₃ | NH₂ | (3-Cl—C₆H₄)OCF₂ | Me | CH | CH | O | H | CH₂CF₂CF₂H | log P |
| I-A-Q2-298 | 3-Cl | H | 5-CF₃ | NH₂ | (2-Cl—C₆H₄)OCF₂ | Me | CH | CH | O | H | CH₂CF₂CF₂H | log P |
| I-A-Q2-299 | 3-Cl | H | 5-CF₃ | NH₂ | PhOCF₂ | Me | CH | CH | O | H | CH₂CF₂CF₂H | log P |
| I-A-Q2-300 | 3-Cl | H | 5-CF₃ | NH₂ | (3,4-Cl,Cl—C₆H₄)OCF₂ | Me | CH | CH | O | H | CH₂CF₂CF₂H | log P |
| I-A-Q2-301 | 3-Cl | H | 5-F | NH₂ | CF₃ | Me | CH | CH | O | H | CH₂CF₂CF₂H | log P |
| I-A-Q2-302 | H | 4-Cl | H | NH₂ | CF₃ | Me | CH | CH | O | H | CH₂CF₂CF₂H | log P |
| I-A-Q2-303 | H | 4-Br | H | NH₂ | CF₃ | Me | CH | CH | O | H | CH₂CF₂CF₂H | log P |
| I-A-Q2-304 | 3-Cl | 4-Cl | H | NH₂ | CF₃ | Me | CH | CH | O | H | CH₂CF₂CF₂H | log P |
| I-A-Q2-305 | 2-Cl | H | 4-Cl | NH₂ | CF₃ | Me | CH | CH | O | H | CH₂CF₂CF₂H | log P |
| I-A-Q2-306 | H | 4-CN | H | NH₂ | CF₃ | Me | CH | CH | O | H | CH₂CF₂CF₂H | log P |
| I-A-Q2-307 | 2-F | 4-F | 6-F | NH₂ | CF₃ | Me | CH | CH | O | H | CH₂CF₂CF₂H | log P |
| I-A-Q2-308 | 2-Cl | H | H | NH₂ | CF₃ | Me | CH | CH | O | H | CH₂CF₂CF₂H | log P |
| I-A-Q2-309 | 3-Cl | 4-F | H | NH₂ | CF₃ | Me | CH | CH | O | H | CH₂CF₂CF₂H | log P |
| I-A-Q2-310 | 3-F | 4-Cl | H | NH₂ | CF₃ | Me | CH | CH | O | H | CH₂CF₂CF₂H | log P |
| I-A-Q2-311 | 3-F | H | H | NH₂ | CF₃ | Me | CH | CH | O | H | CH₂CF₂CF₂H | log P |
| I-A-Q2-312 | 3-F | 4-F | H | NH₂ | CF₃ | Me | CH | CH | O | H | CH₂CF₂CF₂H | log P |
| I-A-Q2-313 | H | 4-I | H | NH₂ | CF₃ | Me | CH | CH | O | H | CH₂CF₂CF₂H | log P |
| I-A-Q2-314 | H | 3-Br | H | NH₂ | CF₃ | Me | CH | CH | O | H | CH₂CF₂CF₂H | log P |
| I-A-Q2-315 | 3-F | H | 5-F | NH₂ | CF₃ | Me | CH | CH | O | H | CH₂CF₂CF₂H | log P |
| I-A-Q2-316 | 3-F | 4-F | 5-F | NH₂ | CF₃ | Me | CH | CH | O | H | CH₂CF₂CF₂H | log P |
| I-A-Q2-317 | 2-Me | H | H | NH₂ | CF₃ | Me | CH | CH | O | H | CH₂CF₂CF₂H | log P |
| I-A-Q2-318 | 3-CF₃ | H | 5-CF₃ | NH₂ | CF₃ | Me | CH | CH | O | H | CH₂CF₂CF₂H | log P |
| I-A-Q2-319 | 2-F | H | 4-F | NH₂ | CF₂H | Me | CH | CH | O | H | CH₂CF₂CF₂H | log P |
| I-A-Q2-320 | H | 4-Cl | H | NH₂ | CF₂H | Me | CH | CH | O | H | CH₂CF₂CF₂H | log P |
| I-A-Q2-321 | H | 4-Cl | H | NH₂ | CF₂CF₃ | Me | CH | CH | O | H | CH₂CF₂CF₂H | log P |
| I-A-Q2-322 | 3-Cl | H | 5-CF₃ | NH₂ | CF₂Cl | Me | CH | CH | O | H | CH₂CF₂CF₂H | log P |
| I-A-Q2-323 | 3-Cl | 4-Cl | H | NH₂ | Me | Me | CH | CH | O | H | CH₂CF₂CF₂H | log P |
| I-A-Q2-324 | H | 4-MeO | H | NH₂ | Me | Me | CH | CH | O | H | CH₂CF₂CF₂H | log P |
| I-A-Q2-325 | H | 4-Cl | H | NH₂ | Me | Me | CH | CH | O | H | CH₂CF₂CF₂H | log P |
| I-A-Q2-326 | H | 4-F | H | NH₂ | Me | Me | CH | CH | O | H | CH₂CF₂CF₂H | log P |
| I-A-Q2-327 | 2-Cl | 4-F | H | NH₂ | Me | Me | CH | CH | O | H | CH₂CF₂CF₂H | log P |
| I-A-Q2-328 | 2-F | 4-F | 6-F | NH₂ | Me | Me | CH | CH | O | H | CH₂CF₂CF₂H | log P |
| I-A-Q2-329 | 2-F | 4-F | H | NH₂ | Me | Me | CH | CH | O | H | CH₂CF₂CF₂H | log P |
| I-A-Q2-330 | H | 4-Cl, | H | NH₂ | Me | Me | CH | CH | O | H | CH₂CF₂CF₂H | log P |
| I-A-Q2-331 | 3-Cl | H | H | NH₂ | Me | Me | CH | CH | O | H | CH₂CF₂CF₂H | log P |
| I-A-Q2-332 | 2-F | 5-F | H | NH₂ | Me | Me | CH | CH | O | H | CH₂CF₂CF₂H | log P |
| I-A-Q2-332 | 2-F | 5-F | H | NH₂ | Me | Me | CH | CH | O | H | CH₂CF₂CF₂H | log P |
| I-A-Q2-333 | 2-Cl | H | H | NH₂ | Me | Me | CH | CH | O | H | CH₂CF₂CF₂H | log P |
| I-A-Q2-334 | 2-Cl | H | 6-F | NH₂ | Me | Me | CH | CH | O | H | CH₂CF₂CF₂H | log P |
| I-A-Q2-335 | 2-Cl | H | 5-Cl | NH₂ | Me | Me | CH | CH | O | H | CH₂CF₂CF₂H | log P |
| I-A-Q2-336 | 2-Cl | H | 6-Cl | NH₂ | Me | Me | CH | CH | O | H | CH₂CF₂CF₂H | log P |
| I-A-Q2-337 | H | 4-Cl | H | NH₂ | Ph | Me | CH | CH | O | H | CH₂CF₂CF₂H | log P |
| I-A-Q2-338 | H | 4-Cl | H | NH₂ | iPr | Me | CH | CH | O | H | CH₂CF₂CF₂H | log P |
| I-A-Q2-339 | H | 4-Cl | H | NH₂ | Et | Me | CH | CH | O | H | CH₂CF₂CF₂H | log P |
| I-A-Q2-340 | H | 4-Cl | H | NH₂ | Pr | Me | CH | CH | O | H | CH₂CF₂CF₂H | log P |
| I-A-Q2-341 | H | 4-Cl | H | NH₂ | Cypr | Me | CH | CH | O | H | CH₂CF₂CF₂H | log P |
| I-A-Q2-342 | H | 4-Cl | H | NH₂ | iPrCH₂ | Me | CH | CH | O | H | CH₂CF₂CF₂H | log P |
| I-A-Q2-343 | 3-CF₃ | H | 5-CF₃ | NH₂ | CF₂CF₃ | Me | CH | CH | O | H | CH₂CF₂CF₂H | log P |
| I-A-Q2-344 | 3-F | H | 5-Cl | NH₂ | CF₃ | Me | CH | CH | O | H | CH₂-2-Pyr | log P |
| I-A-Q2-345 | H | 4-Cl | H | NH₂ | CF₃ | Me | CH | CH | O | H | CH₂-2-Pyr | log P |
| I-A-Q2-346 | H | 4-Br | H | NH₂ | CF₃ | Me | CH | CH | O | H | CH₂-2-Pyr | log P |
| I-A-Q2-347 | 3-Cl | 4-Cl | H | NH₂ | CF₃ | Me | CH | CH | O | H | CH₂-2-Pyr | log P |
| I-A-Q2-348 | 2-Cl | 4-Cl | H | NH₂ | CF₃ | Me | CH | CH | O | H | CH₂-2-Pyr | log P |
| I-A-Q2-349 | H | 4-CN | H | NH₂ | CF₃ | Me | CH | CH | O | H | CH₂-2-Pyr | log P |
| I-A-Q2-350 | 2-F | 4-F | 6-F | NH₂ | CF₃ | Me | CH | CH | O | H | CH₂-2-Pyr | log P |
| I-A-Q2-351 | 2-Cl | H | H | NH₂ | CF₃ | Me | CH | CH | O | H | CH₂-2-Pyr | log P |
| I-A-Q2-352 | 3-Cl | 4-F | H | NH₂ | CF₃ | Me | CH | CH | O | H | CH₂-2-Pyr | log P |
| I-A-Q2-353 | 3-F | 4-Cl | H | NH₂ | CF₃ | Me | CH | CH | O | H | CH₂-2-Pyr | log P |
| I-A-Q2-354 | 3-F | H | H | NH₂ | CF₃ | Me | CH | CH | O | H | CH₂-2-Pyr | log P |
| I-A-Q2-355 | 3-F | 4-F | H | NH₂ | CF₃ | Me | CH | CH | O | H | CH₂-2-Pyr | log P |
| I-A-Q2-356 | H | 4-I | H | NH₂ | CF₃ | Me | CH | CH | O | H | CH₂-2-Pyr | log P |
| I-A-Q2-357 | 3-Br | H | H | NH₂ | CF₃ | Me | CH | CH | O | H | CH₂-2-Pyr | log P |
| I-A-Q2-358 | 3-F | 4-F | 5-F | NH₂ | CF₃ | Me | CH | CH | O | H | CH₂-2-Pyr | log P |
| I-A-Q2-359 | 2-Me | H | H | NH₂ | CF₃ | Me | CH | CH | O | H | CH₂-2-Pyr | log P |
| I-A-Q2-360 | 3-CF₃ | H | 5-CF₃ | NH₂ | CF₃ | Me | CH | CH | O | H | CH₂-2-Pyr | log P |
| I-A-Q2-361 | 2-F | H | 4-F | NH₂ | CF₂H | Me | CH | CH | O | H | CH₂-2-Pyr | log P |
| I-A-Q2-362 | H | 4-Cl | H | NH₂ | CF₂H | Me | CH | CH | O | H | CH₂-2-Pyr | log P |
| I-A-Q2-363 | H | 4-Cl | H | NH₂ | CF₂CF₃ | Me | CH | CH | O | H | CH₂-2-Pyr | log P |
| I-A-Q2-364 | 3-Cl | H | 5-CF₃ | NH₂ | CF₂Cl | Me | CH | CH | O | H | CH₂-2-Pyr | log P |
| I-A-Q2-365 | 3-Cl | 4-Cl | H | NH₂ | Me | Me | CH | CH | O | H | CH₂-2-Pyr | log P |
| I-A-Q2-366 | H | 4-Cl | H | NH₂ | Me | Me | CH | CH | O | H | CH₂-2-Pyr | log P |
| I-A-Q2-367 | H | 4-F | H | NH₂ | Me | Me | CH | CH | O | H | CH₂-2-Pyr | log P |
| I-A-Q2-368 | 2-Cl | 4-F | H | NH₂ | Me | Me | CH | CH | O | H | CH₂-2-Pyr | log P |
| I-A-Q2-369 | 2-F | H | 6-F | NH₂ | Me | Me | CH | CH | O | H | CH₂-2-Pyr | log P |
| I-A-Q2-370 | 2-F | 4-F | H | NH₂ | Me | Me | CH | CH | O | H | CH₂-2-Pyr | log P |
| I-A-Q2-371 | 3-Cl | H | H | NH₂ | Me | Me | CH | CH | O | H | CH₂-2-Pyr | log P |
| I-A-Q2-372 | 2-F | H | 5-F | NH₂ | Me | Me | CH | CH | O | H | CH₂-2-Pyr | log P |
| I-A-Q2-373 | 2-Cl | H | H | NH₂ | Me | Me | CH | CH | O | H | CH₂-2-Pyr | log P |
| I-A-Q2-374 | 2-F | H | 6-Cl | NH₂ | Me | Me | CH | CH | O | H | CH₂-2-Pyr | log P |
| I-A-Q2-375 | 2-Cl | H | 5-Cl | NH₂ | Me | Me | CH | CH | O | H | CH₂-2-Pyr | log P |
| I-A-Q2-376 | 2-Cl | H | 6-Cl | NH₂ | Me | Me | CH | CH | O | H | CH₂-2-Pyr | log P |
| I-A-Q2-377 | H | 4-Cl | H | NH | Ph | Me | CH | CH | O | H | CH₂-2-Pyr | log P |
| I-A-Q2-378 | H | 4-Cl | H | NH₂ | iPr | Me | CH | CH | O | H | CH₂-2-Pyr | log P |
| I-A-Q2-379 | H | 4-Cl | H | NH₂ | Et | Me | CH | CH | O | H | CH₂-2-Pyr | log P |
| I-A-Q2-380 | H | 4-Cl | H | NH₂ | Pr | Me | CH | CH | O | H | CH₂-2-Pyr | log P |
| I-A-Q2-381 | H | 4-Cl | H | NH₂ | Cypr | Me | CH | CH | O | H | CH₂-2-Pyr | log P |
| I-A-Q2-382 | H | 4-Cl | H | NH₂ | iPrCH₂ | Me | CH | CH | O | H | CH₂-2-Pyr | log P |
| I-A-Q2-383 | 3-CF₃ | H | 5-CF₃ | NH₂ | CF₂CF₃ | Me | CH | CH | O | H | CH₂-2-Pyr | log P |
| I-A-Q2-384 | 3-Cl | H | 5-Cl | NH₂ | CF₃ | Cl | CH | CH | O | H | CH₂CF₂CF₂H | log P |
| I-A-Q2-385 | 3-Cl | H | 5-Cl | NH₂ | CF₃ | CF₃ | CH | CH | O | H | CH₂CF₂CF₂H | log P |
| I-A-Q2-386 | 3-Cl | H | 5-Cl | NH₂ | CF₃ | CF₃ | CH | CH | O | H | CH₂-2-Pyr | log P |
| I-A-Q2-387 | 3-Cl | H | 5-Cl | NH₂ | CF₃ | Me | CF | CH | O | H | CH₂CF₂CF₂H | log P |
| I-A-Q2-388 | 3-Cl | H | 5-Cl | NH₂ | CF₃ | Me | CF | CH | O | H | CH₂-2-Pyr | log P |
| I-A-Q2-389 | 3-Cl | H | 5-Cl | NH₂ | CF₃ | Me | CH | CF | O | H | CH₂CF₂CF₂H | log P |
| I-A-Q2-390 | 3-Cl | H | 5-Cl | NH₂ | CF₃ | Me | CH | CF | O | H | CH₂-2-Pyr | log P |
| I-A-Q2-391 | 3-Cl | H | 5-Cl | NH₂ | CF₃ | Cl | CH | CH | O | H | CH₂-2-Pyr | log P |
| I-A-Q2-392 | 3-Cl | H | 5-Cl | NH₂ | CF₃ | Br | CH | CH | O | H | CH₂-2-Pyr | log P |
| I-A-Q2-393 | 3-Cl | H | 5-Cl | NH₂ | CF₃ | NMe₂ | CH | CH | O | H | CH₂CF₂CF₂H | log P |
| I-A-Q2-394 | 3-Cl | H | 5-Cl | NH₂ | CF₃ | NMe₂ | CH | CH | O | H | CH₂-2-Pyr | log P |
| I-A-Q2-395 | 3-Cl | H | 5-Cl | NH₂ | CF₃ | SMe | CH | CH | O | H | CH₂CF₂CF₂H | log P |
| I-A-Q2-396 | 3-Cl | H | 5-Cl | SMe | CF₃ | CF₃ | CH | CH | O | H | CH₂CF₂CF₂H | log P |
| I-A-Q2-397 | 3-Cl | H | 5-Cl | NH₂ | CF₃ | NHMe | CH | CH | O | H | CH₂CF₂CF₂H | log P |
| I-A-Q2-398 | 3-Cl | H | 5-Cl | NH₂ | CF₃ | NHMe | CH | CH | O | H | CH₂-2-Pyr | log P |
| I-A-Q2-399 | 3-Cl | H | 5-Cl | Br | CF₃ | Me | CH | CH | O | H | CH₂CF₂CF₂H | log P |
| I-A-Q2-400 | 3-Cl | H | 5-Cl | 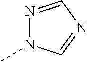 | CF₃ | Me | CH | CH | O | H | CH₂CF₂CF₂H | log P |
| I-A-Q2-401 | 3-Cl | H | 5-Cl | CN | CF₃ | Me | CH | CH | O | H | CH₂CF₂CF₂H | log P |
| I-A-Q2-402 | 3-Cl | H | 5-Cl | NH₂ | CF₃ | Me | CH | N | O | H | CH₂-2-Pyr | log P |
| I-A-Q2-403 | 3-Cl | H | 5-Cl | NH₂ | CF₃ | Me | CH | N | O | H | CHMe-2-Pyr | log P |
| I-A-Q2-404 | 3-Cl | H | 5-Cl | NH₂ | CF₃ | Me | CH | N | O | H | CH₂-Cypr | log P |
| I-A-Q2-405 | 3-Cl | H | 5-Cl | NH₂ | CF₃ | Me | CH | N | O | H | CH₂CF₂CF₂H | log P |
| I-A-Q2-407 | 3-Cl | H | 5-Cl | H | CF₃ | SMe | CH | CH | O | H | CH₂CF₂CF₂H | log P |
| I-A-Q2-408 | 3-Cl | H | 5-Cl | NH₂ | tBu | SMe | CH | CH | O | H | CH₂-2-Pyr | log P |
| I-A-Q2-409 | 3-Cl | H | 5-Cl | NH₂ | CF₃ | SMe | CH | CH | O | H | CH₂-2-Pyr | log P |
| I-A-Q2-410 | 2-Cl | 4-Cl | H | NH₂ | CF₃ | Me | CH | CH | O | H | CH₂Cypr | log P |
| I-A-Q2-411 | 2-Cl | 4-Cl | H | NH₂ | CF₃ | Me | CH | CH | O | H | CH₂-2-Pyrm | log P |
| I-A-Q2-412 | 2-Cl | 4-Cl | H | NH₂ | CF₃ | Me | CH | CH | O | H | CHMe-2-Pyr | log P |
| I-A-Q2-413 | 2-Cl | H | H | NH₂ | CF₃ | Me | CH | CH | O | H | CH₂Cypr | log P |
| I-A-Q2-414 | 2-Cl | H | H | NH₂ | CF₃ | Me | CH | CH | O | H | CH₂-2-Pyrm | log P |
| I-A-Q2-415 | 2-Cl | H | H | NH₂ | CF₃ | Me | CH | CH | O | H | CHMe-2-Pyr | log P |
| I-A-Q2-416 | 3-Br | H | H | NH₂ | CF₃ | Me | CH | CH | O | H | CH₂Cypr | log P |
| I-A-Q2-417 | 3-Br | H | H | NH₂ | CF₃ | Me | CH | CH | O | H | CH₂-2-Pyrm | log P |
| I-A-Q2-418 | 3-Br | H | H | NH₂ | CF₃ | Me | CH | CH | O | H | CHMe-2-Pyr | log P |
| I-A-Q2-419 | 2-Br | H | H | NH₂ | CF₃ | Me | CH | CH | O | H | CH₂-2-Pyrm | log P |
| I-A-Q2-420 | 2-Br | H | H | NH₂ | CF₃ | Me | CH | CH | O | H | CHMe-2-Pyr | log P |
| I-A-Q2-421 | 2-Br | H | H | NH₂ | CF₃ | Me | CH | CH | O | H | CH₂-2-Pyr | log P |
| I-A-Q2-422 | 2-Br | H | H | NH₂ | CF₃ | Me | CH | CH | O | H | CH₂Cypr | log P |
| I-A-Q2-423 | 2-Br | H | H | NH₂ | CF₃ | Me | CH | CH | O | H | CH₂CF₂CF₂H | log P |
| I-A-Q2-424 | 3-Cl | H | 5-Cl | NH₂ | CF₃ | S(O)Me | CH | CH | O | H | CH₂-2-Pyr | log P |
| I-A-Q2-425 | 3-Cl | H | 5-Cl | NH₂ | CF₃ | S(O)₂Me | CH | CH | O | H | CH₂-2-Pyr | log P |
| Characteristic data for synthesis examples: | | | | | | | | | | | | |
| I-A-Q2-001: log P (pH 2): 4.02 | | | | | | | | | | | | |
| log P (pH 7.5): 4.74 | | | | | | | | | | | | |
| ¹H NMR (CDCl₃): 4.78 (d, 2 H, J = 4.6 Hz), 7.22-7.42 (m, 6 H), 7.72-7.78 (m, 2 H), 8.00-8.02 (m, 1 H), 8.11-8.13 (m, 2 H), 8.53 (d, 1 H, J = 4.8 Hz) | | | | | | | | | | | | |
| I-A-Q2-002: spectroscopic data see protocol under general synthesis procedures | | | | | | | | | | | | |
| I-A-Q2-003: ¹H NMR (CDCl₃): 4.81 (d, 2 H, J = 4.6 Hz), 7.28-7.39 (m, 7 H), 7.73-7.78 (m, 2 H), 8.1-8.17 (m, 2 H), 8.45-8.54 (m, 2 H) | | | | | | | | | | | | |
| I-A-Q2-004: ¹H NMR (CDCl₃): 3.86-4.16 (m, 2 H), 7.34-7.42 (m, 3 H), 7.86-8.02 (m, 3 H), 8.15-8.17 (m, 1 H) | | | | | | | | | | | | |
| I-A-Q2-005: ¹H NMR (CDCl₃): 1.43 (d, 3 H, J = 3.5 Hz), 4.86-4.91 (m, 1 H), 6.13-6.20 (m, 1 H), 7.34-7.40 (m, 3 H), 7.59-7.67 (m, 1 H), 7.86-8.15 (m, 3H) | | | | | | | | | | | | |
| I-A-Q2-006: ¹H NMR (CDCl₃): 1.45 (d, 3 H, J = 7.1 Hz), 4.88-4.93 (m, 1 H), 7.36 (t, 1 H, J = 1.8 Hz), 7.74-7.76 (m, 3 H), 8.23-8.29 (m, 4 H), 9.17 (s, 1H) | | | | | | | | | | | | |
| I-A-Q2-008: ¹H NMR (CDCl₃): 4.79 (d, 2 H, J = 4.9 Hz), 7.43-7.77 (m, 9 H), 7.95-854 (m, 4 H) | | | | | | | | | | | | |
| I-A-Q2-009: ¹H NMR (CDCl₃): 4.77 (d, 2 H, J = 4.8 Hz), 7.22-7.42 (m, 3 H), 7.72-7.78 (m, 2 H), 7.93-7.95 (m, 3 H), 8.03-8.05 (m, 1 H), 8.14-8.14 (m, 1 H), 8.27-8.31 (m, 1 H), 8.52 (d, 1 H, J = 4.6 Hz) | | | | | | | | | | | | |
| I-A-Q2-010: ¹H NMR (CDCl₃): 4.10-4.14 (m, 2 H), 6.33-6.35 (m, 1 H), 7.71 (d, 1 H, J = 8.4 Hz), 7.92-7.95 (m, 3 H), 8.03-8.05 (m, 1 H), 8.14-8.14 (m, 1 H), 8.27 (s, 1H) | | | | | | | | | | | | |
| I-A-Q2-011: log P (HCOOH): 4.54 | | | | | | | | | | | | |
| ¹H NMR (DMSO-d₆): 8.77 (d, 1 H, J = 8.8 Hz), 7.95 (s, 1 H), 7.53 (t, 1 H, J = 2.0 Hz), 7.52-7.49 (m, 2 H), 7.34 (d, 2 H, J = 2.0 Hz), 5.58 (s, 2 H), 4.84-4.78 (m, 1 H), 2.42 (s, 3 H), 1.34 (d, 3 H, J = 7.2 Hz) | | | | | | | | | | | | |
| I-A-Q2-012: spectroscopic data see protocol under general synthesis procedures | | | | | | | | | | | | |
| I-A-Q2-013: log P (HCOOH): 3.59 | | | | | | | | | | | | |
| I-A-Q2-014: log P (HCOOH): 4.34 | | | | | | | | | | | | |
| ¹H NMR (DMSO-d₆): 8.86 (t, 1 H, J = 6.4 Hz), 7.54-7.49 (m, 4 H), 7.34 (d, 2 H, J = 2.0 Hz), 5.59 (s, 2 H), 4.12-4.04 (m, 1 H0, 2.43 (s, 3 H) | | | | | | | | | | | | |
| I-A-Q2-015: log P (HCOOH): 3.70 | | | | | | | | | | | | |
| ¹H NMR (DMSO-d6): 8.55-8.52 (m, 2 H), 7.77 (td, 1 H, J = 7.6 and 2.0 Hz), 7.54 (d, 1 H, J = 8.8 Hz), 7.53 (t, 1 H, J = 2.0 Hz), 7.49-7.47 (m, 2 H), 7.46 (d, 1 H, J = 7.6 Hz), 7.34 (d, 2 H, J = 2.0 Hz), 7.25 (dd, 1 H, J = 7.6 and 4.8 Hz), 5.58 (s, 2 H), 5.22-5.12 (m, 1 H), 2.41 (s, 3 H), 1.51 (d, 3 H, J = 6.8 Hz) | | | | | | | | | | | | |
| I-A-Q2-016: log P (HCOOH): 4.79 | | | | | | | | | | | | |
| ¹H NMR (DMSO-d6): 8.59 (d, 1 H, J = 7.6 Hz), 7.53 (t, 1 H, J = 2.0 Hz), 7.49-7.21 (m, 10 H), 5.54 (t, 2 H), 5.19-5.14 (m, 1 H), 2.38 (s, 3 H), 1.48 (d, 3 H, J = 6.8 Hz) | | | | | | | | | | | | |
| I-A-Q2-017: log P (HCOOH): 4.04 | | | | | | | | | | | | |
| ¹H NMR (DMSO-d6): 8.55 (t, 1 H, J = 5.8 Hz), 7.53 (t, 1 H, J = 2.0 Hz), 7.53-7.47 (m, 3 H), 7.34 (d, 2 H, J = 2.0 Hz), 6.12 (tt, 1 H, J = 56.0 and 3.8 Hz), 5.59 (s, 2 H0, 3.73-3.63 (m, 2 H), 2.43 (s, 3 H) | | | | | | | | | | | | |
| I-A-Q2-018: log P (HCOOH): 3.56 | | | | | | | | | | | | |
| ¹H NMR (DMSO-d6): 8.77 (d, 2 H, J = 5.2 Hz), 8.59 (t, 1 H, J = 5.8 Hz), 7.62 (d, 1 H, J = 8.8 Hz), 7.53 (t, 1 H, J = 2.0 Hz), 7.52-7.48 (m, 2 H), 7.40 (t, 1 H, J = 4.8 Hz), 7.35 (d, 2 H, J = 1.6 Hz), 5.59 (s, 2 H), 4.66 (d, 2 H, 4.66 (d, 2 H, J = 6.0 Hz), Me under DMSO signal | | | | | | | | | | | | |
| I-A-Q2-019: log P (HCOOH): 4.57 | | | | | | | | | | | | |
| ¹H NMR (DMSO-d6): 1.36 (d, 3 H); 2.42 (s, 3 H); 4.81 (m, 1 H); 5.51 (br. s, 2 H); 7.32 (dd, 1 H); 7.47-7.57 (m, 4 H); 7.67 (d, 1 H); 7.70 (br. d, 1 H) | | | | | | | | | | | | |
| I-A-Q2-020: spectroscopic data see protocol under general synthesis procedures | | | | | | | | | | | | |
| I-A-Q2-021: log P (HCOOH): 3.48 | | | | | | | | | | | | |
| ¹H NMR (DMSO-d6): 8.85 (d, 1 H, J = 8.8 Hz), 8.69 (s, 1 H), 8.51 (d, 1 H, J = 4.8 Hz), 7.75 (td, 1 H, J = 7.6 and 1.6 Hz), 7.54 (s, 1 H), 7.40 (d, 1 H, J = 7.6 Hz), 7.31 (d, 2 H, J = 2.0 Hz), 7.25 (dd, 1 H, J = 7.6 and 5.6 Hz), 5.14-5.09 (dd, 1 H), 1.45 (d, 3 H, J = 6.8 Hz) | | | | | | | | | | | | |
| I-A-Q2-022: spectroscopic data protocol under general synthesis procedures | | | | | | | | | | | | |
| I-A-Q2-023: log P (HCOOH): 4.91 | | | | | | | | | | | | |
| ¹H NMR (DMSOd6): 8.85 (t, 1 H, J = 5.6 Hz), 8.52 (d, 1 H, J = 4.0 Hz), 7.79 (td, 1 H, J = 7.6 and 1.6 Hz), 7.72 (t, 1 H, J = 1.8 Hz), 7.64 (d, 1 H, J = 8.4 Hz), 7.58-7.53 (m, 2 H), 7.50 (d, 2 H, J = 2.0 Hz), 7.42 (d, 1 H, J = 8.0 Hz), 7.28 (dd, 1 H, J = 6.8 and 5.6 Hz), 4.58 (d, 2 H, J = 6.0 Hz), 2.47 (s, 3 H) | | | | | | | | | | | | |
| I-A-Q2-024: spectroscopic data see protocol under general synthesis procedures | | | | | | | | | | | | |
| I-A-Q2-025: spectroscopic data see protocol under general synthesis procedures | | | | | | | | | | | | |
| I-A-Q2-026: log P (HCOOH): 5.44 | | | | | | | | | | | | |
| ¹H NMR (DMSO-d6): 8.90 (d, 1 H, J = 8.8 Hz), 7.71 (t, 1 H, J = 2.0 Hz), 7.54-7.52 (m, 3 H), 7.45 (d, 2 H, J = 2.0 Hz), 4.85-4.79 (m, 1 H), 2.43 (s, 3 H), 1.34 (d, 3 H, J = 7.2 Hz) | | | | | | | | | | | | |
| I-A-Q2-027: spectroscopic dat asee protocol under general synthesis procedures | | | | | | | | | | | | |
| I-A-Q2-028: log P (HCOOH): 4.35 | | | | | | | | | | | | |
| ¹H NMR (DMSO-d6): 8.83 (t, 1 H, J = 5.6 Hz), 8.52 (d, 1 H, J = 4.0 Hz), 7.78 (td, 1 H, J = 7.6 and 2.0 Hz), 7.69 (t, 1 H, J = 2.0 Hz), 7.63 (d, 1 H, J = 8.0 Hz), 7.58-7.55 (m, 2 H), 7.50 (d, 2 H, J = 2.0 Hz), 7.41 (d, 1 H, J = 8.0 Hz), 7.27 (dd, 1 H, J = 6.8 and 5.2 Hz), 4.58 (d, 2 H, J = 5.6 Hz), 2.47 (s, 3 H), 2.04 (s, 3 H) | | | | | | | | | | | | |
| I-A-Q2-029: log P (HCOOH): 3.24 | | | | | | | | | | | | |
| I-A-Q2-030: log P (HCOOH): 3.68 | | | | | | | | | | | | |
| I-A-Q2-031: log P (HCOOH): 3.33 | | | | | | | | | | | | |
| I-A-Q2-032: log P (HCOOH): 3.63 | | | | | | | | | | | | |
| I-A-Q2-033: log P (HCOOH): 4.51 | | | | | | | | | | | | |
| I-A-Q2-034: log P (HCOOH): 1.94 | | | | | | | | | | | | |
| I-A-Q2-035: log P (HCOOH): 4.23 | | | | | | | | | | | | |
| I-A-Q2-036: log P (HCOOH): 3.68 | | | | | | | | | | | | |
| I-A-Q2-037: log P (HCOOH): 2.42 | | | | | | | | | | | | |
| I-A-Q2-038: log P (HCOOH): 3.11 | | | | | | | | | | | | |
| I-A-Q2-039: log P (HCOOH): 3.94 | | | | | | | | | | | | |
| I-A-Q2-040: log P (HCOOH): 4.57 | | | | | | | | | | | | |
| I-A-Q2-041: log P (HCOOH): 3.94 | | | | | | | | | | | | |
| I-A-Q2-042: log P (HCOOH): 2.93 | | | | | | | | | | | | |
| I-A-Q2-043: log P (HCOOH): 5.65 | | | | | | | | | | | | |
| I-A-Q2-044: log P (HCOOH): 3.42 | | | | | | | | | | | | |
| I-A-Q2-045: log P (HCOOH): 5.85 | | | | | | | | | | | | |
| I-A-Q2-046: log P (HCOOH): 4.51 | | | | | | | | | | | | |
| I-A-Q2-047: log P (HCOOH): 4.94 | | | | | | | | | | | | |
| I-A-Q2-048: log P (HCOOH): 4.70 | | | | | | | | | | | | |
| I-A-Q2-049: log P (HCOOH): 4.28 | | | | | | | | | | | | |
| I-A-Q2-050: log P (HCOOH): 4.64 | | | | | | | | | | | | |
| I-A-Q2-051: log P (HCOOH): 3.06 | | | | | | | | | | | | |
| I-A-Q2-052: log P (HCOOH): 2.34 | | | | | | | | | | | | |
| I-A-Q2-053: log P (HCOOH): 2.04 | | | | | | | | | | | | |
| I-A-Q2-054: log P (HCOOH): 2.31 | | | | | | | | | | | | |
| I-A-Q2-055: log P (HCOOH): 5.79 | | | | | | | | | | | | |
| I-A-Q2-056: log P (HCOOH): 3.93 | | | | | | | | | | | | |
| I-A-Q2-057: log P (HCOOH): 2.72 | | | | | | | | | | | | |
| I-A-Q2-058: log P (HCOOH): 1.94 | | | | | | | | | | | | |
| I-A-Q2-059: log P (HCOOH): 3.63 | | | | | | | | | | | | |
| I-A-Q2-060: log P (HCOOH): 4.34 | | | | | | | | | | | | |
| I-A-Q2-061: log P (HCOOH): 4.45 | | | | | | | | | | | | |
| I-A-Q2-062: log P (HCOOH): 3.94 | | | | | | | | | | | | |
| I-A-Q2-063: log P (HCOOH): 3.11 | | | | | | | | | | | | |
| I-A-Q2-064: log P (HCOOH): 6.44 | | | | | | | | | | | | |
| I-A-Q2-065: log P (HCOOH): 2.72 | | | | | | | | | | | | |
| I-A-Q2-066: log P (HCOOH): 5.15 | | | | | | | | | | | | |
| I-A-Q2-067: log P (HCOOH): 4.65 | | | | | | | | | | | | |
| I-A-Q2-068: log P (HCOOH): 5.35 | | | | | | | | | | | | |
| I-A-Q2-069: log P (HCOOH): 4.83 | | | | | | | | | | | | |
| I-A-Q2-070: log P (HCOOH): 5.25 | | | | | | | | | | | | |
| I-A-Q2-071: log P (HCOOH): 4.95 | | | | | | | | | | | | |
| I-A-Q2-072: log P (HCOOH): 5.22 | | | | | | | | | | | | |
| I-A-Q2-073: log P (HCOOH): 4.35 | | | | | | | | | | | | |
| I-A-Q2-074: log P (HCOOH): 2.76 | | | | | | | | | | | | |
| I-A-Q2-075: log P (HCOOH): 3.94 | | | | | | | | | | | | |
| I-A-Q2-076: log P (HCOOH): 2.89 | | | | | | | | | | | | |
| I-A-Q2-077: log P (HCOOH): 4.17 | | | | | | | | | | | | |
| I-A-Q2-078: log P (HCOOH): 2.94 | | | | | | | | | | | | |
| I-A-Q2-079: log P (HCOOH): 3.37 | | | | | | | | | | | | |
| I-A-Q2-080: log P (HCOOH): 2.38 | | | | | | | | | | | | |
| I-A-Q2-081: log P (HCOOH): 3.58 | | | | | | | | | | | | |
| I-A-Q2-082: log P (HCOOH): 2.46 | | | | | | | | | | | | |
| I-A-Q2-083: log P (HCOOH): 3.68 | | | | | | | | | | | | |
| I-A-Q2-084: log P (HCOOH): 3.89 | | | | | | | | | | | | |
| I-A-Q2-085: log P (HCOOH): 3.47 | | | | | | | | | | | | |
| I-A-Q2-086: log P (HCOOH): 3.68 | | | | | | | | | | | | |
| I-A-Q2-087: log P (HCOOH): 2.80 | | | | | | | | | | | | |
| I-A-Q2-088: log P (HCOOH): 2.99 | | | | | | | | | | | | |
| I-A-Q2-089: log P (HCOOH): 3.89 | | | | | | | | | | | | |
| I-A-Q2-090: log P (HCOOH): 4.11 | | | | | | | | | | | | |
| I-A-Q2-091: log P (HCOOH): 3.09 | | | | | | | | | | | | |
| I-A-Q2-092: log P (HCOOH): 3.27 | | | | | | | | | | | | |
| I-A-Q2-093: log P (HCOOH): 4.00 | | | | | | | | | | | | |
| I-A-Q2-094: log P (HCOOH): 2.76 | | | | | | | | | | | | |
| I-A-Q2-095: log P (HCOOH): 4.51 | | | | | | | | | | | | |
| I-A-Q2-096: log P (HCOOH): 5.35 | | | | | | | | | | | | |
| I-A-Q2-097: log P (HCOOH): 4.64 | | | | | | | | | | | | |
| I-A-Q2-098: log P (HCOOH): 4.36 | | | | | | | | | | | | |
| I-A-Q2-099: log P (HCOOH): 5.42 | | | | | | | | | | | | |
| I-A-Q2-100: log P (HCOOH): 4.96 | | | | | | | | | | | | |
| I-A-Q2-101: log P (HCOOH): 4.71 | | | | | | | | | | | | |
| I-A-Q2-102: log P (HCOOH): 4.06 | | | | | | | | | | | | |
| I-A-Q2-103: log P (HCOOH): 5.19 | | | | | | | | | | | | |
| I-A-Q2-104: log P (HCOOH): 4.46 | | | | | | | | | | | | |
| I-A-Q2-105: log P (HCOOH): 3.61 | | | | | | | | | | | | |
| I-A-Q2-106: log P (HCOOH): 4.30 | | | | | | | | | | | | |
| I-A-Q2-107: log P (HCOOH): 2.95 | | | | | | | | | | | | |
| I-A-Q2-108: log P (HCOOH): 3.80 | | | | | | | | | | | | |
| I-A-Q2-109: log P (HCOOH): 4.35 | | | | | | | | | | | | |
| I-A-Q2-110: log P (HCOOH): 2.69 | | | | | | | | | | | | |
| I-A-Q2-111: log P (HCOOH): 4.17 | | | | | | | | | | | | |
| I-A-Q2-112: log P (HCOOH): 4.34 | | | | | | | | | | | | |
| I-A-Q2-133: log P (HCOOH): 4.44 | | | | | | | | | | | | |
| I-A-Q2-134: log P (HCOOH): 3.11 | | | | | | | | | | | | |
| I-A-Q2-135: log P (HCOOH): 4.06 | | | | | | | | | | | | |
| I-A-Q2-136: log P (HCOOH): 3.80 | | | | | | | | | | | | |
| I-A-Q2-137: log P (HCOOH): 3.40 | | | | | | | | | | | | |
| I-A-Q2-138: log P (HCOOH): 4.21 | | | | | | | | | | | | |
| I-A-Q2-139: log P (HCOOH): 4.25 | | | | | | | | | | | | |
| I-A-Q2-140: log P (HCOOH): 3.93 | | | | | | | | | | | | |
| I-A-Q2-141: log P (HCOOH): 4.21 | | | | | | | | | | | | |
| I-A-Q2-142: log P (HCOOH): 3.77 | | | | | | | | | | | | |
| I-A-Q2-143: log P (HCOOH): 2.59 | | | | | | | | | | | | |
| I-A-Q2-144: log P (HCOOH): 3.93 | | | | | | | | | | | | |
| I-A-Q2-145: log P (HCOOH): 4.34 | | | | | | | | | | | | |
| I-A-Q2-146: log P (HCOOH): 4.54 | | | | | | | | | | | | |
| I-A-Q2-147: log P (HCOOH): 3.47 | | | | | | | | | | | | |
| I-A-Q2-148: log P (HCOOH): 4.12 | | | | | | | | | | | | |
| I-A-Q2-149: log P (HCOOH): 4.15 | | | | | | | | | | | | |
| I-A-Q2-150: log P (HCOOH): 4.65 | | | | | | | | | | | | |
| I-A-Q2-151: log P (HCOOH): 4.06 | | | | | | | | | | | | |
| I-A-Q2-152: log P (HCOOH): 4.45 | | | | | | | | | | | | |
| I-A-Q2-153: log P (HCOOH): 3.89 | | | | | | | | | | | | |
| I-A-Q2-154: log P (HCOOH): 3.75 | | | | | | | | | | | | |
| I-A-Q2-155: log P (HCOOH): 4.11 | | | | | | | | | | | | |
| I-A-Q2-156: log P (HCOOH): 3.89 | | | | | | | | | | | | |
| I-A-Q2-157: log P (HCOOH): 3.51 | | | | | | | | | | | | |
| I-A-Q2-158: log P (HCOOH): 2.85 | | | | | | | | | | | | |
| I-A-Q2-159: log P (HCOOH): 3.65 | | | | | | | | | | | | |
| I-A-Q2-160: log P (HCOOH): 3.23 | | | | | | | | | | | | |
| I-A-Q2-161: log P (HCOOH): 3.37 | | | | | | | | | | | | |
| I-A-Q2-162: log P (HCOOH): 3.51 | | | | | | | | | | | | |
| I-A-Q2-163: log P (HCOOH): 3.13 | | | | | | | | | | | | |
| I-A-Q2-164: log P (HCOOH): 3.27 | | | | | | | | | | | | |
| I-A-Q2-165: log P (HCOOH): 2.72 | | | | | | | | | | | | |
| I-A-Q2-166: log P (HCOOH): 3.84 | | | | | | | | | | | | |
| I-A-Q2-167: log P (HCOOH): 3.19 | | | | | | | | | | | | |
| I-A-Q2-168: log P (HCOOH): 2.61 | | | | | | | | | | | | |
| I-A-Q2-169: log P (HCOOH): 3.78 | | | | | | | | | | | | |
| I-A-Q2-170: log P (HCOOH): 3.15 | | | | | | | | | | | | |
| I-A-Q2-171: log P (HCOOH): 2.53 | | | | | | | | | | | | |
| I-A-Q2-172: log P (HCOOH): 3.73 | | | | | | | | | | | | |
| I-A-Q2-173: log P (HCOOH): 3.11 | | | | | | | | | | | | |
| I-A-Q2-174: log P (HCOOH): 2.50 | | | | | | | | | | | | |
| I-A-Q2-175: log P (HCOOH): 3.47 | | | | | | | | | | | | |
| I-A-Q2-176: log P (HCOOH): 2.89 | | | | | | | | | | | | |
| I-A-Q2-177: log P (HCOOH): 2.27 | | | | | | | | | | | | |
| I-A-Q2-178: log P (HCOOH): 3.89 | | | | | | | | | | | | |
| I-A-Q2-179: log P (HCOOH): 3.24 | | | | | | | | | | | | |
| I-A-Q2-180: log P (HCOOH): 2.61 | | | | | | | | | | | | |
| I-A-Q2-181: log P (HCOOH): 2.98 | | | | | | | | | | | | |
| I-A-Q2-182: log P (HCOOH): 3.33 | | | | | | | | | | | | |
| I-A-Q2-183: log P (HCOOH): 3.11 | | | | | | | | | | | | |
| I-A-Q2-184: log P (HCOOH): 2.76 | | | | | | | | | | | | |
| I-A-Q2-185: log P (HCOOH): 2.14 | | | | | | | | | | | | |
| I-A-Q2-186: log P (HCOOH): 2.42 | | | | | | | | | | | | |
| I-A-Q2-187: log P (HCOOH): 3.42 | | | | | | | | | | | | |
| I-A-Q2-188: log P (HCOOH): 3.78 | | | | | | | | | | | | |
| I-A-Q2-189: log P (HCOOH): 3.53 | | | | | | | | | | | | |
| I-A-Q2-190: log P (HCOOH): 3.15 | | | | | | | | | | | | |
| I-A-Q2-191: log P (HCOOH): 2.57 | | | | | | | | | | | | |
| I-A-Q2-192: log P (HCOOH): 2.89 | | | | | | | | | | | | |
| I-A-Q2-193: log P (HCOOH): 3.42 | | | | | | | | | | | | |
| I-A-Q2-194: log P (HCOOH): 3.37 | | | | | | | | | | | | |
| I-A-Q2-195: log P (HCOOH): 2.96 | | | | | | | | | | | | |
| I-A-Q2-196: log P (HCOOH): 2.84 | | | | | | | | | | | | |
| I-A-Q2-197: log P (HCOOH): 2.76 | | | | | | | | | | | | |
| I-A-Q2-198: log P (HCOOH): 5.40 | | | | | | | | | | | | |
| I-A-Q2-199: log P (HCOOH): 3.48 | | | | | | | | | | | | |

I-A-Q2-200: log P (HCOOH): 4.82
I-A-Q2-201: log P (HCOOH): 5.11
I-A-Q2-202: log P (HCOOH): 4.22
I-A-Q2-203: log P (HCOOH): 3.77
I-A-Q2-204: log P (HCOOH): 4.22
I-A-Q2-205: log P (HCOOH): 4.17
I-A-Q2-206: log P (HCOOH): 4.29
I-A-Q2-207: log P (HCOOH): 4.83
I-A-Q2-208: log P (HCOOH): 3.19
I-A-Q2-209: log P (HCOOH): 3.02
I-A-Q2-210: log P (HCOOH): 4.28
I-A-Q2-211: log P (HCOOH): 2.89
I-A-Q2-212: log P (HCOOH): 2.27
I-A-Q2-213: log P (HCOOH): 4.89
I-A-Q2-214: log P (HCOOH): 4.45
I-A-Q2-215: log P (HCOOH): 4.76
I-A-Q2-216: log P (HCOOH): 2.57
I-A-Q2-217: log P (HCOOH): 4.70
I-A-Q2-218: log P (HCOOH): 3.33
I-A-Q2-219: log P (HCOOH): 4.05
I-A-Q2-220: log P (HCOOH): 4.11
I-A-Q2-221: log P (HCOOH): 4.51
I-A-Q2-222: log P (HCOOH): 4.76
I-A-Q2-223: log P (HCOOH): 4.45
I-A-Q2-224: log P (HCOOH): 3.78
I-A-Q2-225: log P (HCOOH): 4.57
I-A-Q2-226: log P (HCOOH): 2.50
I-A-Q2-227: log P (HCOOH): 4.40
I-A-Q2-228: log P (HCOOH): 3.78
I-A-Q2-229: log P (HCOOH): 4.05
I-A-Q2-230: log P (HCOOH): 4.89
I-A-Q2-231: log P (HCOOH): 2.69
I-A-Q2-232: log P (HCOOH): 4.95
I-A-Q2-233: log P (HCOOH): 4.40
I-A-Q2-234: log P (HCOOH): 2.38
I-A-Q2-235: log P (HCOOH): 4.95
I-A-Q2-236: log P (HCOOH): 4.51
I-A-Q2-237: log P (HCOOH): 4.76
I-A-Q2-238: log P (HCOOH): 3.78
I-A-Q2-239: log P (HCOOH): 4.17
I-A-Q2-240: log P (HCOOH): 4.70
I-A-Q2-241: log P (HCOOH): 4.89
I-A-Q2-242: log P (HCOOH): 5.29
I-A-Q2-243: log P (HCOOH): 3.78
I-A-Q2-244: log P (HCOOH): 3.68
I-A-Q2-245: log P (HCOOH): 3.89
I-A-Q2-246: log P (HCOOH): 5.02
I-A-Q2-247: log P (HCOOH): 4.57
I-A-Q2-248: log P (HCOOH): 4.34
I-A-Q2-249: log P (HCOOH): 4.28
I-A-Q2-250: log P (HCOOH): 3.68
I-A-Q2-251: log P (HCOOH): 3.84
I-A-Q2-252: log P (HCOOH): 3.02
I-A-Q2-253: log P (HCOOH): 4.40
I-A-Q2-254: log P (HCOOH): 3.58
I-A-Q2-255: log P (HCOOH): 4.40
I-A-Q2-256: log P (HCOOH): 4.64
I-A-Q2-257: log P (HCOOH): 5.22
I-A-Q2-258: log P (HCOOH): 3.47
I-A-Q2-259: log P (HCOOH): 3.33
I-A-Q2-260: log P (HCOOH): 3.33
I-A-Q2-261: log P (HCOOH): 4.76
I-A-Q2-262: log P (HCOOH): 3.28
I-A-Q2-263: log P (HCOOH): 5.08
I-A-Q2-264: log P (HCOOH): 1.99
I-A-Q2-265: log P (HCOOH): 3.63
I-A-Q2-266: log P (HCOOH): 4.89
I-A-Q2-267: log P (HCOOH): 6.14
I-A-Q2-268: log P (HCOOH): 4.45
I-A-Q2-269: log P (HCOOH): 4.57
I-A-Q2-270: log P (HCOOH): 3.84
I-A-Q2-271: log P (HCOOH): 4.45
I-A-Q2-272: log P (HCOOH): 3.24
I-A-Q2-273: log P (HCOOH): 2.98
I-A-Q2-274: log P (HCOOH): 4.95
I-A-Q2-275: log P (HCOOH): 4.47
I-A-Q2-276: log P (HCOOH): 4.50
I-A-Q2-277: log P (HCOOH): 4.56
I-A-Q2-278: log P (HCOOH): 4.30
I-A-Q2-279: log P (HCOOH): 4.43
I-A-Q2-280: log P (HCOOH): 3.96
I-A-Q2-281: log P (HCOOH): 3.97
I-A-Q2-282: log P (HCOOH): 5.25
I-A-Q2-283: log P (HCOOH): 4.12
I-A-Q2-284: log P (HCOOH): 4.32
I-A-Q2-285: log P (HCOOH): 4.95
I-A-Q2-286: log P (HCOOH): 4.89
I-A-Q2-287: log P (HCOOH): 5.33
I-A-Q2-288: log P (HCOOH): 4.40
I-A-Q2-289: log P (HCOOH): 4.95
I-A-Q2-290: log P (HCOOH): 4.00
I-A-Q2-291: log P (HCOOH): 3.84
I-A-Q2-292: log P (HCOOH): 3.53
I-A-Q2-293: log P (HCOOH): 3.11
I-A-Q2-294: log P (HCOOH): 4.95
I-A-Q2-295: log P (HCOOH): 5.27
I-A-Q2-296: log P (HCOOH): 5.14
I-A-Q2-297: log P (HCOOH): 4.95
I-A-Q2-298: log P (HCOOH): 4.64
I-A-Q2-299: log P (HCOOH): 4.51
I-A-Q2-300: log P (HCOOH): 5.40
I-A-Q2-301: log P (HCOOH): 3.75
I-A-Q2-302: log P (HCOOH): 3.61
I-A-Q2-303: log P (HCOOH): 3.70
I-A-Q2-304: log P (HCOOH): 4.00
I-A-Q2-305: log P (HCOOH): 3.75
I-A-Q2-306: log P (HCOOH): 3.24
I-A-Q2-307: log P (HCOOH): 3.27
I-A-Q2-308: log P (HCOOH): 3.23
I-A-Q2-309: log P (HCOOH): 3.65
I-A-Q2-310: log P (HCOOH): 3.70
I-A-Q2-311: log P (HCOOH): 3.27
I-A-Q2-312: log P (HCOOH): 3.42
I-A-Q2-313: log P (HCOOH): 3.85
I-A-Q2-314: log P (HCOOH): 3.65
I-A-Q2-315: log P (HCOOH): 3.37
I-A-Q2-316: log P (HCOOH): 3.65
I-A-Q2-317: log P (HCOOH): 3.32
I-A-Q2-318: log P (HCOOH): 4.23
I-A-Q2-319: log P (HCOOH): 2.80
I-A-Q2-320: log P (HCOOH): 3.23
I-A-Q2-321: log P (HCOOH): 4.00
I-A-Q2-322: log P (HCOOH): 4.34
I-A-Q2-323: log P (HCOOH): 4.32
I-A-Q2-324: log P (HCOOH): 2.30
I-A-Q2-325: log P (HCOOH): 2.85
I-A-Q2-326: log P (HCOOH): 2.51
I-A-Q2-327: log P (HCOOH): 2.73
I-A-Q2-328: log P (HCOOH): 2.48
I-A-Q2-329: log P (HCOOH): 2.59
I-A-Q2-330: log P (HCOOH): 3.12
I-A-Q2-331: log P (HCOOH): 2.85
I-A-Q2-332: log P (HCOOH): 2.59
I-A-Q2-333: log P (HCOOH): 2.59
I-A-Q2-334: log P (HCOOH): 2.59
I-A-Q2-335: log P (HCOOH): 3.04
I-A-Q2-336: log P (HCOOH): 2.76
I-A-Q2-337: log P (HCOOH): 3.80
I-A-Q2-338: log P (HCOOH): 3.65
I-A-Q2-339: log P (HCOOH): 3.27
I-A-Q2-340: log P (HCOOH): 3.56
I-A-Q2-341: log P (HCOOH): 3.46
I-A-Q2-342: log P (HCOOH): 3.85
I-A-Q2-343: log P (HCOOH): 4.57
I-A-Q2-344: log P (HCOOH): 2.84
I-A-Q2-345: log P (HCOOH): 2.69
I-A-Q2-346: log P (HCOOH): 2.80
I-A-Q2-347: log P (HCOOH): 3.11
I-A-Q2-348: log P (HCOOH): 2.84
I-A-Q2-349: log P (HCOOH): 2.04
I-A-Q2-350: log P (HCOOH): 2.34
I-A-Q2-351: log P (HCOOH): 2.27
I-A-Q2-352: log P (HCOOH): 2.76
I-A-Q2-353: log P (HCOOH): 2.84
I-A-Q2-354: log P (HCOOH): 2.31
I-A-Q2-355: log P (HCOOH): 2.50
I-A-Q2-356: log P (HCOOH): 2.93
I-A-Q2-357: log P (HCOOH): 2.72
I-A-Q2-358: log P (HCOOH): 2.80
I-A-Q2-359: log P (HCOOH): 2.34
I-A-Q2-360: log P (HCOOH): 3.47
I-A-Q2-361: log P (HCOOH): 1.91
I-A-Q2-362: log P (HCOOH): 2.27
I-A-Q2-363: log P (HCOOH): 3.19
I-A-Q2-364: log P (HCOOH): 3.53
I-A-Q2-365: log P (HCOOH): 2.27
I-A-Q2-366: log P (HCOOH): 1.91
I-A-Q2-367: log P (HCOOH): 1.59
I-A-Q2-368: log P (HCOOH): 1.79
I-A-Q2-369: log P (HCOOH): 1.52
I-A-Q2-370: log P (HCOOH): 1.64
I-A-Q2-371: log P (HCOOH): 1.88
I-A-Q2-372: log P (HCOOH): 1.61
I-A-Q2-373: log P (HCOOH): 1.64
I-A-Q2-374: log P (HCOOH): 1.66
I-A-Q2-375: log P (HCOOH): 2.04
I-A-Q2-376: log P (HCOOH): 1.79
I-A-Q2-377: log P (HCOOH): 2.80
I-A-Q2-378: log P (HCOOH): 2.61
I-A-Q2-379: log P (HCOOH): 2.20
I-A-Q2-380: log P (HCOOH): 2.53
I-A-Q2-381: log P (HCOOH): 2.46
I-A-Q2-382: log P (HCOOH): 2.84
I-A-Q2-383: log P (HCOOH): 3.94
I-A-Q2-384: log P (HCOOH): 4.53
I-A-Q2-385: log P (HCOOH): 4.60
I-A-Q2-386: log P (HCOOH): 3.77
I-A-Q2-387: log P (HCOOH): 4.32
I-A-Q2-388: log P (HCOOH): 3.37
I-A-Q2-389: log P (HCOOH): 4.35
I-A-Q2-390: log P (HCOOH): 3.41
I-A-Q2-391: log P (HCOOH): 3.57
I-A-Q2-392: log P (HCOOH): 3.51
I-A-Q2-393: log P (HCOOH): 4.82
I-A-Q2-394: log P (HCOOH): 3.70
I-A-Q2-395: log P (HCOOH): 4.43
I-A-Q2-396: log P (HCOOH): 4.59
I-A-Q2-397: log P (HCOOH): 4.69
I-A-Q2-398: log P (HCOOH): 3.59
I-A-Q2-399: log P (HCOOH): 5.26
I-A-Q2-400: log P (HCOOH): 4.36
I-A-Q2-401: log P (HCOOH): 4.93
I-A-Q2-402: log P (HCOOH): 3.89
I-A-Q2-403: log P (HCOOH): 4.34
I-A-Q2-404: log P (HCOOH): 4.81
I-A-Q2-405: log P (HCOOH): 4.84
I-A-Q2-407: log P (HCOOH): 5.15
I-A-Q2-408: log P (HCOOH): 3.68
I-A-Q2-409: log P (HCOOH): 3.38
I-A-Q2-410: log P (HCOOH): 3.94
I-A-Q2-411: log P (HCOOH): 3.17
I-A-Q2-412: log P (HCOOH): 3.29
I-A-Q2-413: log P (HCOOH): 3.37
I-A-Q2-414: log P (HCOOH): 2.72
I-A-Q2-415: log P (HCOOH): 2.71
I-A-Q2-416: log P (HCOOH): 3.81
I-A-Q2-417: log P (HCOOH): 3.13
I-A-Q2-418: log P (HCOOH): 3.22
I-A-Q2-419: log P (HCOOH): 2.68
I-A-Q2-420: log P (HCOOH): 2.81
I-A-Q2-421: log P (HCOOH): 2.53
I-A-Q2-422: log P (HCOOH): 3.41
I-A-Q2-423: log P (HCOOH): 3.55
I-A-Q2-424: log P (HCOOH): 2.75
I-A-Q2-425: log P (HCOOH): 2.99

TABLE 3
In accordance with the general methods described above, it is possible to prepare the
compounds of the formula (I-A where Q = Q³) listed in the table below.
(I-A-Q³)

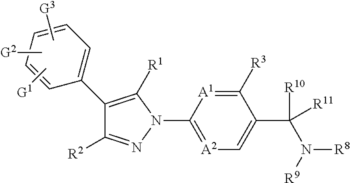

| Example | | | | | | | | | | | | | Phys. |
|---|---|---|---|---|---|---|---|---|---|---|---|---|---|
| no. | G¹ | G² | G³ | R¹ | R² | R³ | A¹ | A² | R⁸ | R⁹ | R¹⁰ | R¹¹ | chem. data |
| I-A-Q3-001 | 3-Cl | H | 5-Cl | H | CF₃ | Me | CH | CH | Ac | H | H | H | |
| I-A-Q3-002 | 3-CF₃ | H | 5-CF₃ | H | CF₃ | CN | CH | CH | CSMe | H | H | H | |
| I-A-Q3-003 | 3-CF₃ | H | H | NH₂ | CF₃ | NO₂ | CH | CH | COEt | H | H | H | |
| I-A-Q3-004 | 3-Br | H | 5-Br | NH₂ | CF₃ | CF₃ | CH | CH | CH₂-2-Pyr | H | H | H | |
| I-A-Q3-005 | 3-Cl | 4-Cl | 5-Cl | Cl | CF₃ | Br | CH | CH | COiPr | H | H | H | |
| I-A-Q3-006 | 3-Br | 4-Cl | 5-Br | Cl | C₂F₅ | Cl | CH | CH | COCyclpr | H | H | H | |
| I-A-Q3-007 | 3-CF₃ | 4-Cl | 5-Cl | Br | C₂F₅ | CH₂OMe | CH | CH | SO₂Me | H | H | H | |
| I-A-Q3-008 | 3-CF₃ | 4-F | H | Br | C₂F₅ | CH₂NHMe | CH | CH | CONMe₂ | H | H | H | |
| I-A-Q3-009 | 3-Cl | 4-CF₃ | 5-Cl | I | C₂F₅ | Me | CH | CH | CHO | H | H | H | |
| I-A-Q3-010 | 3-CF₃ | H | 5-Cl | I | CF₃ | CN | CH | CH | CONHCH₂CF₃ | H | H | H | |
| I-A-Q3-011 | 3-Cl | 4-CF₃ | 5-Cl | CN | CF₃ | NO2 | N | N | COOMe | H | H | H | |
| I-A-Q3-012 | 3-Cl | 4-Br | 5-Cl | CN | CF₃ | CF₃ | N | N | Ac | Ac | H | H | |
| I-A-Q3-013 | 3-F | 4-F | 5-F | NHCH₂Ph | C₂F₅ | Br | CH | CH | Ac | Ac | Me | Me | |
| I-A-Q3-014 | 2-Cl | 3-CF₃ | 4-Cl | NHAc | CF₃ | Cl | CH | CH | COOMe | COOMe | H | H | |
| I-A-Q3-015 | 3-Cl | 4-CF₃ | H | NAc₂ | CF₃ | CH₂OAc | CH | CH | COOMe | COOMe | Me | Me | |
| I-A-Q3-016 | 3-Br | 4-NH₂ | 5-Br | NHMe | CF₂Ph | CH₂NHAc | CH | CH | SO₂Me | H | Me | Me | |
| I-A-Q3-017 | 3-Br | H | H | NMe₂ | CF₂OMe | Me | CH | CH | CH₂-2-Pyr | H | Me | Me | |
| I-A-Q3-018 | 3-Cl | 4-Cl | H | SMe | CF₂OPh | CN | CH | CH | CH₂-2-Pyr | Ac | Me | Me | |
| I-A-Q3-019 | 2-Cl | 3-Cl | 4-Cl | SOMe | CF₂(2-Pyr) | NO₂ | N | CH | Me | H | H | H | |
| I-A-Q3-020 | 3-NO₂ | H | H | SO₂Me | CF₂O(2-Pyr) | CF₃ | CH | N | Ph | H | H | H | |
| I-A-Q3-021 | 3-Cl | H | 5-Cl | NH₂ | CF₃ | Cl | CH | CH | COCH₂CF₃ | H | H | H | log P |
| I-A-Q3-022 | 3-Cl | H | 5-Cl | NH₂ | CF₃ | Cl | CH | CH | COCypr₃ | H | H | H | log P |
| I-A-Q3-023 | 3-Cl | H | 5-Cl | NH₂ | CF₃ | Cl | CH | CH | COCH₃ | H | H | H | log P |
| I-A-Q3-024 | 3-Cl | H | 5-Cl | NH₂ | CF₃ | Cl | CH | CH | CONHEt | H | H | H | log P |

I-A-Q3-021: log P (HCOOH): 4.48
I-A-Q3-022: log P (HCOOH): 4.12
I-A-Q3-023: log P (HCOOH): 3.70
I-A-Q3-024: log P (HCOOH): 3.85

TABLE 4
In accordance with the general methods described above, it is possible to prepare the
compounds of the formula (I-A where Q = Q⁴) listed in the table below.
(I-A-Q⁴)

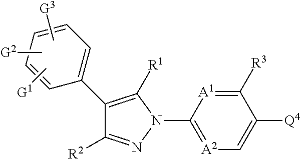

| | | | | | | | | | | Phys. chem. |
|---|---|---|---|---|---|---|---|---|---|---|
| Example no. | G¹ | G² | G³ | R¹ | R² | R³ | A¹ | A² | Q⁴ | data |
| I-A-Q4-001 | 3-Cl | H | 5-Cl | H | CF3 | CF₃ | CH | CH | COOEt | ¹H-NMR |
| I-A-Q4-002 | 3-Cl | H | 5-Cl | H | CF₃ | CF₃ | CH | CH | COOH | solid |
| I-A-Q4-003 | 3-CF₃ | H | 5-CF₃ | H | CF₃ | CF₃ | CH | CH | COOEt | ¹H-NMR |
| I-A-Q4-004 | 3-CF₃ | H | 5-CF₃ | H | CF₃ | CF₃ | CH | CH | COOH | |
| I-A-Q4-005 | 3-Cl | H | 5-Cl | H | CF₃ | NO₂ | CH | CH | COOtBu | ¹H-NMR |
| I-A-Q4-006 | 3-Cl | H | 5-Cl | H | CF₃ | CF₃ | CH | CH | NO₂ | ¹H-NMR |
| I-A-Q4-007 | 3-Cl | H | 5-Cl | H | CF₃ | CF₃ | CH | CH | NH₂ | ¹H-NMR |
| I-A-Q4-008 | 3-Cl | H | 5-Cl | H | H | CF₃ | CH | CH | COOEt | ¹H-NMR |
| I-A-Q4-009 | 3-Cl | H | 5-CF₃ | NH₂ | CF₃ | CN | CH | CH | F | log P |
| | | | | | | | | | | ¹H-NMR |
| I-A-Q4-010 | 3-Cl | H | 5-Cl | NH₂ | CF₃ | Me | CH | CH | COOH | log P |
| | | | | | | | | | | ¹H-NMR |
| I-A-Q4-011 | 3-Cl | H | 5-Cl | NH₂ | CF₃ | Me | CH | CH | COOMe | log P |
| | | | | | | | | | | ¹H-NMR |
| I-A-Q4-012 | 3-Cl | 4-Cl | H | NH₂ | CF₃ | Me | CH | CH | COOH | log P |
| | | | | | | | | | | ¹H-NMR |
| I-A-Q4-013 | 3-Cl | 4-Cl | H | NH₂ | CF₃ | Me | CH | CH | COOEt | log P |
| | | | | | | | | | | ¹H-NMR |
| I-A-Q4-014 | 3-Cl | H | 5-Cl | NH₂ | CF₃ | H | N | CH | Cl | log P |
| I-A-Q4-015 | H | 4-Cl | H | NCHNMe₂ | CF₃ | H | N | N | Cl | log P |
| I-A-Q4-016 | 3-Cl | H | 5-CF₃ | NH₂ | CF₃ | H | N | CH | Cl | log P |
| I-A-Q4-017 | 3-Cl | H | 5-Cl | Br | CF₃ | H | CH | CH | I | log P |
| I-A-Q4-018 | 3-Cl | H | 5-Cl | I | CF₃ | H | CH | CH | I | log P |
| I-A-Q4-019 | 3-Cl | H | 5-Cl | NH₂ | CF₃ | H | CF | CH | SO₂Et | log P |
| I-A-Q4-020 | 3-Cl | 4-Cl | H | SMe | CF₃ | H | N | N | Cl | log P |
| I-A-Q4-021 | 2-Cl | 4-Cl | H | NH₂ | Me | H | N | CH | Cl | log P |
| I-A-Q4-022 | 3-Me | H | 5-Me | NH₂ | CF₃ | H | N | CH | Cl | log P |
| I-A-Q4-023 | 2-Cl | H | H | NH₂ | CF₃ | H | N | CH | Cl | log P |
| I-A-Q4-024 | H | 4-Cl | H | NH₂ | Cypr | H | N | CH | Cl | log P |
| I-A-Q4-025 | H | 4-Cl | H | NH₂ | Pr | H | N | CH | Cl | log P |
| I-A-Q4-026 | H | 4-Cl | H | NH₂ | Et | H | N | CH | Cl | log P |
| I-A-Q4-027 | H | 4-Cl | H | NH₂ | iPr | H | N | CH | Cl | log P |
| I-A-Q4-028 | H | 4-Cl | H | NH₂ | Ph | H | N | CH | Cl | log P |
| I-A-Q4-029 | 2-Cl | H | 6-Cl | NH₂ | Me | H | N | CH | Cl | log P |
| I-A-Q4-030 | 2-Cl | H | 5-Cl | NH₂ | Me | H | N | CH | Cl | log P |
| I-A-Q4-031 | 2-F | H | 6-Cl | NH₂ | Me | H | N | CH | Cl | log P |
| I-A-Q4-032 | 2-Cl | H | H | NH₂ | Me | H | N | CH | Cl | log P |
| I-A-Q4-033 | 2-F | H | 5-F | NH₂ | Me | H | N | CH | Cl | log P |
| I-A-Q4-034 | 3-Cl | H | H | NH₂ | Me | H | N | CH | Cl | log P |
| I-A-Q4-035 | 2-F | 4-F | H | NH₂ | Me | H | N | CH | Cl | log P |
| I-A-Q4-036 | 2-F | H | 6-F | NH₂ | Me | H | N | CH | Cl | log P |
| I-A-Q4-037 | 2-Cl | 4-F | H | NH₂ | Me | H | N | CH | Cl | log P |
| I-A-Q4-038 | H | 4-F | H | NH₂ | Me | H | N | CH | Cl | log P |
| I-A-Q4-039 | H | 4-Cl | H | NH₂ | Me | H | N | CH | Cl | log P |
| I-A-Q4-040 | 3-F | H | 5-Cl | NH₂ | CF₃ | H | N | CH | Cl | log P |
| I-A-Q4-041 | H | 4-Cl | H | NH₂ | CF₃ | H | N | CH | Cl | log P |
| I-A-Q4-042 | H | 4-Br | H | NH₂ | CF₃ | H | N | CH | Cl | log P |
| I-A-Q4-043 | 3-Cl | 4-Cl | H | NH₂ | CF₃ | H | N | CH | Cl | log P |
| I-A-Q4-044 | 2-Cl | 4-Cl | H | NH₂ | CF₃ | H | N | CH | Cl | log P |
| I-A-Q4-045 | H | 4-CN | H | NH₂ | CF₃ | H | N | CH | Cl | log P |
| I-A-Q4-046 | 2-F | 4-F | 6-F | NH₂ | CF₃ | H | N | CH | Cl | log P |
| I-A-Q4-047 | 3-Cl | 4-F | H | NH₂ | CF₃ | H | N | CH | Cl | log P |
| I-A-Q4-048 | 3-F | 4-Cl | H | NH₂ | CF₃ | H | N | CH | Cl | log P |
| I-A-Q4-049 | 3-F | H | H | NH₂ | CF₃ | H | N | CH | Cl | log P |
| I-A-Q4-050 | 3-F | 4-F | H | NH₂ | CF₃ | H | N | CH | Cl | log P |
| I-A-Q4-051 | H | 4-I | H | NH₂ | CF₃ | H | N | CH | Cl | log P |
| I-A-Q4-052 | 3-Br | H | H | NH₂ | CF₃ | H | N | CH | Cl | log P |
| I-A-Q4-053 | 3-F | 4-F | 5-F | NH₂ | CF₃ | H | N | CH | Cl | log P |
| I-A-Q4-054 | 2-Me | H | H | NH₂ | CF₃ | H | N | CH | Cl | log P |
| I-A-Q4-055 | H | 4-Me | H | NH₂ | CF₃ | H | N | CH | Cl | log P |
| I-A-Q4-056 | 2-F | 4-F | H | NH₂ | CHF2 | H | N | CH | Cl | log P |
| I-A-Q4-057 | H | 4-Cl | H | NH₂ | CHF2 | H | N | CH | Cl | log P |
| I-A-Q4-058 | H | 4-Cl | H | NH₂ | C₂F₅ | H | N | CH | CI | log P |
| I-A-Q4-059 | 3-Cl | H | 5-CF₃ | NH₂ | CClF₂ | H | N | CH | Cl | log P |
| I-A-Q4-060 | 3-Cl | 4-Cl | H | NH₂ | Me | H | N | CH | Cl | log P |
| I-A-Q4-061 | H | 4-OMe | H | NH₂ | Me | H | N | CH | Cl | log P |
| I-A-Q4-062 | H | 4-Cl | H | NH₂ | CH₂-iPr | H | N | CH | Cl | log P |
| I-A-Q4-063 | 3-Cl | H | 5-Cl | NH₂ | CF₃ | CF₃ | N | N | COOMe | log P |
| I-A-Q4-064 | 3-Cl | H | 5-Cl | NH₂ | CF₃ | H | N | CH | COOH | log P |
| I-A-Q4-065 | 3-Cl | H | 5-Cl | H | CF₃ | Me | CH | CH | F | |
| I-A-Q4-066 | 3-CF₃ | H | 5-CF₃ | H | CF₃ | CN | CH | CH | Cl | |
| I-A-Q4-067 | 3-CF₃ | H | H | NH₂ | CF₃ | NO₂ | CH | CH | Br | |
| I-A-Q4-068 | 3-Br | H | 5-Br | NH₂ | CF₃ | CF₃ | CH | CH | I | |
| I-A-Q4-069 | 3-CI | 4-Cl | 5-Cl | Cl | CF₃ | Br | CH | CH | CN | |
| I-A-Q4-070 | 3-Br | 4-Cl | 5-Br | Cl | C₂F₅ | Cl | CH | CH | NO₂ | |
| I-A-Q4-071 | 3-CF₃ | 4-Cl | 5-Cl | Br | C₂F₅ | CH₂OMe | CH | CH | SMe | |
| I-A-Q4-072 | 3-CF₃ | 4-F | H | Br | C₂F₅ | CH₂NHMe | CH | CH | SCF₃ | |
| I-A-Q4-073 | 3-Cl | 4-CF₃ | 5-Cl | I | C₂F₅ | Me | CH | CH | SOMe | |
| I-A-Q4-074 | 3-CF₃ | H | 5-Cl | I | CF₃ | CN | CH | CH | SOCF₃ | |
| I-A-Q4-075 | 3-Cl | 4-CF₃ | 5-Cl | CN | CF₃ | NO₂ | N | N | SO₂Me | |
| I-A-Q4-076 | 3-Cl | 4-Br | 5-Cl | CN | CF₃ | CF₃ | N | N | SO₂CF₃ | |
| I-A-Q4-077 | 3-F | 4-F | 5-F | NHCH₂Ph | C₂F₅ | Br | CH | CH | CN | |
| I-A-Q4-078 | 2-Cl | 3-CF₃ | 4-Cl | NHAc | CF₃ | Cl | CH | CH | NO₂ | |
| I-A-Q4-079 | 3-Cl | 4-CF₃ | H | NAc₂ | CF₃ | CH₂OAc | CH | CH | F | |
| I-A-Q4-080 | 3-Br | 4-NH₂ | 5-Br | NHMe | CF₂Ph | CH₂NHAc | CH | CH | Cl | |
| I-A-Q4-081 | 3-Br | H | H | NMe₂ | CF₂OMe | Me | CH | CH | Br | |
| I-A-Q4-082 | 3-Cl | 4-Cl | H | SMe | CF₂OPh | CN | CH | CH | I | |
| I-A-Q4-083 | 2-Cl | 3-Cl | 4-Cl | SOMe | CF₂(2-Pyr) | NO₂ | N | CH | COOH | |
| I-A-Q4-084 | 3-NO₂ | H | H | SO₂Me | CF₂O(2-Pyr) | CF₃ | CH | N | COOEt | |
| I-A-Q4-085 | 2-Cl | 4-Cl | H | NH₂ | CF₃ | H | CH | CH | COOEt | log P |
| I-A-Q4-086 | 3-Cl | H | 5-Cl | NH₂ | CF₃ | H | CH | CH | COOEt | log P |
| I-A-Q4-087 | 3-Cl | H | 5-Cl | NH₂ | CF₃ | H | CH | CH | COOH | log P |
| I-A-Q4-088 | H | 4-Cl | H | NH₂ | CF₃ | H | CH | CH | COOH | log P |
| I-A-Q4-089 | 3-Cl | 4-Cl | H | NH₂ | CF₃ | H | CH | CH | COOH | log P |
| I-A-Q4-090 | 3-Me | H | 5-Me | NH₂ | CF₃ | H | CH | CH | COOH | log P |
| I-A-Q4-091 | 3-Br | H | H | NH₂ | CF₃ | H | CH | CH | COOH | log P |
| I-A-Q4-092 | 3-F | 4-F | H | NH₂ | CF₃ | H | CH | CH | COOH | log P |
| I-A-Q4-093 | 3-F | 4-Cl | H | NH₂ | CF₃ | H | CH | CH | COOH | log P |
| I-A-Q4-094 | 3-Cl | 4-F | H | NH₂ | CF₃ | H | CH | CH | COOH | log P |
| I-A-Q4-095 | 2-Cl | 4-Cl | H | NH₂ | CF₃ | H | CH | CH | COOH | log P |
| I-A-Q4-096 | 3-Cl | H | H | NH₂ | CF₃ | H | CH | CH | COOH | log P |
| I-A-Q4-097 | 3-F | H | H | NH₂ | CF₃ | H | CH | CH | COOH | log P |
| I-A-Q4-098 | H | 4-Br | H | NH₂ | CF₃ | H | CH | CH | COOH | log P |
| I-A-Q4-099 | 3-F | 4-F | 5-F | NH₂ | CF₃ | H | CH | CH | COOH | log P |
| I-A-Q4-100 | 3-F | H | 5-Cl | NH₂ | CF₃ | H | CH | CH | COOH | log P |
| I-A-Q4-101 | 2-Me | H | H | NH₂ | CF₃ | H | CH | CH | COOH | log P |
| I-A-Q4-102 | 3-Cl | H | 5-Cl | NH₂ | CF₃ | CN | CH | CH | I | log P |
| I-A-Q4-103 | 3-Cl | H | 5-Cl | NH₂ | CF₃ | CN | CH | CH | F | log P |
| I-A-Q4-104 | 3-Cl | H | 5-Cl | NH₂ | CF₃ | Cl | CH | CH | COOMe | log P |
| I-A-Q4-105 | 3-Cl | H | 5-Cl | NH₂ | CF₃ | CF₃ | CH | CH | F | log P |
| I-A-Q4-106 | 3-Cl | H | 5-Cl | NH₂ | CF₃ | H | CCF₃ | CH | F | log P |
| I-A-Q4-107 | 3-Cl | H | 5-Cl | NH₂ | CF₃ | H | CF | CF | F | log P |
| I-A-Q4-108 | 3-Cl | H | 5-CF₃ | NH₂ | CF₃ | CF₃ | CH | CH | COOMe | |
| I-A-Q4-109 | 3-Cl | H | 5-Cl | NH₂ | CF₃ | F | CH | CH | F | log P |
| I-A-Q4-110 | 3-Cl | H | 5-Cl | NH₂ | CF₃ | Cl | CH | CH | SO₂NHCypr | log P |
| I-A-Q4-111 | 3-Cl | H | 5-Cl | NH₂ | CF₃ | Br | CH | CH | SO₂NHCypr | log P |
| I-A-Q4-112 | 3-Cl | H | 5-Cl | NH₂ | CF₃ | CF₃ | CH | CH | COOMe | log P |
| I-A-Q4-113 | 3-Cl | H | 5-Cl | NH₂ | CF₃ | Me | CF | CH | COOMe | log P |
| I-A-Q4-114 | 3-Cl | H | 5-Cl | NH₂ | CF₃ | Me | CH | CF | COOMe | log P |
| I-A-Q4-115 | 3-Cl | H | 5-Cl | NH₂ | CF₃ | CF₃ | CH | CH | COOH | log P |
| I-A-Q4-116 | 3-Cl | H | 5-Cl | NH₂ | CF₃ | Me | CF | CH | COOH | log P |
| I-A-Q4-117 | 3-Cl | H | 5-Cl | NH₂ | CF₃ | Me | CH | CF | COOH | log P |
| I-A-Q4-118 | 3-Cl | H | 5-Cl | NH₂ | CF₃ | SMe | CH | CH | COOMe | log P |
| I-A-Q4-119 | 3-Cl | H | 5-Cl | NH₂ | CF₃ | NMe₂ | CH | CH | COOMe | log P |
| I-A-Q4-120 | 3-Cl | H | 5-Cl | NH₂ | CF₃ | NHMe | CH | CH | COOH | log P |
| I-A-Q4-121 | 3-Cl | H | 5-Cl | NH₂ | CF₃ | NMe2 | CH | CH | COOH | log P |
| I-A-Q4-122 | 3-Cl | H | 5-Cl | NH₂ | CF₃ | CN | CH | CH | S(O)₂NHCybu | log P |
| I-A-Q4-123 | 3-Cl | H | 5-Cl | NH₂ | CF₃ | CN | CH | CH | S(O)₂NHCH₂CF₃ | log P |

Characteristic data for synthesis examples:
I-A-Q4-001: spectroscopic data see protocol under general synthesis procedures
I-A-Q4-002: spectroscopic data see protocol under general synthesis procedures
I-A-Q4-003: ¹H NMR (CDCl₃): 8.26-8.18 (m, 2 H), 8.04-7.97 (m, 5 H), 4.44 (q, 2 H, J = 7.1 Hz), 1.42 (t, 3 H, J = 7.1 Hz)
I-A-Q4-005: ¹H NMR (CDCl₃): 8.22 (d, 1H, J = 2.2 Hz), 8.15 (d, 1 H, J = 0.9 Hz), 8.03 (dd, 1 H, J = 8.4 and 2.2 Hz), 7.94-7.91 (m, 1 H), 7.42-7.36 (m, 3 H), 1.58 (s, 9 H)
I-A-Q4-006: ¹H NMR (CDCl₃): 8.27-8.23 (m, 1 H), 8.20-8.17 (m, 1 H), 8.15-8.11 (m, 2 H), 7.44-7.43 (m, 1 H), 7.37-7.37 (m, 2 H)
I-A-Q4-007: ¹H NMR (CDCl₃): 7.92-7.87 (m, 1 H), 7.75-7.73 (m, 1 H), 7.66-7.61 (m, 1 H), 7.34-7.29 (m, 3 H), 6.84 (t, 1 H, J = 8.7 Hz), 4.37-4.25 (m, 2 H)
I-A-Q4-008: ¹H NMR (CDCl₃): 8.25 (d, 1 H, J = 0.5 Hz), 8.16 (s, 1 H), 8.02 (s, 1 H), 7.97 (d, 2 H, J = 1.3 Hz), 7.44 (d, 2 H, J = 1.8 Hz), 7.32-7.30 (m, 1 H), 4.43 (q, 2 H, J= 7.1 Hz), 1.41 (t, 3 H, J= 8.6 Hz)
I-A-Q4-009: spectroscopic data see protocol under general synthesis procedures
I-A-Q4-010: spectroscopic data see protocol under general synthesis procedures
I-A-Q4-011: spectroscopic data see protocol under general synthesis procedures
I-A-Q4-012: spectroscopic data see protocol under general synthesis procedures
I-A-Q4-013: spectroscopic data see protocol under general synthesis procedures
I-A-Q4-014: log P (HCOOH): 5.76
I-A-Q4-015: log P (HCOOH): 3.47
I-A-Q4-016: log P (HCOOH): 5.76
I-A-Q4-017: log P (HCOOH): 6.71
I-A-Q4-018: log P (HCOOH): 6.50
I-A-Q4-019: log P (HCOOH): 3.94
I-A-Q4-020: log P (HCOOH): 5.22
I-A-Q4-021: log P (HCOOH): 4.70
I-A-Q4-022: log P (HCOOH): 5.22
I-A-Q4-023: log P (HCOOH): 4.57
I-A-Q4-024: log P (HCOOH): 5.36
I-A-Q4-025: log P (HCOOH): 5.51
I-A-Q4-026: log P (HCOOH): 5.02
I-A-Q4-027: log P (HCOOH): 5.73
I-A-Q4-028: log P (HCOOH): 5.64
I-A-Q4-029: log P (HCOOH): 4.05
I-A-Q4-030 :log P (HCOOH): 4.57
I-A-Q4-031: log P (HCOOH): 3.78
I-A-Q4-032: log P (HCOOH): 3.89
I-A-Q4-033: log P (HCOOH): 3.84
I-A-Q4-034: log P (HCOOH): 4.40
I-A-Q4-035: log P (HCOOH): 3.78
I-A-Q4-036: log P (HCOOH): 3.58
I-A-Q4-037: log P (HCOOH): 4.05
I-A-Q4-038: log P (HCOOH): 3.84
I-A-Q4-039: log P (HCOOH): 4.40
I-A-Q4-040: log P (HCOOH): 5.29
I-A-Q4-041: log P (HCOOH): 4.95
I-A-Q4-042: log P (HCOOH): 5.22
I-A-Q4-043: log P (HCOOH): 5.44
I-A-Q4-044: log P (HCOOH): 5.02
I-A-Q4-045: log P (HCOOH): 4.11
I-A-Q4-046: log P (HCOOH): 4.46
I-A-Q4-047: log P (HCOOH): 4.95
I-A-Q4-048: log P (HCOOH): 5.02
I-A-Q4-049: log P (HCOOH): 4.51
I-A-Q4-050: log P (HCOOH): 4.64
I-A-Q4-051: log P (HCOOH): 5.29
I-A-Q4-052: log P (HCOOH): 5.02
I-A-Q4-053: log P (HCOOH): 4.89
I-A-Q4-054: log P (HCOOH): 4.64
I-A-Q4-055: log P (HCOOH): 4.83
I-A-Q4-056: log P (HCOOH): 3.84
I-A-Q4-057: log P (HCOOH): 4.45
I-A-Q4-058: log P (HCOOH): 5.44
I-A-Q4-059: log P (HCOOH): 5.81
I-A-Q4-060: log P (HCOOH): 5.02
I-A-Q4-061: log P (HCOOH): 3.58
I-A-Q4-062: log P (HCOOH): 5.97
I-A-Q4-063: log P (HCOOH): 4.66
I-A-Q4-064: log P (HCOOH): 4.23
I-A-Q4-085: log P (HCOOH): 4.57
I-A-Q4-086: log P (HCOOH): 4.93
I-A-Q4-087: log P (HCOOH): 3.79
I-A-Q4-088: log P (HCOOH): 3.28
I-A-Q4-089: log P (HCOOH): 3.69
I-A-Q4-090: log P (HCOOH): 3.42
I-A-Q4-091: log P (HCOOH): 3.29
I-A-Q4-092: log P (HCOOH): 3.06
I-A-Q4-093: log P (HCOOH): 3.39
I-A-Q4-094: log P (HCOOH): 3.31
I-A-Q4-095: log P (HCOOH): 3.44
I-A-Q4-096: log P (HCOOH): 3.20
I-A-Q4-097: log P (HCOOH): 2.88
I-A-Q4-098: log P (HCOOH): 3.35
I-A-Q4-099: log P (HCOOH): 3.32
I-A-Q4-100: log P (HCOOH): 3.32
I-A-Q4-101: log P (HCOOH): 2.94
I-A-Q4-102: log P (HCOOH): 5.94
I-A-Q4-103: log P (HCOOH): 4.44
I-A-Q4-104: log P (HCOOH): 5.07
I-A-Q4-105: log P (HCOOH): 5.25
I-A-Q4-106: log P (HCOOH): 4.68
I-A-Q4-107: log P (HCOOH): 4.45
I-A-Q4-109: log P (HCOOH): 4.77
I-A-Q4-110: log P (HCOOH): 4.49
I-A-Q4-111: log P (HCOOH): 4.77
I-A-Q4-112: log P (HCOOH): 5.21
I-A-Q4-113: log P (HCOOH): 4.78
I-A-Q4-114: log P (HCOOH): 4.84
I-A-Q4-115: log P (HCOOH): 4.22
I-A-Q4-116: log P (HCOOH): 3.91
I-A-Q4-117: log P (HCOOH): 3.96
I-A-Q4-118: log P (HCOOH): 4.93
I-A-Q4-119: log P (HCOOH): 4.71
I-A-Q4-120: log P (HCOOH): 4.11
I-A-Q4-121: log P (HCOOH): 3.07
I-A-Q4-122: log P (HCOOH): 4.69
I-A-Q4-123: log P (HCOOH): 4.41

TABLE 5
In accordance with the general methods described above, it is possible to prepare the
compounds of the formula (I-B where Q = Q¹) listed in the table below.
(I-B-Q¹)

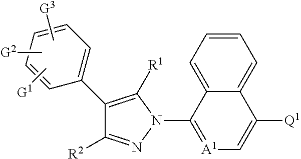

| | | | | | | | | Phys. |
|---|---|---|---|---|---|---|---|---|
| | | | | | | | | chem. |
| Example no. | G¹ | G² | G³ | R¹ | R² | A¹ | Q¹ | data |
| I-B-Q1-001 | 3-Cl | H | 5-Cl | H | CF₃ | CH | 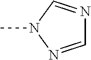 | |
| I-B-Q1-002 | 3-CF₃ | H | 5-CF₃ | H | CF₃ | CH | 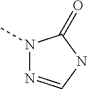 | |
| I-B-Q1-003 | 3-CF₃ | H | H | NH₂ | CF₃ | CH | 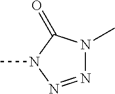 | |
| I-B-Q1-004 | 3-Br | H | 5-Br | NH₂ | CF₃ | CH | 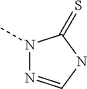 | |
| I-B-Q1-005 | 3-Cl | 4-Cl | 5-Cl | Cl | CF₃ | CH | 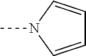 | |
| I-B-Q1-006 | 3-Br | 4-Cl | 5-Br | Cl | C₂F₅ | CH | 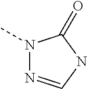 | |
| I-B-Q1-007 | 3-CF₃ | 4-Cl | 5-Cl | Br | C₂F₅ | CH | 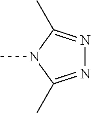 | |
| I-B-Q1-008 | 3-CF₃ | 4-F | H | Br | C₂F₅ | CH | 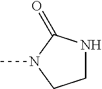 | |
| I-B-Q1-009 | 3-Cl | 4-CF₃ | 5-Cl | I | C₂F₅ | CH | 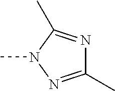 | |
| I-B-Q1-010 | 3-CF₃ | H | 5-Cl | I | CF₃ | CH | 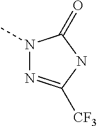 | |
| I-B-Q1-011 | 3-Cl | 4-CF₃ | 5-Cl | CN | CF₃ | N | 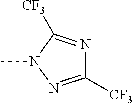 | |
| I-B-Q1-012 | 3-Cl | 4-Br | 5-Cl | CN | CF₃ | N | 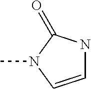 | |
| I-B-Q1-013 | 3-F | 4-F | 5-F | NHCH₂Ph | C₂F₅ | CH | 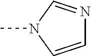 | |
| I-B-Q1-014 | 2-Cl | 3-CF₃ | 4-Cl | NHAc | CF₃ | CH | 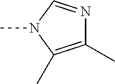 | |
| I-B-Q1-015 | 3-Cl | 4-CF₃ | H | NAc₂ | CF₃ | CH | 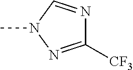 | |
| I-B-Q1-016 | 3-Br | 4-NH₂ | 5-Br | NHMe | CF₂Ph | CH | 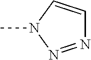 | |
| I-B-Q1-017 | 3-Br | H | H | NMe₂ | CF₂OMe | CH | 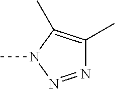 | |
| I-B-Q1-018 | 3-Cl | 4-Cl | H | SMe | CF₂OPh | CH | 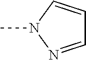 | |
| I-B-Q1-019 | 2-Cl | 3-Cl | 4-Cl | SOMe | CF₂(2-Pyr) | N | 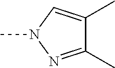 | |
| I-B-Q1-020 | 3-NO₂ | H | H | SO₂Me | CF₂O(2-Pyr) | CH | 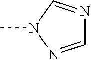 | |

TABLE 6
In accordance with the general methods described above, it is possible to prepare the
compounds of the formula (I-B where Q = Q²) listed in the table below.
(I-B-Q²)

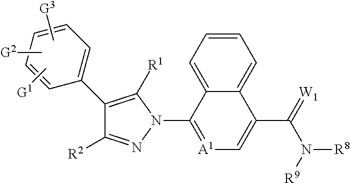

| | | | | | | | | | | Phys. |
|---|---|---|---|---|---|---|---|---|---|---|
| | | | | | | | | | | chem. |
| Example no. | G¹ | G² | G³ | R¹ | R² | A¹ | W¹ | R⁸ | R⁹ | data |
| I-B-Q2-001 | 3-Cl | H | 5-Cl | H | CF₃ | CH | O | H | TFiPr | log P |
| I-B-Q2-002 | 3-CF₃ | H | 5-CF₃ | H | CF₃ | CH | O | H | HFiPr | |
| I-B-Q2-003 | 3-CF₃ | H | H | NH₂ | CF₃ | CH | O | H | CH₂-2-Pyr | |
| I-B-Q2-004 | 3-Br | H | 5-Br | NH₂ | CF₃ | CH | O | Me | CH₂-2-Pyr | |
| I-B-Q2-005 | 3-Cl | 4-Cl | 5-Cl | Cl | CF₃ | CH | O | H | CHMe-2-Pyr | |
| I-B-Q2-006 | 3-Br | 4-Cl | 5-Br | Cl | C₂F₅ | CH | O | Me | CHMe-2-Pyr | |
| I-B-Q2-007 | 3-CF₃ | 4-Cl | 5-Cl | Br | C₂F₅ | CH | O | H | CH₂-3-Pyr | |
| I-B-Q2-008 | 3-CF₃ | 4-F | H | Br | C₂F₅ | CH | O | Me | CH₂-3-Pyr | |
| I-B-Q2-009 | 3-Cl | 4-CF₃ | 5-Cl | I | C₂F₅ | CH | O | H | CHMe-3-Pyr | |
| I-B-Q2-010 | 3-CF₃ | H | 5-Cl | I | CF₃ | CH | O | Me | CHMe-3-Pyr | |
| I-B-Q2-011 | 3-Cl | 4-CF3 | 5-Cl | CN | CF₃ | N | O | H | CH₂-4-Pyr | |
| I-B-Q2-012 | 3-Cl | 4-Br | 5-Cl | CN | CF₃ | N | O | Me | CH₂-4-Pyr | |
| I-B-Q2-013 | 3-F | 4-F | 5-F | NHCH₂Ph | C₂F₅ | CH | O | H | CHMe-4-Pyr | |
| I-B-Q2-014 | 2-Cl | 3-CF₃ | 4-Cl | NHAc | CF₃ | CH | O | Me | CHMe-4-Pyr | |
| I-B-Q2-015 | 3-Cl | 4-CF₃ | H | NAc₂ | CF₃ | CH | O | H | CH₂-2-Pyrm | |
| I-B-Q2-016 | 3-Br | 4-NH₂ | 5-Br | NHMe | CF₂Ph | CH | O | H | CH₂-2-Pyrz | |
| I-B-Q2-017 | 3-Br | H | H | NMe₂ | CF₂OMe | CH | O | H | Me | |
| I-B-Q2-018 | 3-Cl | 4-Cl | H | SMe | CF₂OPh | CH | O | Me | Me | |
| I-B-Q2-019 | 2-Cl | 3-Cl | 4-Cl | SOMe | CF₂(2-Pyr) | N | O | H | 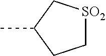 | |
| I-B-Q2-020 | 3-NO₂ | H | H | SO₂Me | CF₂O(2-Pyr) | CH | O | H | CH₂COOMe | |
| I-B-Q2-021 | 3-Cl | H | 5-Cl | H | CF₃ | CH | O | H | CH₂CF₂CF₂H | log P |
| I-B-Q2-022 | 3-Cl | H | 5-Cl | H | CF₃ | CH | O | H | CH₂-2-Pyr | log P |
| I-B-Q2-023 | 3-Cl | H | 5-Cl | H | CF₃ | CH | O | H | CyBu | log P |
| I-B-Q2-024 | 3-Cl | H | 5-Cl | H | CF₃ | CH | O | H | 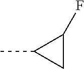 | log P |

I-B-Q2-001: log P (HCOOH): 4.74
I-B-Q2-021: log P (HCOOH): 4.58
I-B-Q2-022: log P (HCOOH): 3.54
I-B-Q2-023: log P (HCOOH): 4.57
I-B-Q2-024: log P (HCOOH): 4.20

TABLE 7
In accordance with the general methods described above, it is possible to prepare the
compounds of the formula (I-B where Q = Q³) listed in the table below.
(I-B-Q³)

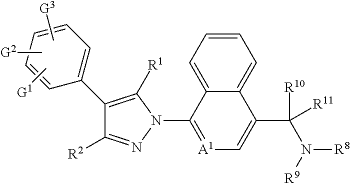

| | | | | | | | | | | | Phys. |
|---|---|---|---|---|---|---|---|---|---|---|---|
| | | | | | | | | | | | chem. |
| Example no. | G¹ | G² | G³ | R¹ | R² | A¹ | R⁸ | R⁹ | R¹⁰ | R¹¹ | data |
| I-B-Q3-001 | 3-Cl | H | 5-Cl | H | CF₃ | CH | Ac | H | H | H | |
| I-B-Q3-002 | 3-CF₃ | H | 5-CF₃ | H | CF₃ | CH | CSMe | H | H | H | |
| I-B-Q3-003 | 3-CF₃ | H | H | NH₂ | CF₃ | CH | COEt | H | H | H | |
| I-B-Q3-004 | 3-Br | H | 5-Br | NH₂ | CF₃ | CH | CH₂-2-Pyr | H | H | H | |
| I-B-Q3-005 | 3-Cl | 4-Cl | 5-Cl | Cl | CF₃ | CH | COiPr | H | H | H | |
| I-B-Q3-006 | 3-Br | 4-Cl | 5-Br | Cl | C₂F₅ | CH | COCyclpr | H | H | H | |
| I-B-Q3-007 | 3-CF₃ | 4-Cl | 5-Cl | Br | C₂F₅ | CH | SO₂Me | H | H | H | |
| I-B-Q3-008 | 3-CF₃ | 4-F | H | Br | C₂F₅ | CH | CONMe₂ | H | H | H | |
| I-B-Q3-009 | 3-Cl | 4-CF₃ | 5-Cl | I | C₂F₅ | CH | CHO | H | H | H | |
| I-B-Q3-010 | 3-CF₃ | H | 5-Cl | I | CF₃ | CH | CONHCH₂CF₃ | H | H | H | |
| I-B-Q3-011 | 3-Cl | 4-CF₃ | 5-Cl | CN | CF₃ | N | COOMe | H | H | H | |
| I-B-Q3-012 | 3-Cl | 4-Br | 5-Cl | CN | CF₃ | N | Ac | Ac | H | H | |
| I-B-Q3-013 | 3-F | 4-F | 5-F | NHCH₂Ph | C₂F₅ | CH | Ac | Ac | Me | Me | |
| I-B-Q3-014 | 2-Cl | 3-CF₃ | 4-Cl | NHAc | CF₃ | CH | COOMe | COOMe | H | H | |
| I-B-Q3-015 | 3-Cl | 4-CF₃ | H | NAc₂ | CF₃ | CH | COOMe | COOMe | Me | Me | |
| I-B-Q3-016 | 3-Br | 4-NH₂ | 5-Br | NHMe | CF₂Ph | CH | SO₂Me | H | Me | Me | |
| I-B-Q3-017 | 3-Br | H | H | NMe₂ | CF₂OMe | CH | CH₂-2-Pyr | H | Me | Me | |
| I-B-Q3-018 | 3-Cl | 4-Cl | H | SMe | CF₂OPh | CH | CH₂-2-Pyr | Ac | Me | Me | |
| I-B-Q3-019 | 2-Cl | 3-Cl | 4-Cl | SOMe | CF₂(2-Pyr) | N | Me | H | H | H | |
| I-B-Q3-020 | 3-NO₂ | H | H | SO₂Me | CF₂O(2-Pyr) | CH | Ph | H | H | H | |

TABLE 8
In accordance with the general methods described above, it is possible to prepare
the compounds of the formula (I-B where Q = Q⁴) listed in the table below.
(I-B-Q⁴)

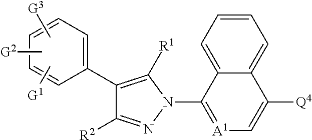

| Table 8 | | | | | | | | Phys. |
|---|---|---|---|---|---|---|---|---|
| Example | | | | | | | | chem. |
| No. | G¹ | G² | G³ | R¹ | R² | A¹ | Q⁴ | data |
| I-B-Q4-001 | 3-Cl | H | 5-Cl | H | CF₃ | CH | F | |
| I-B-Q4-002 | 3-CF₃ | H | 5-CF₃ | H | CF₃ | CH | Cl | |
| I-B-Q4-003 | 3-CF₃ | H | H | NH₂ | CF₃ | CH | Br | |
| I-B-Q4-004 | 3-Br | H | 5-Br | NH₂ | CF₃ | CH | I | |
| I-B-Q4-005 | 3-Cl | 4-Cl | 5-Cl | Cl | CF₃ | CH | CN | |
| I-B-Q4-006 | 3-Br | 4-Cl | 5-Br | Cl | C₂F₅ | CH | NO₂ | |
| I-B-Q4-007 | 3-CF₃ | 4-Cl | 5-Cl | Br | C₂F₅ | CH | SMe | |
| I-B-Q4-008 | 3-CF₃ | 4-F | H | Br | C₂F₅ | CH | SCF₃ | |
| I-B-Q4-009 | 3-Cl | 4-CF₃ | 5-Cl | I | C₂F₅ | CH | SOMe | |
| I-B-Q4-010 | 3-CF₃ | H | 5-Cl | I | CF₃ | CH | SOCF₃ | |
| I-B-Q4-011 | 3-Cl | 4-CF₃ | 5-Cl | CN | CF₃ | N | SO₂Me | |
| I-B-Q4-012 | 3-Cl | 4-Br | 5-Cl | CN | CF₃ | N | SO₂CF₃ | |
| I-B-Q4-013 | 3-F | 4-F | 5-F | NHCH₂Ph | C₂F₅ | CH | CN | |
| I-B-Q4-014 | 2-Cl | 3-CF₃ | 4-Cl | NHAc | CF₃ | CH | NO₂ | |
| I-B-Q4-015 | 3-Cl | 4-CF₃ | H | NAc₂ | CF₃ | CH | F | |
| I-B-Q4-016 | 3-Br | 4-NH₂ | 5-Br | NHMe | CF₂Ph | CH | Cl | |
| I-B-Q4-017 | 3-Br | H | H | NMe₂ | CF₂OMe | CH | Br | |
| I-B-Q4-018 | 3-Cl | 4-Cl | H | SMe | CF₂OPh | CH | I | |
| I-B-Q4-019 | 2-Cl | 3-Cl | 4-Cl | SOMe | CF₂(2-Pyr) | N | COOH | |
| I-B-Q4-020 | 3-NO₂ | H | H | SO₂Me | CF₂O(2-Pyr) | CH | COOEt | |
| I-B-Q4-021 | 3-Cl | H | 5-Cl | NH₂ | CF₃ | CH | COOH | log P |

I-B-Q4-021: log P (HCOOH): 4.12

TABLE 9
In accordance with the general methods
described above, it is possible to prepare the hydrazines of
the formula (VII-A) listed in the table below.
(VII-A)

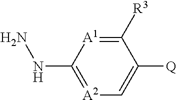

| Table 9 | | | | | Phys. |
|---|---|---|---|---|---|
| Example | | | | | chem. |
| No. | A¹ | A² | R³ | Q | data |
| VII-A-001 | CH | CH | Me | COOH | |
| VII-A-002 | N | CH | Me | COOH | |
| VII-A-003 | CH | N | Me | COOH | |
| VII-A-004 | CH | CH | Me | COOEt | |
| VII-A-005 | N | N | Me | COOMe | |
| VII-A-006 | N | CH | Me | COOMe | |
| VII-A-007 | CH | N | Me | COOMe | |
| VII-A-008 | CH | CH | Me | COOMe | log P |
| | | | | | ¹H-NMR |
| VII-A-009 | N | CH | Me | COOEt | |
| VII-A-010 | CH | N | Me | COOEt | |
| VII-A-011 | CH | CH | CN | COOH | |
| VII-A-012 | N | N | CN | COOH | |
| VII-A-013 | N | CH | CN | COOH | |
| VII-A-014 | CH | N | CN | COOH | |
| VII-A-015 | CH | CH | CN | COOMe | |
| VII-A-016 | N | N | CN | COOMe | |
| VII-A-017 | N | CH | CN | COOMe | |
| VII-A-018 | CH | N | CN | COOMe | |
| VII-A-019 | CH | CH | CN | COOEt | |
| VII-A-020 | N | N | CN | COOEt | |
| VII-A-021 | N | CH | CN | COOEt | |
| VII-A-022 | CH | N | CN | COOEt | |
| VII-A-023 | CH | CH | CF₃ | COOH | |
| VII-A-024 | N | N | CF₃ | COOH | |
| VII-A-025 | N | CH | CF₃ | COOH | |
| VII-A-026 | CH | N | CF₃ | COOH | |
| VII-A-027 | CH | CH | CF₃ | COOMe | |
| VII-A-028 | N | CH | CF₃ | COOMe | |
| VII-A-029 | CH | N | CF₃ | COOMe | |
| VII-A-030 | CH | CH | CF₃ | COOEt | |
| VII-A-031 | N | CH | CF₃ | COOEt | |
| VII-A-032 | CH | N | CF₃ | COOEt | |
| VII-A-033 | N | N | NO₂ | COOH | |
| VII-A-034 | N | CH | NO₂ | COOH | |
| VII-A-035 | CH | N | NO₂ | COOH | |
| VII-A-036 | CH | CH | NO₂ | COOMe | |
| VII-A-037 | N | N | NO₂ | COOMe | |
| VII-A-038 | N | CH | NO₂ | COOMe | |
| VII-A-039 | CH | N | NO₂ | COOMe | |
| VII-A-040 | CH | CH | NO₂ | COOEt | |
| VII-A-041 | N | N | NO₂ | COOEt | |
| VII-A-042 | N | CH | NO₂ | COOEt | |
| VII-A-043 | CH | N | NO₂ | COOEt | |
| VII-A-044 | CH | CH | Br | COOH | |
| VII-A-045 | N | N | Br | COOH | |
| VII-A-046 | N | CH | Br | COOH | |
| VII-A-047 | CH | N | Br | COOH | |
| VII-A-048 | CH | CH | Br | COOMe | |
| VII-A-049 | N | N | Br | COOMe | |
| VII-A-050 | N | CH | Br | COOMe | |
| VII-A-051 | CH | N | Br | COOMe | |
| VII-A-052 | CH | CH | Br | COOEt | |
| VII-A-053 | N | N | Br | COOEt | |
| VII-A-054 | N | CH | Br | COOEt | |
| VII-A-055 | CH | N | Br | COOEt | |
| VII-A-056 | N | N | Cl | COOH | |
| VII-A-057 | N | CH | Cl | COOH | |
| VII-A-058 | CH | N | Cl | COOH | |
| VII-A-059 | N | N | Cl | COOMe | |
| VII-A-060 | N | CH | Cl | COOMe | |
| VII-A-061 | CH | N | Cl | COOMe | |
| VII-A-062 | CH | CH | Cl | COOEt | |
| VII-A-063 | N | N | Cl | COOEt | |
| VII-A-064 | N | CH | Cl | COOEt | |
| VII-A-065 | CH | N | Cl | COOEt | |
| VII-A-066 | CH | CH | I | COOH | |
| VII-A-067 | N | N | I | COOH | |
| VII-A-068 | N | CH | I | COOH | |
| VII-A-069 | CH | N | I | COOH | |
| VII-A-070 | CH | CH | I | COOMe | |
| VII-A-071 | N | N | I | COOMe | |
| VII-A-072 | N | CH | I | COOMe | |
| VII-A-073 | CH | N | I | COOMe | |
| VII-A-074 | CH | CH | I | COOEt | |
| VII-A-075 | N | N | I | COOEt | |
| VII-A-076 | N | CH | I | COOEt | |
| VII-A-077 | CH | N | I | COOEt | |
| VII-A-078 | CH | CH | OCF₃ | COOH | |
| VII-A-079 | N | N | OCF₃ | COOH | |
| VII-A-080 | N | CH | OCF₃ | COOH | |
| VII-A-081 | CH | N | OCF₃ | COOH | |
| VII-A-082 | CH | CH | OCF₃ | COOMe | |
| VII-A-083 | N | N | OCF₃ | COOMe | |
| VII-A-084 | N | CH | OCF₃ | COOMe | |
| VII-A-085 | CH | N | OCF₃ | COOMe | |
| VII-A-086 | CH | CH | OCF₃ | COOEt | |
| VII-A-087 | N | N | OCF₃ | COOEt | |
| VII-A-088 | N | CH | OCF₃ | COOEt | |
| VII-A-089 | CH | N | OCF₃ | COOEt | |
| VII-A-090 | CH | CH | CH₂OMe | COOH | |
| VII-A-091 | N | N | CH₂OMe | COOH | |
| VII-A-092 | N | CH | CH₂OMe | COOH | |
| VII-A-093 | CH | N | CH₂OMe | COOH | |
| VII-A-094 | CH | CH | CH₂OMe | COOMe | |
| VII-A-095 | N | N | CH₂OMe | COOMe | |
| VII-A-096 | N | CH | CH₂OMe | COOMe | |
| VII-A-097 | CH | N | CH₂OMe | COOMe | |
| VII-A-098 | CH | CH | CH₂OMe | COOEt | |
| VII-A-099 | N | N | CH₂OMe | COOEt | |
| VII-A-100 | N | CH | CH₂OMe | COOEt | |
| VII-A-101 | CH | N | CH₂OMe | COOEt | |
| VII-A-102 | CH | CH | Me | 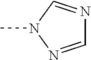 | |
| VII-A-103 | CH | CH | CN | 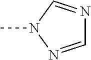 | ¹H-NMR |
| VII-A-104 | CH | CH | CF₃ | 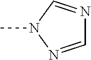 | |
| VII-A-105 | CH | CH | NO₂ | 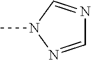 | |
| VII-A-106 | CH | CH | Br | 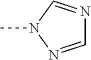 | |
| VII-A-107 | CH | CH | Cl | 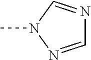 | |
| VII-A-108 | CH | CH | I | 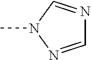 | |
| VII-A-109 | CH | CH | OCF₃ | 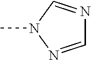 | |
| VII-A-110 | CH | CH | CH₂OMe | 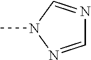 | |
| VII-A-111 | CH | CH | Me | CONH—CH₂-2-Pyr | |
| VII-A-112 | CH | CH | CN | CONH—CH₂-2-Pyr | |
| VII-A-113 | CH | CH | CF₃ | CONH—CH₂-2-Pyr | |
| VII-A-114 | CH | CH | NO₂ | CONH—CH₂-2-Pyr | |
| VII-A-115 | CH | CH | Br | CONH—CH₂-2-Pyr | |
| VII-A-116 | CH | CH | Cl | CONH—CH₂-2-Pyr | |
| VILA-117 | CH | CH | I | CONH—CH₂-2-Pyr | |
| VII-A-118 | CH | CH | OCF₃ | CONH—CH₂-2-Pyr | |
| VII-A-119 | CH | CH | CH₂OMe | CONH—CH₂-2-Pyr | |
| VII-A-120 | CH | CH | Me | CONH—CHMe-2-Pyr | |
| VII-A-121 | CH | CH | CN | CONH—CHMe-2-Pyr | |
| VII-A-122 | CH | CH | CF₃ | CONH—CHMe-2-Pyr | |
| VII-A-123 | CH | CH | NO₂ | CONH—CHMe-2-Pyr | |
| VII-A-124 | CH | CH | Br | CONH—CHMe-2-Pyr | |
| VII-A-125 | CH | CH | Cl | CONH—CHMe-2-Pyr | |
| VII-A-126 | CH | CH | I | CONH—CHMe-2-Pyr | |
| VII-A-127 | CH | CH | OCF₃ | CONH—CHMe-2-Pyr | |
| VII-A-128 | CH | CH | CH₂OMe | CONH—CHMe-2-Pyr | |
| VII-A-129 | CH | CH | Me | CONH-TFiPr | |
| VII-A-130 | CH | CH | CN | CONH-TFiPr | |
| VII-A-131 | CH | CH | CF₃ | CONH-TFiPr | |
| VII-A-132 | CH | CH | NO₂ | CONH-TFiPr | |
| VII-A-133 | CH | CH | Br | CONH-TFiPr | |
| VII-A-134 | CH | CH | Cl | CONH-TFiPr | |
| VII-A-135 | CH | CH | I | CONH-TFiPr | |
| VII-A-136 | CH | CH | OCF₃ | CONH-TFiPr | |
| VII-A-137 | CH | CH | CH₂OMe | CONH-TFiPr | |

### Characteristic Data for Synthesis Examples:

VII-A-008: spectroscopic data see protocol under general synthesis procedures
VII-A-103: spectroscopic data see protocol under general synthesis procedures

TABLE 10

| In accordance with the general methods described | | | |
|---|---|---|---|
| above, it is possible to prepare | | | |
| the hydrazines of the | | | |
| formula (VII-B) listed in the table below. | | | |
| (VII-B) | | | |
| 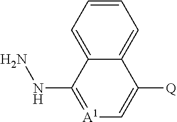 | | | |
| Table 10 | | | Phys. |
| Example | | | chem. |
| No. | A¹ | Q | data |
| VII-B-001 | N | COOH | |
| VII-B-002 | CH | COOMe | |
| VII-B-003 | CH | COOEt | |
| VII-B-004 | N | COOEt | |
| VII-B-005 | CH | COOH | |

TABLE 11
In accordance with the general methods described above,
it is possible to prepare the compounds
of the formula (IX) listed in the table below.
(IX)

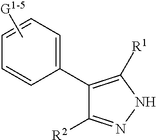

| Table | | | | | | |
|---|---|---|---|---|---|---|
| 11 | | | | | | |
| Ex- | | | | | | Phys. |
| ample | | | | | | chem. |
| No. | G¹ | G² | G³ | R¹ | R² | data |
| IX-001 | 3-Cl | H | 5-Cl | H | CF₃ | |
| IX-002 | 3-CF₃ | H | 5-CF₃ | H | CF₃ | |
| IX-003 | 3-CF₃ | H | H | H | CF₃ | |
| IX-004 | 3-Br | H | 5-Br | H | CF₃ | |
| IX-005 | 3-Cl | 4-Cl | 5-Cl | H | CF₃ | |
| IX-006 | 3-Br | 4-Cl | 5-Br | H | C₂F₅ | |
| IX-007 | 3-CF₃ | 4-Cl | 5-Cl | H | C₂F₅ | |
| IX-008 | 3-CF₃ | 4-F | H | H | C₂F₅ | |
| IX-009 | 3-Cl | 4-CF₃ | 5-Cl | H | C₂F₅ | |
| IX-010 | 3-CF₃ | H | 5-Cl | H | CF₃ | |
| IX-011 | 3-Cl | 4-CF₃ | 5-Cl | H | CF₃ | |
| IX-012 | 3-Cl | 4-Br | 5-Cl | H | CF₃ | |
| IX-013 | 3-F | 4-F | 5-F | H | C₂F₅ | |
| IX-014 | 2-Cl | 3-CF₃ | 4-Cl | H | CF₃ | |
| IX-015 | 3-Cl | 4-CF₃ | H | H | CF₃ | |
| IX-016 | 3-Br | 4-NH₂ | 5-Br | H | CF₂Ph | |
| IX-017 | 3-Br | H | H | H | CF₂OMe | |
| IX-018 | 3-Cl | 4-Cl | H | H | CF₂OPh | |
| IX-019 | 2-Cl | 3-Cl | 4-Cl | H | CF₂ | |
| | | | | | (2-Pyr) | |
| IX-020 | 3-NO₂ | H | H | H | CF₂O | |
| | | | | | (2-Pyr) | |
| IX-021 | 3-Cl | H | 5-Cl | NH₂ | CF₃ | log P |
| | | | | | | ¹H-NMR |
| IX-022 | 3-CF₃ | H | 5-CF₃ | NH₂ | CF₃ | |
| IX-023 | 3-CF₃ | H | H | NH₂ | CF₃ | |
| IX-024 | 3-Br | H | 5-Br | NH₂ | CF₃ | |
| IX-025 | 3-Cl | 4-Cl | 5-Cl | NH₂ | CF₃ | |
| IX-026 | 3-Br | 4-Cl | 5-Br | NH₂ | CF₃ | |
| IX-027 | 3-CF₃ | 4-Cl | 5-Cl | NH₂ | CF₃ | |
| IX-028 | 3-CF₃ | 4-F | H | NH₂ | CF₃ | |
| IX-029 | 3-Cl | 4-CF₃ | 5-Cl | NH₂ | CF₃ | |
| IX-030 | 3-CF₃ | H | 5-Cl | NH₂ | CF₃ | |
| IX-031 | 3-Cl | 4-CF₃ | 5-Cl | NH₂ | CF₃ | |
| IX-032 | 3-Cl | 4-Br | 5-Cl | NH₂ | CF₃ | |
| IX-033 | 3-F | 4-F | 5-F | NH₂ | CF₃ | |
| IX-034 | 2-Cl | 3-CF₃ | 4-Cl | NH₂ | CF₃ | |
| IX-035 | 3-Cl | 4-CF₃ | H | NH₂ | CF₃ | |
| IX-036 | 3-Br | 4-NH₂ | 5-Br | NH₂ | CF₃ | |
| IX-037 | 3-Br | H | H | NH₂ | CF₃ | |
| IX-038 | 3-Cl | 4-Cl | H | NH₂ | CF₃ | log P |
| | | | | | | ¹H-NMR |
| IX-039 | 2-Cl | 3-Cl | 4-Cl | NH₂ | CF₃ | |
| IX-040 | 3-NO₂ | H | H | NH₂ | CF₃ | |
| IX-041 | 3-Cl | H | 5-Cl | NH₂ | C₂F₅ | |
| IX-042 | 3-CF₃ | H | 5-CF₃ | NH₂ | C₂F₅ | |
| IX-043 | 3-CF₃ | H | H | NH₂ | C₂F₅ | |
| IX-044 | 3-Br | H | 5-Br | NH₂ | C₂F₅ | |
| IX-045 | 3-Cl | 4-Cl | 5-Cl | NH₂ | C₂F₅ | |
| IX-046 | 3-Br | 4-Cl | 5-Br | NH₂ | C₂F₅ | |
| IX-047 | 3-CF₃ | 4-Cl | 5-Cl | NH₂ | CF₂Ph | |
| IX-048 | 3-CF₃ | 4-F | H | NH₂ | CF₂OMe | |
| IX-049 | 3-Cl | 4-CF₃ | 5-Cl | NH₂ | CF₂OPh | |
| IX-050 | 3-CF₃ | H | 5-Cl | NH₂ | CF₂ | |
| | | | | | (2-Pyr) | |
| IX-051 | 3-Cl | 4-CF₃ | 5-Cl | NH₂ | CF₂O | |
| | | | | | (2-Pyr) | |
| IX-052 | 3-Cl | 4-Br | 5-Cl | NH₂ | C₂F₅ | |
| IX-053 | 3-F | 4-F | 5-F | NH₂ | C₂F₅ | |
| IX-054 | 2-Cl | 3-CF₃ | 4-Cl | NH₂ | C₂F₅ | |
| IX-055 | 3-Cl | 4-CF₃ | H | NH₂ | C₂F₅ | |
| IX-056 | 3-Br | 4-NH₂ | 5-Br | NH₂ | CF₂Ph | |
| IX-057 | 3-Br | H | H | NH₂ | CF₂OMe | |
| IX-058 | 3-Cl | 4-Cl | H | NH₂ | CF₂OPh | |
| IX-059 | 2-Cl | 3-Cl | 4-Cl | NH₂ | CF₂ | |
| | | | | | (2-Pyr) | |
| IX-060 | 3-NO₂ | H | H | NH₂ | CF₂O | |
| | | | | | (2-Pyr) | |
| IX-061 | 3-Cl | H | 5-Cl | NHAc | CF₃ | |
| IX-062 | 3-CF₃ | H | 5-CF₃ | NAc₂ | CF₃ | |
| IX-063 | 3-CF₃ | H | H | NH(C(O)OMe) | CF₃ | |
| IX-064 | 3-Br | H | 5-Br | N(C(O)OMe)₂ | CF₃ | |
| IX-065 | 3-Cl | 4-Cl | 5-Cl | NHMe | CF₃ | |
| IX-066 | 3-Br | 4-Cl | 5-Br | NMe₂ | C₂F₅ | |
| IX-067 | 3-CF₃ | 4-Cl | 5-Cl | NH(CH₂Ph) | C₂F₅ | |
| IX-068 | 3-CF₃ | 4-F | H | N(CH₂Ph)₂ | C₂F₅ | |
| IX-069 | 3-Cl | 4-CF₃ | 5-Cl | NH(CH₂-2-Pyr) | C₂F₅ | |
| IX-070 | 3-CF₃ | H | 5-Cl | N(CH₂-2-Pyr)₂ | CF₃ | |
| IX-071 | 3-Cl | 4-CF₃ | 5-Cl | NH(SO₂Me) | CF₃ | |
| IX-072 | 3-Cl | 4-Br | 5-Cl | NH(SO₂CF₃) | CF₃ | |
| IX-073 | 3-F | 4-F | 5-F | NHCypr | C₂F₅ | |
| IX-074 | 2-Cl | 3-CF₃ | 4-Cl | NHEt | CF₃ | |
| IX-075 | 3-Cl | 4-CF₃ | H | NHPh | CF₃ | |
| IX-076 | 3-Br | 4-NH₂ | 5-Br | NH(C(O)OtBu) | CF₂Ph | |
| IX-077 | 3-Br | H | H | NH(C(O)tBu | CF₂OMe | |
| IX-078 | 3-Cl | 4-Cl | H | NHtBu | CF₂OPh | |
| IX-079 | 2-Cl | 3-Cl | 4-Cl | NH(CH₂- | CF₂ | |
| | | | | 2-Pyrim) | (2-Pyr) | |
| IX-080 | 3-NO₂ | H | H | NH(CH₂- | CF₂O | |
| | | | | 2-Pyraz) | (2-Pyr) | |
| IX-081 | 3-Cl | H | 5-Cl | NHAc | CF₃ | |
| IX-082 | 3-CF₃ | H | 5-CF₃ | NAc₂ | CF₃ | |
| IX-083 | 3-CF₃ | H | H | NH(C(O)OMe) | CF₃ | |
| IX-084 | 3-Br | H | 5-Br | N(C(O)OMe)₂ | CF₃ | |
| IX-085 | 3-Cl | 4-Cl | 5-Cl | NHMe | CF₃ | |
| IX-086 | 3-Br | 4-Cl | 5-Br | NMe₂ | C₂F₅ | |
| IX-087 | 3-CF₃ | 4-Cl | 5-Cl | NH(CH₂Ph) | C₂F₅ | |
| IX-088 | 3-CF₃ | 4-F | H | N(CH₂Ph)₂ | C₂F₅ | |
| IX-089 | 3-Cl | 4-CF₃ | 5-Cl | NH(CH₂-2-Pyr) | C₂F₅ | |
| IX-090 | 3-CF₃ | H | 5-Cl | N(CH₂-2-Pyr)₂ | CF₃ | |
| IX-091 | 3-Cl | 4-CF₃ | 5-Cl | NH(SO₂Me) | CF₃ | |
| IX-092 | 3-Cl | 4-Br | 5-Cl | NH(SO₂CF₃) | CF₃ | |
| IX-093 | 3-F | 4-F | 5-F | NHCypr | C₂F₅ | |
| IX-094 | 2-Cl | 3-CF₃ | 4-Cl | NHEt | CF₃ | |
| IX-095 | 3-Cl | 4-CF₃ | H | NHPh | CF₃ | |
| IX-096 | 3-Br | 4-NH₂ | 5-Br | NH(C(O)OtBu) | CF₂Ph | |
| IX-097 | 3-Br | H | H | NH(C(O)tBu | CF₂OMe | |
| IX-098 | 3-Cl | 4-Cl | H | NHiPr | CF₂OPh | |
| IX-099 | 2-Cl | 3-Cl | 4-Cl | NH(CH₂- | CF₂ | |
| | | | | 2-Pyrim) | (2-Pyr) | |
| IX-100 | 3-NO₂ | H | H | NH(CH₂- | CF₂O | |
| | | | | 2-Pyraz) | (2-Pyr) | |
| IX-101 | 3-Cl | H | 5-Cl | Cl | CF₃ | |
| IX-102 | 3-CF₃ | H | 5-CF₃ | Cl | CF₃ | |
| IX-103 | 3-CF₃ | H | H | Cl | CF₃ | |
| IX-104 | 3-Br | H | 5-Br | Cl | CF₃ | |
| IX-105 | 3-Cl | 4-Cl | 5-Cl | Cl | CF₃ | |
| IX-106 | 3-Br | 4-Cl | 5-Br | Cl | C₂F₅ | |
| IX-107 | 3-CF₃ | 4-Cl | 5-Cl | Cl | C₂F₅ | |
| IX-108 | 3-CF₃ | 4-F | H | Cl | C₂F₅ | |
| IX-109 | 3-Cl | 4-CF₃ | 5-Cl | Cl | C₂F₅ | |
| IX-110 | 3-CF₃ | H | 5-Cl | Cl | CF₃ | |
| IX-111 | 3-Cl | 4-CF₃ | 5-Cl | Cl | CF₃ | |
| IX-112 | 3-Cl | 4-Br | 5-Cl | Cl | CF₃ | |
| IX-113 | 3-F | 4-F | 5-F | Cl | C₂F₅ | |
| IX-114 | 2-Cl | 3-CF₃ | 4-Cl | Cl | CF₃ | |
| IX-115 | 3-Cl | 4-CF₃ | H | Cl | CF₃ | |
| IX-116 | 3-Br | 4-NH₂ | 5-Br | Cl | CF₂Ph | |
| IX-117 | 3-Br | H | H | Cl | CF₂OMe | |
| IX-118 | 3-Cl | 4-Cl | H | Cl | CF₂OPh | |
| IX-119 | 2-Cl | 3-Cl | 4-Cl | Cl | CF₂ | |
| | | | | | (2-Pyr) | |
| IX-120 | 3-NO₂ | H | H | Cl | CF₂O | |
| | | | | | (2-Pyr) | |
| IX-121 | 3-Cl | H | 5-Cl | Br | CF₃ | |
| IX-122 | 3-CF₃ | H | 5-CF₃ | Br | CF₃ | |
| IX-123 | 3-CF₃ | H | H | Br | CF₃ | |
| IX-124 | 3-Br | H | 5-Br | Br | CF₃ | |
| IX-125 | 3-Cl | 4-Cl | 5-Cl | Br | CF₃ | |
| IX-126 | 3-Br | 4-Cl | 5-Br | Br | C₂F₅ | |
| IX-127 | 3-CF₃ | 4-Cl | 5-Cl | Br | C₂F₅ | |
| IX-128 | 3-CF₃ | 4-F | H | Br | C₂F₅ | |
| IX-129 | 3-Cl | 4-CF₃ | 5-Cl | Br | C₂F₅ | |
| IX-130 | 3-CF₃ | H | 5-Cl | Br | CF₃ | |
| IX-131 | 3-Cl | 4-CF₃ | 5-Cl | Br | CF₃ | |
| IX-132 | 3-Cl | 4-Br | 5-Cl | Br | CF₃ | |
| IX-133 | 3-F | 4-F | 5-F | Br | C₂F₅ | |
| IX-134 | 2-Cl | 3-CF₃ | 4-Cl | Br | CF₃ | |
| IX-135 | 3-Cl | 4-CF₃ | H | Br | CF₃ | |
| IX-136 | 3-Br | 4-NH₂ | 5-Br | Br | CF₂Ph | |
| IX-137 | 3-Br | H | H | Br | CF₂OMe | |
| IX-138 | 3-Cl | 4-Cl | H | Br | CF₂OPh | |
| IX-139 | 2-Cl | 3-Cl | 4-Cl | Br | CF₂ | |
| | | | | | (2-Pyr) | |
| IX-140 | 3-NO₂ | H | H | Br | CF₂O | |
| | | | | | (2-Pyr) | |
| IX-141 | 3-Cl | H | 5-Cl | I | CF₃ | |
| IX-142 | 3-CF₃ | H | 5-CF₃ | I | CF₃ | |
| IX-143 | 3-CF₃ | H | H | I | CF₃ | |
| IX-144 | 3-Br | H | 5-Br | I | CF₃ | |
| IX-145 | 3-Cl | 4-Cl | 5-Cl | I | CF₃ | |
| IX-146 | 3-Br | 4-Cl | 5-Br | I | C₂F₅ | |
| IX-147 | 3-CF₃ | 4-Cl | 5-Cl | I | C₂F₅ | |
| IX-148 | 3-CF₃ | 4-F | H | I | C₂F₅ | |
| IX-149 | 3-Cl | 4-CF₃ | 5-Cl | I | C₂F₅ | |
| IX-150 | 3-CF₃ | H | 5-Cl | I | CF₃ | |
| IX-151 | 3-Cl | 4-CF₃ | 5-Cl | I | CF₃ | |
| IX-152 | 3-Cl | 4-Br | 5-Cl | I | CF₃ | |
| IX-153 | 3-F | 4-F | 5-F | I | C₂F₅ | |
| IX-154 | 2-Cl | 3-CF₃ | 4-Cl | I | CF₃ | |
| IX-155 | 3-Cl | 4-CF₃ | H | I | CF₃ | |
| IX-156 | 3-Br | 4-NH₂ | 5-Br | I | CF₂Ph | |
| IX-157 | 3-Br | H | H | I | CF₂OMe | |
| IX-158 | 3-Cl | 4-Cl | H | I | CF₂OPh | |
| IX-159 | 2-Cl | 3-Cl | 4-Cl | I | CF₂ | |
| | | | | | (2-Pyr) | |
| IX-160 | 3-NO₂ | H | H | I | CF₂O | |
| | | | | | (2-Pyr) | |
| IX-161 | 3-Cl | H | 5-Cl | CN | CF₃ | |
| IX-162 | 3-CF₃ | H | 5-CF₃ | CN | CF₃ | |
| IX-163 | 3-CF₃ | H | H | CN | CF₃ | |
| IX-164 | 3-Br | H | 5-Br | CN | CF₃ | |
| IX-165 | 3-Cl | 4-Cl | 5-Cl | CN | CF₃ | |
| IX-166 | 3-Br | 4-Cl | 5-Br | CN | C₂F₅ | |
| IX-167 | 3-CF₃ | 4-Cl | 5-Cl | CN | C₂F₅ | |
| IX-168 | 3-CF₃ | 4-F | H | CN | C₂F₅ | |
| IX-169 | 3-Cl | 4-CF₃ | 5-Cl | CN | C₂F₅ | |
| IX-170 | 3-CF₃ | H | 5-Cl | CN | CF₃ | |
| IX-171 | 3-Cl | 4-CF₃ | 5-Cl | CN | CF₃ | |
| IX-172 | 3-Cl | 4-Br | 5-Cl | CN | CF₃ | |
| IX-173 | 3-F | 4-F | 5-F | CN | C₂F₅ | |
| IX-174 | 2-Cl | 3-CF₃ | 4-Cl | CN | CF₃ | |
| IX-175 | 3-Cl | 4-CF₃ | H | CN | CF₃ | |
| IX-176 | 3-Br | 4-NH₂ | 5-Br | CN | CF₂Ph | |
| IX-177 | 3-Br | H | H | CN | CF₂OMe | |
| IX-178 | 3-Cl | 4-Cl | H | CN | CF₂OPh | |
| IX-179 | 2-Cl | 3-Cl | 4-Cl | CN | CF₂ | |
| | | | | | (2-Pyr) | |
| IX-180 | 3-NO₂ | H | H | CN | CF₂O | |
| | | | | | (2-Pyr) | |
| IX-181 | 3-Cl | H | 5-Cl | SMe | CF₃ | |
| IX-182 | 3-CF₃ | H | 5-CF₃ | SOMe | CF₃ | |
| IX-183 | 3-CF₃ | H | H | SO₂Me | CF₃ | |
| IX-184 | 3-Br | H | 5-Br | SMe | CF₃ | |
| IX-185 | 3-Cl | 4-Cl | 5-Cl | SOMe | CF₃ | |
| IX-186 | 3-Br | 4-Cl | 5-Br | SO₂Me | C₂F₅ | |
| IX-187 | 3-CF₃ | 4-Cl | 5-Cl | SMe | C₂F₅ | |
| IX-188 | 3-CF₃ | 4-F | H | SOMe | C₂F₅ | |
| IX-189 | 3-Cl | 4-CF₃ | 5-Cl | SO₂Me | C₂F₅ | |
| IX-190 | 3-CF₃ | H | 5-Cl | SMe | CF₃ | |
| IX-191 | 3-Cl | 4-CF₃ | 5-Cl | SOMe | CF₃ | |
| IX-192 | 3-Cl | 4-Br | 5-Cl | SO₂Me | CF₃ | |
| IX-193 | 3-F | 4-F | 5-F | SMe | C₂F₅ | |
| IX-194 | 2-Cl | 3-CF₃ | 4-Cl | SOMe | CF₃ | |
| IX-195 | 3-Cl | 4-CF₃ | H | SO₂Me | CF₃ | |
| IX-196 | 3-Br | 4-NH₂ | 5-Br | SMe | CF₂Ph | |
| IX-197 | 3-Br | H | H | SOMe | CF₂OMe | |
| IX-198 | 3-Cl | 4-Cl | H | SO₂Me | CF₂OPh | |
| IX-199 | 2-Cl | 3-Cl | 4-Cl | SMe | CF₂ | |
| | | | | | (2-Pyr) | |
| IX-200 | 3-NO₂ | H | H | SOMe | CF₂O | |
| | | | | | (2-Pyr) | |
| IX-201 | 3-F | 4-F | H | NH₂ | CF₃ | log P |
| IX-202 | H | 4-F | H | NH₂ | CF₃ | log P |
| IX-203 | 3-Cl | H | 5-Cl | NHAc | CF₃ | log P |
| IX-204 | 2-Cl | 4-Cl | H | NHAc | CF₃ | log P |
| | | | | | | ¹H-NMR |

Characteristic data for synthesis examples:
IX-021: log P (HCOOH): 2.91
¹H NMR (DMSO-d6): δ = 12.32 (s, 1 H), 7.45 (s, 1 H), 7.25 (s, 2), 5.32 (s, 2 H) ppm
IX-38: log P (HCOOH): 2.80
¹H NMR (DMSO-d6): δ = 5.27 (br. s, 2H); 7.25 (dd, 1H); 7.46 (d, 1H); 7.61 (d, 1H) ppm
IX-201: log P (HCOOH): 2.33
IX-202: log P (HCOOH): 2.16
IX-203: log P (HCOOH): 2.98
IX-204: log P (HCOOH): 2.79
¹H NMR (DMSO-d6): δ = 1.99 (s, 3H); 7.32 (d, 1H); 7.47 (d, 1H); 7.67 (s, 1H); 9.98 (s, 1H, NH); 13.53 (s, 1H, NH) ppm

TABLE 12
In accordance with the general methods described above,
it is possible to prepare the compounds
of the formula (XI-A) listed in the table below.
(XI-A)

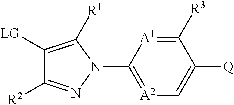

| Table 12 | | | | | | | | Phys. |
|---|---|---|---|---|---|---|---|---|
| Example | | | | | | | | chem. |
| No. | LG | R¹ | R² | R³ | A¹ | A² | Q | data |
| XI-A-001: | I | H | CF₃ | CF₃ | CH | CH | COOEt | ¹H-NMR |
| XI-A-002 | Br | H | CF₃ | Me | CH | CH | COOEt | |
| XI-A-003 | Br | H | CF₃ | CN | CH | CH | COOH | |
| XI-A-004 | Br | NH₂ | CF₃ | NO₂ | CH | CH | Br | |
| XI-A-005 | Br | NH₂ | CF₃ | CF₃ | CH | CH | I | |
| XI-A-006 | Br | Cl | CF₃ | Br | CH | CH | CN | |
| XI-A-007 | Br | Cl | C₂F₅ | Cl | CH | CH | NO₂ | |
| XI-A-008 | Br | Br | C₂F₅ | CH₂OMe | CH | CH | SMe | |
| XI-A-009 | Br | Br | C₂F₅ | CH₂NHMe | CH | CH | SCF₃ | |
| XI-A-010 | Br | I | C₂F₅ | Me | CH | CH | SOMe | |
| XI-A-011 | Br | I | CF₃ | CN | CH | CH | SOCF₃ | |
| XI-A-012 | Br | CN | CF₃ | NO₂ | N | N | SO₂Me | |
| XI-A-013 | Br | CN | CF₃ | CF₃ | N | N | SO₂CF₃ | |
| XI-A-014 | Br | NHCH₂Ph | C₂F₅ | Br | CH | CH | CN | |
| XI-A-015 | Br | NHAc | CF₃ | Cl | CH | CH | NO₂ | |
| XI-A-016 | Br | NAc₂ | CF₃ | CH₂OAc | CH | CH | F | |
| XI-A-017 | Br | NHMe | CF₂Ph | CH₂NHAc | CH | CH | Cl | |
| XI-A-018 | Br | NH₂ | CF₃ | H | CH | CH | COOEt | log P |
| | | | | | | | | ¹H-NMR |
| XI-A-019 | Br | SMe | CF₂Oph | CN | CH | CH | CONH—CH₂-Pyr | |
| XI-A-020 | Br | SOMe | CF₂(2-Pyr) | NO2 | N | CH | CONH—CHMe-2-Pyr | |
| XI-A-021 | Br | SO₂Me | CF₂O(2-Pyr) | CF₃ | CH | N | CONH-TfiPr | |
| XI-A-022 | I | H | CF₃ | Me | CH | CH | COOEt | |
| XI-A-023 | I | H | CF₃ | CN | CH | CH | COOH | |
| XI-A-024 | I | NH₂ | CF₃ | NO₂ | CH | CH | Br | |
| XI-A-025 | I | NH₂ | CF₃ | CF₃ | CH | CH | I | |
| XI-A-026 | I | Cl | CF₃ | Br | CH | CH | CN | |
| XI-A-027 | I | Cl | C₂F₅ | Cl | CH | CH | NO₂ | |
| XI-A-028 | I | Br | C₂F₅ | CH₂OMe | CH | CH | SMe | |
| XI-A-029 | I | Br | C₂F₅ | CH₂NHMe | CH | CH | SCF₃ | |
| XI-A-030 | I | I | C₂F₅ | Me | CH | CH | SOMe | |
| XI-A-031 | I | I | CF₃ | CN | CH | CH | SOCF₃ | |
| XI-A-032 | I | CN | CF₃ | NO₂ | N | N | SO₂Me | |
| XI-A-033 | I | CN | CF₃ | CF₃ | N | N | SO₂CF₃ | |
| XI-A-034 | I | NHCH₂Ph | C₂F₅ | Br | CH | CH | CN | |
| XI-A-035 | I | NHAc | CF₃ | Cl | CH | CH | NO₂ | |
| XI-A-036 | I | NAc₂ | CF₃ | CH₂OAc | CH | CH | F | |
| XI-A-037 | I | NHMe | CF₂Ph | CH₂NHAc | CH | CH | Cl | |
| XI-A-038 | Br | NH₂ | CF₃ | H | CH | CH | COOH | log P |
| | | | | | | | | ¹H-NMR |
| XI-A-039 | I | SMe | CF₂Oph | CN | CH | CH | CONH—CH₂-2-Pyr | |
| XI-A-040 | I | SOMe | CF₂(2-Pyr) | NO2 | N | CH | CONH—CHMe-2-Pyr | |
| XI-A-041 | I | SO₂Me | CF₂O(2-Pyr) | CF₃ | CH | N | CONH-TfiPr | |
| XI-A-042 | Cl | NH₂ | CF₃ | H | CH | CH | COOEt | log P |
| | | | | | | | | ¹H-NMR |
| XI-A-043 | Cl | NH₂ | CF₃ | H | CH | CH | COOH | log P |
| | | | | | | | | ¹H-NMR |
| XI-A-044 | I | NH₂ | CF₃ | H | CH | CH | COOEt | log P |
| | | | | | | | | ¹H-NMR |
| XI-A-045 | I | NH₂ | CF₃ | H | CH | CH | COOH | log P |
| | | | | | | | | ¹H-NMR |
| XI-A-046 | Cl | NH₂ | CF₃ | H | CH | CH | CO—NH—Me | ¹H-NMR |
| XI-A-047 | Cl | NH₂ | CF₃ | H | CH | CH | CO—NMe₂ | ¹H-NMR |
| XI-A-048 | Cl | NH₂ | CF₃ | H | CH | CH | CONH—Et | ¹H-NMR |
| XI-A-049 | Cl | NH₂ | CF₃ | H | CH | CH | CONH—Pr | ¹H-NMR |
| XI-A-050 | Cl | NH₂ | CF₃ | H | CH | CH | CONH—iPr | ¹H-NMR |
| XI-A-051 | Cl | NH₂ | CF₃ | H | CH | CH | CONH-Cypr | ¹H-NMR |
| XI-A-052 | Cl | NH₂ | CF₃ | H | CH | CH | CONH-TFiPr | ¹H-NMR |
| XI-A-053 | Cl | NH₂ | CF₃ | H | CH | CH | CONH—tBu | ¹H-NMR |
| XI-A-054 | Cl | NH₂ | CF₃ | H | CH | CH | CONH—CHMe—tBu | ¹H-NMR |
| XI-A-055 | Cl | NH₂ | CF₃ | H | CH | CH | CONH-3-Pyr | ¹H-NMR |
| XI-A-056 | Cl | NH₂ | CF₃ | H | CH | CH | CONH—CH₂-2-Pyr | ¹H-NMR |
| XI-A-057 | Br | NH₂ | CF₃ | H | CH | CH | CO—NH—Me | ¹H-NMR |
| XI-A-058 | Br | NH₂ | CF₃ | H | CH | CH | CO—NMe₂ | ¹H-NMR |
| XI-A-059 | Br | NH₂ | CF₃ | H | CH | CH | CONH—Et | ¹H-NMR |
| XI-A-060 | Br | NH₂ | CF₃ | H | CH | CH | CONH—Pr | ¹H-NMR |
| XI-A-061 | Br | NH₂ | CF₃ | H | CH | CH | CONH—iPr | ¹H-NMR |
| XI-A-062 | Br | NH₂ | CF₃ | H | CH | CH | CONH-Cypr | ¹H-NMR |
| XI-A-063 | Br | NH₂ | CF₃ | H | CH | CH | CONH-TFiPr | ¹H-NMR |
| XI-A-064 | Br | NH₂ | CF₃ | H | CH | CH | CONH—tBu | ¹H-NMR |
| XI-A-065 | Br | NH₂ | CF₃ | H | CH | CH | CONH—CHMe—tBu | ¹H-NMR |
| XI-A-066 | Br | NH₂ | CF₃ | H | CH | CH | CONH-3-Pyr | ¹H-NMR |
| XI-A-067 | Br | NH₂ | CF₃ | H | CH | CH | CONH—CH₂-2-Pyr | ¹H-NMR |
| XI-A-068 | I | NH₂ | CF₃ | H | CH | CH | CO—NH—Me | ¹H-NMR |
| XI-A-069 | I | NH₂ | CF₃ | H | CH | CH | CO—NMe₂ | ¹H-NMR |
| XI-A-070 | I | NH₂ | CF₃ | H | CH | CH | CONH—Et | ¹H-NMR |
| XI-A-071 | I | NH₂ | CF₃ | H | CH | CH | CONH—Pr | ¹H-NMR |
| XI-A-072 | I | NH₂ | CF₃ | H | CH | CH | CONH—iPr | ¹H-NMR |
| XI-A-073 | I | NH₂ | CF₃ | H | CH | CH | CONH-Cypr | ¹H-NMR |
| XI-A-074 | I | NH₂ | CF₃ | H | CH | CH | CONH-TFiPr | ¹H-NMR |
| XI-A-075 | I | NH₂ | CF₃ | H | CH | CH | CONH—tBu | ¹H-NMR |
| XI-A-076 | I | NH₂ | CF₃ | H | CH | CH | CONH—CHMe—tBu | ¹H-NMR |
| XI-A-077 | I | NH₂ | CF₃ | H | CH | CH | CONH-3-Pyr | ¹H-NMR |
| XI-A-078 | I | NH₂ | CF₃ | H | CH | CH | CONH—CH₂-2-Pyr | ¹H-NMR |
| XI-A-079 | H | NH₂ | CF₃ | CN | CH | CH | 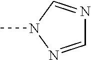 | log P |
| XI-A-080 | H | NH₂ | CF₃ | Me | CH | CH | COOMe | log P |
| XI-A-081 | Cl | NH₂ | CF₃ | Me | CH | CH | COOMe | log P |
| XI-A-082 | Br | NH₂ | CF₃ | Me | CH | CH | COOMe | log P |
| XI-A-083 | I | NH₂ | CF₃ | Me | CH | CH | COOMe | log P |
| XI-A-084 | H | NH₂ | CF₃ | Me | CH | CH | COOH | log P |
| XI-A-085 | Cl | NH₂ | CF₃ | Me | CH | CH | COOH | log P |
| XI-A-086 | Br | NH₂ | CF₃ | Me | CH | CH | COOH | log P |
| XI-A-087 | I | NH₂ | CF₃ | Me | CH | CH | COOH | log P |
| XI-A-088 | H | NH₂ | CF₃ | Me | CH | CH | CONHCH₂CF₂CF₂H | log P |
| XI-A-089 | H | NH₂ | CF₃ | Me | CH | CH | CONHCH₂-2-Pyr | log P |
| XI-A-090 | Cl | NH₂ | CF₃ | Me | CH | CH | CONHCH₂CF₂CF₂H | log P |
| XI-A-091 | Cl | NH₂ | CF₃ | Me | CH | CH | CONHCH₂-2-Pyr | log P |
| XI-A-092 | Br | NH₂ | CF₃ | Me | CH | CH | CONHCH₂CF₂CF₂H | log P |
| XI-A-093 | Br | NH₂ | CF₃ | Me | CH | CH | CONHCH₂-2-Pyr | log P |
| XI-A-094 | I | NH₂ | CF₃ | Me | CH | CH | CONHCH₂CF₂CF₂H | log P |
| XI-A-095 | I | NH₂ | CF₃ | Me | CH | CH | CONHCH₂-2-Pyr | log P |

Characteristic data for synthesis examples:
¹H-NMR data (400 MHz., internal reference: tetramethylsilane δ = 0.00 ppm; s = singlet, br. s = broad singlet, d = doublet, dd = doublet of doublets, m = multiplet, q = quartet, t = triplet)
XI-A-001: spectroscopic data see protocol under general synthesis procedures
XI-A-018: log P (HCOOH): 3.60
¹H NMR (DMSO-d6): δ = 1.35 (t, 3H); 4.36 (q, 2H); 5.88 (br. s, 2H); 7.74 (d, 2H); 8.10 (d, 2H) ppm
XI-A-038: log P (HCOOH): 2.38
¹H NMR (DMSO-d6): δ = 4.70 (br. s, 2H); 7.68 (d, 2H); 8.15 (d, 2H) ppm
XI-A-042: log P (HCOOH): 3.52
¹H NMR (CD₃CN): δ = 1.37 (t, 3H); 4.38 (q, 2H); 4.71 (br.s, 2H); 7.68 (d, 2H); 8.14 (d, 2H) ppm
XI-A-043: log P (HCOOH): 2.34
¹H NMR (CD₃CN): δ = 4.70 (br.s, 2H); 7.68 (d, 2H); 8.15 (d, 2H) ppm
XI-A-044: log P (HCOOH): 3.66
¹H NMR (DMSO-d6): δ = 1.35 (t, 3H); 4.34 (q, 2H); 5.70 (br.s, 2H); 7.73 (d, 2H); 8.09 (d, 2H) ppm
XI-A-045: log P (HCOOH): 2.47
¹H NMR (DMSO-d6): δ =5.69 (br.s, 2H); 7.70 (d, 2H); 8.07 (d, 2H); 12.9 (br. s, 1H) ppm
XI-A-046: ¹H NMR (CD₃CN): δ = 2.89 (d, 3H); 4.66 (br. s, 2H); 6.95 (br. S, 1H); 7.63 (d, 2H); 7.92 (d, 2H) ppm
XI-A-047: ¹H NMR (CD₃CN): δ = 2.99 (br. s, 6H); 4.66 (br. s, 2H); 7.53-7.60 (m, 4H) ppm
XI-A-048: ¹H NMR (CD₃CN): δ = 1.20 (t, 3H); 3.40 (m, 2H); 4.66 (br.s, 2H); 6.99 (br. s, 1H); 7.62 (d, 2H); 7.93 (d, 2H) ppm
XI-A-049: ¹H NMR (CD₃CN): δ = 0.96 (t, 3H); 1.62 (m, 2H); 3.33 (m, 2H); 4.66 (br. s, 2H); 6.99 (br. s, 1H); 7.62 (d, 2H); 7.93 (d, 2H) ppm
XI-A-050: ¹H NMR (CD₃CN): δ = 1.24 (d, 6H); 4.19 (m, 1H); 4.66 (br.s, 2H); 6.80 (br. s, 1H); 7.62 (d, 2H); 7.93 (d, 2H) ppm
XI-A-051: ¹H NMR (CD₃CN): δ = 0.62 (m, 2H); 0.76 (m, 2H); 2.86 (m, 1H); 4.68 (br. s, 2H); 7.06 (br. s, 1H); 7.62 (d, 2H); 7.90 (d, 2H) ppm
XI-A-052: ¹H NMR (CD₃CN): δ = 1.43 (d, 3H); 4.82 (br. s, 2H); 4.93 (m, 1H); 7.40 (br. d, 1H); 7.68 (d, 2H); 7.99 (d, 2H) ppm
XI-A-053: ¹H NMR (CD₃CN): δ = 1.44 (s, 9H); 4.80 (br. s, 2H); 6.70 (br. s, 1H); 7.60 (d, 2H); 7.91 (d, 2H) ppm
XI-A-054: ¹H NMR (CD₃CN): δ = 0.96 (s, 9H); 1.15 (d, 3H); 4.05 (m, 1H); 4.80 (br. s, 2H); 6.78 (br. d, 1H); 7.63 (d, 2H); 7.94 (d, 2H) ppm
XI-A-055: ¹H NMR (CD₃CN): δ = 4.71 (br. s, 2H); 7.35 (m, 1H); 7.73 (d, 2H); 8.10 (d, 2H); 8.14 (m, 1H); 8.34 (m, 1H); 8.85 (br., 2H) ppm
XI-A-056: ¹H NMR (CD₃CN): δ = 4.68 (d, 2H); 4.82 (br. s, 2H); 7.29 (m, 1H); 7.40 (d, 1H); 7.67 (d, 2H); 7.77 (m, 1H); 7.88 (br. m, 1H); 8.02 (d, 2H); 8.53 (m, 1H) ppm
XI-A-057: ¹H NMR (DMSO-d6): δ = 2.82 (d, 3H); 5.80 (br. s, 2H); 7.66 (d, 2H); 7.98 (d, 2H); 8.37 (br. s, 1H) ppm
XI-A-058: ¹H NMR (DMSO-d6): δ = 2.98 (s, 6H); 5.80 (br. s, 2H); 7.54-7.63 (m, 4H) ppm
XI-A-059: ¹H NMR (DMSO-d6): δ = 1.15 (t, 3H); 3.32 (m, 2H); 5.79 (br. s, 2H); 7.65 (d, 2H); 7.99 (d, 2H); 8.40 (br. t, 1H) ppm
XI-A-060: ¹H NMR (DMSO-d6): δ = 0.91 (t, 3H); 1.57 (m, 2H); 3.25 (m, 2H); 5.79 (br. s, 2H); 7.65 (d, 2H); 8.00 (d, 2H); 8.38 (br. t, 1H) ppm
XI-A-061: ¹H NMR (DMSO-d6): δ = 1.20 (d, 6H); 4.12 (m, 1H); 5.78 (br. s, 2H); 7.64 (d, 2H); 8.00 (d, 2H); 8.15 (br. d, 1H) ppm
XI-A-062: ¹H NMR (DMSO-d6): δ = 0.60 (m, 2H); 0.71 (m, 2H); 2.90 (m, 1H); 5.79 (br. s, 2H); 7.64 (d, 2H); 7.97 (d, 2H); 8.38 (br. s, 1H) ppm
XI-A-063: ¹H NMR (DMSO-d6): δ = 1.39 (d, 3H); 4.88 (m, 1H); 5.81 (br. s, 2H); 7.70 (d, 2H); 8.05 (d, 2H); 8.82 (br. d, 1H) ppm
XI-A-064: ¹H NMR (DMSO-d6): δ = 1.40 (s, 9H); 5.76 (br. s, 2H); 7.62 (d, 2H); 7.67 (br. s, 1H); 7.96 (d, 2H) ppm
XI-A-065: ¹H NMR (DMSO-d6): δ = 0.92 (s, 9H); 1.11 (d, 3H); 4.01 (m, 1H); 5.78 (br. s, 2H); 7.64 (d, 2H); 7.87 (br. d, 1H); 8.01 (d, 2H) ppm
XI-A-066: ¹H NMR (DMSO-d6): δ = 5.7-6.0 (br. signal, 2H); 7.55 (m, 1H); 7.78 (d, 2H); 8.17 (d, 2H); 8.34 (m, 1H); 8.40 (m, 1H); 9.06 (br. s, 1H); 10.62 (s, 1H) ppm
XI-A-067: ¹H NMR (DMSO-d6): δ = 4.61 (d, 2H); 5.82 (br. s, 2H); 7.25 (m, 1H); 7.34 (m, 1H); 7.67 (d, 2H); ~7.5 (m, 1H); 8.08 (d, 2H); 8.51 (m, 1H); 9.03 (br. t, 1H) ppm
XI-A-068: ¹H NMR (DMSO-d6): δ = 2.82 (d, 3H); 5.62 (br. s, 2H); 7.64 (d, 2H); 7.98 (d, 2H); 8.36 (br. s, 1H) ppm
XI-A-069: ¹H NMR (DMSO-d6): δ = 2.98 (s, 6H); 5.63 (br. s, 2H); 7.53-7.73 (m, 4H) ppm
XI-A-070: ¹H NMR (DMSO-d6): δ = 1.15 (t, 3H); 3.32 (m, 2H); 5.61 (br. s, 2H); 7.64 (d, 2H); 7.99 (d, 2H); 8.39 (br. s, 1H) ppm
XI-A-071: ¹H NMR (DMSO-d6): δ = 0.91 (t, 3H); 1.55 (m, 2H); 3.26 (m, 2H); 5.61 (br. s, 2H); 7.64 (d, 2H); 7.99 (d, 2H); 8.38 (br. t, 1H) ppm
XI-A-072: ¹H NMR (DMSO-d6): δ = 1.19 (d, 6H); 4.12 (m, 1H); 5.60 (br. s, 2H); 7.63 (d, 2H); 8.00 (d, 2H); 8.15 (br. d, 1H) ppm
XI-A-073: ¹H NMR (DMSO-d6): δ = 0.61 (m, 2H); 0.71 (m, 2H); 2.89 (m, 1H); 5.61 (br. s, 2H); 7.63 (d, 2H); 7.97 (d, 2H); 8.37 (br. s, 1H) ppm
XI-A-074: ¹H NMR (DMSO-d6): δ = 1.39 (d, 3H); 4.87 (m, 1H); 5.64 (br. s, 2H); 7.68 (d, 2H); 8.05 (d, 2H); 8.82 (br. s, 1H) ppm
XI-A-075: ¹H NMR (DMSO-d6): δ = 1.40 (s, 9H); 5.58 (br. s, 2H); 7.60 (d, 2H); 7.66 (br. s, 1H); 7.96 (d, 2H) ppm
XI-A-076: ¹H NMR (DMSO-d6): δ = 0.92 (s, 9H); 1.11 (d, 3H); 4.01 (m, 1H); 7.62 (d, 2H); 7.86 (br. d, 1H); 8.00 (d, 2H) ppm
XI-A-077: ¹H NMR (DMSO-d6): δ = 5.68 (br. s, 2H); 6.9 (m, 1H); 6.98 (m, 1H); 7.39 (m, 1H); 7.75 (d, 2H); 8.15 (d, 2H); 8.33 (m, 1H); 10.40 (s, 1H) ppm
XI-A-078: ¹H NMR (DMSO-d6): δ = 4.60 (d, 2H); 5.64 (br. d, 2H); 7.25 (m, 1H); 7.34 (m, 1H); 7.67 (d, 2H); 7.74 (m, 1H); 8.07 (d, 2H); 8.50 (m, 1H); 9.03 (br. t, 1H) ppm
XI-A-079: log P (HCOOH): 2.80
XI-A-080: log P (HCOOH): 1.97
XI-A-081: log P (HCOOH): 2.76
XI-A-082: log P (HCOOH): 3.52
XI-A-083: log P (HCOOH): 3.48
XI-A-084: log P (HCOOH): 3.53
XI-A-085: log P (HCOOH): 1.95
XI-A-086: log P (HCOOH): 2.54
XI-A-087: log P (HCOOH): 2.58
XI-A-088: log P (HCOOH): 2.63
XI-A-089: log P (HCOOH): 2.45
XI-A-090: log P (HCOOH): 1.42
XI-A-090: log P (HCOOH): 2.99
XI-A-091: log P (HCOOH): 1.94
XI-A-092: log P (HCOOH): 3.03
XI-A-093: log P (HCOOH): 1.98
XI-A-094: log P (HCOOH): 3.08
XI-A-095: log P (HCOOH): 2.03

TABLE 15
In accordance with the general methods described above,
it is possible to prepare the compounds
of the formula (XI-B) listed in the table below.
(XI-B)

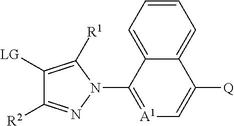

| Table 15 | | | | | | Phys. |
|---|---|---|---|---|---|---|
| Example | | | | | | chem. |
| No. | LG | R¹ | R² | A¹ | Q | data |
| XI-B-001 | Br | H | CF₃ | CH | COOEt | |
| XI-B-002 | Br | H | CF₃ | CH | COOH | |
| XI-B-003 | Br | NH₂ | CF₃ | CH | Br | |
| XI-B-004 | Br | NH₂ | CF₃ | CH | I | |
| XI-B-005 | Br | Cl | CF₃ | CH | CN | |
| XI-B-006 | Br | Cl | C₂F₅ | CH | NO₂ | |
| XI-B-007 | Br | Br | C₂F₅ | CH | SMe | |
| XI-B-008 | Br | Br | C₂F₅ | CH | SCF₃ | |
| XI-B-009 | Br | I | C₂F₅ | CH | SOMe | |
| XI-B-010 | Br | I | CF₃ | CH | SOCF₃ | |
| XI-B-011 | Br | CN | CF₃ | N | SO₂Me | |
| XI-B-012 | Br | CN | CF₃ | N | SO₂CF₃ | |
| XI-B-013 | Br | NHCH₂Ph | C₂F₅ | CH | CN | |
| XI-B-014 | Br | NHAc | CF₃ | CH | NO₂ | |
| XI-B-015 | Br | NAc₂ | CF₃ | CH | F | |
| XI-B-016 | Br | NHMe | CF₂Ph | CH | Cl | |
| XI-B-017 | Br | NMe₂ | CF₂OMe | CH | 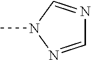 | |
| XI-B-018 | Br | SMe | CF₂OPh | CH | CONH—CH₂—Pyr | |
| XI-B-019 | Br | SOMe | CF₂ | CH | CONH—CHMe-2-Pyr | |
| | | | (2-Pyr) | | | |
| XI-B-020 | Br | SO₂Me | CF₂O | N | CONH-TFiPr | |
| | | | (2-Pyr) | | | |
| XI-B-021 | I | H | CF₃ | CH | COOEt | |
| XI-B-022 | I | H | CF₃ | CH | COOH | |
| XI-B-023 | I | NH₂ | CF₃ | CH | Br | |
| XI-B-024 | I | NH₂ | CF₃ | CH | I | |
| XI-B-025 | I | Cl | CF₃ | CH | CN | |
| XI-B-026 | I | Cl | C₂F₅ | CH | NO₂ | |
| XI-B-027 | I | Br | C₂F₅ | CH | SMe | |
| XI-B-028 | I | Br | C₂F₅ | CH | SCF₃ | |
| XI-B-029 | I | I | C₂F₅ | CH | SOMe | |
| XI-B-030 | I | I | CF₃ | CH | SOCF₃ | |
| XI-B-031 | I | CN | CF₃ | N | SO₂Me | |
| XI-B-032 | I | CN | CF₃ | N | SO₂CF₃ | |
| XI-B-033 | I | NHCH₂Ph | C₂F₅ | CH | CN | |
| XI-B-034 | I | NHAc | CF₃ | CH | NO₂ | |
| XI-B-035 | I | NAc₂ | CF₃ | CH | F | |
| XI-B-036 | I | NHMe | CF₂Ph | CH | Cl | |
| XI-B-037 | I | NMe₂ | CF₂OMe | CH | 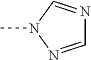 | |
| XI-B-038 | I | SMe | CF₂OPh | CH | CONH—CH₂—Pyr | |
| XI-B-039 | I | SOMe | CF₂ | CH | CONH—CHMe-2-Pyr | |
| | | | (2-Pyr) | | | |
| XI-B-040 | I | SO₂Me | CF₂O | N | CONH-TFiPr | |
| | | | (2-Pyr) | | | |

### BIOLOGICAL EXAMPLES

### Example A

### Phaedon cochleariae Test (PHAECO Spray Treatment)

Solvent: 78.0 parts by weight of acetone
1.5 parts by weight of dimethylformamide
   Emulsifier: 0.5 part by weight of alkylaryl polyglycol ether

To produce a suitable preparation of active compound, 1 part by weight of active compound is mixed with the stated amounts of solvent and emulsifier, and the concentrate is diluted with emulsifier-containing water to the desired concentration.

Discs of Chinese cabbage (Brassica pekinensis) are sprayed with an active compound preparation of the desired concentration and, after drying, populated with larvae of the mustard beetle (Phaedon cochleariae).

After 7 days, the effect in % is determined. 100% means that all beetle larvae have been killed; 0% means that none of the beetle larvae have been killed.

In this test, for example, the following compounds of the Preparation Examples showed, at an application rate of 500 g/ha, an effect of >80%: I-A-Q1-001, I-A-Q1-002, I-A-Q2-011, I-A-Q2-012, I-A-Q2-013, I-A-Q2-014, I-A-Q2-015, I-A-Q2-016, I-A-Q2-017, I-A-Q2-018, I-A-Q2-019, I-A-Q2-020, I-A-Q2-022, I-A-Q2-023, I-A-Q2-024, I-A-Q2-025, I-A-Q2-026, I-A-Q2-027, I-A-Q2-028, I-A-Q2-029, I-A-Q2-030, I-A-Q2-032, I-A-Q2-033, I-A-Q2-034, I-A-Q2-035, I-A-Q2-037, I-A-Q2-039, I-A-Q2-040, I-A-Q2-041, I-A-Q2-042, I-A-Q2-044, I-A-Q2-046, I-A-Q2-047, I-A-Q2-048, I-A-Q2-049, I-A-Q2-050, I-A-Q2-051, I-A-Q2-052, I-A-Q2-053, I-A-Q2-054, I-A-Q2-055, I-A-Q2-056, I-A-Q2-057, I-A-Q2-058, I-A-Q2-059, I-A-Q2-060, I-A-Q2-061, I-A-Q2-062, I-A-Q2-065, I-A-Q2-066, I-A-Q2-069, I-A-Q2-073, I-A-Q2-079, I-A-Q2-080, I-A-Q2-083, I-A-Q2-084, I-A-Q2-085, I-A-Q2-086, I-A-Q2-087, I-A-Q2-088, I-A-Q2-089, I-A-Q2-090, I-A-Q2-092, I-A-Q2-095, I-A-Q2-096, I-A-Q2-098, I-A-Q2-109, I-A-Q2-110, I-A-Q2-111, I-A-Q2-112, I-A-Q2-133, I-A-Q2-134, I-A-Q2-135, I-A-Q2-136, I-A-Q2-137, I-A-Q2-138, I-A-Q2-139, I-A-Q2-140, I-A-Q2-141, I-A-Q2-144, I-A-Q2-145, I-A-Q2-147, I-A-Q2-149, I-A-Q2-150, I-A-Q2-151, I-A-Q2-152, I-A-Q2-153, I-A-Q2-154, I-A-Q2-155, I-A-Q2-157, I-A-Q2-159, I-A-Q2-160, I-A-Q2-162, I-A-Q2-165, I-A-Q2-166, I-A-Q2-167, I-A-Q2-169, I-A-Q2-170, I-A-Q2-171, I-A-Q2-172, I-A-Q2-174, I-A-Q2-175, I-A-Q2-176, I-A-Q2-180, I-A-Q2-182, I-A-Q2-183, I-A-Q2-188, I-A-Q2-202, I-A-Q2-203, I-A-Q2-205, I-A-Q2-207, I-A-Q2-209, I-A-Q2-210, I-A-Q2-211, I-A-Q2-213, I-A-Q2-214, I-A-Q2-215, I-A-Q2-216, I-A-Q2-217, I-A-Q2-218, I-A-Q2-219, I-A-Q2-220, I-A-Q2-221, I-A-Q2-222, I-A-Q2-223, I-A-Q2-224, I-A-Q2-225, I-A-Q2-226, I-A-Q2-227, I-A-Q2-228, I-A-Q2-229, I-A-Q2-230, I-A-Q2-233, I-A-Q2-234, I-A-Q2-236, I-A-Q2-237, I-A-Q2-238, I-A-Q2-239, I-A-Q2-240, I-A-Q2-241, I-A-Q2-242, I-A-Q2-244, I-A-Q2-246, I-A-Q2-247, I-A-Q4-009, I-A-Q4-011, I-A-Q4-040, I-A-Q4-042, I-A-Q4-048, I-A-Q4-058, I-A-Q4-102.

In this test, for example, at an application rate of 500 g/ha, the following compound of the Preparation Examples had an activity of 83%: I-A-Q1-089.

In this test, for example, at an application rate of 500 g/ha, the following compounds of the Preparation Examples had an activity of 100%: I-A-Q1-087, I-A-Q1-090, I-A-Q1-091, I-A-Q2-407, I-A-Q2-412, I-A-Q2-414, I-A-Q2-415, I-A-Q2-417, I-A-Q2-419, I-A-Q2-420, I-A-Q2-421.

### Example B

### Spodoptera frugiperda Test (SPODFR Spray Treatment)

Solvent: 78.0 parts by weight of acetone
1.5 parts by weight of dimethylformamide
   Emulsifier: 0.5 part by weight of alkylaryl polyglycol ether

To produce a suitable preparation of active compound, 1 part by weight of active compound is mixed with the stated amounts of solvent and emulsifier, and the concentrate is diluted with emulsifier-containing water to the desired concentration.

Discs of maize leaves (Zea mays) are sprayed with an active compound preparation of the desired concentration and, after drying, populated with caterpillars of the armyworm (Spodoptera frugiperda).

After 7 days, the effect in % is determined 100% means that all caterpillars have been killed; 0% means that none of the caterpillars have been killed.

In this test, for example, the following compounds of the Preparation Examples showed, at an application rate of 500 g/ha, an effect of >80%: I-A-Q1-001, I-A-Q2-011, I-A-Q2-012, I-A-Q2-014, I-A-Q2-015, I-A-Q2-017, I-A-Q2-018, I-A-Q2-019, I-A-Q2-020, I-A-Q2-023, I-A-Q2-030, I-A-Q2-033, I-A-Q2-035, I-A-Q2-039, I-A-Q2-044, I-A-Q2-048, I-A-Q2-052, I-A-Q2-053, I-A-Q2-054, I-A-Q2-055, I-A-Q2-058, I-A-Q2-060, I A Q2-061, I-A-Q2-062, I-A-Q2-107, I-A-Q2-110, I-A-Q2-133, I-A-Q2-141, I-A-Q2-147, I-A-Q2-155, I-A-Q2-160, I-A-Q2-166, I-A-Q2-202, I-A-Q2-207, I-A-Q2-217, I-A-Q2-218, I-A-Q2-219, I-A-Q2-220, I-A-Q2-221, I-A-Q2-223, I-A-Q2-224, I-A-Q2-227, I-A-Q2-230, I-A-Q2-231, I-A-Q2-236, I-A-Q2-238, I-A-Q2-239, I-A-Q2-240, I-A-Q2-242, I-A-Q2-244, I-A-Q2-246, I-A-Q4-009, I-A-Q4-014, I-A-Q4-016, I-A-Q4-040, I-A-Q4-041, I-A-Q4-042, I-A-Q4-043, I-A-Q4-048, I-A-Q4-051, I-A-Q4-053, I-A-Q4-058, I-A-Q4-102, IX-021, IX-038.

In this test, for example, at an application rate of 500 g/ha, the following compound of the Preparation Examples had an activity of 83%: I-A-Q2-409.

In this test, for example, at an application rate of 500 g/ha, the following compounds of the Preparation Examples had an activity of 100%: I-A-Q1-091, I-A-Q2-407.

### Example C

### Myzus persicae Test (MYZUPE Spray Treatment)

Solvent: 78.0 parts by weight of acetone
1.5 parts by weight of dimethylformamide
   Emulsifier: 0.5 part by weight of alkylaryl polyglycol ether

To produce a suitable preparation of active compound, 1 part by weight of active compound is mixed with the stated amounts of solvent and emulsifier, and the concentrate is diluted with emulsifier-containing water to the desired concentration.

Discs of Chinese cabbage (Brassica pekinensis) infected by all stages of the green peach aphid (Myzus persicae) are sprayed with an active compound preparation of the desired concentration.

After 6 days, the effect in % is determined. 100% means that all of the aphids have been killed; 0% means that none of the aphids have been killed.

In this test, for example, the following compounds of the Preparation Examples showed, at an application rate of 500 g/ha, an effect of >80%: I-A-Q2-053, I-A-Q2-133, I-A-Q2-134, I-A-Q2-0192, I-A-Q4-088, I-A-Q4-089.

In this test, for example, at an application rate of 500 g/ha, the following compound of the Preparation Examples had an activity of 90%: I-A-Q1-089.

### Example D

### Tetranychus Test, OP-Resistant (TETRUR Spray Treatment)

Solvent: 78.0 parts by weight of acetone
1.5 parts by weight of dimethylformamide
   Emulsifier: 0.5 part by weight of alkylaryl polyglycol ether

To produce a suitable preparation of active compound, 1 part by weight of active compound is mixed with the stated amounts of solvent and emulsifier, and the concentrate is diluted with emulsifier-containing water to the desired concentration.

Discs of bean leaves (Phaseolus vulgaris) which are infested by all stages of the greenhouse red spider mite (Tetranychus urticae) are sprayed with an active compound preparation of the desired concentration.

After the desired period of time, the effect in % is determined 100% means that all of the spider mites have been killed; 0% means that none of the spider mites have been killed.

In this test, for example, the following compounds of the Preparation Examples showed, at an application rate of 500 g/ha, an effect of >80%: I-A-Q2-012, I-A-Q2-015, I-A-Q2-020, I-A-Q2-035, I-A-Q2-040, I-A-Q2-041, I-A-Q2-048, I-A-Q2-051, I-A-Q2-052, I-A-Q2-055, I-A-Q2-060, I-A-Q2-112, I-A-Q4-058.

In this test, for example, the following compounds of the Preparation Examples showed, at an application rate of 100 g/ha, an effect of >80%: Nos. I-A-Q2-046, I-A-Q2-110, I-A-Q2-202, 1-A-Q2-214, I-A-Q2-216, I-A-Q2-219, I-A-Q2-220, I-A-Q2-223, I-A-Q2-224, I-A-Q2-227, I-A-Q2-229, I-A-Q2-231, I-A-Q2-239, I-A-Q2-240, I-A-Q2-247

### Example E

### Lucilia cuprina Test (LUCICU)

Solvent: dimethyl sulphoxide

To prepare a suitable active compound preparation, 1 part by weight of active compound is mixed with the stated amount of solvent and the concentrate is diluted with water to the desired concentration.

Vessels containing horse meat treated with the active compound preparation of the desired concentration are populated with Lucilia cuprina larvae.

After the desired period of time, the kill in % is determined. 100% means that all of the larvae have been killed; 0% means that none of the larvae have been killed.

In this test, for example, the following compounds of the Preparation Examples showed, at an application rate of 100 ppm, an effect of >80%: I-A-Q1-001, I-A-Q2-002, I-A-Q2-005, I-A-Q2-010, I-A-Q2-011, I-A-Q2-012, I-A-Q2-014, I-A-Q2-015, I-A-Q2-017, I-A-Q2-018, I-A-Q2-019, I-A-Q2-023, I-A-Q2-134, I-A-Q2-141, I-A-Q2-147, I-A-Q2-149, I-A-Q2-152.

### Example F

### Musca domestica Test (MUSCDO)

Solvent: dimethyl sulphoxide

To prepare a suitable active compound preparation, 1 part by weight of active compound is mixed with the stated amount of solvent and the concentrate is diluted with water to the desired concentration.

Vessels containing a sponge treated with the active compound preparation of the desired concentration are populated with adult Musca domestica.

After the desired period of time, the kill in % is determined 100% means that all of the flies have been killed; 0% means that none of the flies have been killed.

In this test, for example, the following compounds of the Preparation Examples showed, at an application rate of 100 ppm, an effect of >80%: I-A-Q2-002, I-A-Q2-005, I-A-Q2-011, I-A-Q2-014, I-A-Q2-017, I-A-Q2-018.

### Example G

### Ctenocephalides felis; Oral (CTECFE)

Solvent: 1 part by weight of dimethyl sulphoxide

To produce a suitable preparation of active compound, 2 parts by weight of active compound are mixed with the stated amount of solvent. Part of the concentrate is diluted with citrated cattle blood, and the desired concentration is prepared.

20 unfed adult fleas (Ctenocephalides felis) are placed into a chamber which is closed at the top and bottom with gauze. A metal cylinder whose bottom end is closed with a parafilm is placed onto the chamber. The cylinder contains the blood/active compound preparation, which can be taken up by the fleas through the parafilm membrane.

After the desired period of time, the kill in % is determined 100% means that all of the fleas have been killed; 0% means that none of the fleas have been killed.

In this test, for example, the following compounds of the Preparation Examples showed, at an application rate of 100 ppm, an effect of >80%: I-A-Q1-001, I-A-Q2-011, I-A-Q2-012, I-A-Q2-014, I-A-Q2-015, I-A-Q2-016, I-A-Q2-017, I-A-Q2-018, I-A-Q2-023, I-A-Q2-134

### Example H

### Boophilus microplus Test (BOOPMI Injection)

Solvent: dimethyl sulphoxide

To prepare a suitable active compound preparation, 1 part by weight of active compound is mixed with the stated amount of solvent and the concentrate is diluted with water to the desired concentration.

The solution of active compound is injected into the abdomen (Boophilus microplus), and the animals are transferred into dishes and kept in a climatised room. The activity is assessed by position of fertile eggs.

After the desired period of time, the effect in % is determined. 100% means that none of the ticks has laid any fertile eggs.

In this test, for example, the following compounds of the Preparation Examples showed, at an application rate of 20 μg/animal, an effect of >80%: I-A-Q1-001, I-A-Q2-002, I-A-Q2-005, 1-A-Q2-010, I-A-Q2-011, I-A-Q2-012, I-A-Q2-014, I-A-Q2-015, I-A-Q2-016, I-A-Q2-017, I-A-Q2-018, I-A-Q2-019, I-A-Q2-022, I-A-Q2-023, I-A-Q2-024, I-A-Q2-026, I-A-Q2-134, I-A-Q2-141, I-A-Q2-147, I-A-Q2-149, I-A-Q2-150, I-A-Q2-152, I-A-Q2-195.

### Example I

### Spodoptera litura Larvae Test

Solvent: 3 parts by weight of dimethylformamide
Emulsifier: 1 part by weight of polyoxyethylene alkylphenyl ether

To prepare a suitable active compound, 1 part by weight of active compound is mixed with the stated amount of solvent comprising the stated amount of emulsifier, and the mixture is diluted with water to the specified concentration.

Sweet potato leaves are dipped into the sample solution diluted with water to the specified concentration and the leaves treated in this manner are, after the solution adhering to the leaves has dried in air, transferred into a laboratory dish which has a diameter of 9 cm and in which there are 10 stage 3 Spodoptera litura larvae. The dish is then placed in a temperature-controlled room at 25° C., sweet potato leaves are then added to the dish on day two and day four and the number of dead insects is determined after 7 days and used to calculate the insecticidal ratio.

The results are the means of two laboratory dishes per group in this test.

Compounds I-A-Q4-001 and I-A-Q4-006 showed, at an active compound concentration of 500 ppm, an 80% kill of the insect larvae.

Compounds I-A-Q2-001, I-A-Q2-002, I-A-Q2-003, I-A-Q2-005, I-A-Q2-006, and I-A-Q2-010 showed, at an active compound concentration of 500 ppm, a 100% kill of the insect larvae.

### Example J

### Aulacophora femoralis Test (Spray Test)

Solvent: 3 parts by weight of dimethylformamide
Emulsifier: 1 part by weight of polyoxyethylene alkylphenyl ether

To prepare a suitable active compound formulation, 1 part by weight of the active compound is mixed with the stated amount of solvent comprising the stated amount of emulsifier, and the mixture is diluted with water to a specified concentration.

Cucumber leaves are dipped into a dilute aqueous solution of an active compound at the specified concentration prepared in the same manner as in the test described above, air-dried and transferred into a plastic dish with sterilized black soil. 5 stage 2 Aulacophora femoralis larvae are transferred into this dish. The dish is then placed into a temperature-controlled room at 25° C. After 7 days, the number of dead larvae is counted to calculate the mortality.

Compounds I-A-Q2-002, I-A-Q2-005 and I-A-Q2-010 showed, at an active compound concentration of 500 ppm, a 100% kill of the insects.

1. Compounds of the formula (I) 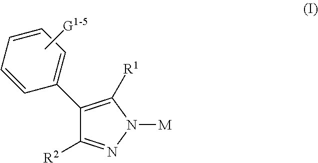
in which
M represents one of the groupings listed below: 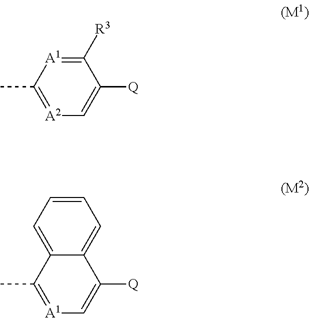
A¹ and A² independently of one another represent nitrogen or C—R⁴;
G¹, G², G³, G⁴ and G⁵ represent hydrogen or a substituent independently of the others selected from the group consisting of: halogen, alkyl, alkenyl, alkynyl, haloalkyl, SF_{S}, hydroxyl, alkoxy, alkoxyalkyl, alkoxyalkoxy, cycloalkyl, alkenyloxy, alkynyloxy, cycloalkylalkoxy, haloalkoxy, haloalkoxyalkyl, alkylsulphanyl, haloalkylsulphanyl, alkylsulphinyl, haloalkylsulphinyl, alkylsulphonyl, haloalkylsulphonyl, cyano, alkylcarbonyl, alkoxycarbonyl, alkoxycarbonylalkyl, carboxyl, aminocarbonyl, aminothiocarbonyl, alkylaminocarbonyl, dialkylaminocarbonyl, trialkylsilyl, nitro, amino, alkylamino, dialkylamino, alkylsulphonylamino, dialkylsulphonylamino, —CR⁵═NO—R⁵, —CR⁵═NO-haloalkyl and formyl, where vicinal alkyl, haloalkyl, alkoxy and/or haloalkoxy groups together with the carbon atoms to which they are attached may form a five- to six-membered cyclic system which contains 0 to 2 oxygen atoms and whose alkyl moiety may optionally be substituted by one or more fluorine atoms;
Q represents a substituent selected from the group consisting of Q¹, Q², Q³ and Q⁴;
Q¹ represents one of the heterocyclic groupings listed below; 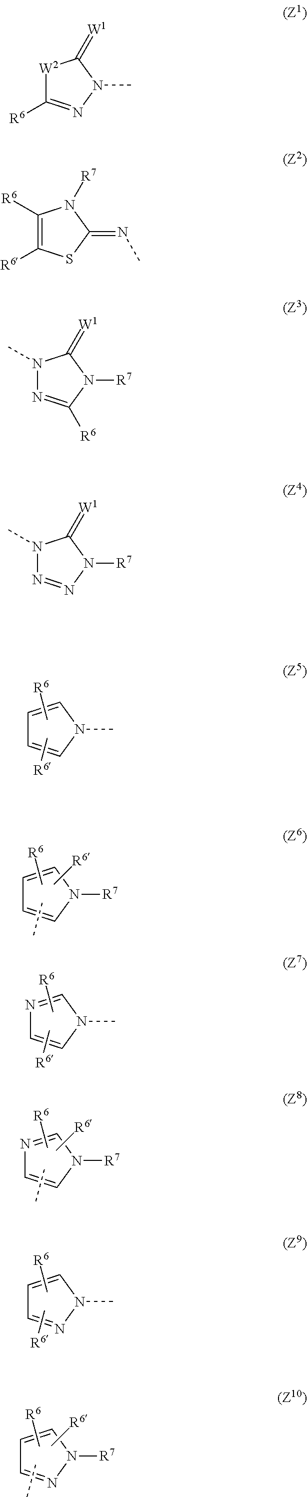 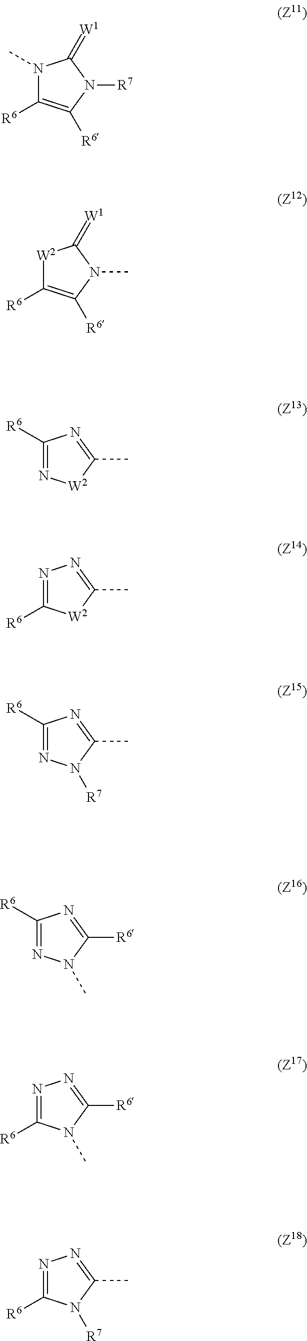 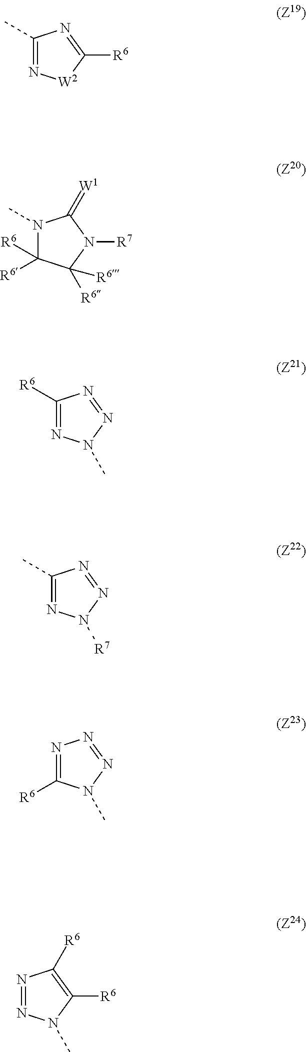
where
W¹ and W² independently of one another represent oxygen or sulphur,
and
R⁶, R^{6′}, R^{6″}, R^{6′″} and R⁷ have the meaning given below;
Q² represents C(W1)NR⁸R⁹;
Q³ represents C(R¹⁰R¹¹)NR⁸R⁹;
Q⁴ represents cyano (where R¹ does not represent amino), nitro, amino, COOH, COOR¹², fluorine (if R³ is different from chlorine), chlorine (if R³ is different from chlorine, COOH, CH₂CH₂OMe and OMe), bromine, iodine, SR¹² (where R¹ does not represent amino if R¹² represents alkyl), S(O)R¹², S(O)₂R¹² or S(O)₂NR⁸R⁹;
R¹ represents hydrogen, halogen, alkylsulfanyl, alkylsulfinyl, alkylsulfonyl, cyano, amino, Z¹⁶ or NR¹³R¹⁴;
R² represents hydrogen, halogen, alkyl, alkenyl, alkynyl, haloalkyl, cycloalkyl, hydroxyalkyl, alkoxyalkyl, haloalkoxyalkyl, alkoxyhaloalkyl, alkylsulphanylalkyl, alkylsulphinylalkyl, alkylsulphonylalkyl, haloalkylsulphanylalkyl, haloalkylsulphinylalkyl, haloalkylsulphonylalkyl cycloalkylalkyl, phenylalkyl, heteroarylalkyl, heterocyclylalkyl, arylhaloalkyl, haloalkyloxyhaloalkyloxyhaloalkyl, heterocyclyl, alkylsulphanyl, alkylsulphinyl, alkylsulphonyl or phenyl;
where a phenyl or heteroaryl radical is optionally mono- or polysubstituted by halogen, alkyl, haloalkyl, alkoxy, nitro or cyano; a heterocyclyl radical is optionally substituted by —OH/═O, —SH/═S, —NH₂, halogen, alkyl, haloalkyl, alkoxy, alkylsulphanyl, alkylsulphinyl, alkylsulphonyl, monoalkylamino, dialkylamino, nitro or cyano; and a phenyl-, heteroaryl- or heterocyclyl-bound alkanediyl radical is optionally mono- or polysubstituted by halogen, alkyl, haloalkyl, alkoxy or cyano;
R³ represents halogen, alkyl, alkenyl, alkynyl, haloalkyl, cycloalkyl, alkoxyalkyl, alkylcarbonyloxyalkyl, alkylsulphanylalkyl, alkylsulphinylalkyl, alkylsulphonylalkyl, hydroxyl, Z¹⁶, alkoxy, acylamino, alkoxycarbonylamino, alkenyloxy, alkinyloxy, cycloalkylalkoxy, haloalkoxy, alkylsulphanyl, haloalkylsulphanyl, alkylsulphinyl, haloalkylsulphinyl, alkylsulphonyl, haloalkylsulphonyl, cyano, alkylcarbonyl, alkoxycarbonyl, alkoxycarbonylalkyl, carboxyl, aminocarbonyl, aminothiocarbonyl, alkylaminocarbonyl, dialkylaminocarbonyl, alkylsulphonylaminocarbonyl, trialkylsilyl, nitro, amino, alkylamino, dialkylamino, alkylsulphonylamino, dialkylsulphonylamino, —CR⁵═NO—R⁵, —CR⁵═NO-haloalkyl or formyl;
R⁴ represents hydrogen, cyano, nitro, alkyl, haloalkoxy, halogen or alkoxy;
R⁵ represents hydrogen or alkyl;
R⁶, R^{6′}, R^{6″}, R^{6′″} independently of one another represent hydrogen, amino, hydroxyl, mercapto, nitro, cyano, carboxyl, halogen, alkyl, haloalkyl, alkenyl, alkynyl, cycloalkyl, alkoxy, haloalkoxy, alkenyloxy, alkynyloxy, alkoxycarbonyl, alkylsulphanyl, haloalkylsulphanyl, alkylsulphinyl, haloalkylsulphinyl, alkylsulphonyl, haloalkylsulphonyl, aminocarbonyl, aminothiocarbonyl, CR⁵═NO—R⁵, —CR⁵═NO-haloalkyl, formyl, alkylamino, dialkylamino, phenyl, heteroaryl, heteroarylalkyl or heterocyclylalkyl;
where a phenyl or heteroaryl radical is optionally mono- or polysubstituted by halogen, alkyl, haloalkyl, alkoxy, nitro or cyano; a heterocyclyl radical is optionally substituted by —OH/═O, —SH/═S, —NH₂, halogen, alkyl, haloalkyl, alkoxy, alkylsulphanyl, alkylsulphinyl, alkylsulphonyl, monoalkylamino, dialkylamino, nitro or cyano; and a phenyl-, heteroaryl- or heterocyclyl-bound alkanediyl radical is optionally mono- or polysubstituted by halogen, alkyl, haloalkyl, alkoxy or cyano;
R⁷ represents hydrogen, amino, hydroxyl, cyano, alkyl, haloalkyl, alkenyl, alkynyl, cycloalkyl, alkoxy, alkenyloxy, alkynyloxy, alkoxycarbonyl, alkylcarbonyl, alkylamino-carbonyl, dialkylaminocarbonyl, phenyl, phenylalkyl, heteroarylalkyl or heterocyclylalkyl;
where a phenyl or heteroaryl radical is optionally mono- or polysubstituted by halogen, alkyl, haloalkyl, alkoxy, nitro or cyano; a heterocyclyl radical is optionally substituted by —OH/═O, —SH/═S, —NH₂, halogen, alkyl, haloalkyl, alkoxy, alkylsulphanyl, alkylsulphinyl, alkylsulphonyl, monoalkylamino, dialkylamino, nitro or cyano; and a phenyl-, heteroaryl- or heterocyclyl-bound alkanediyl radical is optionally mono- or polysubstituted by halogen, alkyl, haloalkyl, alkoxy or cyano and
where optionally two adjacent radicals from the group consisting of R⁶, R^{6′}, R^{6″}, R^{6′″} and R⁷ together represent alkanediyl or alkenediyl, each of which has 2 to 5 carbon atoms and is optionally substituted and/or optionally interrupted at the beginning (or the end) or within the hydrocarbon chain by oxygen or sulphur or a grouping from the group consisting of —S(O)—, —SO₂—, —NH— or —N-alkyl-;
R⁸ and R⁹ independently of one another represent hydrogen, alkyl, haloalkyl, alkenyl (optionally substituted by halogen, alkyl, haloalkyl or cyano), alkynyl, cycloalkyl (optionally mono- or polysubstituted at the cycle by halogen, haloalkyl, alkyl or condensed at an aromatic or heteroaromatic moiety), cycloalkylalkyl (optionally mono- or polysubstituted at the cycle by halogen, haloalkyl, alkyl or condensed at an aromatic or heteroaromatic moiety, optionally mono- or polysubstituted at the alkyl moiety by halogen, alkyl, haloalkyl, alkoxy or cyano), cycloalkylcarbonylheterocyclyl, alkyloxycarbonylheterocyclyl, alkylsulphinylalkyl, alkylsulphanylalkyl, alkylsulphonylalkyl, hydroxyalkyl, heterocyclylcarbonylalkyl, heteroarylcarbonylaminoalkyl, haloalkoxyalkyl, alkylthiocarbonyl, dialkylaminocarbonyl, alkylaminocarbonyl, halo alkylaminocarbonyl, alkoxy, alkenyloxy, alkynyloxy, alkoxyalkyl, alkylcarbonylalkyl, C(R¹⁰R¹¹)CR⁵═NO—R⁵, alkylsulphonyl, alkylcarbonyl, haloalkylcarbonyl, alkenylcarbonyl, alkynylcarbonyl, cycloalkylcarbonyl, alkoxycarbonyl, alkenyloxycarbonyl, alkynyloxycarbonyl, alkoxyalkylcarbonyl, phenylsulphonyl, phenyl, heteroaryl, heterocyclyl, phenylalkyl, heteroarylalkyl, heterocyclylalkyl, phenyl carbonyl, heteroarylcarbonyl, heterocyclylcarbonyl, phenylalkylcarbonyl, heteroarylalkylcarbonyl, heterocyclylalkylcarbonyl, phenoxycarbonyl or phenylalkoxycarbonyl;
where a phenyl or heteroaryl radical is optionally mono- or polysubstituted by halogen, alkyl, haloalkyl, alkoxy, nitro or cyano; a heterocyclyl radical is optionally substituted by —OH/═O, —SH/═S, —NH₂, halogen, alkyl, haloalkyl, alkoxy, alkylsulphanyl, alkylsulphinyl, alkylsulphonyl, monoalkylamino, dialkylamino, nitro or cyano; and a phenyl-, heteroaryl- or heterocyclyl-bound alkanediyl radical is optionally mono- or polysubstituted by halogen, alkyl, haloalkyl, alkoxy or cyano and
where optionally R⁸/R⁹ together represent alkanediyl or alkenediyl, each of which has 2 to 5 carbon atoms and is optionally substituted and/or optionally interrupted at the beginning (or the end) or within the hydrocarbon chain by oxygen or sulphur or a grouping from the group consisting of —S(O)—, —SO₂—, —NH— or —N-alkyl-;
R¹⁰ and R¹¹ independently of one another represent hydrogen, alkyl, haloalkyl or cyano;
R¹² represents alkyl or haloalkyl;
R¹³ and R¹⁴ independently of one another represent hydrogen, alkyl, haloalkyl, alkenyl, alkynyl, alkoxyalkyl, alkylcarbonylalkyl, C(R¹⁰R¹¹)CR⁵═NO—R⁵, alkylsulfonyl, alkylcarbonyl, haloalkylcarbonyl, alkenylcarbonyl, alkynylcarbonyl, cycloalkylcarbonyl, alkoxycarbonyl, alkenyloxycarbonyl, alkynyloxycarbonyl, alkoxyalkylcarbonyl, phenylsulfonyl, phenyl, heteroaryl, heterocyclyl, phenylalkyl, heteroarylalkyl, heterocyclylalkyl, phenylcarbonyl, heteroarylcarbonyl, heterocyclylcarbonyl, phenylalkylcarbonyl, heteroarylalkylcarbonyl, heterocyclylalkylcarbonyl, phenoxycarbonyl or phenylalkoxycarbonyl;
where a phenyl or heteroaryl radical is optionally mono- or polysubstituted by halogen, alkyl, haloalkyl, alkoxy, nitro or cyano; a heterocyclyl radical is optionally substituted by —OH/═O, —SH/═S, —NH₂, halogen, alkyl, haloalkyl, alkoxy, alkylsulphanyl, alkylsulphinyl, alkylsulphonyl, monoalkylamino, dialkylamino, nitro or cyano; and a phenyl-, heteroaryl- or heterocyclyl-bound alkanediyl radical is optionally mono- or polysubstituted by halogen, alkyl, haloalkyl, alkoxy or cyano
or
R¹³ and R¹⁴ as imine form the grouping ═CR⁵—NR¹⁵ or ═CR^{S}—OR¹²;
R¹⁵ and R¹⁶ independently of one another represent alkyl or
R¹⁵ and R¹⁶ together represent alkanediyl or alkenediyl, each of which has 2 to 5 carbon atoms and is optionally substituted and/or optionally interrupted at the beginning (or the end) or within the hydrocarbon chain by oxygen or sulphur or a grouping from the group consisting of —S(O)—, —SO₂—, —NH— or —N-alkyl-;
and the compounds of the general formula (I) furthermore include N-oxides, salts, tautomers, diastereomers and optical isomers.

2. Compounds of the formula (IX) 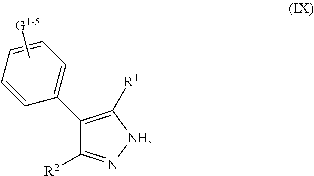
where G¹, G², G³, G⁴, G⁵, R¹ and R² are as defined in claim 1, with the proviso, that R¹ does not represent amino.

3. Compounds of the formula (XI-A) or (XI-B) 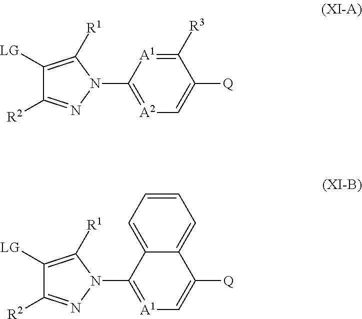
where R¹, R², R³, A¹, A² and Q are as defined in claim 1 and LG represents chlorine, bromine, iodine or alkylsulphonyl.

4. Compounds of the formula (VII-A) or (VII-B) 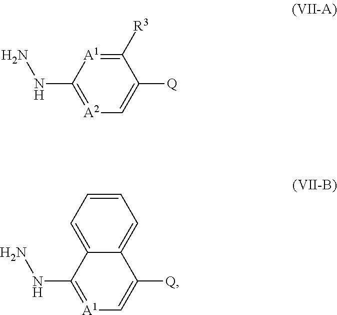
where A¹, A², R³ and Q are as defined in claim 1, but Q does not represent Z⁸ if A¹ represents nitrogen and A² represents CH.

5. Use of compounds of the formula (I) according to claim 1 for controlling animal pests.

6. Use of the compounds of the formula (I) for controlling animal pests, where M represents M¹ represents Q⁴, Q⁴ represents hydrogen and all other radicals are as defined in claim 1, with the proviso that A¹ and A² do not simultaneously represent nitrogen.

7. Use of the compounds of the formula (I) for controlling animal pests, where M represents M¹, R³ represents hydrogen and all other radicals are as defined above, with the proviso that A¹ and A² do not simultaneously represent nitrogen.

8. Use of compounds of the formula (XI-A) or (XI-B) according to claim 3 for controlling animal pests.

9. Method for controlling animal pests, characterized in that compounds of the formula (I) according to claim 1 are allowed to act on animal pests and/or their habitat and/or seed.

10. Process for preparing agrochemical compositions, characterized in that compounds of the formula (I) according to claim 1 are mixed with extenders and/or surfactants.
